SEQUENCE LISTING (1 sequence)

SEQ ID NO: 1 - protein, 4 aa, Artificial Sequence
  Synthetic polypeptide
GSGS

(19) United States
(12) Patent Application Publication
(10) Pub. No.: US 2023/0052528 A1
(43) Pub. Date: Feb. 16, 2023

(54) TASPASE1 INHIBITORS AND USES THEREOF

(71) Applicants: The Regents of the University of California, Oakland, CA (US); WASHINGTON UNIVERSITY IN ST. LOUIS, St. Louis, MO (US); VANDERBILT UNIVERSITY, Nashville, TN (US); SRI INTERNATIONAL, Menlo Park, CA (US); The United States of America, as represented by the Secretary, Department of Health and Human Servic, Bethesda, MD (US)

(72) Inventors: Michelle R. Arkin, San Francisco, CA (US); R. Jeffrey Neitz, San Francisco, CA (US); Adam R. Renslo, Oakland, CA (US); James Hsieh, Englewood Cliffs, NJ (US); Shubhankar Dutta, San Francisco, CA (US); Clifford Bryant, Millbrae, CA (US); William J. Moore, Hagerstown, MD (US); Alex G. Waterson, Murfreesboro, TN (US); Lidia Sambucetti, Redwood City, CA (US); Somnath Jana, Nashville, TN (US); lan M. Romaine, Nashville, TN (US); Alexander P. Lamers, Nashville, TN (US); Yassir Younis Adam, San Francisco, CA (US); Claire Repellin, Menlo Park, CA (US)

(21) Appl. No.: 17/777,971

(22) PCT Filed: Nov. 20, 2020
(86) PCT No.: PCT/US20/61657

§ 371 Date: May 18, 2022

Related U.S. Application Data
(60) Provisional application No. 62/939,258, filed on Nov. 22, 2019.

(51) Int. Cl. C07D 241/04; A61P 35/00; A61P 11/00; C07C 311/11; C07D 295/26; C07D 403/12; C07C 323/41; C12N 9/64

(52) U.S. Cl. CPC C07D 241/04; A61P 35/00; A61P 11/00; C07C 311/11; C07D 295/26; C07D 403/12; C07C 323/41; C12N 9/6421; C12Y 304/25

(57) ABSTRACT

Disclosed herein, inter alia, are compounds and methods for inhibiting Taspase1 and the treatment of cancer.

### CROSS-REFERENCES TO RELATED APPLICATIONS

This application claims the benefit of U.S. Provisional Application No. 62/939,258, filed Nov. 22, 2019, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under contract no. HHSN261200800001E awarded by the National Institutes of Health. The government has certain rights in the invention.

### BACKGROUND

Taspase1 (threonine aspartase) is a protease is overexpressed in numerous liquid and solid malignancies. Indeed, loss of Taspase1 strongly inhibits development of HER2-driven breast tumors and EGFR-driven, drug-resistant and non drug-resistant lung cancer. Identifying inhibitors of Taspase1 has proven to be a challenge. Disclosed herein, inter alia, are solutions to these and other problems known in the art.

### BRIEF SUMMARY

In an aspect is provided a compound having the formula:

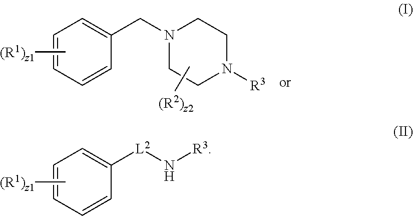

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl.

L² is substituted or unsubstituted alkylene.

R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O) NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents may optionally be joined to form a substituted or unsubstituted cycloalkyl, substituted or unsubstituted heterocycloalkyl, substituted or unsubstituted aryl, or substituted or unsubstituted heteroaryl.

R³ is independently —CN,

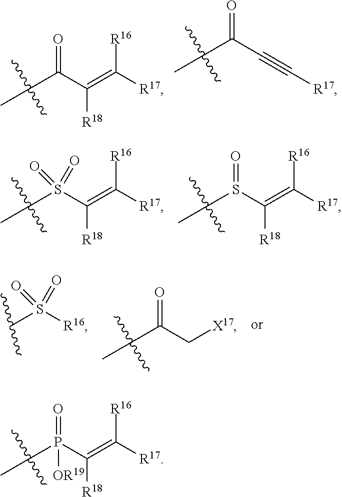

R¹⁶ is independently hydrogen, halogen, —CX¹⁶₃, —CHX¹⁶₂, —CH₂X¹⁶, —CN, —SOₙ₁₆R^{16A}, —SOᵥ₁₆NR^{16A}R^{16B}, —NHNR^{16A}R^{16B}, —ONR^{16A}R^{16B}, —NHC(O)NHNR^{16A}R^{16B}, —NHC(O)NR^{16A}R^{16B}, —N(O)ₘ₁₆, —NR^{16A}R^{16B}, —C(O)R^{16A}, —C(O)—OR^{16A}, —C(O)NR^{16A}R^{16B}, —OR^{16A}, —NR^{16A}SO₂R^{16B}, —NR^{16A}C(O)R^{16B}, —NR^{16A}C(O)OR^{16B}, —NR^{16A}OR^{16B}, —OCX¹⁶₃, —OCHX¹⁶₂, —OCH₂X¹⁶, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R¹⁷ is independently hydrogen, halogen, —CX¹⁷₃, —CHX¹⁷₂, —CH₂X¹⁷, —CN, —SOₙ₁₇R^{17A}, —SOᵥ₁₇NR^{17A}R^{17B}, —NHNR^{17A}R^{17B}, —ONR^{17A}R^{17B}, —NHC(O)NHNR^{17A}R^{17B}, —NHC(O)NR^{17A}R^{17B}, —N(O)ₘ₁₇, —NR^{17A}R^{17B}, —C(O)R^{17A}, —C(O)—OR^{17A}, —C(O)NR^{17A}R^{17B}, —OR^{17A}, —NR^{17A}SO₂R^{17B}, —NR^{17A}C(O)R^{17B}, —NR^{17A}C(O)OR^{17B}, —NR^{17A}OR^{17B}, —OCX¹⁷₃, —OCHX¹⁷₂, —OCH₂X¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R¹⁸ is independently hydrogen,

halogen, —CX¹⁸₃, —CHX¹⁸₂, —CH₂X¹⁸, —CN, —SOₙ₁₈R^{18A}, —SOᵥ₁₈NR^{18A}R^{18B}, —NHNR^{18A}R^{18B}, —ONR^{18A}R^{18B}, —NHC(O)NHNR^{18A}R^{18B},
—NHC(O)NR^{18A}R^{18B}, —N(O)ₘ₁₈, —NR^{18A}R^{18B}, —C(O)R^{18A}, —C(O)—OR^{18A}, —C(O)N^{18A}R^{18B}, —OR^{18A}, —NR^{18A}SO₂R^{18B}, —NR^{18A}C(O)R^{18B}, —NR^{18A}C(O)OR^{18B}, —NR^{18A}OR^{18B}, —OCX¹⁸₃, —OCHX¹⁸₂, —OCH₂X¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R¹⁹ is independently hydrogen, halogen, —CX¹⁹₃, —CHX¹⁹₂, —CH₂X¹⁹, —CN, —SOₙ₁₉R^{19A}, —SOᵥ₁₉NR^{19A}R^{19B}, —NHNR^{19A}R^{19B}, —ONR^{19A}R^{19B}, —NHC(O)NHNR^{19A}R^{19B}, —NHC(O)NR^{19A}R^{19B}, —N(O)ₘ₁₉, —NR^{19A}R^{19B}, —C(O)R^{19A}, —C(O)—OR^{19A}, —C(O)NR^{19A}R^{19B}, —OR^{19A}, —NR^{19A}SO₂R^{19B}, —NR^{19A}C(O)R^{19B}, —NR^{19A}C(O)OR^{19B}, —NR^{19A}OR^{19B}, —OCX¹⁹₃, —OCHX¹⁹₂, —OCH₂X¹⁹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R^{1A}, R^{1B}, R^{1C}, R^{1D}, R^{2A}, R^{2B}, R^{2C}, R^{2D}, R^{16A}, R^{16B}, R^{17A}, R^{17B}, R^{18A}, R^{18B}, R^{19A}, and R^{19B} are independently hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl.

X, X¹, X², X¹⁶, X¹⁷, X¹⁸, and X¹⁹ are independently —F, —Cl, —Br, or —I.

n1, n2, n16, n17, n18, and n19 are independently an integer from 0 to 4.

m1, m2, m16, m17, m18, m19, v1, v2, v16, v17, v18, and v19 are independently 1 or 2.

z1 is an integer from 0 to 5.

z2 is an integer from 0 to 8.

In an aspect is provided a pharmaceutical composition including a compound as described herein, including embodiments, and a pharmaceutically acceptable excipient.

In an aspect is provided a method of inhibiting Taspase1 protein activity, the method including contacting the Taspase1 protein with a compound as described herein.

In an aspect is provided a method of treating cancer, the method including administering to a subject in need thereof an effective amount of a compound as described herein.

### BRIEF DESCRIPTION OF THE DRAWINGS

FIGS. 1A and 1B. Taspase1 crystallography. FIG. 1A: Overlay of split enzyme construct and circularly permuted Taspase 1 covalently bound to inhibitor. FIG. 1B: Close in view of the catalytic site of full-length Taspase1 with superimposed inhibitor in sticks.

FIG. 2. X-ray co-crystal structures suggest new interactions near the piperazine “shoulder”.

FIGS. 3A-3B. Comparison of compound SMDC069 to compound SMDC689 in Taspase1-mediated cleavage assay in cells. FIG. 3A. Dual-fluorescent protease reporter contains the sequence of MLL cleaved by Taspase1, flanked by GFP with a nuclear export signal and RFP with a nuclear import signal (see also FIG. 7). Uncleaved reporter remains in the nucleus, while Taspase1-cleaved reporter translocates to the cytosol. SMDC069 inhibits nuclear export of the reporter through inhibition of Taspase1, with an EC₅₀ of 0.64 μM. SMDC689 inhibits nuclear export of the reporter with an EC₅₀ of 9.2 μM. FIG. 3B. Viability of PC9 cells measured at 72 h of exposure to indicated compounds by CellTiter-Glo® Assay. SMDC723 is not a Taspase1 inhibitor, and therefore acts as a nonspecific control.

FIGS. 4A-4B. Reported activity for taspase1 inhibitors. All data represented in μM. “Taspase1 IC50 (FAM)”=inhibition of cleavage of a Taspase1 peptide substrate; detected by fluorescence resonance energy transfer (FRET). “6.6 mM GSH added”=same assay, in the presence of 6.6 mM glutathione (GSH). “C293A IC50”=same assay, but using Taspase1 C293A mutation, which does not affect enzyme activity but removes the cysteine with which compounds react. DFPR IC50=dual-fluorescent proteolytic reporter cell assay described in FIGS. 3 and 7. “Ki”=Ki determined by kinact/Ki measurements, using the Taspase1/FRET-peptide assay described above.

FIGS. 5A-5C. FIG. 5A: Evolution of Taspase1 inhibitors from disulfide hit (SMDC673). FIG. 5B: X-ray structures of circularly permuted (cp-1) Taspase1 (Tasp1) bound to SMDC967 (monomer A+B). Compounds were soaked into protein crystal. Compound net shows 2Fo−Fc, 1 sigma, 2.5A resolution. FIG. 5C: Overlay X-ray structures of Tasp1 cp1±SMDC967 (monomer A+B), compound soaked into protein crystal (2.6A resolution). Numbered residues highlight contact points. Close contacts (<4A) to selected residues are indicated by dashes. Dash (“MonomerB”)=missing bond between C293 and SMDC967. Apo and +SMDC967 are depicted; definition of protein construct in FIG. 11A.

FIGS. 6A-6C. FIG. 6A: Structures of SMDC689, SMDC069, SMDC203 & SMDC275 with IC50 values (vs wildtype Taspase1), values represent average (avg) of several experiments. FIG. 6B: X-ray structures of split Tasp1 del183+SMDC069 (monomer A+B), obtained by co-crystallization of protein and inhibitor, compound net shows 2Fo−Fc, 1 sigma, 2.6A resolution. FIG. 6C: Overlay X-ray structures of split Tasp1 del183±SMDC069 (monomer A+B), obtained by co-crystallization of protein and inhibitor, 2.6A resolution. Numbered residues highlight contact points. Cys293 sulfur and SMDC069 warhead are indicated as balls. Close contacts (<4A) to selected residues are indicated by dashes. Dash (“MonomerB”)=missing bond between C293 and SMDC069. Apo and +SMDC069 are depicted; definition of protein construct in FIG. 11A.

FIG. 7. Format of DFPR assay and cell efficacy results of compound SMDC203 using DFPR Assay; 24 hours post-treatment.

FIG. 8. Cell efficacy results of compounds SMDC069 using DFPR Assay; 24 hours post-treatment.

FIGS. 9A-9B. FIG. 9A: Chemical structures and IC50 data for compounds SMDC723, SMDC069, SMDC203, and SMDC275. FIG. 9B: Cell viability assays for compounds SMDC723, SMDC069, SMDC203, and SMDC275.

FIGS. 10A-10C. FIG. 10A: Tethering screen & initial biochemistry (selectivity vs. potency) flowchart. FIG. 10B: Tethering screen scatter plot. FIG. 10C: Sample mass spectrum (top: apo; bottom: +SMDC673).

FIGS. 11A-11C. FIG. 11A: Domain diagrams of intact Tasp1, split Tasp1, split Tasp1 del183, and Tasp1 cp1_2-339. The scissile bond (T234) in the inactive zymogen and the deleted residues of the delta183 construct are also indicated. FIG. 11B: Crystal structures of apo split Tasp1 del206 (2A8J.PDB; Khan, 2005), split Tasp1 del183, and Tasp1 cp 12-339. Shown are the alpha and beta domains of the Monomer “B” structures. Residues 1-40 and 417-420 in the del206 and del183 are not observed in the crystal structures. Residues 41 and 416 of the del183 and del206 constructs and the GSGS linker of the circularly permuted structure are shown (bottom left of protein). FIG. 11C: Overlays of the three dimeric structures: del206; del183; and cp1.

FIGS. 12A-12C. FIG. 12A: Structures of SMDC673, SMDC208, SMDC714, and SMDC967. FIG. 12B: LC-MS selectivity data for wildtype (WT) Taspase1 vs. Taspase1 C293A and Caspase6. FIG. 12C: LC-MS selectivity data for Taspase1 WT, T234A, T234S, or T234V constructs.

FIGS. 13A-13D. FIG. 13A: Structures of compounds SMDC689, SMDC069, SMDC203 and SMDC275. FIG. 13B: Representative IC₅₀ plots for compounds SMDC689, SMDC069, SMDC203 and SMDC727, using Taspase1 inhibition assay with FRET substrate, with or without 6.6 mM GSH. FIG. 13C: LC-MS selectivity data for wildtype (WT) Taspase1 vs. Taspase1 C293A and Caspase6. FIG. 13D: LC-MS selectivity data for Taspase1 WT, T234A, T234S, or T234V constructs.

FIGS. 14A-14C. FIG. 14A: X-ray structures of Tasp1 cp1+SMDC689 (monomer A+B), co-crystal, compound net shows 2Fo-Fc, 1 sigma, 2.45 Å resolution. FIG. 14B: Overlay of apo cp1 and cp1+SMDC689. Close contact (≤4 Å, dashes) between SMDC689 and indicated residues. FIG. 14C: Chemical structure of SMDC689. Protein construct defined in FIG. 11A.

FIGS. 15A-15D. Tasp1 cp1 structures+SMDC967 or SMDC689. FIG. 15A: Overlays of cp1+SMDC967 and cp1+SMDC689 focusing on the substituted phenyl ring of the inhibitors; Monomer A (left) and Monomer B (right). Close contacts between the inhibitor and A48, Y61, and C378 of compounds SMDC967 and SMDC689 are indicated as dashes. FIGS. 15B and 15C: Surface representation of Tasp1 cp1+SMDC967 (b) and +SMDC678 (c), focusing on the substituted phenyl ring. The 3-fluoro group of SMDC689 fills an empty space in the binding pocket not occupied by SMDC967. FIG. 15D: Chemical structures of SMDC967 and SMDC689. Protein construct defined in FIG. 11A.

FIGS. 16A-16C. split Tasp1 delta183+SMDC689 co-crystal, 2.3 Å resolution. FIG. 16A: X-ray structures of split Tasp1 delta183+SMDC689 (monomer A+B), co-crystal, 2Fo-Fc, 1 sigma, 2.3 Å resolution. FIG. 16B: Overlay of apo delta183 and delta183+SMDC689. Close contact (≤4 Å, dashes) between SMDC689 and indicated residues. The C293 thiol sulfur and the SMDC689 warhead are indicated as balls, respectively. FIG. 16C: Chemical structure of SMDC689. Protein construct defined in FIG. 11A.

FIGS. 17A-17C. FIG. 17A: X-ray structures of split Tasp1 delta183+SMDC556 (monomer A+B), co-crystal, 2Fo-Fc, 1 sigma, 2.5 Å resolution. FIG. 17B: Overlay of apo delta183 and delta183+SMDC556. Close contact (≤4 Å, dashes) between SMDC556 and indicated residues. The C293 thiol sulfur and the SMDC556 warhead are indicated as balls, respectively. FIG. 17C: Chemical structure of SMDC556. Protein construct defined in FIG. 11A.

FIGS. 18A-18C. FIG. 18A: X-ray structures of Tasp1 cp1+SMDC883 (monomer A+B), co-crystal, 2Fo-Fc, 1 sigma, 2.15 Å resolution. FIG. 18B: Overlay of apo cp1 and cp1+SMDC883. Close contact (≤4 Å, dashes) between SMDC883 and indicated residues. The C293 thiol sulfur and the SMDC883 warhead are indicated as balls, respectively. FIG. 18C: Chemical structure of SMDC883. Protein construct defined in FIG. 11A.

FIG. 19. Overlay of circularly permuted & split Taspase structures and compounds (compounds not shown).

FIG. 20. Crystallography parameters for split Tasp1 delta 183.

FIG. 21. Crystallography parameters for Tasp1 cp-1_2-339.

FIG. 22. Dual Fluorescent Proteolytic Reporter assay results. Representative dose response curves for SMDC967, SMDC069, SMDC203, and SMDC275.

FIG. 23. Biochemical and Cell-Activity Data for Selected Compounds and Cancer Cell Line Cytotoxicity Data (average values+std dev). The data in FIG. 23 are averaged numbers, based on multiple experiments, and including experiments as set forth in FIGS. 3A-3B, FIGS. 4A-4B, FIG. 8, and FIG. 22.

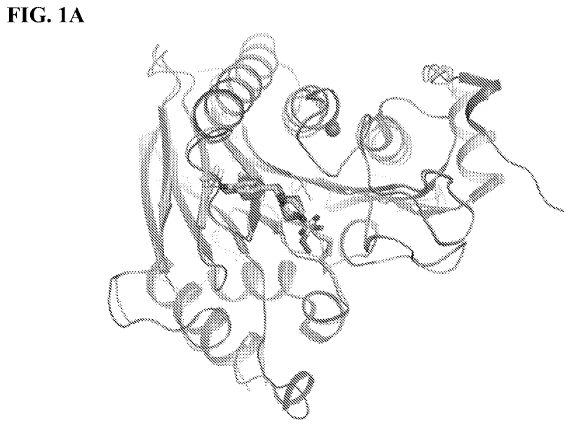

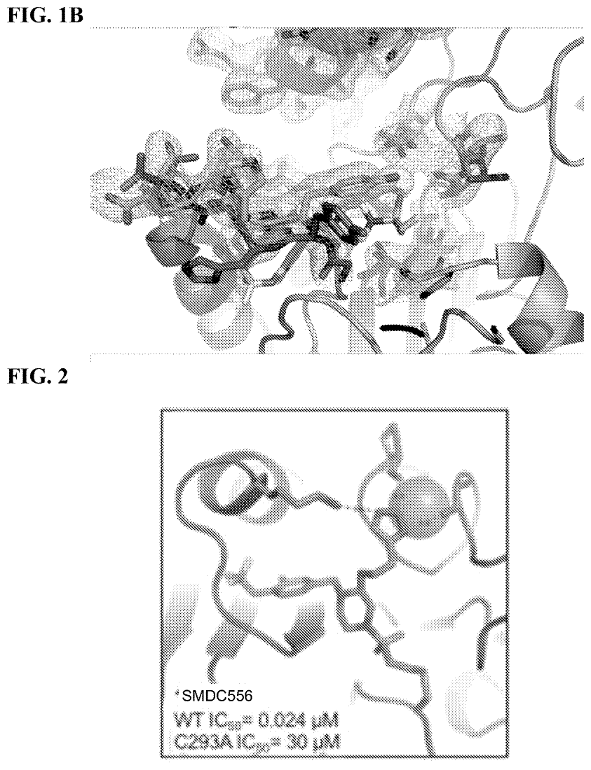

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1 and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds. In embodiments, the alkyl is fully saturated. In embodiments, the alkyl is monounsaturated. In embodiments, the alkyl is polyunsaturated.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. In embodiments, a bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, a bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings. In embodiments, monocyclic cycloalkenyl ring systems are cyclic hydrocarbon groups containing from 3 to 8 carbon atoms, where such groups are unsaturated (i.e., containing at least one annular carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, the term “heterocycloalkyl” means a monocyclic, bicyclic, or a multicyclic heterocycloalkyl ring system. In embodiments, heterocycloalkyl groups are fully saturated. In embodiments, a bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings. In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. In embodiments, a fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). In embodiments, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl, benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4 pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocycloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

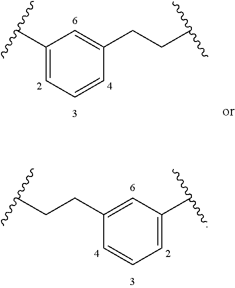

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —Cl₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″—CN, —NO₂, —NR′SO₂R″—NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″—NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₂₀ alkyl, C₁-C₁₂ alkyl, C₁-C₆ alkyl, C₁-C₆ alkyl, C₁-C₄ alkyl, or C₁-C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃-C₁₀ cycloalkyl, C₃-C₈ cycloalkyl, C₃-C₆ cycloalkyl, C₄-C₆ cycloalkyl, or C₅-C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆-C₁₂ aryl, C₆-C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₆ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₂₀ alkyl, C₁-C₁₂ alkyl, C₁-C₈ alkyl, C₁-C₆ alkyl, C₁-C₄ alkyl, or C₁-C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃-C₁₀ cycloalkyl, C₃-C₈ cycloalkyl, C₃-C₆ cycloalkyl, C₄-C₆ cycloalkyl, or C₅-C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆-C₁₂ aryl, C₆-C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —N₁-12, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₂₀ alkyl, C₁-C₁₂ alkyl, C₁-C₈ alkyl, C₁-C₆ alkyl, C₁-C₄ alkyl, or C₁-C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃-C₁₀ cycloalkyl, C₃-C₈ cycloalkyl, C₃-C₆ cycloalkyl, C₄-C₆ cycloalkyl, or C₅-C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆-C₁₂ aryl, C₆-C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₆ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkyl ene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the term “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH₂, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding;
(m) metal bonding to reactive phosphorus groups (e.g. phosphines) to form, for example, phosphate diester bonds;
(n) azides coupled to alkynes using copper catalyzed cycloaddition click chemistry; and
(o) biotin conjugate can react with avidin or strepavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.1}, R^{13.2}, R^{13.3}, R^{13.4}, etc., wherein each of R^{13.1}, R^{13.2}, R^{13.3}, R^{13.4} etc. is defined within the scope of the definition of R¹³ and optionally differently.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As^{, 86}Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

The term “lipid moiety” is used in accordance with its ordinary meaning in chemistry and refers to a hydrophobic molecule which is typically characterized by an aliphatic hydrocarbon chain. In embodiments, the lipid moiety includes a carbon chain of 3 to 100 carbons. In embodiments, the lipid moiety includes a carbon chain of 5 to 50 carbons. In embodiments, the lipid moiety includes a carbon chain of 5 to 25 carbons. In embodiments, the lipid moiety includes a carbon chain of 8 to 525 carbons. Lipid moieties may include saturated or unsaturated carbon chains, and may be optionally substituted. In embodiments, the lipid moiety is optionally substituted with a charged moiety at the terminal end. In embodiments, the lipid moiety is an alkyl or heteroalkyl optionally substituted with a carboxylic acid moiety at the terminal end.

A charged moiety refers to a functional group possessing an abundance of electron density (i.e. electronegative) or is deficient in electron density (i.e. electropositive). Non-limiting examples of a charged moiety includes carboxylic acid, alcohol, phosphate, aldehyde, and sulfonamide. In embodiments, a charged moiety is capable of forming hydrogen bonds.

The terms “bind” and “bound” as used herein is used in accordance with its plain and ordinary meaning and refers to the association between atoms or molecules. The association can be covalent (e.g., covalent bond or covalent linker (e.g., a first linker or second linker)) or non-covalent (e.g., non-covalent bond (e.g., electrostatic interactions (e.g., ionic bond, hydrogen bond, halogen bond), van der Waals interactions (e.g. dipole-dipole, dipole-induced dipole, London dispersion), ring stacking (pi effects), hydrophobic interactions and the like)).

The term “capable of binding” as used herein refers to a moiety (e.g. a compound as described herein) that is able to measurably bind to a target (e.g., a NF-κB, a Toll-like receptor protein). Ki is the binding constant that describes the energy of association for the non-covalent portion of the molecule. Kinact is the rate at which the covalent bond is formed between the small molecule and the protein. In embodiments, where a moiety is capable of binding a target, the moiety is capable of binding with a Ki of less than about 150 μM, 125 μM, 110 μM, 100 μM, 75 μM, 50 μM, 20 μM, 10 μM, 5 μM, 1 μM, 500 nM, 250 nM, 100 nM, 75 nM, 50 nM, 25 nM, 15 nM, 10 nM, 5 nM, 1 nM, or about 0.1 nM. In embodiments, the Kinact is less than about, 1 s⁻¹, 0.5 s⁻¹, 0.1 s⁻¹, 0.05 s⁻¹, 0.01 s⁻¹, 0.005 s⁻¹, or about 0.001 s⁻¹.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, propionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

A “synergistic amount” as used herein refers to the sum of a first amount (e.g., an amount of a Taspase1 inhibitor) and a second amount (e.g., a therapeutic agent) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of the Taspase1 inhibitor in combination with a second agent (e.g., an anticancer agent) where the measured effect is greater than the sum of the individual effects of the Taspase1 inhibitor provided herein and the second agent (e.g., anticancer agent) administered alone as a single agent.

In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the Taspase1 inhibitor provided herein when used separately from the therapeutic agent. In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the therapeutic agent when used separately from the Taspase1 inhibitor provided herein.

The term “EC50” or “half maximal effective concentration” as used herein refers to the concentration of a molecule (e.g., antibody, chimeric antigen receptor or bispecific antibody) capable of inducing a response which is halfway between the baseline response and the maximum response after a specified exposure time. In embodiments, the EC50 is the concentration of a molecule (e.g., antibody, chimeric antigen receptor or bispecific antibody) that produces 50% of the maximal possible effect of that molecule.

The term “IC50” or “half maximal inhibitory concentration” as used herein refers to the concentration of an inhibitory molecule (e.g., small molecule, antibody, chimeric antigen receptor or bispecific antibody) that is required to inhibit a given biologal process or biological component by 50%.

The term “small molecule” is used in accordance with its well understood meaning and refers to a low molecular weight organic compound that may regulate a biological process. In embodiments, the small molecule is a compound that weighs less than 1000 daltons. In embodiments, the small molecule is a compound that weighs less than 900 daltons. In embodiments, the small molecule weighs less than 800 daltons. In embodiments, the small molecule weighs less than 700 daltons. In embodiments, the small molecule weighs less than 600 daltons. In embodiments, the small molecule weighs less than 500 daltons. In embodiments, the small molecule weighs less than 450 daltons. In embodiments, the small molecule weighs less than 400 daltons.

An “Taspase1 inhibitor” refers to a compound (e.g. a compound described herein) that reduces the activity of Taspase1 when compared to a control, such as absence of the compound or a compound with known inactivity.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation). A “Taspase1 inhibitor” is a compound that negatively affects (e.g. decreases) the activity or function of Taspase1 relative to the activity or function of Taspase1 in the absence of the inhibitor.

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The terms “Taspase1” and “Taspase 1” and “TASP1” and “Tasp1” and “Threonine aspartase 1” refer to a protein (including homologs, isoforms, and functional fragments thereof) which cleaves substrates following aspartate residues. The term includes any recombinant or naturally-occurring form of Taspase1 variants thereof that maintain Taspase1 activity (e.g. within at least 30%, 40%, 50%, 60%, 70%, 80%, 90%, 95%, or 100% activity compared to wild-type Taspase1). In embodiments, the Taspase1 protein encoded by the TASP1 gene has the amino acid sequence set forth in or corresponding to Entrez 55617, UniProt Q9H6P5, RefSeq (protein) NP 001310531, RefSeq (protein) NP 001310532, RefSeq (protein) NP 001310533, or RefSeq (protein) NP 060184. In embodiments, the TASP1 gene has the nucleic acid sequence set forth in RefSeq (mRNA) NM 017714, RefSeq (mRNA) NM 001323602, RefSeq (mRNA) NM 001323603, or RefSeq (mRNA) NM 001323604. In embodiments, the amino acid sequence or nucleic acid sequence is the sequence known at the time of filing of the present application. In embodiments, the Taspase1 protein sequence corresponds to NP 001310531.1. In embodiments, the Taspase1 protein sequence corresponds to NP 001310532.1. In embodiments, the Taspase1 protein sequence corresponds to NP 001310533.1. In embodiments, the Taspase1 protein sequence corresponds to NP 060184. In embodiments, the Taspase1 protein sequence corresponds to NP 060184.2. In embodiments, the Taspase1 is a human Taspase1, such as a human cancer causing Taspase1.

The terms “TASP1 gene” as used herein refer to the any of the recombinant or naturally-occurring forms of the TASP1 gene or variants or homologs thereof that code for a Taspase1 polypeptide capable of maintaining the activity of the Taspase1 polypeptide (e.g., within at least 50%, 80%, 90%, 95%, 96%, 97%, 98%, 99% or 100% activity compared to Taspase1 polypeptide). In embodiments, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring TASP1 gene.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to a composition that increases or decreases the level of a target molecule or the function of a target molecule or the physical state of the target of the molecule relative to the absence of the modulator. In some embodiments, a Taspase1 associated disease modulator is a compound that reduces the severity of one or more symptoms of a disease associated with Taspase1 (e.g. cancer). A Taspase1 modulator is a compound that increases or decreases the activity or function or level of activity or level of function of Taspase1.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease, a cancer associated with Taspase1 activity, Taspase1 associated cancer, Taspase1 associated disease (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. For example, a cancer associated with Taspase1 activity or function may be a cancer that results (entirely or partially) from aberrant Taspase1 function (e.g. enzyme activity, protein-protein interaction, signaling pathway) or a cancer wherein a particular symptom of the disease is caused (entirely or partially) by aberrant Taspase1 activity or function. As used herein, what is described as being associated with a disease, if a causative agent, could be a target for treatment of the disease. For example, a cancer associated with Taspase1 activity or function or a Taspase1 associated disease (e.g., cancer, inflammatory disease, autoimmune disease, or infectious disease), may be treated with a Taspase1 modulator or Taspase1 inhibitor, in the instance where increased Taspase1 activity or function (e.g. signaling pathway activity) causes the disease (e.g., cancer, inflammatory disease, autoimmune disease, or infectious disease).

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components. For example, binding of a Taspase1 with a compound as described herein may reduce the level of a product of the Taspase1 catalyzed reaction or the level of a downstream derivative of the product or binding may reduce the interactions between the Taspase1 enzyme or an Taspase1 reaction product and downstream effectors or signaling pathway components (e.g., epigenetic regulatory proteins MLL and the transcription factor (TF) IIA family of nuclear proteins), resulting in changes in cell growth, proliferation, or survival.

In this disclosure, “comprises,” “comprising,” “containing” and “having” and the like can have the meaning ascribed to them in U.S. patent law and can mean “includes,” “including,” and the like. “Consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “inflammatory disease” refers to a disease or condition characterized by aberrant inflammation (e.g. an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “inflammatory disease” refers to a disease or condition characterized by aberrant inflammation (e.g. an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

As used herein, the term “neurodegenerative disorder” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment (e.g., the patient has a disease, the patient suffers from a disease).

The term “prevent” refers to a decrease in the occurrence of Taspase1 disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

Cancer model organism, as used herein, is an organism exhibiting a phenotype indicative of cancer, or the activity of cancer-causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

An “anticancer agent” as used herein refers to a molecule (e.g. compound, peptide, protein, or nucleic acid) used to treat cancer through destruction or inhibition of cancer cells or tissues. Anticancer agents may be selective for certain cancers or certain tissues. In embodiments, anticancer agents herein may include epigenetic inhibitors and multi-kinase inhibitors.

“Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK 1, MEK2, or MEK 1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rIL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), Vincristine sulfate, Cryptophycin 52 (i.e. LY-355703), Vitilevuamide, Tubulysin A, Canadensol, Centaureidin (i.e. NSC-106969), Oncocidin A1 (i.e. BTO-956 and DIME), Fijianolide B, Laulimalide, Narcosine (also known as NSC-5366), Nascapine, Hemiasterlin, Vanadocene acetylacetonate, Monsatrol, lnanocine (i.e. NSC-698666), Eleutherobins (such as Desmethyleleutherobin, Desaetyleleutherobin, lsoeleutherobin A, and Z Eleutherobin), Caribaeoside, Caribaeolin, Halichondrin B, Diazonamide A, Taccalonolide A, Diozostatin, (−)-Phenylahistin (i.e. NSCL-96F037), Myoseverin B, Resverastatin phosphate sodium, steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In ⁹⁰Y or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinibBIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

An “epigenetic inhibitor” as used herein, refers to an inhibitor of an epigenetic process, such as DNA methylation (a DNA methylation Inhibitor) or modification of histones (a Histone Modification Inhibitor). An epigenetic inhibitor may be a histone-deacetylase (HDAC) inhibitor, a DNA methyltransferase (DNMT) inhibitor, a histone methyltransferase (HMT) inhibitor, a histone demethylase (HDM) inhibitor, or a histone acetyltransferase (HAT). Examples of HDAC inhibitors include Vorinostat, romidepsin, CI-994, Belinostat, Panobinostat, Givinostat, Entinostat, Mocetinostat, SRT501, CUDC-101, JNJ-26481585, or PCI24781. Examples of DNMT inhibitors include azacitidine and decitabine. Examples of HMT inhibitors include EPZ-5676. Examples of HDM inhibitors include pargyline and tranylcypromine. Examples of HAT inhibitors include CCT077791 and garcinol.

A “multi-kinase inhibitor” is a small molecule inhibitor of at least one protein kinase, including tyrosine protein kinases and serine/threonine kinases. A multi-kinase inhibitor may include a single kinase inhibitor. Multi-kinase inhibitors may block phosphorylation. Multi-kinases inhibitors may act as covalent modifiers of protein kinases. Multi-kinase inhibitors may bind to the kinase active site or to a secondary or tertiary site inhibiting protein kinase activity. A multi-kinase inhibitor may be an anti-cancer multi-kinase inhibitor. Exemplary anti-cancer multi-kinase inhibitors include dasatinib, sunitinib, erlotinib, bevacizumab, vatalanib, vemurafenib, vandetanib, cabozantinib, poatinib, axitinib, ruxolitinib, regorafenib, crizotinib, bosutinib, cetuximab, gefitinib, imatinib, lapatinib, lenvatinib, mubritinib, nilotinib, panitumumab, pazopanib, trastuzumab, or sorafenib.

The term “irreversible covalent bond” is used in accordance with its plain ordinary meaning in the art and refers to the resulting association between atoms or molecules of (e.g., electrophilic chemical moiety and nucleophilic moiety) wherein the probability of dissociation is low. In embodiments, the irreversible covalent bond does not easily dissociate under normal biological conditions. In embodiments, the irreversible covalent bond is formed through a chemical reaction between two species (e.g., electrophilic chemical moiety and nucleophilic moiety).

The term “electrophilic moiety” is used in accordance with its plain ordinary chemical meaning and refers to a chemical group (e.g., monovalent chemical group) that is electrophilic. In embodiments, the electrophilic chemical moiety is referred to herein as a “warhead” or “E.” In embodiments, E is:

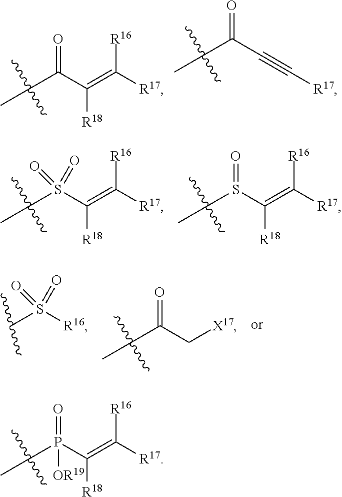

wherein R¹⁶, R¹⁷, R¹⁸, R¹⁹, and X¹⁷ are as described herein, including in embodiments. In embodiments, an electrophilic moiety is a covalent cysteine modifier moiety.

The term “covalent cysteine modifier moiety” as used herein refers to a monovalent electrophilic moiety that is able to measurably bind to a cysteine amino acid. In embodiments, the covalent cysteine modifier moiety binds via an irreversible covalent bond. In embodiments, the covalent cysteine modifier moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM. In embodiments, the covalent cysteine modifier moiety binds via a covalent bond.

The term “nucleophilic moiety” is used in accordance with its plain ordinary chemical meaning and refers to a chemical group (e.g., monovalent chemical group) that is nucleophilic.

An amino acid residue in a protein “corresponds” to a given residue when it occupies the same essential structural position within the protein as the given residue. Instead of a primary sequence alignment, a three-dimensional structural alignment can also be used, e.g., where the structure of the selected protein is aligned for maximum correspondence with the human protein and the overall structures compared. In this case, an amino acid that occupies the same essential position as a specified amino acid in the structural model is said to correspond to the specified residue. For example, a selected residue in a selected protein corresponds to C293 of a Taspase1 protein (e.g., human Taspase1 protein) when the selected residue occupies the same essential spatial or other structural relationship as C293 in the Taspase1 protein (e.g., human Taspase1 protein). In some embodiments, where a selected protein is aligned for maximum homology with the Taspase1 protein (e.g., human Taspase1 protein), the position in the aligned selected protein aligning with C293 is said to correspond to C293 of the Taspase1 protein (e.g., human Taspase1 protein). Instead of a primary sequence alignment, a three-dimensional structural alignment can also be used, e.g., where the structure of the selected protein is aligned for maximum correspondence with the Taspase1 protein (e.g., human Taspase1 protein) and the overall structures compared. In this case, an amino acid that occupies the same essential position as C293 of a Taspase1 protein (e.g., human Taspase1 protein) in the structural model is said to correspond to the C293 residue. Another example is wherein a selected residue in a selected protein corresponds to C293 in a Taspase1 protein (e.g., human Taspase1 protein) when the selected residue (e.g., cysteine residue) occupies essential the same sequence, spatial, or other structural position within the protein as C293 in the Taspase1 protein (e.g., human Taspase 1 protein).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

### II. Compounds

In an aspect is provided a compound having the formula:

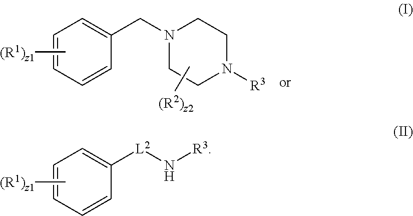

R¹ is independently halogen, —CX¹³, —CHX¹², —CH₂X¹, —OCX¹³, —OCH₂X¹, —OCHX¹², —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl.

L² is substituted or unsubstituted alkylene.

R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR²CNR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O) NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents may optionally be joined to form a substituted or unsubstituted cycloalkyl, substituted or unsubstituted heterocycloalkyl, substituted or unsubstituted aryl, or substituted or unsubstituted heteroaryl.

R³ is independently —CN,

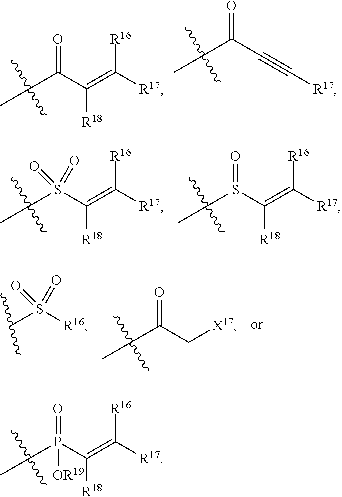

In an alternative aspect, R³ is an electrophilic moiety. In embodiments, R³ is a covalent cysteine modifier moiety.

R¹⁶ is independently hydrogen, halogen, —CX¹⁶₃, —CHX¹⁶₂, —CH₂X¹⁶, —CN, —SOₙ₁₆R^{16A}, —SOᵥ₁₆NR^{16A}R^{16B}, —NHNR^{16A}R^{16B}, —ONR^{16A}R^{16B}, —NHC(O)NHNR^{16A}R^{16B}, —NHC(O)NR^{16A}R^{16B}, —N(O)ₘ₁₆, —NR^{16A}R^{16B}, —C(O)R^{16A}, —C(O)—OR^{16A}, —C(O)NR^{16A}R^{16B}, —OR^{16A}, —NR^{16A}SO₂R^{16B}, —NR^{16A}C(O)R^{16B}, —NR^{16A}C(O)OR^{16B}, —NR^{16A}OR^{16B}, —OCX¹⁶₃, —OCHX¹⁶₂, —OCH₂X¹⁶, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R¹⁷ is independently hydrogen,

halogen, —CX¹⁷³, —CHX¹⁷², —CH₂X¹⁷, —CN, —SOₙ₁₇R^{17A}, —SOᵥ₁₇NR^{17A}R^{17B}, —NHNR^{17A}R^{17B}, —ONR^{17A}R^{17B}, —NHC(O)NHNR^{17A}R^{17B}, —NHC(O)NR^{17A}R^{17B}, —N(O)ₘ₁₇, —NR^{17A}R^{17B}, —C(O)R^{17A}, —C(O)—OR^{17A}, —C(O)NR^{17A}R^{17B}, —OR^{17A}, —NR^{17A}SO₂R^{17B}, —NR^{17A}C(O)R^{17B}, —NR^{17A}C(O)OR^{17B}, —NR^{17A}OR^{17B}, —OCX¹⁷₃, —OCHX¹⁷₂, —OCH₂X¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R¹⁸ is independently hydrogen,

halogen, —CX¹⁸₃, —CHX¹⁸₂, —CH₂X¹⁸, —CN, —SOₙ₁₈R^{18A}, —SOᵥ₁₈ NR^{18A}R^{18B}, —NHNR^{18A}R^{18B}, —ONR^{18A}R^{18B}, —NHC(O)NHNR^{18A}R^{18B}, —NHC(O)NR^{18A}R^{18B}, —N(O)ₘ₁₈, —NR^{18A}R^{8B}, —C(O)R^{18A}, —C(O)—OR^{18A}, —C(O)NR^{18A}R^{18B}, —OR^{18A}, —NR^{18A}SO₂R^{18B}, —NR^{18A}C(O)R^{18B}, —NR^{18A}C(O)OR^{18B}, —NR^{18A}OR^{18B}, —OCX¹⁸₃, —OCHX¹⁸₂, —OCH₂X¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R¹⁹ is independently hydrogen,

halogen, —CX¹⁹₃, —CHX¹⁹₂, —CH₂X¹⁹, —CN, —SOₙ₁₉R^{19A}, —SOᵥ₁₉NR^{19A}R^{19B}, —NHNR^{9A}R^{19B}, —ONR^{19A}R^{19B}, —NHC(O)NHNR^{19A}R^{19B}, —NHC(O)NR^{19A}R^{19B}, —N(O)ₘ₁₉, —NR^{9A}R^{19B}, —C(O)R^{19A}, —C(O)—OR^{19A}, —C(O)NR^{19A}R^{19B}, —OR^{19A}, —NR^{19A}SO₂R^{19B}, —NR^{19A}C(O)R^{19B}, —NR^{19A}C(O)OR^{19B}, —NR^{19A}OR^{19B}, —OCX¹⁹₃, —OCHX¹⁹₂, —OCH₂X¹⁹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R^{1A}, R^{1B}, R^{1C}, R^{1D}, R^{2A}, R^{2B}, R^{2C}, R^{2D}, R^{16A}, R^{16B}, R^{17A}, R^{17B}, R^{18A}, R^{18B}, R^{19A}, and R^{19B} are independently hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl.

X, X¹, X², X¹⁶, X¹⁷, X¹⁸, and X¹⁹ are independently —F, —Cl, —Br, or —I.

n1, n2, n16, n17, n18, and n19 are independently an integer from 0 to 4.

m1, m2, m16, m17, m18, m19, v1, v2, v16, v17, v18, and v19 are independently 1 or 2.

z1 is an integer from 0 to 5.

z2 is an integer from 0 to 8.

In embodiments, the compound has the formula:

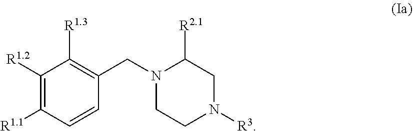

R^{1.1} is independently hydrogen or any value of R¹ as described herein.

R^{1.2} is independently hydrogen or any value of R² as described herein.

R^{1.3} is independently hydrogen or any value of R³ as described herein.

R^{2.1} is independently hydrogen or any value of R² as described herein.

In embodiments, R^{1.1}, R^{1.2}, and R^{1.3} are independently hydrogen, halogen, —CX¹₃, —CHX¹₂, —CH₂X¹, —OCX¹₃, —OCH₂X¹, —OCHX¹₂, —CN, —SR^{1D}, —SCX¹₃, —SCH₂X¹, —SCHX¹₂, —SOₙ₁R^{1D}, —NR^{1A}R^{1B}, —OR^{1D}, —SF₅, substituted or unsubstituted C₁-C₆ alkyl, substituted or unsubstituted 2 to 6 membered heteroalkyl, or substituted or unsubstituted 5 to 6 membered heteroaryl.

In embodiments, R^{2.1} is independently hydrogen, oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl.

In embodiments, the compound has the formula:

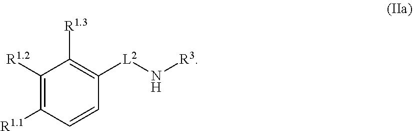

R^{1.1}, R^{1.2}, R³, and L² are as described herein

L² is unsubstituted C₁-C₆ alkylene.

In embodiments, R^{1.1}, R^{1.2}, and R^{1.3} are independently hydrogen,

halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, —SH, —SCH₃, —SCF₃, —SCHF₂, —SCH₂F, —SCCl ₃, —SCHCl₂, —SCH₂Cl, —SCBr₃, —SCHBr₂, —SCH₂Br, —SCl₃, —SCHI₂, —SCH₂I, —SOCH₃, —SO₂CH₃, —NH₂, —NHCH₃, —OH, —SF₅, substituted or unsubstituted C₁-C₆ alkyl, substituted or unsubstituted 2 to 6 membered heteroalkyl, or substituted or unsubstituted 5 to 6 membered heteroaryl.

In embodiments, R^{1.1}, R^{1.2}, and R^{1.3} are independently hydrogen,

halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, —SH, —OH, substituted or unsubstituted C₁-C₆ alkyl, or substituted or unsubstituted 2 to 6 membered heteroalkyl. In embodiments, R¹ 1 is independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —O CHF₂, —OCH₂Cl, —OCH₂Br, —OCH₂I, —OCH₂F, —CN, —SH, —OH, substituted or unsubstituted C₁-C₆ alkyl, or substituted or unsubstituted 2 to 6 membered heteroalkyl. In embodiments, R^{1.2} is independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —O CHF₂, —OCH₂Cl, —OCH₂Br, —OCH₂I, —OCH₂F, —CN, —SH, —OH, substituted or unsubstituted C₁-C₆ alkyl, or substituted or unsubstituted 2 to 6 membered heteroalkyl. In embodiments, R^{1.3} is independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —O CHF₂, —OCH₂Cl, —OCH₂Br, —OCH₂I, —OCH₂F, —CN, —SH, —OH, substituted or unsubstituted C₁-C₆ alkyl, or substituted or unsubstituted 2 to 6 membered heteroalkyl.

In embodiments, R^{2.1} is independently hydrogen, oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R²⁰-substituted or unsubstituted C₁-C₆ alkyl (e.g., C₁-C₆ alkyl, C₁-C₄ alkyl, or C₁-C₂ alkyl), R²⁰-substituted or unsubstituted 2 to 6 membered heteroalkyl (e.g., 2 to 6 membered heteroalkyl, 2 to 4 membered heteroalkyl, or 2 to 3 membered heteroalkyl), R²⁰-substituted or unsubstituted C₃-C₆ cycloalkyl (e.g., C₃-C₆ cycloalkyl, C₃-C₅ cycloalkyl, or C₅-C₆ cycloalkyl), R²⁰-substituted or unsubstituted 3 to 6 membered heterocycloalkyl (e.g., 3 to 6 membered heterocycloalkyl, 3 to 5 membered heterocycloalkyl, or 5 to 6 membered heterocycloalkyl), R²⁰-substituted or unsubstituted C₆-C₁₂ aryl (e.g., C₆-C₁₂ aryl, C₆-C₁₀ aryl, C₁₀ aryl, or phenyl), or R²⁰-substituted or unsubstituted 5 to 12 membered heteroaryl (e.g., 5 to 12 membered heteroaryl, 5 to 10 membered heteroaryl, 5 to 9 membered heteroaryl, or 5 to 6 membered heteroaryl).

In embodiments, R^{2.1} is independently hydrogen, R²⁰-substituted or unsubstituted C₁-C₆ alkyl (e.g., C₁-C₆ alkyl, C₁-C₄ alkyl, or C₁-C₂ alkyl), R²⁰-substituted or unsubstituted 2 to 6 membered heteroalkyl (e.g., 2 to 6 membered heteroalkyl, 2 to 4 membered heteroalkyl, or 2 to 3 membered heteroalkyl), R²⁰-substituted or unsubstituted C₃-C₆ cycloalkyl (e.g., C₃-C₆ cycloalkyl, C₃-C₅ cycloalkyl, or C₅-C₆ cycloalkyl), R²⁰-substituted or unsubstituted 3 to 6 membered heterocycloalkyl (e.g., 3 to 6 membered heterocycloalkyl, 3 to 5 membered heterocycloalkyl, or 5 to 6 membered heterocycloalkyl), R²⁰-substituted or unsubstituted C₆-C₁₂ aryl (e.g., C₆-C₁₂ aryl, C₆-C₁₀ aryl, C₁₀ aryl, or phenyl), or R²⁰-substituted or unsubstituted 5 to 12 membered heteroaryl (e.g., 5 to 12 membered heteroaryl, 5 to 10 membered heteroaryl, 5 to 9 membered heteroaryl, or 5 to 6 membered heteroaryl).

R²⁰ is independently oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, R²¹-substituted or unsubstituted alkyl (e.g., C₁-C₈ alkyl, C₁-C₆ alkyl, or C₁-C₄ alkyl), R²¹-substituted or unsubstituted heteroalkyl (e.g., 2 to 8 membered heteroalkyl, 2 to 6 membered heteroalkyl, or 2 to 4 membered heteroalkyl), R²¹-substituted or unsubstituted cycloalkyl (e.g., C₃-C₈ cycloalkyl, C₃-C₆ cycloalkyl, or C₅-C₆ cycloalkyl), R²¹-substituted or unsubstituted heterocycloalkyl (e.g., 3 to 8 membered heterocycloalkyl, 3 to 6 membered heterocycloalkyl, or 5 to 6 membered heterocycloalkyl), R²¹-substituted or unsubstituted aryl (e.g., C₆-C₁₂ aryl, C₆-C₁₀ aryl, C₁₀ aryl, or phenyl), or R²¹-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R²⁰ is independently oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI ₂, —OCHF₂, —OCH₂Cl, —OCH₂Br, —OCH₂I, —OCH₂F, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

R²¹ is independently oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2.1} is independently hydrogen, unsubstituted C₁-C₆ alkyl (e.g., C₁-C₆ alkyl, C₁-C₄ alkyl, or C₁-C₂ alkyl), unsubstituted 2 to 6 membered heteroalkyl (e.g., 2 to 6 membered heteroalkyl, 2 to 4 membered heteroalkyl, or 2 to 3 membered heteroalkyl), unsubstituted C₃-C₆ cycloalkyl (e.g., C₃-C₆ cycloalkyl, C₃-C₅ cycloalkyl, or C₅-C₆ cycloalkyl), unsubstituted 3 to 6 membered heterocycloalkyl (e.g., 3 to 6 membered heterocycloalkyl, 3 to 5 membered heterocycloalkyl, or 5 to 6 membered heterocycloalkyl), unsubstituted C₆-C₁₂ aryl (e.g., C₆-C₁₂ aryl, C₆-C₁₀ aryl, C₁₀ aryl, or phenyl), or unsubstituted 5 to 12 membered heteroaryl (e.g., 5 to 12 membered heteroaryl, 5 to 10 membered heteroaryl, 5 to 9 membered heteroaryl, or 5 to 6 membered heteroaryl).

In embodiments, R^{2.1} is independently hydrogen, substituted or unsubstituted C₁-C₆ alkyl, substituted or unsubstituted 2 to 6 membered heteroalkyl, substituted or unsubstituted C₆-C₁₂ aryl, or substituted or unsubstituted 5 to 12 membered heteroaryl.

In embodiments, R^{2.1} is independently —CH₂O—CH₂CCH, —CH₂O—CH₂CN, —CH₂O—CH₂-heterocycloalkyl, substituted or unsubstituted C₆-C₁₂ aryl, or substituted or unsubstituted 5 to 12 membered heteroaryl.

In embodiments, R^{2.1} is independently hydrogen, substituted or unsubstituted C₁-C₆ alkyl, or substituted or unsubstituted 2 to 6 membered heteroalkyl.

In embodiments, R^{2.1} is independently hydrogen, R²⁰-substituted or unsubstituted C₁-C₆ alkyl, or R²⁰-substituted or unsubstituted 2 to 6 membered heteroalkyl. In embodiments, R²⁰ is independently —OH, R²¹-substituted or unsubstituted 5 to 6 membered heterocycloalkyl or R²¹-substituted or unsubstituted 5 to 6 membered heteroaryl. In embodiments, R²¹ is independently oxo.

In embodiments, R^{2.1} is independently

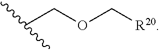

In embodiments, R^{2.1} is independently

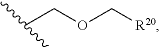

wherein R²⁰ is unsubstituted C₁-C₆ alkyl. In embodiments, R^{2.1} is independently

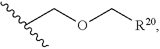

wherein R²⁰ is unsubstituted C₁-C₂ alkyl. In embodiments, R^{2.1} is independently

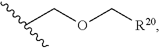

wherein R²⁰ is unsubstituted C₂ alkenyl. In embodiments, R^{2.1} is independently

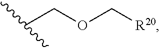

wherein R²⁰ is unsubstituted C₂ alkynyl. In embodiments, R^{2.1} is independently

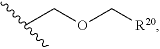

wherein R²⁰ is R²¹-substituted or unsubstituted 5 to 6 membered heterocycloalkyl. In embodiments, R^{2.1} is independently

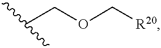

wherein R²⁰ is unsubstituted 5 to 6 membered heterocycloalkyl. In embodiments, R^{2.1} is independently

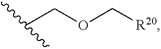

wherein R²⁰ is R²¹-substituted 5 to 6 membered heterocycloalkyl and R²¹ is oxo. In embodiments, R^{2.1} is independently

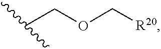

wherein R²⁰ is unsubstituted 5 to 6 membered heteroaryl. In embodiments, R^{2.1} is independently

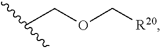

wherein R²⁰ is unsubstituted pyrazolyl. In embodiments, R^{2.1} is independently

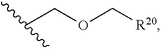

wherein R²⁰ is unsubstituted triazolyl. In embodiments, R²¹ is independently:

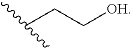 [SMILES: *CCO]

In embodiments, R^{2.1} is independently:

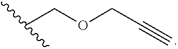 [SMILES: *COCC#C]

In embodiments, R^{2.1} is independently:

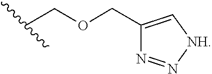 [SMILES: *COCc1c[nH]nn1]

In embodiments, R^{2.1} is independently:

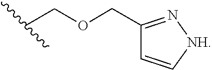 [SMILES: *COCc1cc[nH]n1]

In embodiments, R²¹ is independently:

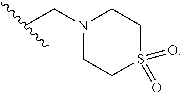 [SMILES: *CN1CCS(=O)(=O)CC1]

In embodiments, R³ is independently —CN,

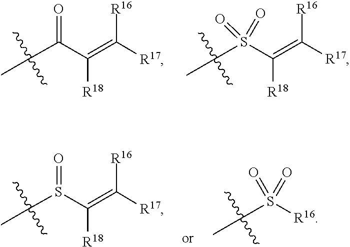

In embodiments, R³ is independently

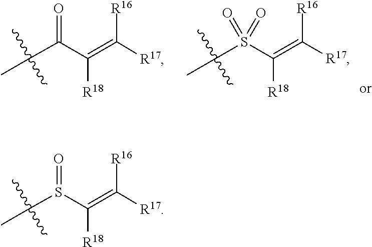

In embodiments, R¹⁶, R¹⁷, and R¹⁸ are independently hydrogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₇ cycloalkyl, or substituted or unsubstituted C₆-C₁₂ aryl. In embodiments, R¹⁶ is independently hydrogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₇ cycloalkyl, or substituted or unsubstituted C₆-C₁₂ aryl. In embodiments, R¹⁷ is independently hydrogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₇ cycloalkyl, or substituted or unsubstituted C₆-C₁₂ aryl. In embodiments, R¹⁸ is independently hydrogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₇ cycloalkyl, or substituted or unsubstituted C₆-C₁₂ aryl.

In embodiments, R¹⁶, R¹⁷, and R¹⁸ are independently hydrogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₆ cycloalkyl, or substituted C₆ aryl.

In embodiments, R¹⁶, R¹⁷, and R¹⁸ are independently hydrogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl.

In embodiments, R^{1A}, R^{1B}, R^{1C}, and R^{1D} are independently hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl.

In embodiments, R^{1A}, R^{1B}, and R^{1D} are independently hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl.

In embodiments, X is independently —F, —Cl, —Br, or —I.

In embodiments, L² is unsubstituted n-propylene or unsubstituted n-butylene.

In embodiments, R^{1.1} is independently hydrogen, —OCCl₃, —OCF₃, —OCBr₃, —OCI₃, —OCHCl₂, —OCHBr₂, —OCHI₂, —OCHF₂, —OCH₂Cl, —O CH₂Br, —OCH₂I, —OCH₂F, —CN, —SH, —SCH₃, —SCF₃, —SCHF₂, —SCH₂F, —SCCl₃, —SCHCl₂, —SCH₂Cl, —SCBr₃, —SCHBr₂, —SCH₂Br, —SCl₃, —SCHI₂, —SCH₂I, —SOCH₃, —SO₂CH₃, —NH₂, —NHCH₃, —OH, —SF₅, alkenyl, alkynyl, unsubstituted methoxy, unsubstituted ethoxy, unsubstituted n-propoxy, unsubstituted isopropoxy, unsubstituted n-butoxy, unsubstituted t-butoxy, unsubstituted sec-butoxy, unsubstituted isobutoxy, or unsubstituted pyrazolyl; R¹² is independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, or unsubstituted C₁-C₄ alkyl; and R^{1.3} is independently hydrogen, halogen, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, unsubstituted methoxy, unsubstituted ethoxy, unsubstituted n-propoxy, unsubstituted isopropoxy, unsubstituted n-butoxy, unsubstituted t-butoxy, unsubstituted sec-butoxy, or unsubstituted isobutoxy.

In embodiments, R^{1.1} is independently hydrogen, —OCCl₃, —OCF₃, —OCBr₃, —OCI₃, —OCHCl₂, —OCHBr₂, —OCHI₂, —OCHF₂, —OCH₂Cl, —O CH₂Br, —OCH₂I, —OCH₂F, —CN, —SH, —SCH₃, —SCF₃, —SCHF₂, —SCH₂F, —SCCl₃, —SCHCl₂, —SCH₂Cl, —SCBr₃, —SCHBr₂, —SCH₂Br, —SCl₃, —SCHI₂, —SCH₂I, —SOCH₃, —SO₂CH₃, —NH₂, —NHCH₃, —OH, —SF₅, alkenyl, alkynyl, unsubstituted methoxy, unsubstituted ethoxy, unsubstituted n-propoxy, unsubstituted isopropoxy, unsubstituted n-butoxy, unsubstituted t-butoxy, unsubstituted sec-butoxy, unsubstituted isobutoxy, or unsubstituted pyrazolyl.

In embodiments, R^{1.2} is independently hydrogen, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, or unsubstituted C₁-C₄ alkyl.

In embodiments, R^{1.3} is independently hydrogen,

halogen, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, unsubstituted methoxy, unsubstituted ethoxy, unsubstituted n-propoxy, unsubstituted isopropoxy, unsubstituted n-butoxy, unsubstituted t-butoxy, unsubstituted sec-butoxy, or unsubstituted isobutoxy.

In embodiments, R^{1.1} is independently

hydrogen, —OCF₃, —CN, —SCH₃, —SCF₃, —SOCH₃, —SO₂CH₃, —NHCH₃, —SF₅, unsubstituted C₂-C₄ alkenyl, unsubstituted C₂-C₄ alkynyl, unsubstituted isopropoxy, or unsubstituted pyrazolyl; R¹² is independently hydrogen, —F, —Br, or —CF₃; and R^{1.3} is independently hydrogen, —F, or —OCF₃.

In embodiments, R^{1.1} is independently

hydrogen, —OCF₃, —CN, —SCH₃, —SCF₃, —SOCH₃, —SO₂CH₃, —NHCH₃, —SF₅, unsubstituted C₂-C₄ alkenyl, unsubstituted C₂-C₄ alkynyl, unsubstituted isopropoxy, or unsubstituted pyrazolyl.

In embodiments, R^{1.2} is independently hydrogen, —F, —Br, or —CF₃.

In embodiments, R^{1.3} is independently hydrogen, —F or —OCF₃.

In embodiments, R³ is independently —CN.

In embodiments, R³ is independently

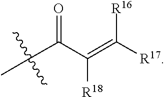

In embodiments, R³ is independently

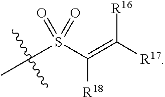

In embodiments, R³ is independently

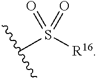

In embodiments, R³ is independently

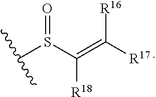

In embodiments, R¹⁶ is hydrogen; R¹⁷ is independently hydrogen, unsubstituted C₁-C₄ alkyl, or unsubstituted C₃-C₆ cycloalkyl; and R¹⁸ is independently hydrogen, unsubstituted C₁-C₄ alkyl, or unsubstituted C₃-C₆ cycloalkyl.

In embodiments, R¹⁶ is hydrogen; R¹⁷ is independently hydrogen or unsubstituted C₁-C₄ alkyl; and R¹⁸ is independently hydrogen or unsubstituted C₁-C₄ alkyl.

In embodiments, R¹⁶ is hydrogen; R¹⁷ is independently hydrogen, unsubstituted methyl, or unsubstituted cyclopropyl; and R¹⁸ is independently hydrogen, unsubstituted methyl, or unsubstituted cyclopropyl.

In embodiments, R¹⁶ is hydrogen; R¹⁷ is independently hydrogen or unsubstituted methyl; and R¹⁸ is independently hydrogen or unsubstituted methyl.

In embodiments, R¹⁶, R¹⁷ and R¹⁸ are hydrogen.

In embodiments, R¹⁶ is hydrogen. In embodiments, R¹⁶ is unsubstituted C₁-C₄ alkyl. In embodiments, R¹⁶ is unsubstituted methyl. In embodiments, R¹⁶ is unsubstituted ethyl. In embodiments, R¹⁶ is unsubstituted n-propyl. In embodiments, R¹⁶ is unsubstituted iso-propyl. In embodiments, R¹⁶ is unsubstituted n-butyl. In embodiments, R¹⁶ is unsubstituted t-butyl. In embodiments, R¹⁷ is hydrogen. In embodiments, R¹⁷ is unsubstituted C₁-C₄ alkyl. In embodiments, R¹⁷ is unsubstituted methyl. In embodiments, R¹⁷ is unsubstituted ethyl. In embodiments, R¹⁷ is unsubstituted n-propyl. In embodiments, R¹⁷ is unsubstituted iso-propyl. In embodiments, R¹⁷ is unsubstituted n-butyl. In embodiments, R¹⁷ is unsubstituted t-butyl. In embodiments, R¹⁸ is hydrogen. In embodiments, R¹⁸ is unsubstituted C₁-C₄ alkyl. In embodiments, R¹⁸ is unsubstituted methyl. In embodiments, R¹⁸ is unsubstituted ethyl. In embodiments, R¹⁸ is unsubstituted n-propyl. In embodiments, R¹⁸ is unsubstituted iso-propyl. In embodiments, R¹⁸ is unsubstituted n-butyl. In embodiments, R¹⁸ is unsubstituted t-butyl.

In embodiments, R¹⁶ is hydrogen. In embodiments, R¹⁶ is C₁-C₄ alkyl. In embodiments, R¹⁶ is methyl. In embodiments, R¹⁶ is substituted or unsubstituted aryl. In embodiments, R¹⁶ is substituted phenyl. In embodiments, R¹⁶ is halo substituted phenyl. In embodiments, R¹⁶ is fluoro substituted phenyl. In embodiments, R¹⁶ is unsubstituted phenyl. In embodiments, R¹⁶ is

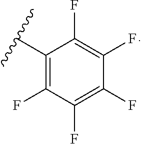 [SMILES: *c1c(F)c(F)c(F)c(F)c1F]

In embodiments, R¹⁶, R¹⁷, and R¹⁸ are independently hydrogen, —CN, substituted or unsubstituted C₁-C₁₂ alkyl, substituted or unsubstituted 2 to 12 membered heteroalkyl; or substituted or unsubstituted C₆-C₁₀ aryl. In embodiments, R¹⁶, R¹⁷, and R¹⁸ are independently hydrogen, substituted or unsubstituted C₁-C₁₂ alkyl, substituted or unsubstituted 2 to 12 membered heteroalkyl; or substituted or unsubstituted C₆-C₁₀ aryl. In embodiments, R¹⁶, R¹⁷, and R¹⁸ are hydrogen.

In embodiments, R¹⁶ is independently hydrogen, —CN, unsubstituted C₁-C₁₂ alkyl (e.g., C₁-C₁₀, alkyl, C₁-C₈ alkyl, C₁-C₆ alkyl, or C₁-C₄ alkyl), unsubstituted 2 to 12 membered heteroalkyl (e.g., 2 to 10 membered heteroalkyl, 2 to 8 membered heteroalkyl, 2 to 6 membered heteroalkyl, or 2 to 4 membered heteroalkyl), or unsubstituted C₆-C₁₀ aryl (e.g., C₆-C₁₂ aryl, C₆-C₁₀ aryl, C₁₀ aryl, or phenyl).

In embodiments, R¹⁶ is independently hydrogen, —CN, R²⁶-substituted or unsubstituted C₁-C₁₂ alkyl (e.g., C₁-C₁₀, alkyl, C₁-C₈ alkyl, C1-C₆ alkyl, or C₁-C₄ alkyl), R²⁶-substituted or unsubstituted 2 to 12 membered heteroalkyl (e.g., 2 to 10 membered heteroalkyl, 2 to 8 membered heteroalkyl, 2 to 6 membered heteroalkyl, or 2 to 4 membered heteroalkyl), or R²⁶-substituted or unsubstituted C₆-C₁₀ aryl (e.g., C₆-C₁₂ aryl, C₆-C₁₀ aryl, C₁₀ aryl, or phenyl).

R²⁶ is independently oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, —SF₅, R³⁶-substituted or unsubstituted alkyl (e.g., C₁-C₈ alkyl, C₁-C₆ alkyl, or C₁-C₄ alkyl), R³⁶-substituted or unsubstituted heteroalkyl (e.g., 2 to 8 membered heteroalkyl, 2 to 6 membered heteroalkyl, or 2 to 4 membered heteroalkyl), R³⁶-substituted or unsubstituted cycloalkyl (e.g., C₃-C₈ cycloalkyl, C₃-C₆ cycloalkyl, or C5-C6 cycloalkyl), R³⁶-substituted or unsubstituted heterocycloalkyl (e.g., 3 to 8 membered heterocycloalkyl, 3 to 6 membered heterocycloalkyl, or 5 to 6 membered heterocycloalkyl), R³⁶-substituted or unsubstituted aryl (e.g., C₆-C₁₂ aryl, C₆-C₁₀ aryl, C₁₀ aryl, or phenyl), or R³⁶-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R²⁶ is independently oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C5-C6 cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

R³⁶ is independently oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI ₂, —OCHF₂, —OCH₂Cl, —OCH₂Br, —OCH₂I, —OCH₂F, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R¹⁷ is independently hydrogen, unsubstituted C₁-C₄ alkyl, or unsubstituted C₃-C₆ cycloalkyl. In embodiments, R¹⁷ is hydrogen or unsubstituted C₁-C₄ alkyl. In embodiments, R¹⁷ is hydrogen, unsubstituted methyl, or unsubstituted cyclopropyl. In embodiments, R¹⁷ is hydrogen or unsubstituted methyl. In embodiments, R¹⁷ is hydrogen. In embodiments, R¹⁷ is unsubstituted methyl.

In embodiments, R¹⁷ is independently hydrogen, —CN, unsubstituted C₁-C₁₂ alkyl (e.g., C₁-C₁₀, alkyl, C₁-C₈ alkyl, C₁-C₆ alkyl, or C₁-C₄ alkyl), unsubstituted 2 to 12 membered heteroalkyl (e.g., 2 to 10 membered heteroalkyl, 2 to 8 membered heteroalkyl, 2 to 6 membered heteroalkyl, or 2 to 4 membered heteroalkyl), or unsubstituted C₆-C₁₀ aryl (e.g., C₆-C₁₂ aryl, C₆-C₁₀ aryl, C₁₀ aryl, or phenyl).

In embodiments, R¹⁷ is independently hydrogen, —CN, R²⁷-substituted or unsubstituted C₁ C₁₂ alkyl (e.g., C₁-C₁₀, alkyl, C₁-C₈ alkyl, C₁-C₆ alkyl, or C₁-C₄ alkyl), R²⁷-substituted or unsubstituted 2 to 12 membered heteroalkyl (e.g., 2 to 10 membered heteroalkyl, 2 to 8 membered heteroalkyl, 2 to 6 membered heteroalkyl, or 2 to 4 membered heteroalkyl), or R²⁷-substituted or unsubstituted C₆-C₁₀ aryl (e.g., C₆-C₁₂ aryl, C₆-C₁₀ aryl, C₁₀ aryl, or phenyl).

R²⁷ is independently oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, —SF₅, R³⁷-substituted or unsubstituted alkyl (e.g., C₁-C₈ alkyl, C₁-C₆ alkyl, or C₁-C₄ alkyl), R³⁷-substituted or unsubstituted heteroalkyl (e.g., 2 to 8 membered heteroalkyl, 2 to 6 membered heteroalkyl, or 2 to 4 membered heteroalkyl), R³⁷-substituted or unsubstituted cycloalkyl (e.g., C₃-C₅ cycloalkyl, C₃-C₆ cycloalkyl, or C₅-C₆ cycloalkyl), R³⁷-substituted or unsubstituted heterocycloalkyl (e.g., 3 to 8 membered heterocycloalkyl, 3 to 6 membered heterocycloalkyl, or 5 to 6 membered heterocycloalkyl), R³⁷-substituted or unsubstituted aryl (e.g., C₆-C₁₂ aryl, C₆-C₁₀ aryl, C₁₀ aryl, or phenyl), or R³⁷-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R²⁷ is independently oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

R³⁷ is independently oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI 2, —OCHF₂, —OCH₂Cl, —OCH₂Br, —OCH₂I, —OCH₂F, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R¹⁸ is independently hydrogen, unsubstituted C₁-C₄ alkyl, or unsubstituted C₃-C₆ cycloalkyl. In embodiments, R¹⁸ is independently hydrogen or unsubstituted C₁-C₄ alkyl. In embodiments, R¹⁸ is independently hydrogen or unsubstituted C₃-C₆ cycloalkyl. In embodiments, R¹⁸ is independently hydrogen, unsubstituted methyl, or unsubstituted cyclopropyl. In embodiments, R¹⁸ is independently hydrogen or unsubstituted methyl. In embodiments, R¹⁸ is hydrogen. In embodiments, R¹⁸ is unsubstituted methyl. In embodiments, R¹⁸ is unsubstituted cyclopropyl. In embodiments, R¹⁸ is —CN.

In embodiments, R¹⁸ is independently hydrogen, —CN, unsubstituted C₁-C₁₂ alkyl (e.g., C₁-C₁₀, alkyl, C₁-C₈ alkyl, C₁-C₆ alkyl, or C₁-C₄ alkyl), unsubstituted 2 to 12 membered heteroalkyl (e.g., 2 to 10 membered heteroalkyl, 2 to 8 membered heteroalkyl, 2 to 6 membered heteroalkyl, or 2 to 4 membered heteroalkyl), or unsubstituted C₆-C₁₀ aryl (e.g., C₆-C₁₂ aryl, C₆-C₁₀ aryl, C₁₀ aryl, or phenyl).

In embodiments, R¹⁸ is independently hydrogen, —CN, R²⁸-substituted or unsubstituted C₁-C₁₂ alkyl (e.g., C₁-C₁₀, alkyl, C₁-C₈ alkyl, C₁-C₆ alkyl, or C₁-C₄ alkyl), R²⁸-substituted or unsubstituted 2 to 12 membered heteroalkyl (e.g., 2 to 10 membered heteroalkyl, 2 to 8 membered heteroalkyl, 2 to 6 membered heteroalkyl, or 2 to 4 membered heteroalkyl), or R²⁸-substituted or unsubstituted C₆-C₁₀ aryl (e.g., C₆-C₁₂ aryl, C₆-C₁₀ aryl, C₁₀ aryl, or phenyl).

R²⁸ is independently oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, —SF₅, R³⁸-substituted or unsubstituted alkyl (e.g., C₁-C₈ alkyl, C₁-C₆ alkyl, or C₁-C₄ alkyl), R³⁸-substituted or unsubstituted heteroalkyl (e.g., 2 to 8 membered heteroalkyl, 2 to 6 membered heteroalkyl, or 2 to 4 membered heteroalkyl), R³⁸-substituted or unsubstituted cycloalkyl (e.g., C₃-C₈ cycloalkyl, C₃-C₆ cycloalkyl, or C₅-C₆ cycloalkyl), R³⁸-substituted or unsubstituted heterocycloalkyl (e.g., 3 to 8 membered heterocycloalkyl, 3 to 6 membered heterocycloalkyl, or 5 to 6 membered heterocycloalkyl), R³⁸-substituted or unsubstituted aryl (e.g., C₆-C₁₂ aryl, C₆-C₁₀ aryl, C₁₀ aryl, or phenyl), or R³⁸-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R²⁸ is independently oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI ₂, —OCHF₂, —OCH₂Cl, —OCH₂Br, —OCH₂I, —OCH₂F, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

R³⁸ is independently oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI ₂, —OCHF₂, —OCH₂Cl, —OCH₂Br, —OCH₂I, —OCH₂F, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R¹⁶ is independently hydrogen or R²⁶-substituted or unsubstituted C₁-C₄ alkyl.

In embodiments, R¹⁷ is independently hydrogen, R²⁷-substituted or unsubstituted C₁-C₄ alkyl, or R²⁷-substituted or unsubstituted C₃-C₆ cycloalkyl. In embodiments, R¹⁷ is independently hydrogen or R²⁷-substituted or unsubstituted C₁-C₄ alkyl.

In embodiments, R¹⁸ is independently hydrogen, R²⁸-substituted or unsubstituted C₁-C₄ alkyl, or R²⁸-substituted or unsubstituted C₃-C₆ cycloalkyl. In embodiments, R¹⁸ is independently hydrogen or R²⁸-substituted or unsubstituted C₁-C₄ alkyl.

In embodiments, R²⁶ is independently —F, —Cl, —Br, or —I. In embodiments, R²⁶ is independently-F. In embodiments, R²⁶ is independently —Cl. In embodiments, R²⁶ is independently-Br. In embodiments, R²⁶ is independently —I.

In embodiments, the compound has the formula:

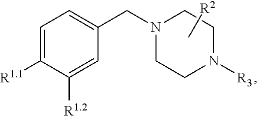

wherein R^{1.1}, R^{1.2}, R², and R³ are as described herein. In embodiments, the compound has the formula:

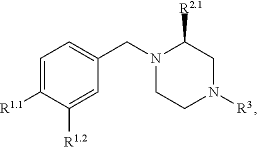

wherein R^{1.1}, R^{1.2}, R^{2.1}, and R³ are as described herein. In embodiments, the compound has the formula:

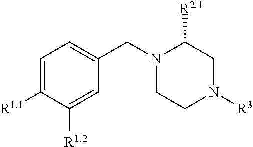

wherein R^{1.1}, R^{1.2}, R^{2.1}, and R³ are as described herein. In embodiments, the compound has the formula:

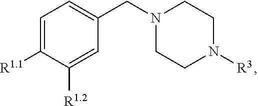

R^{1.1}, wherein R^{1.1}, R^{1.2}, and R³ are as described herein. In embodiments, the compound has the formula:

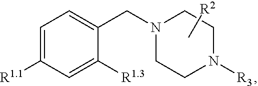

wherein R^{1.1}, R^{1.3}, R², and R³ are as described herein. In embodiments, the compound has the formula:

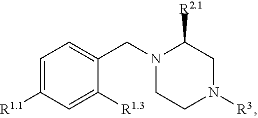

wherein R^{1.1}, R^{1.3}, R^{2.1}, and R³ are as described herein. In embodiments, the compound has the formula:

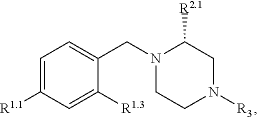

wherein R^{1.1}, R^{1.3}, R^{2.1}, and R³ are as described herein. In embodiments, the compound has the formula:

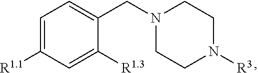

wherein R^{1.1}, R^{1.3}, and R³ are as described herein. In embodiments, the compound has the formula:

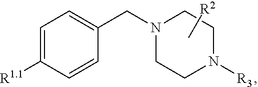

wherein R^{1.1}, R² and R³ are as described herein. In embodiments, the compound has the formula:

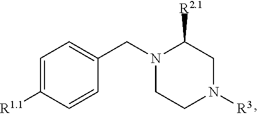

wherein R^{1.1}, R^{2.1}, and R³ are as described herein. In embodiments, the compound has the formula:

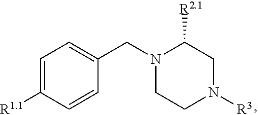

wherein R^{1.1}, R^{2.1}, and R³ are as described herein. In embodiments, the compound has the formula:

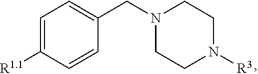

wherein R^{1.1} and R³ are as described herein. In embodiments, the compound has the formula:

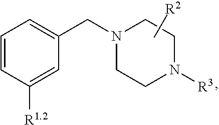

wherein R^{1.2}, R², and R³ are as described herein. In embodiments, the compound has the formula:

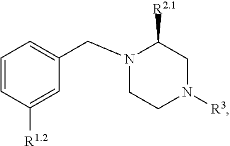

wherein, R^{1.2}, R^{2.1}, and R³ are as described herein. In embodiments, the compound has the formula:

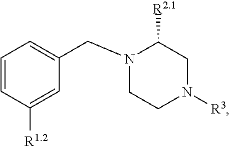

wherein R^{1.2}, R^{2.1}, and R³ are as described herein. In embodiments, the compound has the formula:

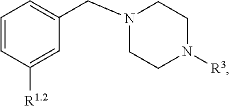

wherein R^{1.2} and R³ are as described herein. In embodiments, the compound has the formula:

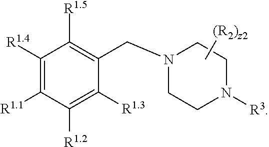

R^{1.1}, R^{1.2}, R^{1.3}, R², z2, and R³ are as described herein. R^{1.4} and R^{1.5} may each independently be hydrogen or any value of R¹ as described herein.

In embodiments, the compound has the formula:

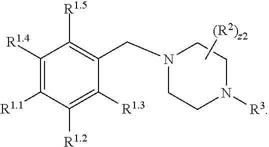

R^{1.1}, R^{1.2}, R^{1.3}, R², z2, and R³ are as described herein. R^{1.4} and R^{1.5} may each independently be hydrogen or any value of R¹ as described herein.

In embodiments, the compound has the formula:

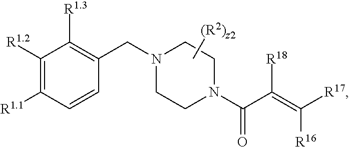

wherein R^{1.1}, R^{1.2}, R^{1.3}, R², R¹⁶, R¹⁷, R¹⁸, and z2 are as described herein. In embodiments, the compound has the formula:

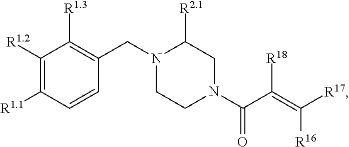

wherein R^{1.1}, R^{1.2}, R^{1.3}, R^{2.1}, R^{1.6}, R¹⁷, and R¹⁸ are as described herein. In embodiments, the compound has the formula:

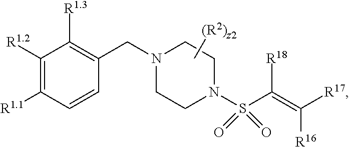

wherein R^{1.1}, R^{1.2}, R^{1.3}, R², R¹⁶, R¹⁷, R¹⁸, and z2 are as described herein. In embodiments, the compound has the formula:

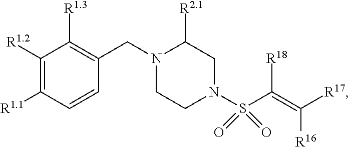

wherein R^{1.1}, R^{1.2}, R^{1.3}, R^{2.1}, R¹⁶, R¹⁷, and R¹⁸ are as described herein. In embodiments, the compound has the formula:

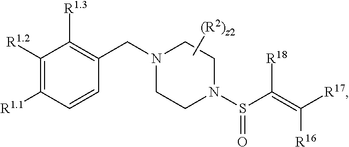

wherein R^{1.1}, R^{1.2}, R^{1.3}, R², R¹⁶, R¹⁷, R¹⁸, and z2 are as described herein. In embodiments, the compound has the formula:

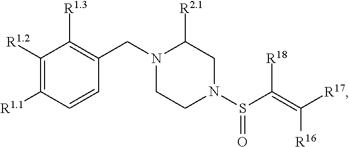

wherein R^{1.1}, R^{1.2}, R^{1.3}, R^{2.1}, R¹⁶, R¹⁷, and R¹⁸ are as described herein. In embodiments, the compound has the formula:

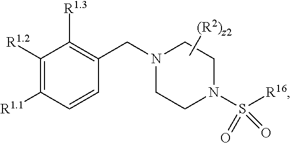

wherein R^{1.1}, R^{1.2}, R^{1.3}, R², R¹⁶, and z2 are as described herein. In embodiments, the compound has the formula:

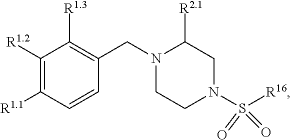

wherein R^{1.1}, R^{1.2}, R^{1.3}, R^{2.1}, and R¹⁶ are as described herein.

In embodiments, the compound has the formula:

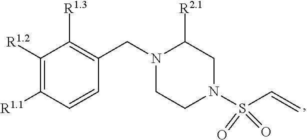

wherein R^{1.1}, R^{1.2}, R^{1.3}, and R^{2.1} are as described herein. In embodiments, the compound has the formula:

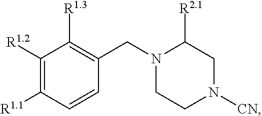

wherein R^{1.1}, R^{1.2}, R^{1.3}, and R^{2.1} are as described herein. In embodiments, the compound has the formula:

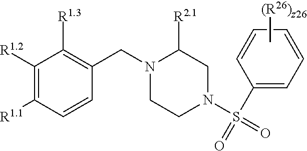

wherein R^{1.1}, R^{1.2}, R^{1.3}, R^{2.1}, and R²⁶, are as described herein. The symbol z26 is an integer from 0 to 5. In embodiments, the compound has the formula:

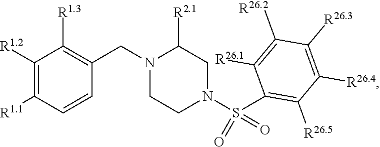

wherein R^{1.1}, R^{1.2}, R^{1.3}, and R^{2.1} are as described herein. R^{26.1}, R^{26.2}, R^{26.3}, R^{26.4}, and R^{26.5} may each independently be hydrogen or any value of R²⁶ as described herein. In embodiments, the compound has the formula:

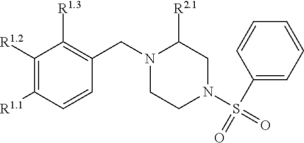

wherein R^{1.1}, R^{1.2}, R^{1.3}, and R^{2.1} are as described herein. In embodiments, the compound has the formula:

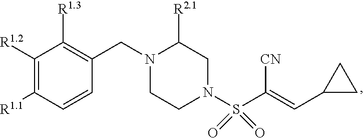

wherein R^{1.1}, R^{1.2}, R^{1.3}, and R^{2.1} are as described herein. In embodiments, the compound has the formula:

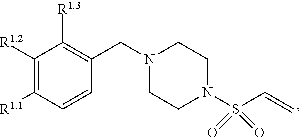

wherein R^{1.1}, R^{1.2} and R^{1.3} are as described herein. In embodiments, the compound has the formula:

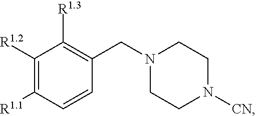

wherein R^{1.1}, R^{1.2}, and R^{1.3} are as described herein. In embodiments, the compound has the formula:

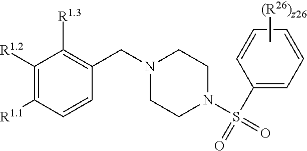

wherein R^{1.1}, R^{1.2}, R^{1.3}, R²⁶, and z26 are as described herein. In embodiments, the compound has the formula:

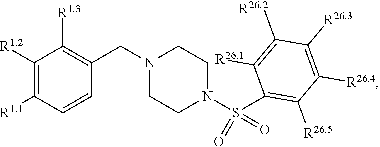

wherein R^{1.1}, R^{1.2}, R^{1.3}, R^{26.1}, R^{26.2}, R^{26.3}, R^{26.4}, and R^{26.5} are as described herein. In embodiments, the compound has the formula:

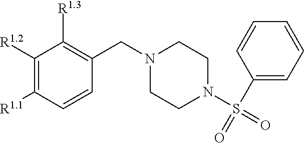

wherein R^{1.1}, R^{1.2}, and R^{1.3} are as described herein. In embodiments, the compound has the formula:

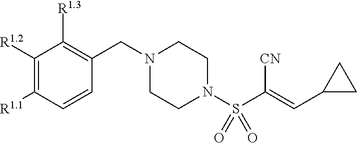

wherein R^{1.1}, R^{1.2}, and R¹³ are as described herein.

In embodiments, the compound has the formula:

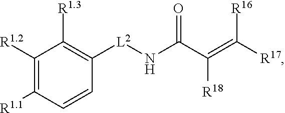

wherein R^{1.1}, R^{1.2}, R^{1.3}, L², R¹⁶, R¹⁷, and R¹⁸ are as described herein. In embodiments, the compound has the formula:

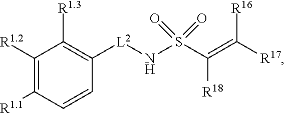

wherein R^{1.1}, R^{1.2}, R^{1.3}, L², R¹⁶, R¹⁷, and R¹⁸ are as described herein. In embodiments, the compound has the formula:

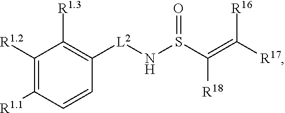

wherein R^{1.1}, R^{1.2}, R^{1.3}, L², R¹⁶, R¹⁷, and R¹⁸ are as described herein. In embodiments, the compound has the formula:

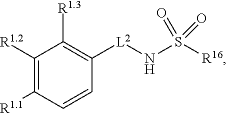

wherein R^{1.1}, R^{1.2}, R^{1.3}, L², and R¹⁶ are as described herein.

In embodiments, R^{26.1} is hydrogen. In embodiments, R^{26.2} is hydrogen. In embodiments, R^{26.3} is hydrogen. In embodiments, R^{26.4} is hydrogen. In embodiments, R^{26.5} is hydrogen.

In embodiments, R^{26.1} is-F, —Cl, —Br, or —I. In embodiments, R^{26.1} is-F. In embodiments, R^{26.1} is —Cl. In embodiments, R^{26.1} is-Br. In embodiments, R^{26.1} is —I. In embodiments, R^{26.2} is —F, —Cl, —Br, or —I. In embodiments, R^{26.2} is-F. In embodiments, R^{26.2} is —Cl. In embodiments, R^{26.2} is-Br. In embodiments, R^{26.2} is —I. In embodiments, R^{26.3} is —F, —Cl, —Br, or —I. In embodiments, R^{26.3} is-F. In embodiments, R^{26.3} is —Cl. In embodiments, R^{26.3} is-Br. In embodiments, R^{26.3} is —I. In embodiments, R^{26.4} is —F, —Cl, —Br, or —I. In embodiments, R^{26.4} is-F. In embodiments, R^{26.4} is —Cl. In embodiments, R^{26.4} is-Br. In embodiments, R^{26.4} is —I. In embodiments, R^{26.5} is —F, —Cl, —Br, or —I. In embodiments, R^{26.5} is-F. In embodiments, R^{26.5} is —Cl. In embodiments, R^{26.5} is-Br. In embodiments, R^{26.5} is —I.

In embodiments, R¹ is independently oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R¹ is independently oxo, halogen, —CX¹³, —CHX¹², —CH₂X¹, —OCX¹³, —OCH₂X¹, —OCHX¹², —CN, —SR^{1D}, —SOR^{1D}, —SO₂R^{1D}, —SO₃R^{1D}, —SO₄R^{1D}, —SONR^{1A}R^{1B}, —SO₂NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O), —N(O)₂, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A} C(O)OR^{1C}, —NR^{1A}OR^{1C}, —SF₅, —N₃, R¹⁰-substituted or unsubstituted alkyl (e.g., C₁-C₈ alkyl, C₁-C₆ alkyl, or C₁-C₄ alkyl), R¹⁰-substituted or unsubstituted heteroalkyl (e.g., 2 to 8 membered heteroalkyl, 2 to 6 membered heteroalkyl, or 2 to 4 membered heteroalkyl), R¹⁰-substituted or unsubstituted cycloalkyl (e.g., C₃-C₈ cycloalkyl, C₃-C₆ cycloalkyl, or C₅-C₆ cycloalkyl), R¹⁰-substituted or unsubstituted heterocycloalkyl (e.g., 3 to 8 membered heterocycloalkyl, 3 to 6 membered heterocycloalkyl, or 5 to 6 membered heterocycloalkyl), R¹⁰-substituted or unsubstituted aryl (e.g., C₆-C₁₂ aryl, C₆-C₁₀ aryl, C₁₀ aryl, or phenyl), or R¹⁰-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R¹ is independently oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, R¹⁰-substituted or unsubstituted alkyl (e.g., C₁-C₈ alkyl, C₁-C₆ alkyl, or C₁-C₄ alkyl), R¹⁰-substituted or unsubstituted heteroalkyl (e.g., 2 to 8 membered heteroalkyl, 2 to 6 membered heteroalkyl, or 2 to 4 membered heteroalkyl), R¹⁰-substituted or unsubstituted cycloalkyl (e.g., C₃-C₈ cycloalkyl, C₃-C₆ cycloalkyl, or C₅-C₆ cycloalkyl), R¹⁰-substituted or unsubstituted heterocycloalkyl (e.g., 3 to 8 membered heterocycloalkyl, 3 to 6 membered heterocycloalkyl, or 5 to 6 membered heterocycloalkyl), R¹⁰-substituted or unsubstituted aryl (e.g., C₆-C₁₂ aryl, C₆-C₁₀ aryl, C₁₀ aryl, or phenyl), or R¹⁰-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R¹ is independently oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

R¹⁰ is independently oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, —SF₅, R¹¹-substituted or unsubstituted alkyl (e.g., C₁-C₈ alkyl, C₁-C₆ alkyl, or C₁-C₄ alkyl), R¹¹-substituted or unsubstituted heteroalkyl (e.g., 2 to 8 membered heteroalkyl, 2 to 6 membered heteroalkyl, or 2 to 4 membered heteroalkyl), R¹¹-substituted or unsubstituted cycloalkyl (e.g., C₃-C₈ cycloalkyl, C₃-C₆ cycloalkyl, or C₅-C₆ cycloalkyl), R¹¹-substituted or unsubstituted heterocycloalkyl (e.g., 3 to 8 membered heterocycloalkyl, 3 to 6 membered heterocycloalkyl, or 5 to 6 membered heterocycloalkyl), R¹¹-substituted or unsubstituted aryl (e.g., C₆-C₁₂ aryl, C₆-C₁₀ aryl, C₁₀ aryl, or phenyl), or R¹¹-substituted or unsubstituted heteroaryl (e.g., 5 to 12 membered heteroaryl, 5 to 10 membered heteroaryl, 5 to 9 membered heteroaryl, or 5 to 6 membered heteroaryl).

R¹¹ is independently oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R¹⁰ is independently oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, two adjacent R¹ substituents are joined to form a substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, two adjacent R¹ substituents are joined to form a R¹⁰-substituted or unsubstituted cycloalkyl (e.g., C₃-C₈ cycloalkyl, C₃-C₆ cycloalkyl, or C₅-C₆ cycloalkyl), R¹⁰-substituted or unsubstituted heterocycloalkyl (e.g., 3 to 8 membered heterocycloalkyl, 3 to 6 membered heterocycloalkyl, or 5 to 6 membered heterocycloalkyl), R¹⁰-substituted or unsubstituted aryl (e.g., C₆-C₁₂ aryl, C₆-C₁₀ aryl, C₁₀ aryl, or phenyl), or R¹⁰-substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, two adjacent R¹ substituents are joined to form a unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{1A}, R^{1B}, R^{1C}, and R^{1D} are independently

hydrogen, —CX₃, —CN, —COOH, —CONH₂, —CHX₂, —CH₂X, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R^{1A}, R^{1B}, R^{1C}, and R^{1D} are independently hydrogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —CN, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₅ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{1A}, R^{1B}, R^{1C}, and R^{1D} are independently

hydrogen, —CX₃, —CN, —COOH, —CONH₂, —CHX₂, —CH₂X, R¹⁰-substituted or unsubstituted alkyl (e.g., C₁-C₈ alkyl, C₁-C₆ alkyl, or C₁-C₄ alkyl), R¹⁰-substituted or unsubstituted heteroalkyl (e.g., 2 to 8 membered heteroalkyl, 2 to 6 membered heteroalkyl, or 2 to 4 membered heteroalkyl), R¹⁰-substituted or unsubstituted cycloalkyl (e.g., C₃-C₈ cycloalkyl, C₃-C₆ cycloalkyl, or C₅-C₆ cycloalkyl), R¹⁰-substituted or unsubstituted heterocycloalkyl (e.g., 3 to 8 membered heterocycloalkyl, 3 to 6 membered heterocycloalkyl, or 5 to 6 membered heterocycloalkyl), R¹⁰-substituted or unsubstituted aryl (e.g., C₆-C₁₂ aryl, C₆-C₁₀ aryl, C₁₀ aryl, or phenyl), or R¹⁰-substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R^{1A}, R^{1B}, R^{1C}, and R^{1D} are independently hydrogen, —CF₃, —CBr₃, —CCl₃, —Cl₃, —CHF₂, —CHBr₂, —CHCl₂, —CHI₂, —CH₂F, —CH₂Br, —CH₂Cl, —C H₂I, —CN, —COOH, —CONH₂, R¹⁰-substituted or unsubstituted alkyl (e.g., C₁-C₈ alkyl, C₁-C₆ alkyl, or C₁-C₄ alkyl), R¹⁰-substituted or unsubstituted heteroalkyl (e.g., 2 to 8 membered heteroalkyl, 2 to 6 membered heteroalkyl, or 2 to 4 membered heteroalkyl), R¹⁰-substituted or unsubstituted cycloalkyl (e.g., C₃-C₅ cycloalkyl, C₃-C₆ cycloalkyl, or C₅-C₆ cycloalkyl), R¹⁰-substituted or unsubstituted heterocycloalkyl (e.g., 3 to 8 membered heterocycloalkyl, 3 to 6 membered heterocycloalkyl, or 5 to 6 membered heterocycloalkyl), R¹⁰-substituted or unsubstituted aryl (e.g., C₆-C₁₂ aryl, C₆-C₁₀ aryl, C₁₀ aryl, or phenyl), or R¹⁰-substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R^{1A}, R^{1B}, R^{1C}, and R^{1D} are independently hydrogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —CN, —COOH, —CONH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R^{1A} and R^{1B} substituents bonded to the same nitrogen atom may optionally be joined to form an R¹⁰-substituted or unsubstituted heterocycloalkyl (e.g., 3 to 8 membered heterocycloalkyl, 3 to 6 membered heterocycloalkyl, or 5 to 6 membered heterocycloalkyl) or R¹⁰-substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R^{1A} and R^{1B} substituents bonded to the same nitrogen atom may optionally be joined to form an unsubstituted heterocycloalkyl (e.g., 3 to 8 membered heterocycloalkyl, 3 to 6 membered heterocycloalkyl, or 5 to 6 membered heterocycloalkyl) or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R¹ is independently —OCH₃. In embodiments, R¹ is independently —OCH₂CH₃. In embodiments, R¹ is independently —OCH₂CH₂CH₃. In embodiments, R¹ is independently unsubstituted C₁-C₄ alkoxy. In embodiments, R¹ is independently —OCF₃. In embodiments, R¹ is independently —OCF₂CF₃. In embodiments, R¹ is independently unsubstituted C₁-C₄ haloalkoxy. In embodiments, R¹ is independently-Br. In embodiments, R¹ is independently —Cl. In embodiments, R¹ is independently-F. In embodiments, R¹ is independently halogen. In embodiments, R¹ is independently —CH₃. In embodiments, R¹ is independently —CH₂CH₃. In embodiments, R¹ is independently unsubstituted C₁-C₄ alkyl. In embodiments, R¹ is independently —CF₃. In embodiments, R¹ is independently —CF₂CF₃. In embodiments, R¹ is independently-CX¹₃. In embodiments, R¹ is independently unsubstituted C₁-C₄ haloalkyl. In embodiments, R¹ is independently —OCH₃ or —F. In embodiments, R¹ is independently unsubstituted C₁-C₄ alkoxy or halogen. In embodiments, R¹ is —CN. In embodiments, R¹ is independently-SH. In embodiments, R¹ is independently —SCH₃. In embodiments, R¹ is independently —SCF₃. In embodiments, R¹ is independently —SOCH₃. In embodiments, R¹ is independently —SO₂CH₃. In embodiments, R¹ is independently —SOCF₃. In embodiments, R¹ is independently —SO₂CF₃. In embodiments, R¹ is independently —SOₙ₁R^{1D}. In embodiments, R^{1D} is independently hydrogen. In embodiments, R^{1D} is independently unsubstituted C₁-C₄ alkyl. In embodiments, R^{1D} is independently unsubstituted C₁-C₄ haloalkyl. In embodiment, n1 is 0. In embodiment, n1 is 1. In embodiment, n1 is 2. In embodiments, R¹ is independently —SF₅. In embodiments, R¹ is independently unsubstituted 2 to 6 membered heteroalkyl. In embodiments, R¹ is independently unsubstituted 5 to 6 membered heteroaryl. In embodiments, R¹ is independently pyrazolyl. In embodiments, R¹ is independently pyrrolyl. In embodiments, R¹ is independently pyridazinyl. In embodiments, R¹ is independently triazinyl. In embodiments, R¹ is independently pyrimidinyl. In embodiments, R¹ is independently imidazolyl. In embodiments, R¹ is independently pyrazinyl. In embodiments, R¹ is independently purinyl. In embodiments, R¹ is independently oxazolyl. In embodiments, R¹ is independently isoxazolyl. In embodiments, R¹ is independently thiazolyl. In embodiments, R¹ is independently isothiazolyl. In embodiments, R¹ is independently furyl. In embodiments, R¹ is independently thienyl. In embodiments, R¹ is independently pyridyl. In embodiments, R¹ is independently pyrimidyl. In embodiments, R¹ is independently —C(O)NH₂. In embodiments, R¹ is independently —C(O)NR^{1A}R^{1B}. In embodiments, R^{1A} is independently hydrogen. In embodiments, R^{1A} is independently unsubstituted C₁-C₄ alkyl. In embodiments, R^{1A} is independently-CH₃. In embodiments, R^{1A} is independently —CH₂CH₃. In embodiments, R^{1B} is independently hydrogen. In embodiments, R^{1B} is independently unsubstituted C₁-C₄ alkyl.

In embodiments, R^{1B} is independently —CH₃. In embodiments, R^{1B} is independently —CH₂CH₃. In embodiments, R^{1C} is independently hydrogen. In embodiments, R^{1C} is independently unsubstituted C₁-C₄ alkyl. In embodiments, R^{1C} is independently —CH₃. In embodiments, R^{1C} is independently —CH₂CH₃. In embodiments, R^{1D} is independently hydrogen. In embodiments, R^{1D} is independently unsubstituted C₁-C₄ alkyl. In embodiments, R^{1D} is independently —CH₃. In embodiments, R^{1D} is independently —CH₂CH₃.

In embodiments, R¹ is independently halogen. In embodiments, R¹ is independently —CCl₃. In embodiments, R¹ is independently —CBr₃. In embodiments, R¹ is independently —CF₃. In embodiments, R¹ is independently —Cl₃. In embodiments, R¹ is independently —CHCl₂. In embodiments, R¹ is independently —CHBr₂. In embodiments, R¹ is independently —CHF₂. In embodiments, R¹ is independently —CHI₂. In embodiments, R¹ is independently —CH₂Cl. In embodiments, R¹ is independently —CH₂Br. In embodiments, R¹ is independently —CH₂F. In embodiments, R¹ is independently —CH₂I. In embodiments, R¹ is independently —CN. In embodiments, R¹ is independently-OH. In embodiments, R¹ is independently —NH₂. —NHCH₃. In embodiments, R¹ is independently-COOH. In embodiments, R¹ is independently —CONH₂. In embodiments, R¹ is independently —NO₂. In embodiments, R¹ is independently-SH. In embodiments, R¹ is independently —SO₃H. In embodiments, R¹ is independently —SO₄H. In embodiments, R¹ is independently —SO₂NH₂. In embodiments, R¹ is independently —SCH₃. In embodiments, R¹ is independently —SCF₃. In embodiments, R¹ is independently —SCHF₂. In embodiments, R¹ is independently —SCH₂F. In embodiments, R¹ is independently —SCCl₃. In embodiments, R¹ is independently —SCHCl₂. In embodiments, R¹ is independently —SCH₂Cl. In embodiments, R¹ is independently —SCBr₃. In embodiments, R¹ is independently —SCHBr₂. In embodiments, R¹ is independently —SCH₂Br. In embodiments, R¹ is independently —SCl₃. In embodiments, R¹ is independently —SCHI₂. In embodiments, R¹ is independently —SCH₂I. In embodiments, R¹ is independently —SOCH₃. In embodiments, R¹ is independently —SO₂CH₃. In embodiments, R¹ is independently —SF₅. In embodiments, R¹ is independently —NHNH₂. In embodiments, R¹ is independently —ONH₂. In embodiments, R¹ is independently —NHC(O)NHNH₂. In embodiments, R¹ is independently —NHC(O)NH₂. In embodiments, R¹ is independently —NHSO₂H. In embodiments, R¹ is independently —NHC(O)H. In embodiments, R¹ is independently —NHC(O)OH. In embodiments, R¹ is independently-NHOH. In embodiments, R¹ is independently —OCCl₃. In embodiments, R¹ is independently —OCF₃. In embodiments, R¹ is independently —OCBr₃. In embodiments, R¹ is independently —OCl₃. In embodiments, R¹ is independently —OCHCl₂. In embodiments, R¹ is independently —OCHBr₂. In embodiments, R¹ is independently —OCHI₂. In embodiments, R¹ is independently —OCHF₂. In embodiments, R¹ is independently —OCH₂Cl. In embodiments, R¹ is independently —OCH₂Br. In embodiments, R¹ is independently —OCH₂I. In embodiments, R¹ is independently —OCH₂F. In embodiments, R¹ is independently —N₃.

In embodiments, R¹ is independently halogen, —CF₃, —CHF₂, —CH₂F, —OCF₃, —OCH₂F, —OCHF₂, —SCF₃, —SCH₂F, —SCHF₂, —CN, —SR^{1D}, —SO₂R^{1D}—SO₂NR^{1A}R^{1B}, —NHC(O)NR^{1A}R^{1B}, —N(O)₂, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, substituted or unsubstituted C₁-C₈ alkyl, substituted or unsubstituted 2 to 5 membered heteroalkyl, substituted or unsubstituted C₃-C₆ cycloalkyl, substituted or unsubstituted 3 to 6 membered heterocycloalkyl, substituted or unsubstituted aryl, or substituted or unsubstituted 5 to 6 membered heteroaryl.

In embodiments, R¹ is independently unsubstituted C₄-C₆ alkyl, unsubstituted C₅-C₆ cycloalkyl, unsubstituted 5 to 6 membered heterocycloalkyl, or substituted or unsubstituted phenyl.

In embodiments, R^{1.1} is independently hydrogen, halogen, —CF₃, —CHF₂, —CH₂F, or substituted or unsubstituted C₁-C₈ alkyl. In embodiments, R^{1.1} is independently hydrogen. In embodiments, R^{1.1} is independently —CF₃. In embodiments, R^{1.1} is halogen or —OCF₃. In embodiments, R^{1.1} is independently-Br. In embodiments, R^{1.1} is independently —Cl. In embodiments, R^{1.1} is independently-F. In embodiments, R^{1.1} is —OCF₃. In embodiments, R^{1.1} is independently —OCF₂CF₃. In embodiments, R^{1.1} is independently unsubstituted C₁-C₄ haloalkoxy. In embodiments, R^{1.1} is independently —OCH₃. In embodiments, R^{1.1} is independently —OCH₂CH₃. In embodiments, R^{1.1} is independently —OCH₂CH₂CH₃. In embodiments, R^{1.1} is independently unsubstituted C₁-C₄ alkoxy. In embodiments, R^{1.1} is —CN. In embodiments, R¹ is independently-SH. In embodiments, R^{1.1} is independently —SCH₃. In embodiments, R^{1.1} is independently —SCF₃. In embodiments, R^{1.1} is independently —SOCH₃. In embodiments, R^{1.1} is independently —SO₂CH₃. In embodiments, R^{1.1} is independently —SOCF₃. In embodiments, R^{1.1} is independently —SO₂CF₃. In embodiments, R^{1.1} is independently —SOₙ₁R^{1D}. In embodiments, R^{1D} is independently hydrogen. In embodiments, R^{1D} is independently unsubstituted C₁-C₄ alkyl. In embodiments, R^{1D} is independently unsubstituted C₁-C₄ haloalkyl. In embodiment, n1 is 0. In embodiment, n1 is 1. In embodiment, n1 is 2. In embodiments, R^{1.1} is independently —SF₅. In embodiments, R^{1.1} is independently unsubstituted 5 to 6 membered heteroaryl. In embodiments, R^{1.1} is independently pyrazolyl. In embodiments, R^{1.1} is independently pyrrolyl. In embodiments, R^{1.1} is independently pyridazinyl. In embodiments, R^{1.1} is independently triazinyl. In embodiments, R^{1.1} is independently pyrimidinyl. In embodiments, R^{1.1} is independently imidazolyl. In embodiments, R¹ is independently pyrazinyl. In embodiments, R^{1.1} is independently purinyl. In embodiments, R¹ is independently oxazolyl. In embodiments, R^{1.1} is independently isoxazolyl. In embodiments, R^{1.1} is independently thiazolyl. In embodiments, R^{1.1} is independently isothiazolyl. In embodiments, R^{1.1} is independently furyl. In embodiments, R^{1.1} is independently thienyl. In embodiments, R^{1.1} is independently pyridyl. In embodiments, R^{1.1} is independently pyrimidyl.

In embodiments, R^{1.1} is independently hydrogen, halogen, —CF₃, —CHF₂, —CH₂F, —OCF₃, —OCH₂F, —OCHF₂, —SCF₃, —SCH₂F, —SCHF₂, —CN, —SR^{1D}, —SO₂R^{1D}—SO₂NR^{1A}R^{1B}, —C(O)R^{1C}, —C(O)—OR^{1C}, —OR^{1D}, —SF₅, substituted or unsubstituted C₁-C₈ alkyl, substituted or unsubstituted 2 to 5 membered heteroalkyl, substituted or unsubstituted C₃-C₆ cycloalkyl, substituted or unsubstituted 3 to 6 membered heterocycloalkyl, substituted or unsubstituted aryl, or substituted or unsubstituted 5 to 6 membered heteroaryl. In embodiments, R^{1.1} is independently —CF₃, —CHF₂, or —CH₂F. In embodiments, R^{1.1} is independently —OCF₃, —OCH₂F, or —OCHF₂. In embodiments, R^{1.1} is independently hydrogen. In embodiments, R^{1.1} is independently-Br. In embodiments, R^{1.1} is independently —Cl. In embodiments, R^{1.1} is independently-F. In embodiments, R^{1.1} is independently halogen. In embodiments, R^{1.1} is independently —OCF₃. In embodiments, R^{1.1} is independently —OCF₂CF₃. In embodiments, R¹¹ is independently unsubstituted C₁-C₄ haloalkoxy.

In embodiments, R^{1.2} is independently hydrogen, halogen, —CF₃, —CHF₂, —CH₂F, —OCF₃, —OCH₂F, —OCHF₂, —SCF₃, —SCH₂F, —SCHF₂, substituted or unsubstituted C₁-C₈ alkyl, or substituted or unsubstituted 2 to 5 membered heteroalkyl. In embodiments, R¹² is hydrogen or halogen. In embodiments, R^{1.2} is independently —CF₃, —CHF₂, or —CH₂F. In embodiments, R¹² is independently —OCF₃, —OCH₂F, or —OCHF₂. In embodiments, R^{1.2} is independently hydrogen. In embodiments, R¹² is independently-Br. In embodiments, R^{1.2} is independently —Cl. In embodiments, R¹² is independently-F. In embodiments, R^{1.2} is independently halogen. In embodiments, R¹² is independently —OCF₃. In embodiments, R^{1.2} is independently —OCF₂CF₃. In embodiments, R^{1.2} is independently unsubstituted C₁-C₄ haloalkoxy.

In embodiments, R^{1.2} is independently hydrogen, halogen, —CF₃, —CHF₂, —CH₂F, —OCF₃, —OCH₂F, —OCHF₂, —SCF₃, —SCH₂F, —SCHF₂, —CN, —SR^{1D}, —SO₂R^{1D}—SO₂NR^{1A}R^{1B}, —C(O)R^{1C}, —C(O)—OR^{1C}, —OR^{1D}, —SF₅, substituted or unsubstituted C₁-C₈ alkyl, substituted or unsubstituted 2 to 5 membered heteroalkyl, substituted or unsubstituted C₃-C₆ cycloalkyl, substituted or unsubstituted 3 to 6 membered heterocycloalkyl, substituted or unsubstituted aryl, or substituted or unsubstituted 5 to 6 membered heteroaryl. In embodiments, R^{1.2} is independently —CF₃, —CHF₂, or —CH₂F. In embodiments, R^{1.2} is independently —OCF₃, —OCH₂F, or —OCHF₂. In embodiments, R^{1.2} is independently hydrogen. In embodiments, R^{1.2} is independently-Br. In embodiments, R^{1.2} is independently —Cl. In embodiments, R¹² is independently-F. In embodiments, R^{1.2} is independently halogen. In embodiments, R^{1.2} is independently —OCF₃. In embodiments, R^{1.2} is independently —OCF₂CF₃. In embodiments, R^{1.2} is independently unsubstituted C₁-C₄ haloalkoxy.

In embodiments, R^{1.3} is independently hydrogen, halogen, —CF₃, —CHF₂, —CH₂F, —OCF₃, —OCH₂F, —OCHF₂, —SCF₃, —SCH₂F, —SCHF₂, —CN, —SR^{1D}, —SO₂R^{1D}—SO₂NR^{1A}R^{1B}, —C(O)R^{1C}, —C(O)—OR^{1C}, —OR^{1D}, —SF₅, substituted or unsubstituted C₁-C₈ alkyl, substituted or unsubstituted 2 to 5 membered heteroalkyl, substituted or unsubstituted C₃-C₆ cycloalkyl, substituted or unsubstituted 3 to 6 membered heterocycloalkyl, substituted or unsubstituted aryl, or substituted or unsubstituted 5 to 6 membered heteroaryl. In embodiments, R^{1.3} is independently —CF₃, —CHF₂, or —CH₂F. In embodiments, R^{1.3} is independently —OCF₃, —OCH₂F, or —OCHF₂. In embodiments, R^{1.3} is independently hydrogen. In embodiments, R^{1.3} is independently-Br. In embodiments, R^{1.3} is independently —Cl. In embodiments, R¹³ is independently-F. In embodiments, R^{1.3} is independently halogen. In embodiments, R^{1.3} is independently —OCF₃. In embodiments, R^{1.3} is independently —OCF₂CF₃. In embodiments, R^{1.3} is independently unsubstituted C₁-C₄ haloalkoxy.

In embodiments, R^{1.4} is independently hydrogen,

halogen, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, unsubstituted methoxy, unsubstituted ethoxy, unsubstituted n-propoxy, unsubstituted isopropoxy, unsubstituted n-butoxy, unsubstituted t-butoxy, unsubstituted sec-butoxy, or unsubstituted isobutoxy.

In embodiments, R^{1.4} is independently hydrogen, halogen, —CF₃, —CHF₂, —CH₂F, —OCF₃, —OCH₂F, —OCHF₂, —SCF₃, —SCH₂F, —SCHF₂, substituted or unsubstituted C₁-C₈ alkyl, or substituted or unsubstituted 2 to 5 membered heteroalkyl. In embodiments, R^{1.4} is hydrogen or halogen. In embodiments, R^{1.4} is independently —CF₃, —CHF₂, or —CH₂F. In embodiments, R^{1.4} is independently —OCF₃, —OCH₂F, or —OCHF₂. In embodiments, R^{1.4} is independently hydrogen. In embodiments, R^{1.4} is independently-Br. In embodiments, R^{1.4} is independently —Cl. In embodiments, R^{1.4} is independently-F. In embodiments, R^{1.4} is independently halogen. In embodiments, R^{1.4} is independently —OCF₃. In embodiments, R^{1.4} is independently —OCF₂CF₃. In embodiments, R^{1.4} is independently unsubstituted C₁-C₄ haloalkoxy.

In embodiments, R^{1.5} is independently hydrogen,

halogen, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, unsubstituted methoxy, unsubstituted ethoxy, unsubstituted n-propoxy, unsubstituted isopropoxy, unsubstituted n-butoxy, unsubstituted t-butoxy, unsubstituted sec-butoxy, or unsubstituted isobutoxy.

In embodiments, R^{1.5} is independently hydrogen, halogen, —CF₃, —CHF₂, —CH₂F, —OCF₃, —OCH₂F, —OCHF₂, —SCF₃, —SCH₂F, —SCHF₂, —CN, —SR^{1D}, —SO₂R^{1D}—SO₂NR^{1A}R^{1B}, —C(O)R^{1C}, —C(O)—OR^{1C}, —OR^{1D}, —SF₅, substituted or unsubstituted C₁-C₈ alkyl, substituted or unsubstituted 2 to 5 membered heteroalkyl, substituted or unsubstituted C₃-C₆ cycloalkyl, substituted or unsubstituted 3 to 6 membered heterocycloalkyl, substituted or unsubstituted aryl, or substituted or unsubstituted 5 to 6 membered heteroaryl. In embodiments, R^{1.5} is independently —CF₃, —CHF₂, or —CH₂F. In embodiments, R¹⁵ is independently —OCF₃, —OCH₂F, or —OCHF₂. In embodiments, R^{1.5} is independently hydrogen. In embodiments, R^{1.5} is independently-Br. In embodiments, R^{1.5} is independently —Cl. In embodiments, R^{1.5} is independently-F. In embodiments, R^{1.5} is independently halogen. In embodiments, R^{1.5} is independently —OCF₃. In embodiments, R^{1.5} is independently —OCF₂CF₃. In embodiments, R^{1.5} is independently unsubstituted C₁-C₄ haloalkoxy.

In embodiments, R¹ and R^{1.2} are independently hydrogen, halogen, —CF₃, —CHF₂, —CH₂F, —OCF₃, —OCH₂F, —OCHF₂, —SCF₃, —SCH₂F, —SCHF₂, or unsubstituted C₁-C₈ alkyl. In embodiments, R^{1.1} is independently hydrogen, halogen, or —OCF₃; and R^{1.2} is independently hydrogen, halogen, or —OCF₃. In embodiments, R^{1.1} is independently hydrogen, —F, or —OCF₃; and R^{1.2} is independently hydrogen, —F, or —OCF₃. In embodiments, R^{1.1} is hydrogen and R¹² is hydrogen, halogen, or —OCF₃. In embodiments, R^{1.1} is hydrogen and R¹² is —OCF₃. In embodiments, R^{1.1} is independently —OCF₃; and R¹² is independently hydrogen, halogen, or —OCF₃. In embodiments, R^{1.1} is independently —OCF₃ and R^{1.2} is independently hydrogen. In embodiments, R^{1.1} is independently —OCF₃; and R¹² is independently halogen. In embodiments, R^{1.1} is independently —OCF₃; and R^{1.2} is independently-F. In embodiments, R^{1.1} is independently —OCF₃; and R^{1.2} is independently —OCF₃.

In embodiments, two adjacent R¹ substituents are joined to form a substituted or unsubstituted 5 to 6 membered heterocycloalkyl.

In embodiments, R¹ is independently halogen, —CF₃, —CHF₂, —CH₂F, —OCF₃, —OCH₂F, —OCHF₂, —SCF₃, —SCH₂F, —SCHF₂, —CN, —SR^{1D}, —SO₂R^{1D}—SO₂NR^{1A}R^{1B}, —NHC(O)NR^{1A}R^{1B}, —N(O)₂, —NR^{1A}R^{1B}, —C(O)R^{1C}, —C(O)—OR^{1C}, —C(O)NR^{1A}R^{1B}, —OR^{1D}, —NR^{1A}SO ₂R^{1D}, —NR^{1A}C(O)R^{1C}, —NR^{1A}C(O)OR^{1C}, —NR^{1A}OR^{1C}, —SF₅, substituted or unsubstituted C₁-C₈ alkyl, substituted or unsubstituted 2 to 5 membered heteroalkyl, substituted or unsubstituted C₃-C₆ cycloalkyl, substituted or unsubstituted 3 to 6 membered heterocycloalkyl, substituted or unsubstituted aryl, or substituted or unsubstituted 5 to 6 membered heteroaryl; two adjacent R¹ substituents may optionally be joined to form a substituted or unsubstituted 5 to 6 membered heterocycloalkyl.

In embodiments, R¹ is independently —OH, —OCH₃, —OCH₂CH₃, —OCH₂CH₂CH₃, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —F, —Cl, —Br, —I, —CF₃, —CCl₃, —CBr₃, —Cl₃, —OCH₂CF₃, —OCH₂CCl₃, —OCH₂CBr₃, —OCH₂Cl₃, —SF₅, —CF₂CF₃, —SCH₃, —SCH₂CH₃, —SCF₃, —SCCl₃, —SCBr₃, —SCI₃, —SOCH₃, —SO₂CH₃, —SOCF₃, —SO₂CF₃, —CH₂CN, —CN,

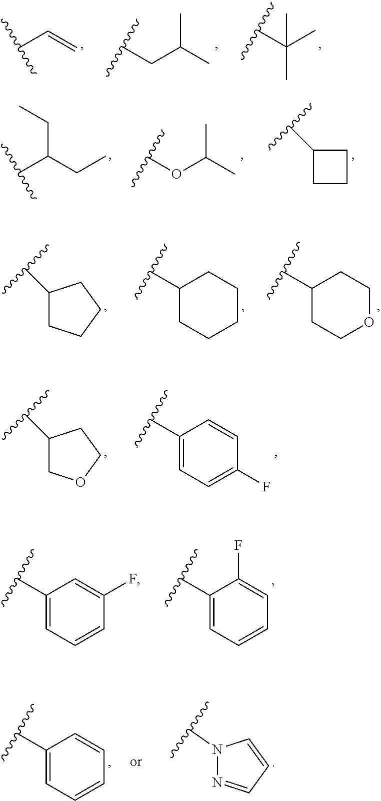

In embodiments, R¹ is independently —OH, —CH₃, —OCH₃, —OCH₂CH₃, —OCH₂CH₂CH₃, —CH₂OH, —F, —Cl, —Br, —I, —OCF₃, —OCHF₂, —OCH₂CF₃, —SF₅, —CF₂CF₃, —SCH₃, —SCF₃, —SOCH₃, —SO₂CH₃, —SOCF₃, —SO₂CF₃, —CH₂CN, —CN, —CF₃,

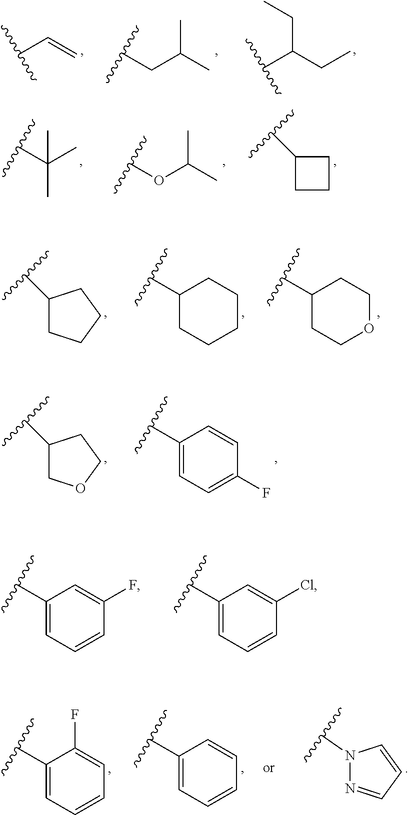

In embodiments, R¹ is independently-OH. In embodiments, R¹ is independently —OCH₃. In embodiments, R¹ is independently —OCH₂CH₃. In embodiments, R¹ is independently —OCH₂CH₂CH₃. In embodiments, R¹ is independently —OCCl₃. In embodiments, R¹ is independently —OCF₃. In embodiments, R¹ is independently —OCBr₃. In embodiments, R¹ is independently —OCl₃. In embodiments, R¹ is independently —OCHCl₂. In embodiments, R¹ is independently —OCHBr₂. In embodiments, R¹ is independently —OCHI₂. In embodiments, R¹ is independently —OCHF₂. In embodiments, R¹ is independently —OCH₂Cl. In embodiments, R¹ is independently —OCH₂Br. In embodiments, R¹ is independently —OCH₂I. In embodiments, R¹ is independently —OCH₂F. In embodiments, R¹ is independently-F. In embodiments, R¹ is independently —Cl. In embodiments, R¹ is independently-Br. In embodiments, R¹ is independently —I. In embodiments, R¹ is independently —CF₃. In embodiments, R¹ is independently —CCl₃. In embodiments, R¹ is independently —CBr₃. In embodiments, R¹ is independently —Cl₃. In embodiments, R¹ is independently —OCH₂CF₃. In embodiments, R¹ is independently —OCH₂CCl₃. In embodiments, R¹ is independently —OCH₂CBr₃. In embodiments, R¹ is independently —OCH₂Cl₃. In embodiments, R¹ is independently —SF₅. In embodiments, R¹ is independently —CF₂CF₃. In embodiments, R¹ is independently —SCH₃. In embodiments, R¹ is independently —SCH₂CH₃. In embodiments, R¹ is independently —SCF₃. In embodiments, R¹ is independently —SCCl₃. In embodiments, R¹ is independently —SCBr₃. In embodiments, R¹ is independently —SCl₃. In embodiments, R¹ is independently —SOCH₃. In embodiments, R¹ is independently —SO₂CH₃. In embodiments, R¹ is independently —SOCF₃. In embodiments, R¹ is independently —SO₂CF₃. In embodiments, R¹ is independently —CH₂CN. In embodiments, R¹ is independently —CN. In embodiments, R¹ is independently

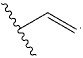 [SMILES: *C=C]

In embodiments, R¹ is independently

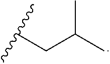 [SMILES: *CC(C)C]

In embodiments, R¹ is independently

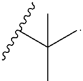 [SMILES: *C(C)(C)C]

In embodiments, R¹ is independently

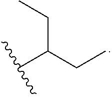 [SMILES: *C(CC)CC]

In embodiments, R¹ is independently

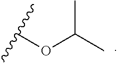 [SMILES: *OC(C)C]

In embodiments, R¹ is independently

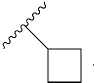 [SMILES: *C1CCC1]

In embodiments, R¹ is independently

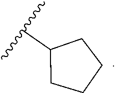 [SMILES: *C1CCCC1]

In embodiments, R¹ is independently

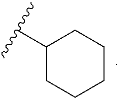 [SMILES: *C1CCCCC1]

In embodiments, R¹ is independently

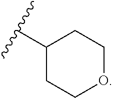 [SMILES: *C1CCOCC1]

In embodiments, R¹ is independently

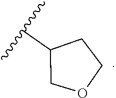 [SMILES: *C1CCOC1]

In embodiments, R¹ is independently

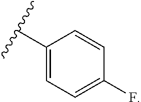 [SMILES: *c1ccc(F)cc1]

In embodiments, R¹ is independently

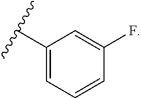 [SMILES: *c1cccc(F)c1]

In embodiments, R¹ is independently

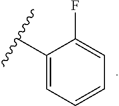 [SMILES: *c1ccccc1F]

In embodiments, R¹ is independently

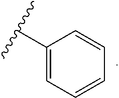 [SMILES: *c1ccccc1]

In embodiments, R¹ is independently

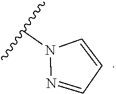 [SMILES: *n1cccn1]

In embodiments, R^{1.1} is independently-OH. In embodiments, R^{1.1} is independently —OCH₃. In embodiments, R^{1.1} is independently —OCH₂CH₃. In embodiments, R^{1.1} is independently —OCH₂CH₂CH₃. In embodiments, R^{1.1} is independently —OCCl₃. In embodiments, R^{1.1} is independently —OCF₃. In embodiments, R^{1.1} is independently —OCBr₃. In embodiments, R^{1.1} is independently —OCl₃. In embodiments, R^{1.1} is independently —OCHCl₂. In embodiments, R^{1.1} is independently —OCHBr₂. In embodiments, R^{1.1} is independently —OCHI₂. In embodiments, R^{1.1} is independently —OCHF₂. In embodiments, R^{1.1} is independently —OCH₂Cl. In embodiments, R^{1.1} is independently —OCH₂Br. In embodiments, R^{1.1} is independently —OCH₂I. In embodiments, R^{1.1} is independently —OCH₂F. In embodiments, R^{1.1} is independently-F. In embodiments, R^{1.1} is independently —Cl. In embodiments, R^{1.1} is independently-Br. In embodiments, R^{1.1} is independently —I. In embodiments, R^{1.1} is independently —CF₃. In embodiments, R^{1.1} is independently —CCl₃. In embodiments, R^{1.1} is independently —CBr₃. In embodiments, R^{1.1} is independently —Cl₃. In embodiments, R^{1.1} is independently —OCH₂CF₃. In embodiments, R^{1.1} is independently —OCH₂CCl₃. In embodiments, R^{1.1} is independently —OCH₂CBr₃. In embodiments, R^{1.1} is independently —OCH₂Cl₃. In embodiments, R^{1.1} is independently —SF₅. In embodiments, R^{1.1} is independently —CF₂CF₃. In embodiments, R^{1.1} is independently —SCH₃. In embodiments, R^{1.1} is independently —SCH₂CH₃. In embodiments, R^{1.1} is independently —SCF₃. In embodiments, R^{1.1} is independently —SCCl₃. In embodiments, R^{1.1} is independently —SCBr₃. In embodiments, R^{1.1} is independently —SCl₃. In embodiments, R^{1.1} is independently —SOCH₃. In embodiments, R^{1.1} is independently —SO₂CH₃. In embodiments, R^{1.1} is independently —SOCF₃. In embodiments, R^{1.1} is independently —SO₂CF₃. In embodiments, R^{1.1} is independently —CH₂CN. In embodiments, R¹ is independently —CN. In embodiments, R^{1.1} is independently

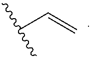 [SMILES: *C=C]

In embodiments, R^{1.1} is independently

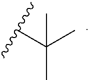 [SMILES: *C(C)(C)C]

_In embodiments, R^{1.1} is independently

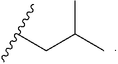 [SMILES: *CC(C)C]

In embodiments, R¹ is independently

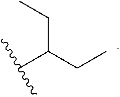 [SMILES: *C(CC)CC]

In embodiments, R^{1.1} is independently

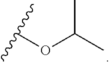 [SMILES: *OC(C)C]

In embodiments, R^{1.1} is independently

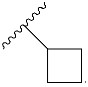 [SMILES: *C1CCC1]

In embodiments, R^{1.1} is independently

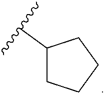 [SMILES: *C1CCCC1]

In embodiments, R^{1.1} is independently

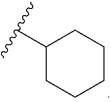 [SMILES: *C1CCCCC1]

In embodiments, R^{1.1} is independently

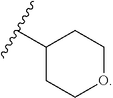 [SMILES: *C1CCOCC1]

In embodiments, R^{1.1} is independently

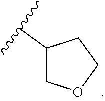 [SMILES: *C1CCOC1]

In embodiments, R^{1.1} is independently

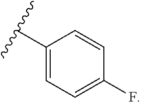 [SMILES: *c1ccc(F)cc1]

In embodiments, R^{1.1} is independently

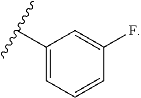 [SMILES: *c1cccc(F)c1]

In embodiments, R^{1.1} is independently

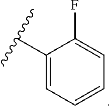 [SMILES: *c1ccccc1F]

In embodiments, R^{1.1} is independently

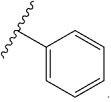 [SMILES: *c1ccccc1]

In embodiments, R^{1.1} is independently

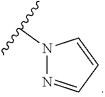 [SMILES: *n1cccn1]

In embodiments, R^{1.2} is independently-OH. In embodiments, R^{1.2} is independently —OCH₃. In embodiments, R^{1.2} is independently —OCH₂CH₃. In embodiments, R^{1.2} is independently —OCH₂CH₂CH₃. In embodiments, R^{1.2} is independently —OCCl₃. In embodiments, R^{1.2} is independently —OCF₃. In embodiments, R^{1.2} is independently —OCBr₃. In embodiments, R^{1.2} is independently —OCl₃. In embodiments, R^{1.2} is independently —OCHCl₂. In embodiments, R^{1.2} is independently —OCHBr₂. In embodiments, R^{1.2} is independently —OCHI₂. In embodiments, R^{1.2} is independently —OCHF₂. In embodiments, R^{1.2} is independently —OCH₂Cl. In embodiments, R^{1.2} is independently —OCH₂Br. In embodiments, R^{1.2} is independently —OCH₂I. In embodiments, R^{1.2} is independently —OCH₂F. In embodiments, R^{1.2} is independently-F. In embodiments, R^{1.2} is independently —Cl. In embodiments, R^{1.2} is independently-Br. In embodiments, R^{1.2} is independently —I. In embodiments, R^{1.2} is independently —CF₃. In embodiments, R^{1.2} is independently —CCl₃. In embodiments, R^{1.2} is independently —CBr₃. In embodiments, R^{1.2} is independently —Cl₃. In embodiments, R^{1.2} is independently —OCH₂CF₃. In embodiments, R^{1.2} is independently —OCH₂CCl₃. In embodiments, R^{1.2} is independently —OCH₂CBr₃. In embodiments, R^{1.2} is independently —OCH₂Cl₃. In embodiments, R^{1.2} is independently —SF₅. In embodiments, R^{1.2} is independently —CF₂CF₃. In embodiments, R^{1.2} is independently —SCH₃. In embodiments, R^{1.2} is independently —SCH₂CH₃. In embodiments, R^{1.2} is independently —SCF₃. In embodiments, R^{1.2} is independently —SCCl₃. In embodiments, R^{1.2} is independently —SCBr₃. In embodiments, R^{1.2} is independently —SCl₃. In embodiments, R^{1.2} is independently —SOCH₃. In embodiments, R^{1.2} is independently —SO₂CH₃. In embodiments, R^{1.2} is independently —SOCF₃. In embodiments, R^{1.2} is independently —SO₂CF₃. In embodiments, R^{1.2} is independently —CH₂CN. In embodiments, R is independently —CN. In embodiments, R^{1.2} is independently

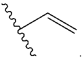 [SMILES: *C=C]

In embodiments, R^{1.2} is independently

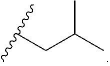 [SMILES: *CC(C)C]

In embodiments, R^{1.2} is independently

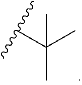 [SMILES: *C(C)(C)C]

In embodiments, R^{1.2} is independently

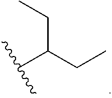 [SMILES: *C(CC)CC]

In embodiments, R^{1.2} is independently

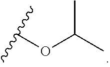 [SMILES: *OC(C)C]

In embodiments, R^{1.2} is independently

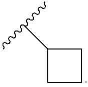 [SMILES: *C1CCC1]

In embodiments, R^{1.2} is independently

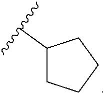 [SMILES: *C1CCCC1]

In embodiments, R^{1.2} is independently

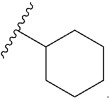 [SMILES: *C1CCCCC1]

In embodiments, R^{1.2} is independently

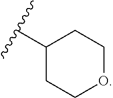 [SMILES: *C1CCOCC1]

In embodiments, R^{1.2} is independently

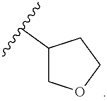 [SMILES: *C1CCOC1]

In embodiments, R^{1.2} is independently

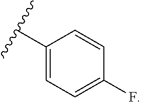 [SMILES: *c1ccc(F)cc1]

In embodiments, R^{1.2} is independently

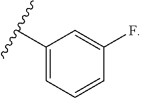 [SMILES: *c1cccc(F)c1]

In embodiments, R^{1.2} is independently

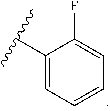 [SMILES: *c1ccccc1F]

In embodiments, R^{1.2} is independently

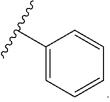 [SMILES: *c1ccccc1]

In embodiments, R^{1.2} is independently

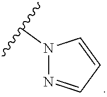 [SMILES: *n1cccn1]

In embodiments, R^{1.3} is independently-OH. In embodiments, R^{1.3} is independently —OCH₃. In embodiments, R^{1.3} is independently —OCH₂CH₃. In embodiments, R^{1.3} is independently —OCH₂CH₂CH₃. In embodiments, R^{1.3} is independently —OCCl₃. In embodiments, R^{1.3} is independently —OCF₃. In embodiments, R^{1.3} is independently —OCBr₃. In embodiments, R^{1.3} is independently —OCl₃. In embodiments, R^{1.3} is independently —OCHCl₂. In embodiments, R^{1.3} is independently —OCHBr₂. In embodiments, R^{1.3} is independently —OCHI₂. In embodiments, R^{1.3} is independently —OCHF₂. In embodiments, R^{1.3} is independently —OCH₂Cl. In embodiments, R^{1.3} is independently —OCH₂Br. In embodiments, R^{1.3} is independently —OCH₂I. In embodiments, R^{1.3} is independently —OCH₂F. In embodiments, R^{1.3} is independently-F. In embodiments, R¹³ is independently —Cl. In embodiments, R^{1.3} is independently-Br. In embodiments, R^{1.3} is independently —I. In embodiments, R^{1.3} is independently —CF₃. In embodiments, R^{1.3} is independently —CCl₃. In embodiments, R^{1.3} is independently —CBr₃. In embodiments, R^{1.3} is independently —Cl₃. In embodiments, R^{1.3} is independently —OCH₂CF₃. In embodiments, R^{1.3} is independently —OCH₂CCl₃. In embodiments, R^{1.3} is independently —OCH₂CBr₃. In embodiments, R^{1.3} is independently —OCH₂Cl₃. In embodiments, R^{1.3} is independently —SF₅. In embodiments, R^{1.3} is independently —CF₂CF₃. In embodiments, R^{1.3} is independently —SCH₃. In embodiments, R^{1.3} is independently —SCH₂CH₃. In embodiments, R^{1.3} is independently —SCF₃. In embodiments, R^{1.3} is independently —SCCl₃. In embodiments, R^{1.3} is independently —SCBr₃. In embodiments, R^{1.3} is independently —SCl₃. In embodiments, R^{1.3} is independently —SOCH₃. In embodiments, R^{1.3} is independently —SO₂CH₃. In embodiments, R^{1.3} is independently —SOCF₃. In embodiments, R^{1.3} is independently —SO₂CF₃. In embodiments, R^{1.3} is independently —CH₂CN. In embodiments, R^{1.3} is independently —CN. In embodiments, R^{1.3} is independently

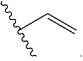 [SMILES: *C=C]

In embodiments, R^{1.3} is independently

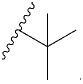 [SMILES: *C(C)(C)C]

In embodiments, R^{1.3} is independently

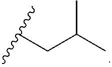 [SMILES: *CC(C)C]

In embodiments, R^{1.3} is independently

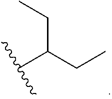 [SMILES: *C(CC)CC]

In embodiments, R^{1.3} is independently

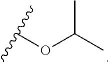 [SMILES: *OC(C)C]

In embodiments, R^{1.3} is independently

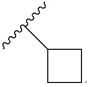 [SMILES: *C1CCC1]

In embodiments, R^{1.3} is independently

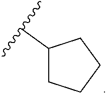 [SMILES: *C1CCCC1]

In embodiments, R^{1.3} is independently

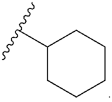 [SMILES: *C1CCCCC1]

In embodiments, R^{1.3} is independently

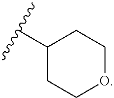 [SMILES: *C1CCOCC1]

In embodiments, R^{1.3} is independently

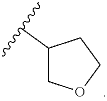 [SMILES: *C1CCOC1]

In embodiments, R^{1.3} is independently

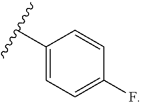 [SMILES: *c1ccc(F)cc1]

In embodiments, R^{1.3} is independently

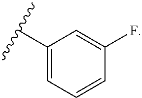 [SMILES: *c1cccc(F)c1]

In embodiments, R^{1.3} is independently

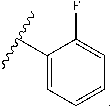 [SMILES: *c1ccccc1F]

In embodiments, R^{1.3} is independently

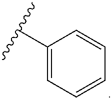 [SMILES: *c1ccccc1]

In embodiments, R^{1.3} is independently

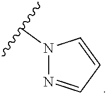 [SMILES: *n1cccn1]

In embodiments, R^{1.4} is independently-OH. In embodiments, R^{1.4} is independently —OCH₃. In embodiments, R^{1.4} is independently —OCH₂CH₃. In embodiments, R^{1.4} is independently —OCH₂CH₂CH₃. In embodiments, R^{1.4} is independently —OCCl₃. In embodiments, R^{1.4} is independently —OCF₃. In embodiments, R^{1.4} is independently —OCBr₃. In embodiments, R^{1.4} is independently —OCl₃. In embodiments, R^{1.4} is independently —OCHCl₂. In embodiments, R^{1.4} is independently —OCHBr₂. In embodiments, R^{1.4} is independently —OCHI₂. In embodiments, R^{1.4} is independently —OCHF₂. In embodiments, R^{1.4} is independently —OCH₂Cl. In embodiments, R^{1.4} is independently —OCH₂Br. In embodiments, R^{1.4} is independently —OCH₂I. In embodiments, R^{1.4} is independently —OCH₂F. In embodiments, R^{1.4} is independently-F. In embodiments, R^{1.4} is independently —Cl. In embodiments, R^{1.4} is independently-Br. In embodiments, R^{1.4} is independently —I. In embodiments, R^{1.4} is independently —CF₃. In embodiments, R^{1.4} is independently —CCl₃. In embodiments, R^{1.4} is independently —CBr₃. In embodiments, R^{1.4} is independently —Cl₃. In embodiments, R^{1.4} is independently —OCH₂CF₃. In embodiments, R^{1.4} is independently —OCH₂CCl₃. In embodiments, R^{1.4} is independently —OCH₂CBr₃. In embodiments, R^{1.4} is independently —OCH₂Cl₃. In embodiments, R^{1.4} is independently —SF₅. In embodiments, R^{1.4} is independently —CF₂CF₃. In embodiments, R^{1.4} is independently —SCH₃. In embodiments, R^{1.4} is independently —SCH₂CH₃. In embodiments, R^{1.4} is independently —SCF₃. In embodiments, R^{1.4} is independently —SCCl₃. In embodiments, R^{1.4} is independently —SCBr₃. In embodiments, R^{1.4} is independently —SCl₃. In embodiments, R^{1.4} is independently —SOCH₃. In embodiments, R^{1.4} is independently —SO₂CH₃. In embodiments, R^{1.4} is independently —SOCF₃. In embodiments, R^{1.4} is independently —SO₂CF₃. In embodiments, R^{1.4} is independently —CH₂CN. In embodiments, R^{1.4} is independently —CN. In embodiments, R^{1.4} is independently

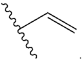 [SMILES: *C=C]

In embodiments, R^{1.4} is independently

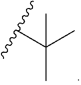 [SMILES: *C(C)(C)C]

In embodiments, R^{1.4} is independently

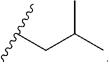 [SMILES: *CC(C)C]

In embodiments, R^{1.4} is independently

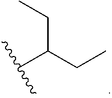 [SMILES: *C(CC)CC]

In embodiments, R^{1.4} is independently

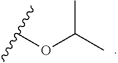 [SMILES: *OC(C)C]

In embodiments, R^{1.4} is independently

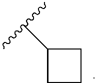 [SMILES: *C1CCC1]

In embodiments, R^{1.4} is independently

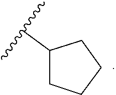 [SMILES: *C1CCCC1]

In embodiments, R¹⁴ is independently

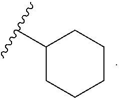 [SMILES: *C1CCCCC1]

In embodiments, R^{1.4} is independently

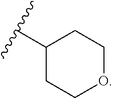 [SMILES: *C1CCOCC1]

In embodiments, R^{1.4} is independently

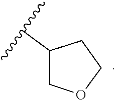 [SMILES: *C1CCOC1]

In embodiments, R^{1.4} is independently

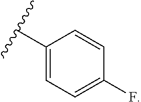 [SMILES: *c1ccc(F)cc1]

In embodiments, R^{1.4} is independently

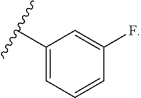 [SMILES: *c1cccc(F)c1]

In embodiments, R^{1.4} is independently

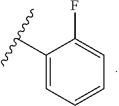 [SMILES: *c1ccccc1F]

In embodiments, R^{1.4} is independently

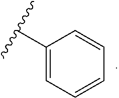 [SMILES: *c1ccccc1]

In embodiments, R^{1.4} is independently

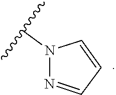 [SMILES: *n1cccn1]

In embodiments, R^{1.5} is independently-OH. In embodiments, R^{1.5} is independently —OCH₃. In embodiments, R¹⁵ is independently —OCH₂CH₃. In embodiments, R^{1.5} is independently —OCH₂CH₂CH₃. In embodiments, R^{1.5} is independently —OCCl₃. In embodiments, R^{1.5} is independently —OCF₃. In embodiments, R^{1.5} is independently —OCBr₃. In embodiments, R^{1.5} is independently —OCl₃. In embodiments, R^{1.5} is independently —OCHCl₂. In embodiments, R^{1.5} is independently —OCHBr₂. In embodiments, R^{1.5} is independently —OCHI₂. In embodiments, R^{1.5} is independently —OCHF₂. In embodiments, R^{1.5} is independently —OCH₂Cl. In embodiments, R^{1.5} is independently —OCH₂Br. In embodiments, R^{1.5} is independently —OCH₂I. In embodiments, R^{1.5} is independently —OCH₂F. In embodiments, R^{1.5} is independently-F. In embodiments, R^{1.5} is independently —Cl. In embodiments, R^{1.5} is independently-Br. In embodiments, R^{1.5} is independently —I. In embodiments, R^{1.5} is independently —CF₃. In embodiments, R^{1.5} is independently —CCl₃. In embodiments, R^{1.5} is independently —CBr₃. In embodiments, R^{1.5} is independently —Cl₃. In embodiments, R^{1.5} is independently —OCH₂CF₃. In embodiments, R^{1.5} is independently —OCH₂CCl₃. In embodiments, R^{1.5} is independently —OCH₂CBr₃. In embodiments, R^{1.5} is independently —OCH₂Cl₃. In embodiments, R^{1.5} is independently —SF₅. In embodiments, R^{1.5} is independently —CF₂CF₃. In embodiments, R^{1.5} is independently —SCH₃. In embodiments, R^{1.5} is independently —SCH₂CH₃. In embodiments, R^{1.5} is independently —SCF₃. In embodiments, R^{1.5} is independently —SCCl₃. In embodiments, R^{1.5} is independently —SCBr₃. In embodiments, R^{1.5} is independently —SCl₃. In embodiments, R^{1.5} is independently —SOCH₃. In embodiments, R^{1.5} is independently —SO₂CH₃. In embodiments, R^{1.5} is independently —SOCF₃. In embodiments, R^{1.5} is independently —SO₂CF₃. In embodiments, R^{1.5} is independently —CH₂CN. In embodiments, R^{1.5} is independently —CN. In embodiments, R^{1.5} is independently

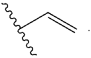 [SMILES: *C=C]

In embodiments, R⁵ is independently

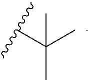 [SMILES: *C(C)(C)C]

In embodiments, R^{1.5} is independently

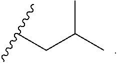 [SMILES: *CC(C)C]

In embodiments, R^{1.5} is independently

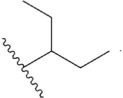 [SMILES: *C(CC)CC]

In embodiments, R^{1.5} is independently

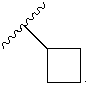 [SMILES: *C1CCC1]

In embodiments, R^{1.5} is independently

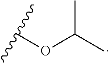 [SMILES: *OC(C)C]

In embodiments, R^{1.5} is independently

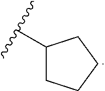 [SMILES: *C1CCCC1]

In embodiments, R^{1.5} is independently

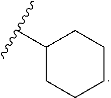 [SMILES: *C1CCCCC1]

In embodiments, R^{1.5} is independently

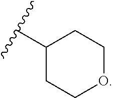 [SMILES: *C1CCOCC1]

In embodiments, R^{1.5} is independently

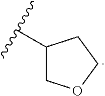 [SMILES: *C1CCOC1]

In embodiments, R^{1.5} is independently

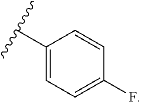 [SMILES: *c1ccc(F)cc1]

In embodiments, R^{1.5} is independently

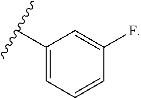 [SMILES: *c1cccc(F)c1]

In embodiments, R^{1.5} is independently

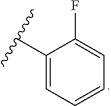 [SMILES: *c1ccccc1F]

In embodiments, R^{1.5} is independently

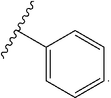 [SMILES: *c1ccccc1]

In embodiments, R^{1.5} is independently

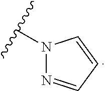 [SMILES: *n1cccn1]

In embodiments, R² is independently oxo, halogen, —CX²³, —CHX²², —CH₂X², —OCX²³, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR²CNR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O) NR^{2A}R^{2B}, —OR^{2D}, —NR^{2A}SO₂R^{2D}, —NR^{2A}C(O)R^{2C}, —NR^{2A}C(O)OR²C, —NR^{2A}OR^{2C}, —SF₅, —N₃, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R² is independently, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR²CNR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O) NR^{2A}R^{2B}, —OR^{2D}, —NR^{2A}SO₂R^{2D}, —NR^{2A}C(O)R^{2C}, —NR^{2A}C(O)OR²C, —NR^{2A}OR²C, —SF₅, —N₃, R²⁰-substituted or unsubstituted alkyl (e.g., C₁-C₈ alkyl, C₁-C₆ alkyl, or C₁-C₄ alkyl), R²⁰-substituted or unsubstituted heteroalkyl (e.g., 2 to 8 membered heteroalkyl, 2 to 6 membered heteroalkyl, or 2 to 4 membered heteroalkyl), R²⁰-substituted or unsubstituted cycloalkyl (e.g., C₃-C₈ cycloalkyl, C₃-C₆ cycloalkyl, or C₅-C₆ cycloalkyl), R²⁰-substituted or unsubstituted heterocycloalkyl (e.g., 3 to 8 membered heterocycloalkyl, 3 to 6 membered heterocycloalkyl, or 5 to 6 membered heterocycloalkyl), R²⁰-substituted or unsubstituted aryl (e.g., C₆-C₁₂ aryl, C₆-C₁₀ aryl, C₁₀ aryl, or phenyl), or R²⁰-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R² is independently R²⁰-substituted or unsubstituted alkyl (e.g., C₁-C₈ alkyl, C₁-C₆ alkyl, or C₁-C₄ alkyl), R²⁰-substituted or unsubstituted heteroalkyl (e.g., 2 to 8 membered heteroalkyl, 2 to 6 membered heteroalkyl, or 2 to 4 membered heteroalkyl), R²⁰-substituted or unsubstituted cycloalkyl (e.g., C₃-C₈ cycloalkyl, C₃-C₆ cycloalkyl, or C₅-C₆ cycloalkyl), R²⁰-substituted or unsubstituted heterocycloalkyl (e.g., 3 to 8 membered heterocycloalkyl, 3 to 6 membered heterocycloalkyl, or 5 to 6 membered heterocycloalkyl), R²⁰-substituted or unsubstituted aryl (e.g., C₆-C₁₂ aryl, C₆-C₁₀ aryl, C₁₀ aryl, or phenyl), or R²⁰-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR²CNR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O) NR^{2A}R^{2B}, —OR^{2D}, —NR^{2A}SO₂R^{2D}, —NR^{2A}C(O)R^{2C}, —NR^{2A}C(O)OR²C, —NR^{2A}OR²C, —SF₅, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R² is independently unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or to 6 membered heteroaryl).

In embodiments, R^{2A}, R^{2B}, R^{2C}, and R^{D} are independently

hydrogen, —CX₃, —CHX₂, —CH₂X, —CN, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R^{2A}, R^{2B}, R^{2C}, and R^{2D} are independently hydrogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —CN, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2A} is independently

hydrogen, —CX₃, —CHX₂, —CH₂X, —CN, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R^{2A} is independently
hydrogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —CN, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2B} is independently

hydrogen, —CX₃, —CHX₂, —CH₂X, —CN, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R^{2B} is independently
hydrogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —CN, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2C} is independently

hydrogen, —CX₃, —CHX₂, —CH₂X, —CN, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R^{2C} is independently
hydrogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —CN, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2D} is independently

hydrogen, —CX₃, —CHX₂, —CH₂X, —CN, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R^{2D} is independently
hydrogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —CN, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₂ aryl, C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2A}, R^{2B}, R^{2C}, and R^{D} are independently

hydrogen, —CX₃, —CHX₂, —CH₂X, —CN, —COOH, —CONH₂, R²⁰-substituted or unsubstituted alkyl (e.g., C₁-C₈ alkyl, C₁-C₆ alkyl, or C₁-C₄ alkyl), R²⁰-substituted or unsubstituted heteroalkyl (e.g., 2 to 8 membered heteroalkyl, 2 to 6 membered heteroalkyl, or 2 to 4 membered heteroalkyl), R²⁰-substituted or unsubstituted cycloalkyl (e.g., C₃-C₈ cycloalkyl, C₃-C₆ cycloalkyl, or C₅-C₆ cycloalkyl), R²⁰-substituted or unsubstituted heterocycloalkyl (e.g., 3 to 8 membered heterocycloalkyl, 3 to 6 membered heterocycloalkyl, or to 6 membered heterocycloalkyl), R²⁰-substituted or unsubstituted aryl (e.g., C₆-C₁₂ aryl, C₆-C₁₀ aryl, C₁₀ aryl, or phenyl), or R²⁰-substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R^{2A}, R^{2B}, R^{2C}, and R^{2D} are independently
hydrogen, —CF₃, —CBr₃, —CCl₃, —Cl₃, —CHF₂, —CHBr₂, —CHCl₂, —CHI₂, —CH₂F, —CH₂Br, —CH₂Cl, —C H₂I, —CN, —COOH, —CONH₂, R²⁰-substituted or unsubstituted alkyl (e.g., C₁-C₈ alkyl, C₁-C₆ alkyl, or C₁-C₄ alkyl), R²⁰-substituted or unsubstituted heteroalkyl (e.g., 2 to 8 membered heteroalkyl, 2 to 6 membered heteroalkyl, or 2 to 4 membered heteroalkyl), R²⁰-substituted or unsubstituted cycloalkyl (e.g., C₃-C₈ cycloalkyl, C₃-C₆ cycloalkyl, or C₅-C₆ cycloalkyl), R²⁰-substituted or unsubstituted heterocycloalkyl (e.g., 3 to 8 membered heterocycloalkyl, 3 to 6 membered heterocycloalkyl, or 5 to 6 membered heterocycloalkyl), R²⁰-substituted or unsubstituted aryl (e.g., C₆-C₁₂ aryl, C₆-C₁₀ aryl, C₁₀ aryl, or phenyl), or R²⁰-substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R^{2A}, R^{2B}, R^{2C}, and R^{2D} are independently hydrogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —CN, —COOH, —CONH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R^{2A} and R^{2B} substituents bonded to the same nitrogen atom may optionally be joined to form an R²⁰-substituted or unsubstituted heterocycloalkyl (e.g., 3 to 8 membered heterocycloalkyl, 3 to 6 membered heterocycloalkyl, or 5 to 6 membered heterocycloalkyl) or R²⁰-substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R^{2A} and R^{2B} substituents bonded to the same nitrogen atom may optionally be joined to form an unsubstituted heterocycloalkyl (e.g., 3 to 8 membered heterocycloalkyl, 3 to 6 membered heterocycloalkyl, or to 6 membered heterocycloalkyl) or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2A} is independently hydrogen, —CX₃, —CHX₂, —CH₂X, —CN, —COOH, —CONH₂, R²⁰-substituted or unsubstituted alkyl (e.g., C₁-C₈ alkyl, C₁-C₆ alkyl, or C₁-C₄ alkyl), R²⁰-substituted or unsubstituted heteroalkyl (e.g., 2 to 8 membered heteroalkyl, 2 to 6 membered heteroalkyl, or 2 to 4 membered heteroalkyl), R²⁰-substituted or unsubstituted cycloalkyl (e.g., C₃-C₈ cycloalkyl, C₃-C₆ cycloalkyl, or C₅-C₆ cycloalkyl), R²⁰-substituted or unsubstituted heterocycloalkyl (e.g., 3 to 8 membered heterocycloalkyl, 3 to 6 membered heterocycloalkyl, or to 6 membered heterocycloalkyl), R²⁰-substituted or unsubstituted aryl (e.g., C₆-C₁₂ aryl, C₆-C₁₀ aryl, C₁₀ aryl, or phenyl), or R²⁰-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2A} is independently

hydrogen, —CF₃, —CBr₃, —CCl₃, —Cl₃, —CHF₂, —CHBr₂, —CHCl₂, —CHI₂, —CH₂F, —CH₂Br, —CH₂Cl, —C H₂I, —CN, —COOH, —CONH₂, R²⁰-substituted or unsubstituted alkyl (e.g., C₁-C₈ alkyl, C₁-C₆ alkyl, or C₁-C₄ alkyl), R²⁰-substituted or unsubstituted heteroalkyl (e.g., 2 to 8 membered heteroalkyl, 2 to 6 membered heteroalkyl, or 2 to 4 membered heteroalkyl), R²⁰-substituted or unsubstituted cycloalkyl (e.g., C₃-C₈ cycloalkyl, C₃-C₆ cycloalkyl, or C₅-C₆ cycloalkyl), R²⁰-substituted or unsubstituted heterocycloalkyl (e.g., 3 to 8 membered heterocycloalkyl, 3 to 6 membered heterocycloalkyl, or 5 to 6 membered heterocycloalkyl), R²⁰-substituted or unsubstituted aryl (e.g., C₆-C₁₂ aryl, C₆-C₁₀ aryl, C₁₀ aryl, or phenyl), or R²⁰-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2A} is independently

hydrogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —CN, —COOH, —CONH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2B} is independently hydrogen, —CX₃, —CHX₂, —CH₂X, —CN, —COOH, —CONH₂, R²⁰-substituted or unsubstituted alkyl (e.g., C₁-C₈ alkyl, C₁-C₆ alkyl, or C₁-C₄ alkyl), R²⁰-substituted or unsubstituted heteroalkyl (e.g., 2 to 8 membered heteroalkyl, 2 to 6 membered heteroalkyl, or 2 to 4 membered heteroalkyl), R²⁰-substituted or unsubstituted cycloalkyl (e.g., C₃-C₈ cycloalkyl, C₃-C₆ cycloalkyl, or C₅-C₆ cycloalkyl), R²⁰-substituted or unsubstituted heterocycloalkyl (e.g., 3 to 8 membered heterocycloalkyl, 3 to 6 membered heterocycloalkyl, or to 6 membered heterocycloalkyl), R²⁰-substituted or unsubstituted aryl (e.g., C₆-C₁₂ aryl, C₆-C₁₀ aryl, C₁₀ aryl, or phenyl), or R²⁰-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2B} is independently

hydrogen, —CF₃, —CBr₃, —CCl₃, —Cl₃, —CHF₂, —CHBr₂, —CHCl₂, —CHI₂, —CH₂F, —CH₂Br, —CH₂Cl, —C H₂I, —CN, —COOH, —CONH₂, R²⁰-substituted or unsubstituted alkyl (e.g., C₁-C₈ alkyl, C₁-C₆ alkyl, or C₁-C₄ alkyl), R²⁰-substituted or unsubstituted heteroalkyl (e.g., 2 to 8 membered heteroalkyl, 2 to 6 membered heteroalkyl, or 2 to 4 membered heteroalkyl), R²⁰-substituted or unsubstituted cycloalkyl (e.g., C₃-C₈ cycloalkyl, C₃-C₆ cycloalkyl, or C₅-C₆ cycloalkyl), R²⁰-substituted or unsubstituted heterocycloalkyl (e.g., 3 to 8 membered heterocycloalkyl, 3 to 6 membered heterocycloalkyl, or 5 to 6 membered heterocycloalkyl), R²⁰-substituted or unsubstituted aryl (e.g., C₆-C₁₂ aryl, C₆-C₁₀ aryl, C₁₀ aryl, or phenyl), or R²⁰-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2B} is independently

hydrogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —CN, —COOH, —CONH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2A} and R^{2B} substituents bonded to the same nitrogen atom may optionally be joined to form an R²⁰-substituted or unsubstituted heterocycloalkyl (e.g., 3 to 8 membered heterocycloalkyl, 3 to 6 membered heterocycloalkyl, or 5 to 6 membered heterocycloalkyl) or R²⁰-substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R^{2A} and R^{2B} substituents bonded to the same nitrogen atom may optionally be joined to form an unsubstituted heterocycloalkyl (e.g., 3 to 8 membered heterocycloalkyl, 3 to 6 membered heterocycloalkyl, or 5 to 6 membered heterocycloalkyl) or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2C} is independently hydrogen, —CX₃, —CHX₂, —CH₂X, —CN, —COOH, —CONH₂, R²⁰-substituted or unsubstituted alkyl (e.g., C₁-C₈ alkyl, C₁-C₆ alkyl, or C₁-C₄ alkyl), R²⁰-substituted or unsubstituted heteroalkyl (e.g., 2 to 8 membered heteroalkyl, 2 to 6 membered heteroalkyl, or 2 to 4 membered heteroalkyl), R²⁰-substituted or unsubstituted cycloalkyl (e.g., C₃-C₈ cycloalkyl, C₃-C₆ cycloalkyl, or C₅-C₆ cycloalkyl), R²⁰-substituted or unsubstituted heterocycloalkyl (e.g., 3 to 8 membered heterocycloalkyl, 3 to 6 membered heterocycloalkyl, or to 6 membered heterocycloalkyl), R²⁰-substituted or unsubstituted aryl (e.g., C₆-C₁₂ aryl, C₆-C₁₀ aryl, C₁₀ aryl, or phenyl), or R²⁰-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2C} is independently

hydrogen, —CF₃, —CBr₃, —CCl₃, —Cl₃, —CHF₂, —CHBr₂, —CHCl₂, —CHI₂, —CH₂F, —CH₂Br, —CH₂Cl, —C H₂I, —CN, —COOH, —CONH₂, R²⁰-substituted or unsubstituted alkyl (e.g., C₁-C₈ alkyl, C₁-C₆ alkyl, or C₁-C₄ alkyl), R²⁰-substituted or unsubstituted heteroalkyl (e.g., 2 to 8 membered heteroalkyl, 2 to 6 membered heteroalkyl, or 2 to 4 membered heteroalkyl), R²⁰-substituted or unsubstituted cycloalkyl (e.g., C₃-C₈ cycloalkyl, C₃-C₆ cycloalkyl, or C₅-C₆ cycloalkyl), R²⁰-substituted or unsubstituted heterocycloalkyl (e.g., 3 to 8 membered heterocycloalkyl, 3 to 6 membered heterocycloalkyl, or 5 to 6 membered heterocycloalkyl), R²⁰-substituted or unsubstituted aryl (e.g., C₆-C₁₂ aryl, C₆-C₁₀ aryl, C₁₀ aryl, or phenyl), or R²⁰-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2C} is independently

hydrogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —CN, —COOH, —CONH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2D} is independently hydrogen, —CX₃, —CHX₂, —CH₂X, —CN, —COOH, —CONH₂, R²⁰-substituted or unsubstituted alkyl (e.g., C₁-C₈ alkyl, C₁-C₆ alkyl, or C₁-C₄ alkyl), R²⁰-substituted or unsubstituted heteroalkyl (e.g., 2 to 8 membered heteroalkyl, 2 to 6 membered heteroalkyl, or 2 to 4 membered heteroalkyl), R²⁰-substituted or unsubstituted cycloalkyl (e.g., C₃-C₈ cycloalkyl, C₃-C₆ cycloalkyl, or C₅-C₆ cycloalkyl), R²⁰-substituted or unsubstituted heterocycloalkyl (e.g., 3 to 8 membered heterocycloalkyl, 3 to 6 membered heterocycloalkyl, or to 6 membered heterocycloalkyl), R²⁰-substituted or unsubstituted aryl (e.g., C₆-C₁₂ aryl, C₆-C₁₀ aryl, C₁₀ aryl, or phenyl), or R²⁰-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2D} is independently

hydrogen, —CF₃, —CBr₃, —CCl₃, —Cl₃, —CHF₂, —CHBr₂, —CHCl₂, —CHI₂, —CH₂F, —CH₂Br, —CH₂Cl, —C H₂I, —CN, —COOH, —CONH₂, R²⁰-substituted or unsubstituted alkyl (e.g., C₁-C₈ alkyl, C₁-C₆ alkyl, or C₁-C₄ alkyl), R²⁰-substituted or unsubstituted heteroalkyl (e.g., 2 to 8 membered heteroalkyl, 2 to 6 membered heteroalkyl, or 2 to 4 membered heteroalkyl), R²⁰-substituted or unsubstituted cycloalkyl (e.g., C₃-C₈ cycloalkyl, C₃-C₆ cycloalkyl, or C₅-C₆ cycloalkyl), R²⁰-substituted or unsubstituted heterocycloalkyl (e.g., 3 to 8 membered heterocycloalkyl, 3 to 6 membered heterocycloalkyl, or 5 to 6 membered heterocycloalkyl), R²⁰-substituted or unsubstituted aryl (e.g., C₆-C₁₂ aryl, C₆-C₁₀ aryl, C₁₀ aryl, or phenyl), or R²⁰-substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R^{2′} is independently

hydrogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —CN, —COOH, —CONH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₂ aryl, C₆-C₁₀ aryl, C₁₀ aryl, or phenyl), or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R² is independently substituted or unsubstituted C₁-C₈ alkyl, substituted or unsubstituted 2 to 5 membered heteroalkyl, substituted or unsubstituted phenyl, or substituted or unsubstituted 5 to 6 membered heteroaryl.

In embodiments, R² is independently substituted or unsubstituted C₁-C₈ alkyl, substituted or unsubstituted 2 to 5 membered heteroalkyl. In embodiments, R² is independently substituted or unsubstituted C₁-C₈ alkyl or substituted or unsubstituted 2 to 5 membered heteroalkyl. In embodiments, R² is independently substituted C₁-C₈ alkyl or substituted 2 to 5 membered heteroalkyl. In embodiments, R² is independently R²⁰-substituted or unsubstituted C₁-C₈ alkyl or R²⁰-substituted or unsubstituted 2 to 5 membered heteroalkyl. In embodiments, R² is independently R²⁰-substituted C₁-C₈ alkyl or R²⁰-substituted 2 to 5 membered heteroalkyl. In embodiments, R² is independently R²⁰-substituted C₁-C₈ alkyl or R²⁰-substituted 2 to 5 membered heteroalkyl; wherein R²⁰ is substituted or unsubstituted 5 to 6 membered heterocycloalkyl or substituted or unsubstituted 5 to 6 membered heteroaryl. In embodiments, R² is independently R²⁰-substituted C₁-C₈ alkyl; wherein R²⁰ is substituted or unsubstituted 5 to 6 membered heterocycloalkyl or substituted or unsubstituted 5 to 6 membered heteroaryl. In embodiments, R² is independently R²⁰-substituted 2 to 5 membered heteroalkyl; wherein R²⁰ is substituted or unsubstituted 5 to 6 membered heterocycloalkyl or substituted or unsubstituted 5 to 6 membered heteroaryl. In embodiments, R² is independently unsubstituted C₁-C₈ alkyl or unsubstituted 2 to 5 membered heteroalkyl. In embodiments, R² is independently unsubstituted C₁-C₈ alkyl. In embodiments, R² is independently unsubstituted 2 to 5 membered heteroalkyl.

In embodiments, R² is hydrogen.

In embodiments, R¹⁶ is independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

In embodiments, R¹⁷ is independently hydrogen, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

In embodiments, R¹⁸ is independently hydrogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

In embodiments, R¹⁹ is independently hydrogen, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

In embodiments, each R^{16A}, R^{16B}, R^{17A}, R^{17B}, R^{18A}, R^{8B}, R^{19A}, and R^{19B}, is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl; each X, X¹⁶, X¹⁷, X¹⁸ and X¹⁹ is independently-F, —Cl, —Br, or —I; the symbols n16, n17, n18, n19, v16, v17, v18, and v19 are independently and integer from 0 to 4; and the symbols m16, m17, m18 and m19 are independently and integer between 1 and 2. In embodiments, the symbols v16, v17, v18, and v19 are independently 1 or 2. In embodiments, the symbols m16, m17, m18 and m19 are independently 1 or 2.

In embodiments, each R^{16A}, R^{16B}, R^{17A}, R^{17B}, R^{18A}, R^{18B}, R^{9A}, and R^{19B}, is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl; each X, X¹⁶, X¹⁷, X¹⁸ and X¹⁹ is independently-F, —Cl, —Br, or —I; the symbols n16, n17, n18, n19, v16, v17, v18, and v19 are independently and integer from 0 to 4; the symbols v16, v17, v18, and v19 are independently 1 or 2; and the symbols m16, m17, m18 and m19 are independently 1 or 2.

In embodiments, X¹ is-F. In embodiments, X¹ is —Cl. In embodiments, X¹ is-Br. In embodiments, X¹ is —I. In embodiments, X² is-F. In embodiments, X² is —Cl. In embodiments, X² is-Br. In embodiments, X² is —I. In embodiments, X¹⁶ is-F. In embodiments, X¹⁶ is —Cl. In embodiments, X¹⁶ is-Br. In embodiments, X¹⁶ is —I. In embodiments, X¹⁷ is-F. In embodiments, X¹⁷ is —Cl. In embodiments, X¹⁷ is-Br. In embodiments, X¹⁷ is —I. In embodiments, X¹⁸ is-F. In embodiments, X¹⁸ is —Cl. In embodiments, X¹⁸ is-Br. In embodiments, X¹⁸ is —I. In embodiments, X¹⁹ is-F. In embodiments, X¹⁹ is —Cl. In embodiments, X¹⁹ is-Br. In embodiments, X¹⁹ is —I. In embodiments, X is-F. In embodiments, X is —Cl. In embodiments, X is-Br. In embodiments, X is —I.

In embodiments, z1 is an integer from 0 to 5. In embodiments, z1 is an integer from 0 to 2. In embodiments, z1 is 0. In embodiments, z1 is 1. In embodiments, z1 is 2. In embodiments, z1 is 3. In embodiments, z1 is 4. In embodiments, z1 is 5. In embodiments, z2 is an integer from 0 to 8. In embodiments, z2 is an integer from 0 to 2. In embodiments, z2 is 0. In embodiments, z2 is 1. In embodiments, z2 is 2. In embodiments, z2 is 3. In embodiments, z2 is 4. In embodiments, z2 is 5. In embodiments, z2 is 6. In embodiments, z2 is 7. In embodiments, z2 is 8. In embodiments, z26 is 0. In embodiments, z26 is 1. In embodiments, z26 is 2. In embodiments, z26 is 3. In embodiments, z26 is 4. In embodiments, z26 is 5. In embodiments, n1 is 0. In embodiments, n1 is 1. In embodiments, n1 is 2. In embodiments, n1 is 3. In embodiments, n1 is 4. In embodiments, m1 is 1. In embodiments, m1 is 2. In embodiments, v1 is 1. In embodiments, v1 is 2. In embodiments, n2 is 0. In embodiments, n2 is 1. In embodiments, n2 is 2. In embodiments, n2 is 3. In embodiments, n2 is 4. In embodiments, m2 is 1. In embodiments, m2 is 2. In embodiments, v2 is 1. In embodiments, v2 is 2. In embodiments, n16 is 0. In embodiments, n16 is 1. In embodiments, n16 is 2. In embodiments, n16 is 3. In embodiments, n16 is 4. In embodiments, m16 is 1. In embodiments, m16 is 2. In embodiments, v16 is 1. In embodiments, v16 is 2. In embodiments, n17 is 0. In embodiments, n17 is 1. In embodiments, n17 is 2. In embodiments, n17 is 3. In embodiments, n17 is 4. In embodiments, m17 is 1. In embodiments, m17 is 2. In embodiments, v17 is 1. In embodiments, v16 is 2. In embodiments, n18 is 0. In embodiments, n18 is 1. In embodiments, n18 is 2. In embodiments, n18 is 3. In embodiments, n18 is 4. In embodiments, m18 is 1. In embodiments, m18 is 2. In embodiments, v18 is 1. In embodiments, v18 is 2. In embodiments, n19 is 0. In embodiments, n19 is 1. In embodiments, n19 is 2. In embodiments, n19 is 3. In embodiments, n19 is 4. In embodiments, m19 is 1. In embodiments, m19 is 2. In embodiments, v19 is 1. In embodiments, v19 is 2.

In embodiments, R¹ is independently hydrogen, halogen, —CX¹³, —CHX¹², —CH₂X¹, —OCX¹₃, —OCH₂X¹, —OCHX¹₂, —CN, —SR^{1D}, —SCX¹₃, —SCH₂X¹, —SCHX¹₂, —SOₙ₁R^{1D}, —NR^{1A}R^{1B}, —OR^{1D}, —SF₅, substituted (e.g., substituted with at least one substituent group, size-limited substituent group, or lower substituent group) or unsubstituted C₁-C₆ alkyl, substituted (e.g., substituted with at least one substituent group, size-limited substituent group, or lower substituent group) or unsubstituted 2 to 6 membered heteroalkyl, or substituted (e.g., substituted with at least one substituent group, size-limited substituent group, or lower substituent group) or unsubstituted 5 to 6 membered heteroaryl.

In embodiments, a substituted R¹ (e.g., substituted alkyl, substituted heteroalkyl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R^{1.2} is independently hydrogen, halogen, —CX¹³, —CHX¹², —CH₂X¹, —OCX¹³, —OCH₂X¹, —OCHX¹², —CN, —SR¹D, —SCX¹³, —SCH₂X¹, —SCHX¹², —SOₙ₁R^{1D}, —NR^{1A}R^{1B}, —OR^{1D}, —SF₅, substituted (e.g., substituted with at least one substituent group, size-limited substituent group, or lower substituent group) or unsubstituted C₁-C₆ alkyl, substituted (e.g., substituted with at least one substituent group, size-limited substituent group, or lower substituent group) or unsubstituted 2 to 6 membered heteroalkyl, or substituted (e.g., substituted with at least one substituent group, size-limited substituent group, or lower substituent group) or unsubstituted 5 to 6 membered heteroaryl.

In embodiments, a substituted R^{1.2} (e.g., substituted alkyl, substituted heteroalkyl, and/or substituted heteroaryl) is substituted with at least one substituent group, size-limited substituent group, or lower substituent group; wherein if the substituted R^{1.2} is substituted with a plurality of groups selected from substituent groups, size-limited substituent groups, and lower substituent groups; each substituent group, size-limited substituent group, and/or lower substituent group may optionally be different. In embodiments, when R^{1.2} is substituted, it is substituted with at least one substituent group. In embodiments, when R^{1.2} is substituted, it is substituted with at least one size-limited substituent group. In embodiments, when R¹² is substituted, it is substituted with at least one lower substituent group.

In embodiments, R^{1.3} is independently hydrogen, halogen, —CX¹³, —CHX¹², —CH₂X¹, —OCX¹³, —OCH₂X¹, —OCHX¹², —CN, —SR¹D, —SCX¹³, —SCH₂X¹, —SCHX¹², —SOₙ₁R^{1D}, —NR^{1A}R^{1B}, —OR^{1D}, —SF₅, substituted (e.g., substituted with at least one substituent group, size-limited substituent group, or lower substituent group) or unsubstituted C₁-C₆ alkyl, substituted (e.g., substituted with at least one substituent group, size-limited substituent group, or lower substituent group) or unsubstituted 2 to 6 membered heteroalkyl, or substituted (e.g., substituted with at least one substituent group, size-limited substituent group, or lower substituent group) or unsubstituted 5 to 6 membered heteroaryl.

In embodiments, a substituted R^{1.3} (e.g., substituted alkyl, substituted heteroalkyl, and/or substituted heteroaryl) is substituted with at least one substituent group, size-limited substituent group, or lower substituent group; wherein if the substituted R^{1.3} is substituted with a plurality of groups selected from substituent groups, size-limited substituent groups, and lower substituent groups; each substituent group, size-limited substituent group, and/or lower substituent group may optionally be different. In embodiments, when R^{1.3} is substituted, it is substituted with at least one substituent group. In embodiments, when R^{1.3} is substituted, it is substituted with at least one size-limited substituent group. In embodiments, when R^{1.3} is substituted, it is substituted with at least one lower substituent group.

In embodiments, R^{1.4} is independently halogen, —CX¹³, —CHX¹², —CH₂X¹, —OCX¹³, —OCH₂X¹, —OCHX¹², —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹ 4 (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1.4} is substituted with a plurality of groups selected from substituent groups, size-limited substituent groups, and lower substituent groups; each substituent group, size-limited substituent group, and/or lower substituent group may optionally be different. In embodiments, when R^{1.4} is substituted, it is substituted with at least one substituent group. In embodiments, when R^{1.4} is substituted, it is substituted with at least one size-limited substituent group. In embodiments, when R^{1.4} is substituted, it is substituted with at least one lower substituent group.

In embodiments, R^{1.5} is independently halogen, —CX¹³, —CHX¹², —CH₂X¹, —OCX¹³, —OCH₂X¹, —OCHX¹², —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁵ is substituted with a plurality of groups selected from substituent groups, size-limited substituent groups, and lower substituent groups; each substituent group, size-limited substituent group, and/or lower substituent group may optionally be different. In embodiments, when R¹⁵ is substituted, it is substituted with at least one substituent group. In embodiments, when R¹⁵ is substituted, it is substituted with at least one size-limited substituent group. In embodiments, when R¹⁵ is substituted, it is substituted with at least one lower substituent group.

In embodiments, R¹⁶ is independently hydrogen,

halogen, —CX¹⁶₃, —CHX¹⁶₂, —CH₂X¹⁶, —CN, —SOₙ₁₆R^{16A}, —SOᵥ₁₆NR^{16A}R^{16B}, —NHNR^{16A}R^{16B}, —ONR^{16A}R^{16B}, —NHC(O)NHNR^{16A}R^{16B}, —NHC(O)NR^{16A}R^{16B}, —N(O)ₘ₁₆, —NR^{16A}R^{16B}, —C(O)R^{16A}, —C(O)—OR^{16A}, —C(O)NR^{16A}R^{16B}, —OR^{16A}, —NR^{16A}SO₂R^{16B}, —NR^{16A}C(O)R^{16B}, —NR^{16A}C(O)OR^{16B}, —NR^{16A}OR^{16B}, —OCX¹⁶₃, —OCHX¹⁶₂, —OCH₂X¹⁶, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁶ is substituted with a plurality of groups selected from substituent groups, size-limited substituent groups, and lower substituent groups; each substituent group, size-limited substituent group, and/or lower substituent group may optionally be different. In embodiments, when R¹⁶ is substituted, it is substituted with at least one substituent group. In embodiments, when R¹⁶ is substituted, it is substituted with at least one size-limited substituent group. In embodiments, when R¹⁶ is substituted, it is substituted with at least one lower substituent group.

In embodiments, R¹⁷ is independently hydrogen,

halogen, —CX¹⁷₃, —CHX¹⁷₂, —CH₂X¹⁷, —CN, —SOₙ₁₇R^{17A}, —SOᵥ₁₇NR^{17A}R^{17B}, —NHNR^{17A}R^{17B}, —ONR^{17A}R^{17B}, —NHC(O)NHNR^{17A}R^{17B}, —NHC(O)NR^{17A}R^{17B}, —N(O)ₘ₁₇, —NR^{17A}R^{17B}, —C(O)R^{17A}, —C(O)—OR^{17A}, —C(O)NR^{17A}R^{17B}, —OR^{17A}, —NR^{17A}SO₂R^{17B}, —NR^{17A}C(O)R^{17B}, —NR^{17A}C(O)OR^{17B}, —NR^{17A}OR^{17B}, —OCX¹⁷₃, —OCHX¹⁷₂, —OCH₂X¹⁷, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁷ is substituted with a plurality of groups selected from substituent groups, size-limited substituent groups, and lower substituent groups; each substituent group, size-limited substituent group, and/or lower substituent group may optionally be different. In embodiments, when R¹⁷ is substituted, it is substituted with at least one substituent group. In embodiments, when R¹⁷ is substituted, it is substituted with at least one size-limited substituent group. In embodiments, when R¹⁷ is substituted, it is substituted with at least one lower substituent group.

In embodiments, R¹⁸ is independently hydrogen, halogen, —CX¹⁸₃, —CHX¹⁸₂, —CH₂X¹⁸, —CN, —SOₙ₁₈R^{18A}, —SOᵥ₁₈NR^{18A}R^{18B}, —NHNR^{18A}R^{18B}, —ONR^{18A}R^{18B}, —NHC(O)NHNR^{18A}R^{18B}, —NHC(O)NR^{18A}R^{18B}, —N(O)ₘ₁₈, —NR^{18A}R^{8B}, —C(O)R^{18A}, —C(O)—OR^{18A}, —C(O)NR^{18A}R^{18B}, —OR^{18A}, —NR^{18A}SO₂R^{18B}, —NR^{18A}C(O)R^{18B}, —NR^{18A}C(O)OR^{18B}, —NR^{18A}OR^{18B}, —OCX¹⁸₃, —OCHX¹⁸₂, —OCH₂X¹⁸, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁸ is substituted with a plurality of groups selected from substituent groups, size-limited substituent groups, and lower substituent groups; each substituent group, size-limited substituent group, and/or lower substituent group may optionally be different. In embodiments, when R¹⁸ is substituted, it is substituted with at least one substituent group. In embodiments, when R¹⁸ is substituted, it is substituted with at least one size-limited substituent group. In embodiments, when R¹⁸ is substituted, it is substituted with at least one lower substituent group.

In embodiments, R¹⁹ is independently hydrogen,

halogen, —CX¹⁹₃, —CHX¹⁹₂, —CH₂X¹⁹, —CN, —SOₙ₁₉R^{19A}, —SOᵥ₁₉NR^{19A}R^{19B}, —NHNR^{19A}R^{19B}, —ONR^{19A}R^{19B}, —NHC(O)NHNR^{19A}R^{19B} —NHC(O)NR^{19A}R^{19B}, —N(O)ₘ₁₉, —NR^{9A}R^{19B}, —C(O)R^{19A}, —C(O)—OR¹⁹A —C(O)NR^{19A}R^{19B}, —OR^{19A}, —NR^{19A}SO₂R^{19B}, —NR^{19A}C(O)R^{19B}, —NR^{19A}C(O)OR^{19B}, —NR^{19A}OR^{19B}, —OCX¹⁹₃, —OCHX¹⁹₂, —OCH₂X¹⁹, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹⁹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁹ is substituted with a plurality of groups selected from substituent groups, size-limited substituent groups, and lower substituent groups; each substituent group, size-limited substituent group, and/or lower substituent group may optionally be different. In embodiments, when R¹⁹ is substituted, it is substituted with at least one substituent group. In embodiments, when R¹⁹ is substituted, it is substituted with at least one size-limited substituent group. In embodiments, when R¹⁹ is substituted, it is substituted with at least one lower substituent group.

In embodiments, R^{16A} is independently

hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{16A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{16A} is substituted with a plurality of groups selected from substituent groups, size-limited substituent groups, and lower substituent groups; each substituent group, size-limited substituent group, and/or lower substituent group may optionally be different. In embodiments, when R^{16A} is substituted, it is substituted with at least one substituent group. In embodiments, when R^{16A} is substituted, it is substituted with at least one size-limited substituent group. In embodiments, when R^{16A} is substituted, it is substituted with at least one lower substituent group.

In embodiments, R^{16B} is independently

hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{16B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{16B} is substituted with a plurality of groups selected from substituent groups, size-limited substituent groups, and lower substituent groups; each substituent group, size-limited substituent group, and/or lower substituent group may optionally be different. In embodiments, when R^{16B} is substituted, it is substituted with at least one substituent group. In embodiments, when R^{16B} is substituted, it is substituted with at least one size-limited substituent group. In embodiments, when R^{16B} is substituted, it is substituted with at least one lower substituent group.

In embodiments, R^{17A} is independently

hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{17A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{17A} is substituted with a plurality of groups selected from substituent groups, size-limited substituent groups, and lower substituent groups; each substituent group, size-limited substituent group, and/or lower substituent group may optionally be different. In embodiments, when R^{17A} is substituted, it is substituted with at least one substituent group. In embodiments, when R^{17A} is substituted, it is substituted with at least one size-limited substituent group. In embodiments, when R^{17A} is substituted, it is substituted with at least one lower substituent group.

In embodiments, R^{17B} is independently

hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{17B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{17B} is substituted with a plurality of groups selected from substituent groups, size-limited substituent groups, and lower substituent groups; each substituent group, size-limited substituent group, and/or lower substituent group may optionally be different. In embodiments, when R^{17B} is substituted, it is substituted with at least one substituent group. In embodiments, when R^{17B} is substituted, it is substituted with at least one size-limited substituent group. In embodiments, when R^{17B} is substituted, it is substituted with at least one lower substituent group.

In embodiments, R^{18A} is independently

hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{18A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{18A} is substituted with a plurality of groups selected from substituent groups, size-limited substituent groups, and lower substituent groups; each substituent group, size-limited substituent group, and/or lower substituent group may optionally be different. In embodiments, when R^{18A} is substituted, it is substituted with at least one substituent group. In embodiments, when R^{18A} is substituted, it is substituted with at least one size-limited substituent group. In embodiments, when R^{18A} is substituted, it is substituted with at least one lower substituent group.

In embodiments, R^{8B} is independently

hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{18B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{18B} is substituted with a plurality of groups selected from substituent groups, size-limited substituent groups, and lower substituent groups; each substituent group, size-limited substituent group, and/or lower substituent group may optionally be different. In embodiments, when R^{18B} is substituted, it is substituted with at least one substituent group. In embodiments, when R^{18B} is substituted, it is substituted with at least one size-limited substituent group. In embodiments, when R^{8B} is substituted, it is substituted with at least one lower substituent group.

In embodiments, R^{19A} is independently

hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{19A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{19A} is substituted with a plurality of groups selected from substituent groups, size-limited substituent groups, and lower substituent groups; each substituent group, size-limited substituent group, and/or lower substituent group may optionally be different. In embodiments, when R^{19A} is substituted, it is substituted with at least one substituent group. In embodiments, when R^{19A} is substituted, it is substituted with at least one size-limited substituent group. In embodiments, when R^{19A} is substituted, it is substituted with at least one lower substituent group.

In embodiments, R^{19B} is independently

hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{19B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{19B} is substituted with a plurality of groups selected from substituent groups, size-limited substituent groups, and lower substituent groups; each substituent group, size-limited substituent group, and/or lower substituent group may optionally be different. In embodiments, when R^{19B} is substituted, it is substituted with at least one substituent group. In embodiments, when R^{19B} is substituted, it is substituted with at least one size-limited substituent group. In embodiments, when R^{19B} is substituted, it is substituted with at least one lower substituent group.

In embodiments, R^{16A} and R^{16B} substituents bonded to the same nitrogen atom may optionally be joined to form a substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered) or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R^{17A} and R^{17B} substituents bonded to the same nitrogen atom may optionally be joined to form a substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered) or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R^{18A} and R^{18B} substituents bonded to the same nitrogen atom may optionally be joined to form a substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered) or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R^{19A} and R^{19B} substituents bonded to the same nitrogen atom may optionally be joined to form a substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered) or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, the compound is:

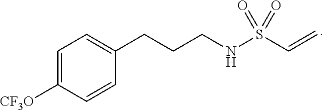

In embodiments, the compound is:

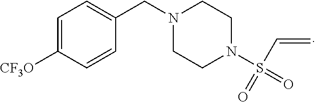

In embodiments, the compound is:

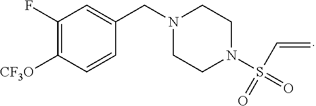

In embodiments, the compound is:

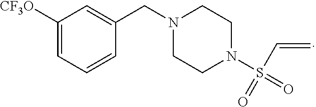

In embodiments, the compound is:

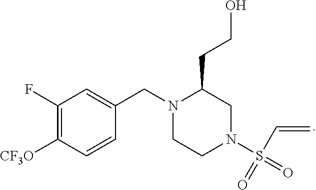

In embodiments, the compound is:

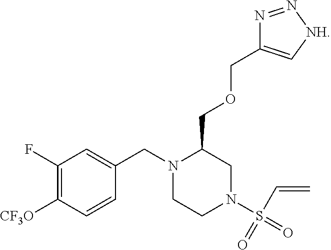

In embodiments, the compound is:

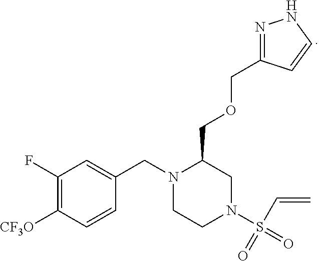

In embodiments, the compound is:

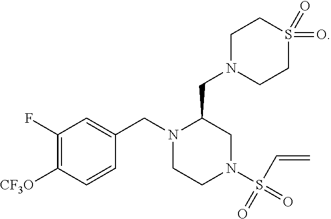

In embodiments, the compound is:

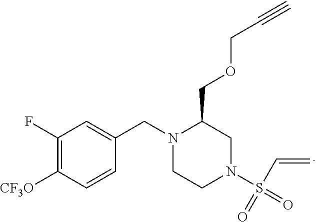

In embodiments, the compound is:

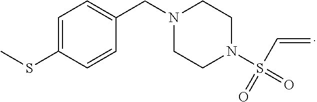 [SMILES: C=CS(=O)(=O)N1CCN(Cc2ccc(SC)cc2)CC1]

In embodiments, the compound is:

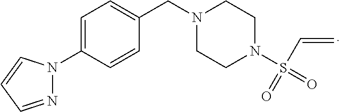 [SMILES: C=CS(=O)(=O)N1CCN(Cc2ccc(-n3cccn3)cc2)CC1]

In embodiments, the compound is:

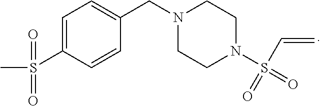 [SMILES: C=CS(=O)(=O)N1CCN(Cc2ccc(S(C)(=O)=O)cc2)CC1]

In embodiments, the compound is:

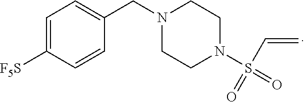

In embodiments, the compound is:

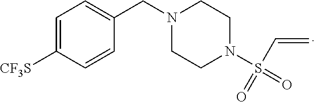

In embodiments, the compound is useful as a comparator compound. In embodiments, the comparator compound can be used to assess the activity of a test compound in an assay (e.g., an assay as described herein, for example in the examples section, figures, or tables).

In embodiments, the compound is a compound described herein (e.g., in an aspect, embodiment, example, table, figure, or claim).

### III. Pharmaceutical Compositions

In an aspect is provided a pharmaceutical composition including a compound as described herein, including embodiments, and a pharmaceutically acceptable excipient. In embodiments, the compound as described herein is included in a therapeutically effective amount.

In embodiments of the pharmaceutical compositions, the compound, or pharmaceutically acceptable salt thereof, is included in a therapeutically effective amount.

In embodiments of the pharmaceutical compositions, the pharmaceutical composition includes a second agent (e.g. therapeutic agent). In embodiments of the pharmaceutical compositions, the pharmaceutical composition includes a second agent (e.g. therapeutic agent) in a therapeutically effective amount. In embodiments of the pharmaceutical compositions, the second agent is an agent for treating cancer. In embodiments, the second agent is an anti-cancer agent. In embodiments, the second agent is a chemotherapeutic. In embodiments, the second agent is an anti-inflammatory agent. In embodiments, the administering does not include administration of any active agent other than the recited active agent (e.g., a compound described herein).

### IV. Methods of Use

In an aspect is provided a method of inhibiting Taspase1 protein activity, the method including contacting the Taspase1 protein with a compound as described herein.

In an aspect is provided a method of treating cancer, the method including administering to a subject in need thereof an effective amount of a compound as described herein. In embodiments, the cancer is glioblastoma, bladder, kidney, liver, pancreatic, melanoma, leukemia, lymphoma, ovarian cancer, renal cancer, colon cancer, prostate cancer, lung cancer, brain cancer, or breast cancer. In embodiments, the cancer is glioblastoma. In embodiments, the cancer is bladder cancer. In embodiments, the cancer is kidney cancer. In embodiments, the cancer is liver cancer. In embodiments, the cancer is pancreatic cancer. In embodiments, the cancer is melanoma. In embodiments, the cancer is leukemia. In embodiments, the cancer is lymphoma. In embodiments, the cancer is ovarian cancer. In embodiments, the cancer is renal cancer. In embodiments, the cancer is colon cancer. In embodiments, the cancer is prostate cancer. In embodiments, the cancer is lung cancer. In embodiments, the cancer is brain cancer. In embodiments, the cancer is breast cancer.

In embodiments, the cancer is colorectal cancer. In embodiments, the cancer is liver cancer. In embodiments, the cancer is hepatocellular cancer. In embodiments, the cancer is breast cancer. In embodiments, the cancer is estrogen receptor positive breast cancer. In embodiments, the cancer is estrogen receptor (ER) negative breast cancer. In embodiments, the cancer is tamoxifen resistant breast cancer. In embodiments, the cancer is HER2 negative breast cancer. In embodiments, the cancer is HER2 positive breast cancer. In embodiments, the cancer is low grade (well differentiated) breast cancer. In embodiments, the cancer is intermediate grade (moderately differentiated) breast cancer. In embodiments, the cancer is high grade (poorly differentiated) breast cancer. In embodiments, the cancer is stage 0 breast cancer. In embodiments, the cancer is stage I breast cancer. In embodiments, the cancer is stage II breast cancer. In embodiments, the cancer is stage III breast cancer. In embodiments, the cancer is stage IV breast cancer. In embodiments, the cancer is triple negative breast cancer.

In embodiments, the cancer is sensitive to Taspase1 inhibition as determined using techniques known in the art (e.g., a screening assay).

In embodiments, the method includes administering a second agent (e.g. therapeutic agent). In embodiments, the method includes administering a second agent (e.g. therapeutic agent) in a therapeutically effective amount. In embodiments, the second agent is an agent for treating cancer. In embodiments, the second agent is an anti-cancer agent. In embodiments, the second agent is a chemotherapeutic. In embodiments, the second agent is an anti-inflammatory agent.

In an aspect is provided a method of inhibiting Taspase1 protein activity, the method including: contacting the Taspase1 protein with a compound described herein.

### V. Taspase1 Protein

In an aspect is provided a Taspase1 protein covalently bonded to a compound as described herein. In embodiments, the compound is bonded (e.g., covalently bonded) to a cysteine residue of the protein.

In an aspect is provided a Taspase protein covalently bonded to a portion of a compound as described herein.

Where the compound covalently binds to the Taspase1, a Taspase1 protein (e.g., human Taspase1) covalently bonded to a Taspase1 inhibitor is formed (also referred to herein as a “Taspase1-compound adduct”), as described below. In embodiments, the resulting covalent bond is reversible. Where the resulting covalent bond is reversible, the bonding reverses upon denaturation of the protein. Thus, in embodiments, the reversibility of a covalent bond between the compound and the Taspase1 upon denaturation of the Taspase1 avoids or decreases autoimmune response in a subject subsequent to administration of the compound (relative to irreversibility).

In embodiments, the Taspase1 protein (e.g., human Taspase1) is covalently bonded to a Taspase1 inhibitor (e.g., compound described herein or a portion of a compound described herein). In embodiments, the Taspase1 protein (e.g., human Taspase1) is irreversibly covalently bonded to a Taspase1 inhibitor (e.g., compound described herein or a portion of a compound described herein). In embodiments, the Taspase1 protein (e.g., human Taspase1) is reversibly covalently bonded to a Taspase1 inhibitor (e.g., compound described herein or a portion of a compound described herein). In embodiments, the Taspase1 protein (e.g., human Taspase1) is covalently bonded to a portion of a Taspase1 inhibitor (e.g., compound described herein). In embodiments, the Taspase1 protein (e.g., human Taspase1) is irreversibly covalently bonded to a portion of a Taspase1 inhibitor (e.g., compound described herein). In embodiments, the Taspase1 protein (e.g., human Taspase1) is reversibly covalently bonded to a portion of a Taspase1 inhibitor (e.g., compound described herein). In embodiments, the Taspase1 inhibitor (e.g., compound described herein) is bonded to a cysteine residue (e.g., Cys293 of human Taspase1 or cysteine corresponding to Cys293 of human Taspase1) of the Taspase1 protein (e.g., human Taspase1).

In embodiments, the Taspase1 protein covalently bonded to a Taspase1 inhibitor or compound described herein is the product of a reaction between the Taspase1 protein and a Taspase1 inhibitor or compound described herein. It will be understood that the covalently bonded Taspase1 protein and Taspase1 inhibitor (e.g., compound described herein) are the remnants of the reactant Taspase1 protein and Taspase1 inhibitor or compound, wherein each reactant now participates in the covalent bond between the Taspase1 protein and Taspase1 inhibitor or compound. In embodiments of the covalently bonded Taspase1 protein and compound described herein, the remnant of the E substituent is a linker including a covalent bond between the Taspase1 protein and the remainder of the compound described herein. It will be understood by a person of ordinary skill in the art that when a Taspase1 protein is covalently bonded to a Taspase1 inhibitor (e.g., compound described herein), the Taspase1 inhibitor (e.g., compound described herein) forms a remnant of the pre-reacted Taspase1 inhibitor (e.g., compound described herein) wherein a bond connects the remnant of the Taspase1 inhibitor (e.g., compound described herein) to the remnant of the Taspase1 protein (e.g., cysteine sulfur, sulfur of amino acid corresponding to C293 of human Taspase1, sulfur of C293 of human Taspase1). The remnant of the Taspase1 inhibitor (compound described herein) may also be called a portion of the Taspase1 inhibitor. In embodiments, the remnant of the electrophilic moiety (e.g., R³) substituent is a linker selected from a bond, —S(O)₂—, —NH—, —O—, —S—, —C(O)—, —C(O)NH—, —NHC(O)—, —NHC(O)NH—, —NHC(O)NH—, —C(O)O—, —OC(O)—, —CH₂NH—, substituted (e.g., substituted with a substituent group, a size-limited substituent group, or lower substituent group) or unsubstituted alkylene (e.g., C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₀ or phenyl), or substituted (e.g., substituted with a substituent group, a size-limited substituent group, or lower substituent group) or unsubstituted heteroarylene (e.g., 5 to 10 membered, 5 to 9 membered, or 5 to 6 membered).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### VI. Embodiments

Embodiment P1. A compound having the formula:

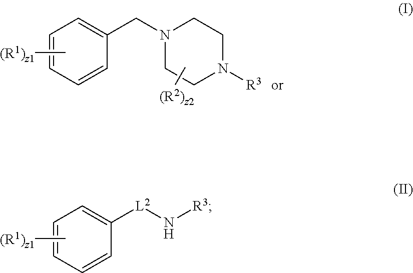

wherein,
R¹ is independently halogen, —CX¹³, —CHX¹², —CH₂X¹, —OCX¹³, —OCH₂X¹, —OCHX¹², —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
L² is substituted or unsubstituted alkylene;
R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR²CNR^{2A}R^{2B}, —ONR^{2A}R^{2B}, —NHC(O)NR²CNR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O) NR^{2A}R^{2B}, —OR^{2D}, —NR^{2A}SO₂R^{2D}, —NR^{2A}C(O)R^{2C}, —NR^{2A}C(O)OR²C, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents may optionally be joined to form a substituted or unsubstituted cycloalkyl, substituted or unsubstituted heterocycloalkyl, substituted or unsubstituted aryl, or substituted or unsubstituted heteroaryl;
R³ is independently —CN,

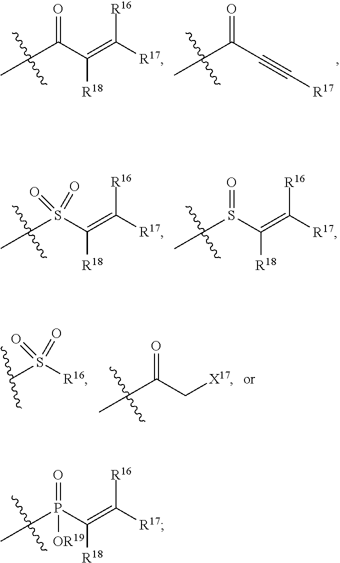

wherein R¹⁶ is independently hydrogen, halogen, —CX¹⁶₃, —CHX¹⁶₂, —CH₂X¹⁶, —CN, —SOₙ₁₆R^{16A}, —SOᵥ₁₆NR^{16A}R^{16B}, —NHNR^{16A}R^{16B}, —ONR^{16A}R^{16B}, —NHC(O)NHNR^{16A}R^{16B}, —NHC(O)NR^{16A}R^{16B}, —N(O)ₘ₁₆, —NR^{16A}R^{16B} C(O)R^{16A}, —C(O)—OR^{16A}, —C(O)NR^{16A}R^{16B}, —OR^{16A}, —NR^{16A}SO₂R^{16B}, —NR^{1A}C(O)R^{16B}, —NR^{16A}C(O)OR^{16B}, —NR^{16A}OR^{16B}, —OCX¹⁶₃, —OCHX¹⁶₂, —OCH₂X¹⁶, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁷ is independently hydrogen,
halogen, —CX¹⁷₃, —CHX¹⁷₂, —CH₂X¹⁷, —CN, —SOₙ₁₇R^{17A}, —SOᵥ₁₇NR^{17A}R^{17B}, —NHNR^{17A}R^{17B}, —ONR^{17A}R^{17B}, —NHC(O)NHNR^{17A}R^{17B}, —NHC(O)NR^{17A}R^{17B}, —N(O)ₘ₁₇, —NR^{17A}R^{17B}, —C(O)R^{17A}, —C(O)—OR^{17A}, —C(O)NR^{17A}R^{17B}, —OR^{17A}, —NR^{17A}SO₂R^{17B}, —NR^{17A}C(O)R^{17B}, —NR^{17A}C(O)OR^{17B}, —NR^{17A}OR^{17B}, —OCX¹⁷₃, —OCHX¹⁷₂, —OCH₂X¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁸ is independently hydrogen,
halogen, —CX¹⁸₃, —CHX¹⁸₂, —CH₂X¹⁸, —CN, —SOₙ₁₈R^{18A}, —SOᵥ₁₈NR^{18A}R^{18B}, —NHNR^{18A}R^{18B}, —ONR^{18A}R^{18B}, —NHC(O)NHNR^{18A}R^{18B}, —NHC(O)NR^{18A}R^{18B}, —N(O)ₘ₁₈, —NR^{18A}R^{8B}, —C(O)R^{18A}, —C(O)—OR^{18A}, —C(O)NR^{18A}R^{18B}, —OR^{18A}, —NR^{18A}SO₂R^{18B}, —NR^{18A}C(O)R^{18B}, —NR^{18A}C(O)OR^{18B}, —NR^{18A}OR^{18B}, —OCX¹⁸₃, —OCHX¹⁸₂, —OCH₂X¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁹ is independently hydrogen,
halogen, —CX¹93, —CHX¹⁹₂, —CH₂X¹⁹, —CN, —SOₙ₁₉R^{19A}, —SOᵥ₁₉NR^{19A}R^{19B}, —NHNR^{19A}R^{19B}, —ONR^{19A}R^{19B}, —NHC(O)NHNR^{19A}R^{19B}, —NHC(O)NR^{19A}R^{19B}, —N(O)ₘ₁₉, —NR^{19A}R^{19B} C(O)R^{19A}, —C(O)—OR¹⁹A —C(O)NR^{19A}R^{19B}, —OR^{19A}, —NR^{19A}SO₂R^{19B}, —NR^{19A}C(O)R^{19B}, —NR^{19A}C(O)OR^{19B}, —NR^{19A}OR^{19B}, —OCX¹⁹₃, —OCHX¹⁹₂, —OCH₂X¹⁹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R^{1A}, R^{1B}, R^{1C}, R^{1D}, R^{2A}, R^{2B}, R^{2C}, R^{2D}, R^{16A}, R^{16B}, R^{17A}, R^{17B}, R^{18A}, R^{18B}, R^{19A}, and R^{19B} are independently hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl;
X, X¹, X², X¹⁶, X¹⁷, X¹⁸, and X¹⁹ are independently —F, —Cl, —Br, or —I;
n1, n2, n16, n17, n18, and n19 are independently an integer from 0 to 4;
m1, m2, m16, m17, m18, m19, v1, v2, v16, v17, v18, and v19 are independently 1 or 2;
z1 is an integer from 0 to 5; and
z2 is an integer from 0 to 8.

Embodiment P2. The compound of embodiment P1, having the formula:

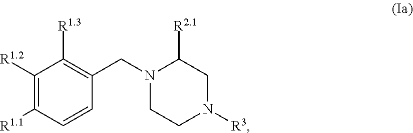

wherein,
R^{1.1}, R^{1.2}, and R^{1.3} are independently hydrogen, halogen, —CX¹₃, —CHX¹₂, —CH₂X¹, —OCX¹³, —OCH₂X¹, —OCHX¹², —CN, —SOₙ₁R^{1D}, —NR^{1A}R^{1B}, —OR^{1D}, —SF₅, substituted or unsubstituted C₁-C₆ alkyl, substituted or unsubstituted 2 to 6 membered heteroalkyl, or substituted or unsubstituted 5 to 6 membered heteroaryl;
R^{2.1} is independently hydrogen, oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
—NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl; R³ is independently —CN,

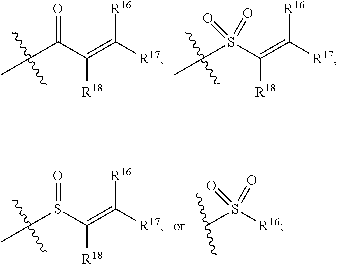

R¹⁶, R¹⁷, and R¹⁸ are independently hydrogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₇ cycloalkyl, or substituted or unsubstituted C₆-C₁₂ aryl;
R^{1A}, R^{1B}, and R^{1D} are independently
hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; and
X is independently —F, —Cl, —Br, or —I.

Embodiment P3. The compound of embodiments P1 to P2, having the formula:

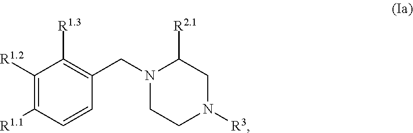

wherein,
R^{1.1}, R^{1.2}, and R^{1.3} are independently hydrogen, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, —SH, —SCH₃, —SCF₃, —SCHF₂, —SCH₂F, —SCCl 3, —SCHCl₂, —SCH₂Cl, —SCBr₃, —SCHBr₂, —SCH₂Br, —SCl₃, —SCHI₂, —SCH₂I, —SOCH₃, —SO₂CH₃, —NH₂, —NHCH₃, —OH, —SF₅, substituted or unsubstituted C₁-C₆ alkyl, substituted or unsubstituted 2 to 6 membered heteroalkyl, or substituted or unsubstituted 5 to 6 membered heteroaryl;
R^{2.1} is independently hydrogen, oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
—NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl;
R³ is independently —CN,

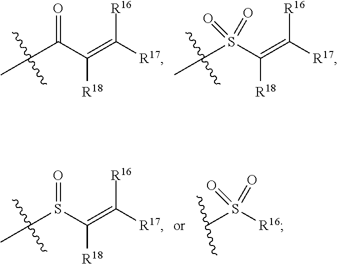

and
R¹⁶, R¹⁷, and R¹⁸ are independently hydrogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₆ cycloalkyl, or substituted C₆ aryl.

Embodiment P4. The compound of one of embodiments P1 to P3, wherein R^{2.1} is independently hydrogen, substituted or unsubstituted C₁-C₆ alkyl, substituted or unsubstituted 2 to 6 membered heteroalkyl, substituted or unsubstituted C₆-C₁₂ aryl, or substituted or unsubstituted 5 to 12 membered heteroaryl.

Embodiment P5. The compound of embodiments P1 to P3, having the formula:

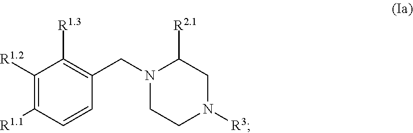

wherein,
R^{2.1} is independently —CH₂O—CH₂CCH, —CH₂O—CH₂CN, —CH₂O—CH₂-heterocycloalkyl, substituted or unsubstituted C₆-C₁₂ aryl, or substituted or unsubstituted 5 to 12 membered heteroaryl.

Embodiment P6. The compound of one of embodiments P1 to P3, wherein R^{2.1} is independently hydrogen, substituted or unsubstituted C₁-C₆ alkyl, or substituted or unsubstituted 2 to 6 membered heteroalkyl.

Embodiment P7. The compound of one of embodiments P1 to P3, wherein R^{2.1} is independently hydrogen, R²⁰-substituted or unsubstituted C₁-C₆ alkyl, or R²⁰-substituted or unsubstituted 2 to 6 membered heteroalkyl;

R²⁰ is independently —OH, R²¹-substituted or unsubstituted 5 to 6 membered heterocycloalkyl or
R²¹-substituted or unsubstituted 5 to 6 membered heteroaryl; and
R²¹ is independently oxo.

Embodiment P8. The compound of embodiment P1, having the formula:

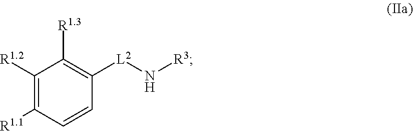

wherein,
R^{1.1}, R^{1.2}, and R^{1.3} are independently hydrogen, halogen, —CX¹³, —CHX¹², —CH₂X¹, —OCX¹³, —OCH₂X¹, —OCHX¹², —CN, —SOₙ₁R^{1D}, —NR^{1A}R^{1B}, —OR^{1D}, —SF₅, substituted or unsubstituted C₁-C₆ alkyl, substituted or unsubstituted 2 to 6 membered heteroalkyl, or substituted or unsubstituted 5 to 6 membered heteroaryl;
L² is unsubstituted C₁-C₆ alkylene;
R³ is independently —CN,

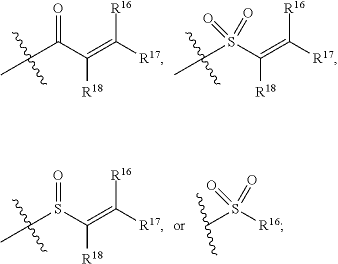

R¹⁶, R¹⁷, and R¹⁸ are independently hydrogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₇ cycloalkyl, or substituted or unsubstituted C₆-C₁₂ aryl;
R^{1A}, R^{1B}, and R^{1D} are independently
hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; and
X is independently —F, —Cl, —Br, or —I.

Embodiment P9. The compound of embodiments P1 and P8, having the formula:

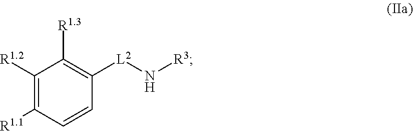

wherein,
R^{1.1}, R^{1.2}, and R^{1.3} are independently hydrogen, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, —SH, —SCH₃, —SCF₃, —SCHF₂, —SCH₂F, —SCCl ₃, —SCHCl₂, —SCH₂Cl, —SCBr₃, —SCHBr₂, —SCH₂Br, —SCl₃, —SCHI₂, —SCH₂I, —SOCH₃, —SO₂CH₃, —NH₂, —NHCH₃, —OH, —SF₅, substituted or unsubstituted C₁-C₆ alkyl, substituted or unsubstituted 2 to 6 membered heteroalkyl, or substituted or unsubstituted 5 to 6 membered heteroaryl;
L² is unsubstituted C₁-C₆ alkylene;
R³ is independently —CN,

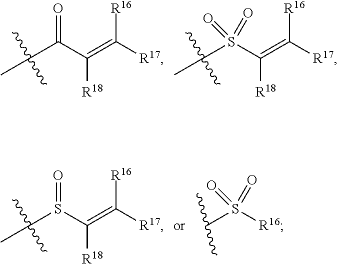

and
R¹⁶, R¹⁷, and R¹⁸ are independently hydrogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₆ cycloalkyl, or substituted C₆ aryl.

Embodiment P10. The compound of embodiment P9, wherein L² is unsubstituted n-propylene or unsubstituted n-butylene.

Embodiment P11. The compound of embodiments P1 or P10, wherein R^{1.1} is independently

hydrogen, —OCCl₃, —OCF₃, —OCBr₃, —OCl₃, —OCHCl₂, —OCHBr₂, —OCHI₂, —OCHF₂, —OCH₂Cl, —O CH₂Br, —OCH₂I, —OCH₂F, —CN, —SH, —SCH₃, —SCF₃, —SCHF₂, —SCH₂F, —SCCl₃, —SCHCl₂, —SCH₂ Cl, —SCBr₃, —SCHBr₂, —SCH₂Br, —SCl₃, —SCHI₂, —SCH₂I, —SOCH₃, —SO₂CH₃, —NH₂, —NHCH₃, —OH, —SF₅, alkenyl, alkynyl, unsubstituted methoxy, unsubstituted ethoxy, unsubstituted n-propoxy, unsubstituted isopropoxy, unsubstituted n-butoxy, unsubstituted t-butoxy, unsubstituted sec-butoxy, unsubstituted isobutoxy, or unsubstituted pyrazolyl;
R^{1.2} is independently hydrogen, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, or unsubstituted C₁-C₄ alkyl; and
R^{1.3} is independently hydrogen,
halogen, —OCCl₃, —OCF₃, —OCBr₃, —OCl₃, —OCHCl₂, —OCHBr₂, —OCHI₂, —OCHF₂, —OCH₂Cl, —OC H₂Br, —OCH₂I, —OCH₂F, —CN, unsubstituted methoxy, unsubstituted ethoxy, unsubstituted n-propoxy, unsubstituted isopropoxy, unsubstituted n-butoxy, unsubstituted t-butoxy, unsubstituted sec-butoxy, or unsubstituted isobutoxy.

Embodiment P12. The compound of embodiments P1 or P10, wherein

R^{1.1} is independently hydrogen, —OCF₃, —CN, —SCH₃, —SCF₃, —SOCH₃, —SO₂CH₃, —NHCH₃, —SF₅, unsubstituted C₂-C₄ alkenyl, unsubstituted C₂-C₄ alkynyl, unsubstituted isopropoxy, or unsubstituted pyrazolyl;
R^{1.2} is independently hydrogen, —F, —Br, or —CF₃; and
R^{1.3} is independently hydrogen, —F, or —OCF₃.

Embodiment P13. The compound of one of embodiments P1 to P12, wherein

R³ is independently —CN.

Embodiment P14. The compound of one of embodiments P1 to P12, wherein

R³ is independently

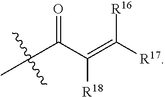

Embodiment P15. The compound of one of embodiments P1 to P12, wherein

R³ is independently

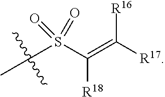

Embodiment P16. The compound of one of embodiments P1 to P12, wherein

R³ is independently

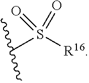

Embodiment P17. The compound of one of embodiments P1 to P12, wherein

R³ is independently

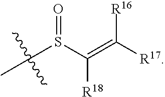

Embodiment P18. The compound of one of embodiments P1 to P17, wherein

R¹⁶ is hydrogen;
R¹⁷ is independently hydrogen, unsubstituted C₁-C₄ alkyl, or unsubstituted C₃-C₆ cycloalkyl; and
R¹⁸ is independently hydrogen, unsubstituted C₁-C₄ alkyl, or unsubstituted C₃-C₆ cycloalkyl.

Embodiment P19. The compound of one of embodiments P1 to P17, wherein

R¹⁶ is hydrogen;
R¹⁷ is independently hydrogen or unsubstituted C₁-C₄ alkyl; and
R¹⁸ is independently hydrogen or unsubstituted C₁-C₄ alkyl.

Embodiment P20. The compound of one of embodiments P1 to P17, wherein

R¹⁶ is hydrogen;
R¹⁷ is independently hydrogen, unsubstituted methyl, or unsubstituted cyclopropyl; and
R¹⁸ is independently hydrogen, unsubstituted methyl, or unsubstituted cyclopropyl.

Embodiment P21. The compound of one of embodiments P1 to P17, wherein

R¹⁶ is hydrogen;
R¹⁷ is independently hydrogen or unsubstituted methyl; and
R¹⁸ is independently hydrogen or unsubstituted methyl.

Embodiment P2₂. The compound of one of embodiments P1 to P17, wherein

R¹⁶, R¹⁷ and R¹⁸ are hydrogen.

Embodiment P2₃. A pharmaceutical composition comprising the compound of any one of embodiments P1 to P2₂ and a pharmaceutically acceptable excipient.

Embodiment P24. A method of inhibiting Taspase1 protein activity, said method comprising: contacting the Taspase1 protein with a compound of one of embodiments P1 to P2₂.

Embodiment P25. A method of treating cancer, said method comprising administering to a subject in need thereof an effective amount of a compound of one of embodiments P1 to P2₂.

Embodiment P26. The method of embodiment P25, wherein the cancer is glioblastoma, melanoma, leukemia, lymphoma, ovarian cancer, renal cancer, colon cancer, prostate cancer, lung cancer, brain cancer, or breast cancer.

Embodiment P27. The method of embodiment P25, wherein the cancer is sensitive to Taspase1 inhibition.

Embodiment P28. A Taspase1 protein covalently bonded to a compound of one of embodiments P1 to P2₂.

Embodiment P29. The Taspase1 protein of embodiment P28, wherein the compound is bonded to a cysteine residue of the protein.

Embodiment P30. A Taspase protein covalently bonded to a portion of a compound of one of embodiments P1 to P2₂.

### VII. Additional Embodiments

Embodiment 1. A compound having the formula:

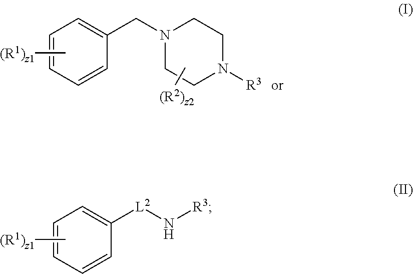

R¹ is independently halogen, —CX¹³, —CHX¹², —CH₂X¹, —OCX¹³, —OCH₂X¹, —OCHX¹², —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
L² is substituted or unsubstituted alkylene;
R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR²CNR^{2A}R^{2B}, —ONR^{2A}R^{2B}, —NHC(O)NR²CNR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O) NR^{2A}R^{2B}, —OR^{2D}, —NR^{2A}SO₂R^{2D}, —NR^{2A}C(O)R^{2C}, —NR^{2A}C(O)OR²C, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents may optionally be joined to form a substituted or unsubstituted cycloalkyl, substituted or unsubstituted heterocycloalkyl, substituted or unsubstituted aryl, or substituted or unsubstituted heteroaryl;
R³ is independently —CN,

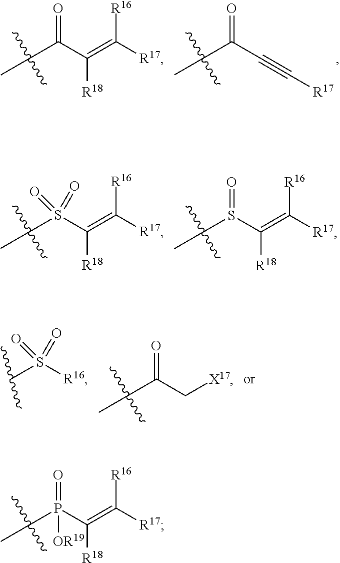

wherein R¹⁶ is independently hydrogen, halogen, —CX¹⁶₃, —CHX¹⁶₂, —CH₂X¹⁶, —CN, —SOₙ₁₆R^{16A}, —SOᵥ₁₆NR^{16A}R^{16B}, —NHNR^{16A}R^{16B}, —ONR^{16A}R^{16B}, —NHC(O)NHNR^{16A}R^{16B}, —NHC(O)NR^{16A}R^{16B}, —N(O)ₘ₁₆, —NR^{16A}R^{16B} C(O)R^{6A}, —C(O)—OR^{16A}, —C(O)NR^{16A}R^{16B}, —OR^{16A}, —NR^{16A}SO₂R^{16B}, —NR^{16A}C(O)R^{16B}, —NR^{16A}C(O)OR^{16B}, —NR^{16A}OR^{16B}, —OCX¹⁶₃, —OCHX¹⁶₂, —OCH₂X¹⁶, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁷ is independently hydrogen,
halogen, —CX¹⁷₃, —CHX¹⁷₂, —CH₂X¹⁷, —CN, —SOₙ₁₇R^{17A}, —SOᵥ₁₇NR^{17A}R^{17B}, —NHNR^{17A}R^{17B}, —ONR^{17A}R^{17B}, —NHC(O)NHNR^{17A}R^{17B}, —NHC(O)NR^{17A}R^{17B}, —N(O)ₘ₁₇, —NR^{17A}R^{17B}, —C(O)R^{17A}, —C(O)—OR^{17A}, —C(O)NR^{17A}R^{17B}, —OR^{17A}, —NR^{17A}SO₂R^{17B}, —NR^{17A}C(O)R^{17B}, —NR^{17A}C(O)OR^{17B}, —NR^{17A}OR^{17B}, —OCX¹⁷₃, —OCHX¹⁷₂, —OCH₂X¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁸ is independently hydrogen,
halogen, —CX¹83, —CHX¹82, —CH₂X¹⁸, —CN, —SOₙ₁₁₈R^{8A}, —SOᵥ₁₈NR^{18A}R^{18B}, —NHNR^{18A}R^{18B}, —ONR^{18A}R^{18B}, —NHC(O)NHNR^{18A}R^{18B},
—NHC(O)NR^{18A}R^{18B}, —N(O)ₘ₁₈, —NR^{18A}R^{8B}, —C(O)R^{18A}, —C(O)—OR^{18A}, —C(O)NR^{18A}R^{18B}, —OR^{18A}, —NR^{18A}SO₂R^{18B}, —NR^{18A}C(O)R^{18B}, —NR^{18A}C(O)OR^{18B}, —NR^{18A}OR^{18B}, —OCX¹⁸₃, —OCHX¹⁸₂, —OCH₂X¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁹ is independently hydrogen,
halogen, —CX¹⁹₃, —CHX¹⁹₂, —CH₂X¹⁹, —CN, —SOₙ₁₉R^{19A}, —SOᵥ₁₉NR^{19A}R^{19B}, —NHNR^{9A}R^{19B}, —ONR^{19A}R^{19B}, —NHC(O)NHNR^{19A}R^{19B},
—NHC(O)NR^{19A}R^{19B}, —N(O)ₘ₁₉, —NR^{19A}R^{19B}, —C(O)R^{19A}, —C(O)—OR¹⁹A —C(O)NR^{19A}R^{19B}, —OR^{19A}, —NR^{19A}SO₂R^{19B}, —NR^{19A}C(O)R^{19B}, —NR^{19A}C(O)OR^{19B}, —NR^{19A}OR^{19B}, —OCX¹⁹₃, —OCHX¹⁹₂, —OCH₂X¹⁹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R^{1A}, R^{1B}, R^{1C}, R^{1D}, R^{2A}, R^{2B}, R^{2C}, R^{2D}, R^{16A}, R^{16B}, R^{17A}, R^{17B}, R^{18A}, R^{18B}, R^{19A}, and R^{19B} are independently hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl;
X, X¹, X², X¹⁶, X¹⁷, X¹⁸, and X¹⁹ are independently —F, —Cl, —Br, or —I;
n1, n2, n16, n17, n18, and n19 are independently an integer from 0 to 4;
m1, m2, m16, m17, m18, m19, v1, v2, v16, v17, v18, and v19 are independently 1 or 2;
z1 is an integer from 0 to 5; and
z2 is an integer from 0 to 8.

Embodiment 2. The compound of embodiment 1, having the formula:

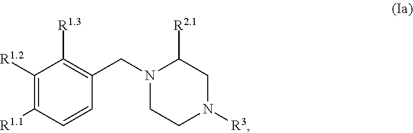

wherein,
R^{1.1}, R^{1.2}, and R^{1.3} are independently hydrogen, halogen, —CX¹³, —CHX¹², —CH₂X¹, —OCX¹³, —OCH₂X¹, —OCHX¹², —CN, —SOₙ₁R^{1D}, —NR^{1A}R^{1B}, —OR^{1D}, —SF₅, substituted or unsubstituted C₁-C₆ alkyl, substituted or unsubstituted 2 to 6 membered heteroalkyl, or substituted or unsubstituted 5 to 6 membered heteroaryl;
R^{2.1} is independently hydrogen, oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl;
R³ is independently —CN,

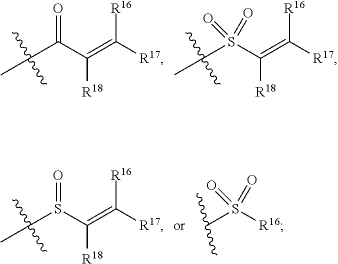

R¹⁶, R¹⁷, and R¹⁸ are independently hydrogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₇ cycloalkyl, or substituted or unsubstituted C₆-C₁₂ aryl;
R^{1A}, R^{1B}, and R^{1D} are independently
hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; and
X is independently —F, —Cl, —Br, or —I.

Embodiment 3. The compound of one of embodiments 1 to 2, having the formula:

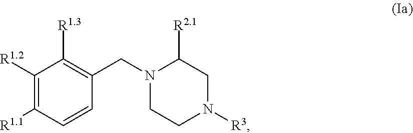

wherein,
R^{1.1}, R^{1.2}, and R^{1.3} are independently hydrogen, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, —SH, —SCH₃, —SCF₃, —SCHF₂, —SCH₂F, —SCCl ₃, —SCHCl₂, —SCH₂Cl, —SCBr₃, —SCHBr₂, —SCH₂Br, —SCl₃, —SCHI₂, —SCH₂I, —SOCH₃, —SO₂CH₃, —NH₂, —NHCH₃, —OH, —SF₅, substituted or unsubstituted C₁-C₆ alkyl, substituted or unsubstituted 2 to 6 membered heteroalkyl, or substituted or unsubstituted 5 to 6 membered heteroaryl;
R^{2.1} is independently hydrogen, oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
—NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl;
R³ is independently —CN,

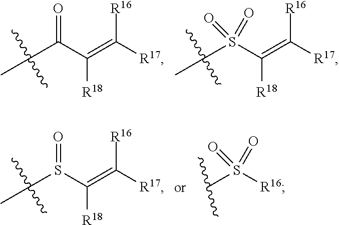

and
R¹⁶, R¹⁷, and R¹⁸ are independently hydrogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₆ cycloalkyl, or substituted C₆ aryl.

Embodiment 4. The compound of one of embodiments 1 to 3, wherein

R^{2.1} is independently hydrogen, substituted or unsubstituted C₁-C₆ alkyl, substituted or unsubstituted 2 to 6 membered heteroalkyl, substituted or unsubstituted C₆-C₁₂ aryl, or substituted or unsubstituted 5 to 12 membered heteroaryl.

Embodiment 5. The compound of one of embodiments 1 to 3, having the formula:

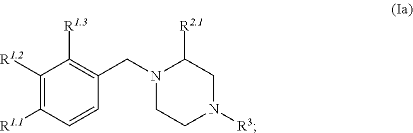

wherein,
R^{2.1} is independently —CH₂O—CH₂CCH, —CH₂O—CH₂CN, —CH₂O—CH₂-heterocycloalkyl, substituted or unsubstituted C₆-C₁₂ aryl, or substituted or unsubstituted 5 to 12 membered heteroaryl.

Embodiment 6. The compound of one of embodiments 1 to 3, wherein

R^{2.1} is independently hydrogen, substituted or unsubstituted C₁-C₆ alkyl, or substituted or unsubstituted 2 to 6 membered heteroalkyl.

Embodiment 7. The compound of one of embodiments 1 to 3, wherein

R^{2.1} is independently hydrogen, R²⁰-substituted or unsubstituted C₁-C₆ alkyl, or R²⁰-substituted or unsubstituted 2 to 6 membered heteroalkyl;
R²⁰ is independently —OH, R²¹-substituted or unsubstituted 5 to 6 membered heterocycloalkyl or R²¹-substituted or unsubstituted 5 to 6 membered heteroaryl; and
R²¹ is independently oxo.

Embodiment 8. The compound of embodiment 1, having the formula:

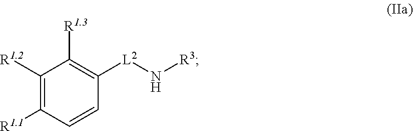

wherein,
R^{1.1}, R^{1.2}, and R^{1.3} are independently hydrogen, halogen, —CX¹³, —CHX¹², —CH₂X¹, —OCX¹³, —OCH₂X¹, —OCHX¹², —CN, —SOₙ₁R^{1D}, —NR^{1A}R^{1B}, —OR^{1D}, —SF₅, substituted or unsubstituted C₁-C₆ alkyl, substituted or unsubstituted 2 to 6 membered heteroalkyl, or substituted or unsubstituted 5 to 6 membered heteroaryl;
L² is unsubstituted C₁-C₆ alkylene;
R³ is independently —CN,

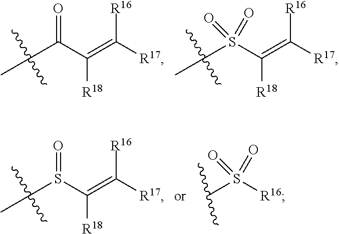

R¹⁶, R¹⁷, and R¹⁸ are independently hydrogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₇ cycloalkyl, or substituted or unsubstituted C₆-C₁₂ aryl;
R^{1A}, R^{1B}, and R^{1D} are independently
hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; and
X is independently —F, —Cl, —Br, or —I.

Embodiment 9. The compound of embodiments 1 or 8, having the formula:

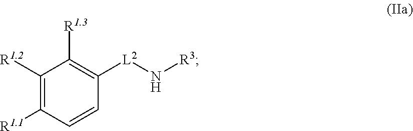

wherein,
R^{1.1}, R^{1.2}, and R^{1.3} are independently hydrogen, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, —SH, —SCH₃, —SCF₃, —SCHF₂, —SCH₂F, —SCCl ₃, —SCHCl₂, —SCH₂Cl, —SCBr₃, —SCHBr₂, —SCH₂Br, —SCl₃, —SCHI₂, —SCH₂I, —SOCH₃, —SO₂CH₃, —NH₂, —NHCH₃, —OH, —SF₅, substituted or unsubstituted C₁-C₆ alkyl, substituted or unsubstituted 2 to 6 membered heteroalkyl, or substituted or unsubstituted 5 to 6 membered heteroaryl;
L² is unsubstituted C₁-C₆ alkylene;
R³ is independently —CN, R

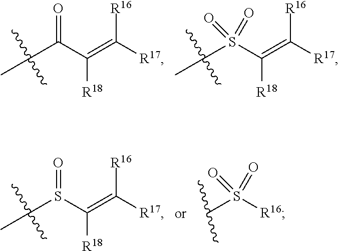

and
R¹⁶, R¹⁷, and R¹⁸ are independently hydrogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₆ cycloalkyl, or substituted C₆ aryl.

Embodiment 10. The compound of embodiment 9, wherein L² is unsubstituted n-propylene or unsubstituted n-butylene.

Embodiment 11. The compound of embodiments 2 or 10, wherein

R^{1.1} is independently
hydrogen, —OCCl₃, —OCF₃, —OCBr₃, —OCI₃, —OCHCl₂, —OCHBr₂, —OCHI₂, —OCHF₂, —OCH₂Cl, —O CH₂Br, —OCH₂I, —OCH₂F, —CN, —SH, —SCH₃, —SCF₃, —SCHF₂, —SCH₂F, —SCCl₃, —SCHCl₂, —SCH₂ Cl, —SCBr₃, —SCHBr₂, —SCH₂Br, —SCl₃, —SCHI₂, —SCH₂I, —SOCH₃, —SO₂CH₃, —NH₂, —NHCH₃, —OH, —SF₅, alkenyl, alkynyl, unsubstituted methoxy, unsubstituted ethoxy, unsubstituted n-propoxy, unsubstituted isopropoxy, unsubstituted n-butoxy, unsubstituted t-butoxy, unsubstituted sec-butoxy, unsubstituted isobutoxy, or unsubstituted pyrazolyl;
R^{1.2} is independently hydrogen, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, or unsubstituted C₁-C₄ alkyl; and
R^{1.3} is independently hydrogen,
halogen, —OCCl₃, —OCF₃, —OCBr₃, —OCI₃, —OCHCl₂, —OCHBr₂, —OCHI₂, —OCHF₂, —OCH₂Cl, —OC H₂Br, —OCH₂I, —OCH₂F, —CN, unsubstituted methoxy, unsubstituted ethoxy, unsubstituted n-propoxy, unsubstituted isopropoxy, unsubstituted n-butoxy, unsubstituted t-butoxy, unsubstituted sec-butoxy, or unsubstituted isobutoxy.

Embodiment 12. The compound of embodiments 2 or 10, wherein

R^{1.1} is independently hydrogen, —OCF₃, —CN, —SCH₃, —SCF₃, —SOCH₃, —SO₂CH₃, —NHCH₃, —SF₅, unsubstituted C₂-C₄ alkenyl, unsubstituted C₂-C₄ alkynyl, unsubstituted isopropoxy, or unsubstituted pyrazolyl;
R^{1.2} is independently hydrogen, —F, —Br, or —CF₃; and
R^{1.3} is independently hydrogen, —F, or —OCF₃.

Embodiment 13. The compound of one of embodiments 1 to 12, wherein

R³ is independently —CN.

Embodiment 14. The compound of one of embodiments 1 to 12, wherein

R³ is independently R

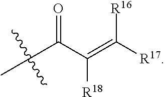

Embodiment 15. The compound of one of embodiments 1 to 12, wherein

R³ is independently

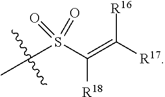

Embodiment 16. The compound of one of embodiments 1 to 12, wherein

R³ is independently

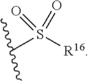

Embodiment 17. The compound of one of embodiments 1 to 12, wherein

R³ is independently

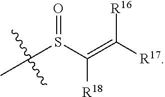

Embodiment 18. The compound of one of embodiments 1 to 17, wherein

R¹⁶ is hydrogen;
R¹⁷ is independently hydrogen, unsubstituted C₁-C₄ alkyl, or unsubstituted C₃-C₆ cycloalkyl; and
R¹⁸ is independently hydrogen, unsubstituted C₁-C₄ alkyl, or unsubstituted C₃-C₆ cycloalkyl.

Embodiment 19. The compound of one of embodiments 1 to 17, wherein

R¹⁶ is hydrogen;
R¹⁷ is independently hydrogen or unsubstituted C₁-C₄ alkyl; and
R¹⁸ is independently hydrogen or unsubstituted C₁-C₄ alkyl.

Embodiment 20. The compound of one of embodiments 1 to 17, wherein

R¹⁶ is hydrogen;
R¹⁷ is independently hydrogen, unsubstituted methyl, or unsubstituted cyclopropyl; and
R¹⁸ is independently hydrogen, unsubstituted methyl, or unsubstituted cyclopropyl.

Embodiment 21. The compound of one of embodiments 1 to 17, wherein

R¹⁶ is hydrogen;
R¹⁷ is independently hydrogen or unsubstituted methyl; and
R¹⁸ is independently hydrogen or unsubstituted methyl.

Embodiment 2₂. The compound of one of embodiments 1 to 17, wherein

R¹⁶, R¹⁷ and R¹⁸ are hydrogen.

Embodiment 2₃. The compound of embodiment 1, having the formula:

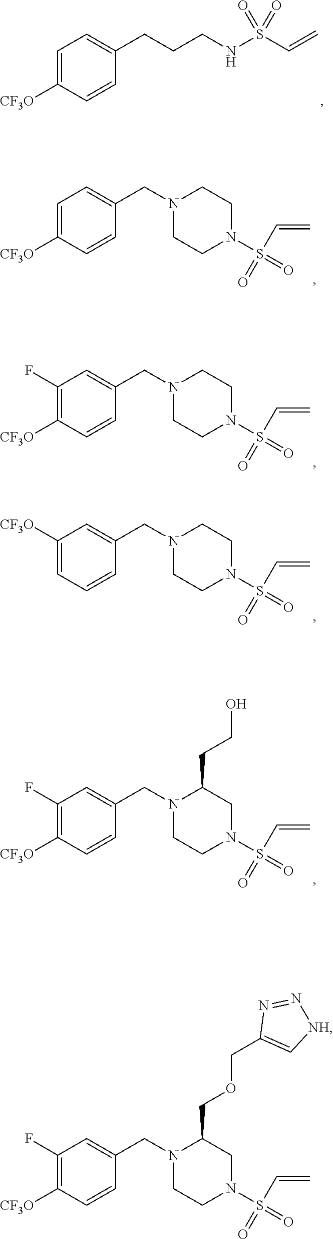

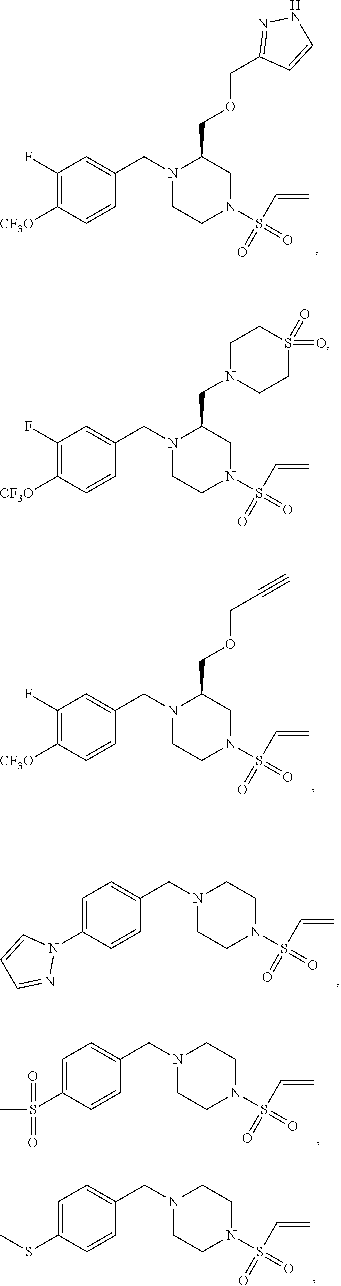

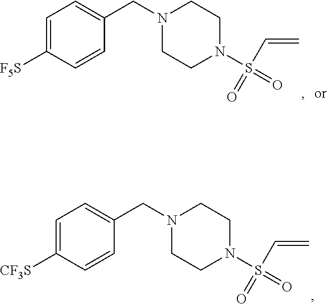

Embodiment 24. A pharmaceutical composition comprising the compound of any one of embodiments 1 to 2₃ and a pharmaceutically acceptable excipient.

Embodiment 25. A method of inhibiting Taspase1 protein activity, said method comprising: contacting the Taspase1 protein with a compound of one of embodiments 1 to 2₃.

Embodiment 26. A method of treating cancer, said method comprising administering to a subject in need thereof an effective amount of a compound of one of embodiments 1 to 2₃.

Embodiment 27. The method of embodiment 26, wherein the cancer is glioblastoma, melanoma, leukemia, lymphoma, ovarian cancer, renal cancer, colon cancer, prostate cancer, lung cancer, brain cancer, or breast cancer.

Embodiment 28. The method of embodiment 26, wherein the cancer is sensitive to Taspase1 inhibition.

Embodiment 29. A Taspase1 protein covalently bonded to a compound of one of embodiment 1 to 23.

Embodiment 30. The Taspase1 protein of embodiment 29, wherein the compound is bonded to a cysteine residue of the protein.

Embodiment 31. A Taspase protein covalently bonded to a portion of a compound of one of embodiments 1 to 23.

Embodiment 32. A compound of any one of embodiments 1 to 23, or a pharmaceutically acceptable salt thereof, for use in a method of treating cancer, comprising administering to a subject in need thereof an effective amount of the compound.

Embodiment 33. A compound for the use of embodiment 32, wherein the cancer is glioblastoma, melanoma, leukemia, lymphoma, ovarian cancer, renal cancer, colon cancer, prostate cancer, lung cancer, brain cancer, or breast cancer.

Embodiment 34. A compound for the use of embodiment 32, wherein the cancer is sensitive to Taspase1 inhibition.

### EXAMPLES

### Example 1: Optimization of Inhibitors of Taspase1

Taspase1 is a unique protease, first characterized by Professor James Hsieh, that contains a threonine residue as the active site nucleophile and cleaves substrates after an aspartate residue.^{3,4}. It is overexpressed in numerous liquid and solid malignancies and has been termed a ‘non-oncogene addiction’protease.^{1,2} Major substrates of Taspase1 include the master cell, epigenetic regulatory proteins MLL and the transcription factor (TF) IIA family of nuclear proteins that regulate the cell cycle.^{5,6} Loss of Taspase1 disrupts proliferation of human cancer cell lines in vitro and reduces growth of tumor xenograft models of several aggressive tumor types. Taspase1 is overexpressed in multiple cancer cell lines and loss of Taspase1 sensitizes glioblastoma and melanoma cells to chemotherapy-induced apoptosis.⁷ Other evidence points to a key role of Taspase1 in invasion and metastasis via proteolysis of MLL in HepG2 hepatocellular carcinoma metastasis models in vitro and in vivo.⁸ Loss of Taspase1 strongly inhibits development of HER2-driven breast tumors and EGFR-driven lung cancer (including drug-resistant, EGFR-T790M mutant tumors). Thus, growth factor-driven, drug-resistant cancers represent promising clinical indications for Taspase1 inhibitors.

An alternative hit-finding approach using a tethering screen, inspired by successful lead compounds targeting KRAS G12C,^{9,10} to target selective covalent modification of a noncatalytic cysteine residue in the Taspase1 substrate-binding groove was accompanied by expanded protein crystallography efforts to drive a structure-based design optimization strategy. This approach produced the first and only known potent Taspase1 inhibitors.

The iterative process of chemical optimization and biological testing has led to the discovery of 21 covalent inhibitors with biochemical IC₅₀<100 nM and increased cell-based potency, now enabling studies of Taspase1 inhibition in cell lines that represent clinically-relevant models.

Designs for Taspase1 inhibitors have benefited from X-ray co-structures that describe the binding modes of inhibitors, yielding molecules with improved on-target cellular efficacy, including inhibition of proliferation.

Crystallography efforts have supported structure-based drug design with co-crystal structures of inhibitors (both covalent and non-covalent) bound to truncated Taspase1 as either a 2-chain or single-chain circularly permuted protein, informing the SAR strategy by identifying regions where additional interactions might increase potency.

Warhead-bearing piperazine compounds that display ˜20 nM Taspase1 inhibition have been shown to exhibit on-mechanism cellular activity. Mass spectrometry-based tethering assays confirmed specific, concentration dependent covalent modification of the enzyme at C293. Most inhibitors show minimal inhibition of a Taspase1 mutant lacking the targeted cysteine (C293A), indicating that a large portion of the potency is due to covalency. Despite this, specific SAR trends demonstrate that binding site interactions effectively modulate inhibition. A primary optimization objective has been to increase pocket occupation and thus reduce reliance on the reactive vinyl sulfonamide warhead. This has been substantially guided by the available co-crystal structures of inhibitors bound to Taspase1 adjacent to the catalytic threonine (T234).

In parallel with the efforts to optimize binding interactions, the team has undertaken a concerted effort to explore less reactive chemical moieties used in approved drugs and clinical candidates. New electrophiles were designed to provide appropriate spacing to react with Cys293, or the catalytic Threonine and were evaluated using piperazine substitutions known to enable potent Taspase1 inhibition. No compounds intended to interact covalently with the catalytic Threonine have yet shown inhibition of Taspase1, but compounds with alternative Cysteine-reactive warheads have demonstrated low micromolar inhibition of the enzyme.

The most potent covalent inhibitors approach the enzymatic assay sensitivity limits. To extend that assay, compounds are also tested in the presence of 6.6 mM GSH as a non-specific, but physiologically relevant, thiol competitor. Selected analogs, notably those with piperazine substitutions (SMDC128), exhibit a smaller drop-off in the presence of 6.6 mM GSH, predicting less sensitivity to intracellular antioxidants and the prospect for better cellular activity.

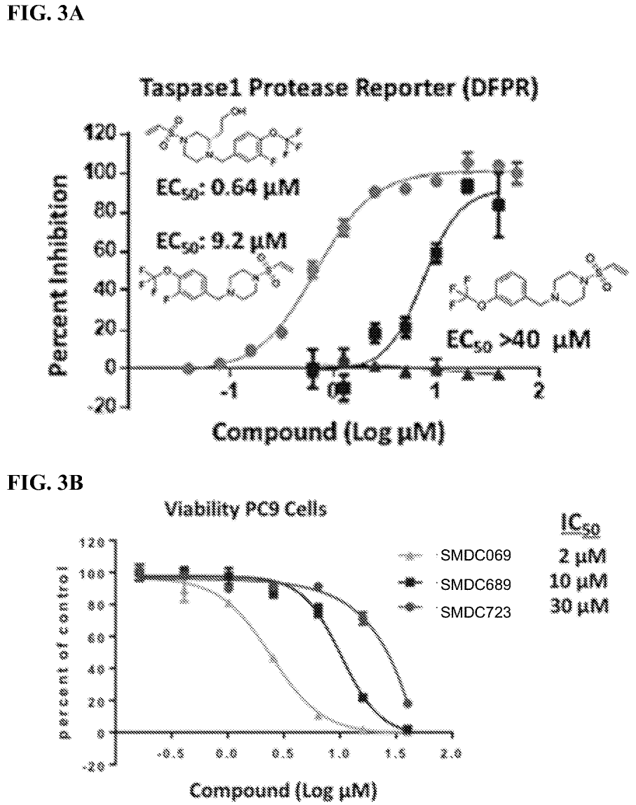

An additional 30 compounds in the series were shown to exhibit dose-dependent activity (EC₅₀ less than 40 μM) in a cell-based assay that monitors cleavage of a Taspase1 substrate (FIG. 3A). Compounds such as SMDC069 are superior; the fluorescence measured activity has been confirmed by immunoblotting in cells. A closely related Taspase1 inactive control analog that retains the vinylsulfonamide electrophile, SMDC723, shows no inhibition of substrate cleavage in cells, demonstrating that the cell-based activity is not driven solely by the warhead, but requires specific binding interactions with Taspase1 in cells, as it does in the biochemical enzyme assay.

The ratio of cell to biochemical potency can vary, possibly due to reaction of the vinyl sulfonamide warhead with cytosolic components such as GSH. We have reduced the gap between enzyme and cell-based activity with analogs such as SMDC069, with a ˜30-fold ratio of cellular to biochemical potency compared to the ˜200 fold drop-off with SMDC689.

Using compounds with increased on-target cellular activity assay, it is now feasible to examine inhibition of cell growth. Taspase1 shRNA knockdown produces target-specific cell growth reduction in specific cancer cell lines. We tested whether inhibitors could phenocopy these results by treating cell monolayers with varying doses of SMDC069, SMDC689, and an inactive control, with the same warhead. FIG. 3B shows that SMDC069 reduced viability of EGFR mutant PC9 lung cancer cells after 72h with an IC50 of ˜2 μM and demonstrates improvement over weaker Taspase1 inhibitors.

In vitro experiments showed that electrophilic piperazine compounds have uniformly excellent solubility and permeability, with no efflux liability. Most examples showed moderate to good microsomal stability. Taken together with the improved cellular data, this suggests a promising profile for discovering future inhibitors worthy of testing in vivo.

TABLE 1
Activity table of Taspase1 inhibitors, as described herein.

| | *Biochemical IC₅₀ (nM) | | LC-MS | | | | |
|---|---|---|---|---|---|---|---|
| | | +6.6 mM | DR₅₀ | | Kᵢ | k_{inact} | DFPR EC₅₀ |
| Cmpd | no GSH | GSH | (nM) | | (uM) | (s⁻¹) | (uM) |
| SMDC967 | 168 | n/a | 637 | (3) | 6.02 (1) | 0.011 (1) | X |
| | (22) +/− 56 | | | | | | |
| SMDC687 | 51 | 661 | 129 | (3) | 32.9 | 0.028 | 11.8 |
| | (73) +/− 48 | (14) +/− 158 | | | (5) +/− 11.2 | (5) +/− 0.002 | (18) +/− 6.2 |
| SMDC069 | 17 | 273 | 47 | (3) | 23.2 (1) | 0.082 (1) | 2.8 |
| | (6) +/− 6 | (3) +/− 83 | | | | | (4) +/− 2.6 |
| SMDC203 | 22 | 237 | 287 | (3) | 27.7 (1) | 0.048 (1) | 1.8 |
| | (3) +/− 10 | (3) +/− 49 | | | | | (2) +/− 0.7 |
| SMDC883 | 26 | 340 | 84 | (3) | 64.3 (1) | 0.037 (1) | 7.5 (1) |
| | (6) +/− 9 | (2) +/− 42 | | | | | |
| SMDC275 | 27 | 420 | 76 | (3) | X | X | X |
| | (3) +/− 9 | (3) +/− 98 | | | | | |
| **SMDC723 | 10 × 103 | n/a | 73,620 | (3) | X | X | >40 (1) |
| | (1) | | | | | | |

*3FAM/QXL520 fluorescent dyes; *
**SMDC723 = negative control for cytotoxicity studies

TABLE 2
Additional activity of Taspase1 inhibitors, as described herein.

| | | | |
|---|---|---|---|
| | PC9 | H1975 | MDA-MB-231 |
| | [Lung] | [Lung] | [Breast] |
| Cmpd | (uM) | (uM) | (uM) |

| | | | | | | |
|---|---|---|---|---|---|---|
| SMDC069 | 2.5 | (L3) | 2.1 | (L3) | 3.2 | (L3) |
| | 8.5 | (L4) | 7.4 | (L4) | ~13 | (L4) |
| SMDC203 | 10.5 | (L2) | 8.1 | (L2) | ~13 | (L2) |
| SMDC275 | 3.5 | (L1) | 2.6 | (L1) | 4.3 | (L1) |
| SMDC723 | ~22 | (L1) | ~20 | (L1) | ~37 | (L1) |
| Neg. control | | | | | | |

We propose to further optimize the current covalent and non-covalent scaffolds using new structural information, particularly co-crystal structures of compounds bound to full-length Taspase 1, to build in non-covalent interactions and decrease reactivity through use of alternative warheads. In the future we plan to further improve cell-based potency over our current best-in-class inhibitors, retain acceptable solubility, permeability and microsomal stability properties and begin evaluating optimized compounds in POC in vivo models reported above to be Taspase sensitive.

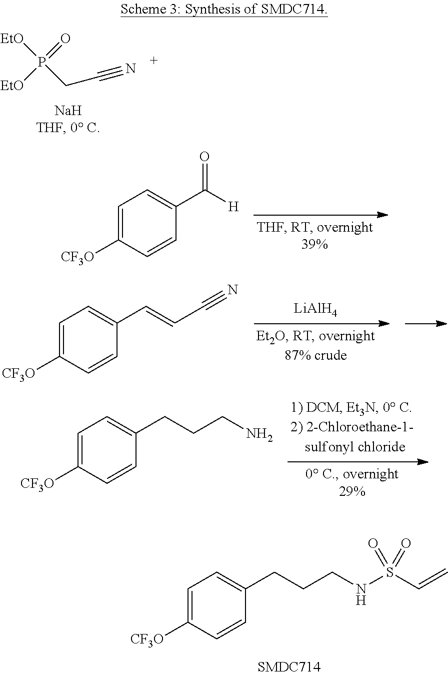

[4-(trifluoromethoxy)phenyl]prop-2-enenitrile: By the procedure of the cited reference (applies to parts 1 and 2), Sodium hydride 60% dispersion in mineral oil (0.558 g, 14.0 mmol 1.00 eq) was dissolved in dry Tetrahydrofuran (17 ml) and stirred in an ice bath under dry Argon. Diethyl cyanomethyl phosphonate (2.25 ml, 14.0 mmol, 1.00 eq) was added. 4-(Trifluoromethoxy)benzaldehyde (1.99 ml, 14.0 mmol, 1.00 eq) was added as a solution in Tetrahydrofuran (5 ml). The reaction was stirred and allowed to warm to room temperature overnight. The reaction was quenched with water and the reaction mixture was partitioned between water and Ethyl acetate with the water layer extracted twice. The combined organic layer was organics were dried with brine and Sodium sulfate, decanted, concentrated to afford a residue which was dissolved in 10:1 hexane ether and hexane was added until 20:1 when crystallization afforded pure intermediate (1.18 g, 5.55 mmol) 39% yield which was used directly in the next reaction. ¹H NMR (300 MHz, CHLOROFORM-d) δ ppm 5.88 (d, J=16.58 Hz, 1H) 7.26 (d, J=8.67 Hz, 2H) 7.40 (d, J=16.58 Hz, 1H) 7.45-7.55 (m, 2H).

3-[4-(trifluoromethoxy)phenyl]propan-1-amine: Lithium aluminum hydride (304 mg, 8.02 mmol, 3.20 eq) was suspended in dry diethyl ether (15 ml) with stirring under dry argon. This was heated to 50° C. and a solution of [4-(trifluoromethoxy)phenyl]prop-2-enenitrile (534 mg, 2.51 mmol, 1 eq) in diethyl ether (5 ml) was added dropwise with a syringe. Heating was continued for 1.5 hours, the reaction was cooled to room temperature and stirred overnight. The reaction was quenched by slow addition of water (5 ml) in an ice bath followed by 1M NaOH (10 ml). The mixture was vigorously stirred with Ethyl acetate for 0.5 hours, partitioned and the water layer was extracted twice more with Ethyl acetate. The combined organics were dried with brine then sodium sulfate, filtered and concentrated to afford crude 3-[4-(trifluoromethoxy)phenyl]propan-1-amine (481 mg, 2.19 mmol) 87% yield, crude which was used crude in the next step. ¹H NMR (300 MHz, CHLOROFORM-d) δ ppm 1.64-1.76 (m, 2H) 2.55-2.63 (m, 2H) 2.66 (t, J=7.06 Hz, 2H) 7.03-7.11 (m, 2H) 7.11-7.17 (m, 2H).

[4-(trifluoromethoxy)phenyl]propyl}ethene-1-Sulfonamide (SMDC714): 3-[4-(trifluoromethoxy)phenyl]propan-1-amine (74.1 mg, 0.385 mmol, 1.00 eq) was dissolved in DCM (4 mL) and cooled to 0° C. The reaction mixture was treated with trimethylamine (2.00 equiv). To it was added 98.0% 2-Chloroethanesulfonyl chloride (1.0 equiv.). The reaction mixture was stirred for 2 h at cold. Then was added Trimethylamine (2 equiv.) at 0° C. The reaction was stirred for 2 h. The reaction mixture was evaporated and loaded on a 12 g Silicycle cartridge in a gradient of Ethyl acetate 0-30% in Hexane to afford the product (34.8 mg, 0.113 mmol) 29% yield. ¹H NMR (300 MHz, CHLOROFORM-d) δ ppm 1.79-1.94 (m, 2H) 2.63-2.73 (m, 2H) 3.04 (q, J=6.84 Hz, 2H) 4.68 (t, J=5.93 Hz, 1H) 5.94 (d, J=9.80 Hz, 1H) 6.23 (d, J=16.58 Hz, 1H) 6.51 (dd, J=16.48, 9.89 Hz, 1H) 7.08-7.16 (m, 2H) 7.16-7.24 (m, 2H); 13C NMR (75 MHz, CHLOROFORM-d) δ ppm 31.45, 31.99, 42.33, 121.10, 122.20, 126.89, 129.66, 135.76, 139.63, 147.62; 19 F NMR (282 MHz, METHANOL-d4) δ ppm −57.93 (s, 1 F); LCMS (ESI+) m/z 310.30 (M+H+).

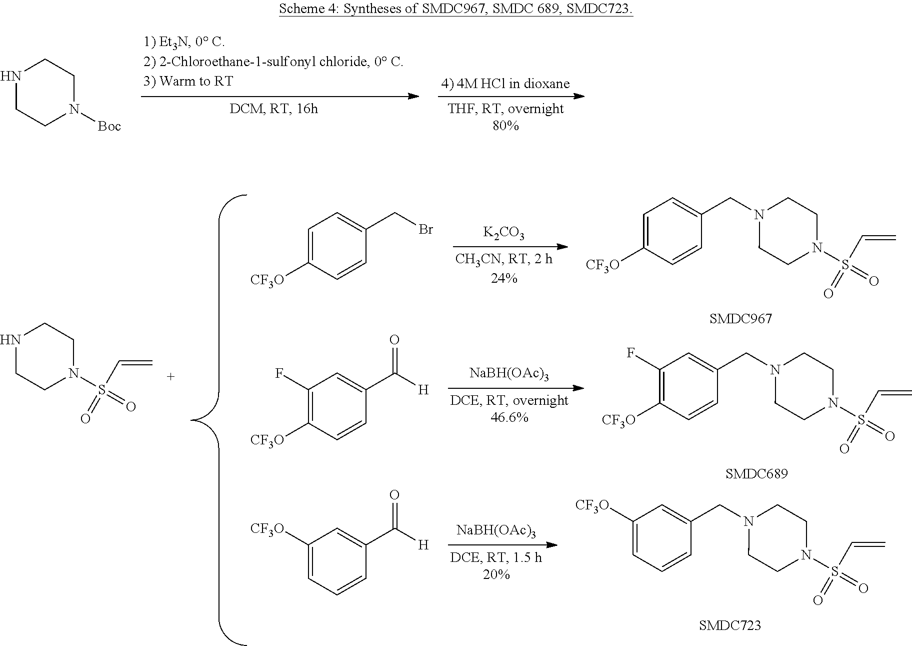

1-(Ethenesulfonyl)piperazine hydrochloride: N-Boc piperazine (10.0 g, 53.7 mmol, 1.0 eq), was dissolved in CH₂Cl₂ (400 mL, 0.13 M), and Et₃N (20 mL, 214 mmol, 4.0 eq) was added. The mixture was cooled to 0° C. in an ice bath and 2-chloroethane sulfonyl chloride (7.86 mL, 75.2 mmol, 1.4 eq) was added. After addition was complete, the reaction mixture was allowed to warm to room temperature and stirred for 16 hours. The reaction was diluted with H₂O and extracted with CH₂Cl₂ (×3). The combined organic layers were washed with saturated aqueous NaCl, dried over MgSO₄, filtered, and concentrated in vacuo. The crude material was purified on silica (0-70% gradient of EtOAc in Hexanes). ¹H NMR (400 MHz, MeOD): δ 6.64 (dd, 1H, J=10.0, 16.5 Hz), 6.19 (d, 1H, J=16.5 Hz), 6.13 (d, 1H, J=10.0 Hz), 3.52 (4H, t, J=4.78 Hz), 3.10 (4H, t, J=5.2 Hz), 1.46 (s, 9H).

This material was dissolved in THE (˜200 mL) and 4 M HCl in dioxane was added (134 mL, 537 mmol, 10 eq) and the reaction was allowed to stir overnight at room temperature. The reaction mixture was then concentrated in vacuo and dried under low pressure to give 9.11 g (80% yield over 2 steps) of 1-(ethenesulfonyl)piperazine hydrochloride as an off-white powder, which was used without further purification. ¹H NMR (400 MHz, MeOD): δ 6.72 (dd, 1H, J=10.0, 16.5 Hz), 6.27 (d, 1H, J=16.5 Hz), 6.21 (d, 1H, J=10.0 Hz), 3.44-3.40 (m, 4H), 3.36-3.30 (m, 4H); MS (ES): m/z 177.1 [M+H]+

1-(ethenesulfonyl)-4-{[4-(trifluoromethoxy)phenyl]methyl}piperazine (SMDC967): 1-(ethenesulfonyl)piperazine hydrochloride (122 mg, 0.6 mmol, 1.0 equiv.) was suspended in a septum-capped 20 vial in ordinary non-dried reagent grade acetonitrile (3 ml), potassium carbonate (159 mg, 1.1 mmol, 2.0 equiv.) was added, followed by 4-(Trifluoromethoxy)benzyl bromide (97 ul, 0.6 mmol, 1.0 equiv.). The reaction mixture was stirred at room temperature for 2 hours, filtered through celite, concentrated, dissolved in a small amount dichloromethane and eluted on a 12 g Silicycle cartridge in a gradient of Ethyl Acetate 0-100% in Hexane. The product was further purified by HPLC 30-80% Methanol (0.05% Formic acid both) in water over 12 min 18 min total, to afford the product (49 mg, 0.1 mmol) 24% yield as a clear oil. ¹H NMR (300 MHz, CHLOROFORM-d) δ ppm 2.39-2.72 (m, 4H) 3.10-3.27 (m, 4H) 3.55 (s, 2H) 6.06 (d, J=9.98 Hz, 1H) 6.25 (d, J=16.58 Hz, 1H) 6.44 (dd, J=16.58, 9.80 Hz, 1H) 7.17 (m, J=8.29 Hz, 2H) 7.34 (m, J=8.29 Hz, 2H), 19F NMR (282 MHz, CHLOROFORM-d) δ ppm 57.87 (s, 1 F) 13C NMR (75 MHz, CHLOROFORM-d) δ ppm 45.62 (s, 1 C) 52.21 (s, 1 C) 61.68 (s, 1 C) 76.58 (s, 1 C) 77.43 (s, 1 C) 120.89 (s, 1 C) 128.92 (s, 1C) 130.18 (s, 1 C) 132.13 (s, 1 C) 136.38 (s, 1 C); LCMS (ESI+) m/z 351.1 (M+H+).

1-(Ethenesulfonyl)-4-{[3-fluoro-4-(trifluoromethoxy)phenyl]methyl}piperazine (SMDC689) (2): 3-Fluoro-4-(trifluoromethoxy)benzaldehyde (78 mg, 0.392 mmol, 1.0 eq.) and 1 (100 mg, 0.47 mmol, 1.2 eq) were dissolved in dichloroethane (2.5 mL, 0.16 M). NaBH(OAc)₃ was added, and the reaction was allowed to stir overnight at room temperature. After confirmation of product formation by LC/MS, the reaction was quenched with 2 M NaOH, then extracted with CH₂Cl₂ (×3). The combined organic layers were then washed with saturated aqueous NaCl, dried over MgSO₄, and concentrated in vacuo. The crude residue was dissolved in MeOH and purified by HPLC to give 66.1 mg (45.6% yield) of the desired product. ¹H NMR (400 MHz, CDCl₃): δ 7.40-7.37 (m, 2H), 7.32 (d, 1H, J=8.0 Hz), 6.44 (dd, 1H, J=9.8, 16.5 Hz), 6.29 (d, 1H, J=16.5 Hz), 6.15 (d, 1H, J=9.8 Hz), 4.15 (s, 1H), 3.57 (bs, 4H), 3.22 (bs, 4H); ¹³C NMR (100 MHz, CDCl₃): δ 42.84, 51.23, 59.64, 120.37 (q, J=249, 518), 120.25 (d, J=20 Hz), 124.56, 127.57 (d, J=2 Hz), 128.69 (d, 7 Hz), 130.98, 131.55, 138.16 (d, 12 Hz), 154.56 (d, J=255 Hz); DEPT-135 (100 MHz, CDCl₃): δ 42.74 (CH2), 51.14 (CH2), 59.56 (CH2), 120.15 (d, J=19 Hz, CH), 124.47 (CH), 127.46 (d, J=4 Hz, CH), 130.88 (CH2), 131.46 (CH); MS (ES) m/z 369.3 [M+H]+.

1-(3-(Trifluoromethoxy)benzyl)-4-(vinyl sulfonyl)piperazine (SMDC723): To 100 mg of 1 (100 mg, 0.47 mmol) in dichloroethane (4.0 mL) was added 3-(trifluoromethoxy)benzaldehyde (70 μL, 0.56 mmol) followed by NaBH(OAc)₃ (350 mg, 1.61 mmol). The reaction vessel capped, purged with Argon, and stirred for 1.5 h at rt. The reaction solution was then diluted with CH₂Cl₂ (20 mL) and washed with 1 M aqueous NaOH (15 mL). The aqueous layer was then extracted 3× with CH₂Cl₂ (20 mL). The combined organics were then washed with brine and dried with MgSO₄. The crude product was absorbed onto celite and purified on a C18 column with 0-50% water (0.1 TFA): MeCN to afford 1-(3-(trifluoromethoxy)benzyl)-4-(vinyl sulfonyl)piperazine (33.5 mg, 20%).¹H NMR (400 MHz; MeOD): δ 7.64-7.60 (m, 1H), 7.54-7.51 (m, 2H), 7.45 (d, J=8.2 Hz, 1H), 6.71 (dd, J=16.6, 10.0 Hz, 1H), 6.28 (d, J=16.6 Hz, 1H), 6.22 (d, J=10.0 Hz, 1H), 4.38 (s, 2H), 3.44 (bs, 4H), 3.33 (bs, 4H); 13C NMR (100 MHz, CDCl₃): δ 42.76, 51.22, 60.24, 120.38 (q, J=171, 343 Hz), 123.00, 123.51, 129.43, 129.76, 131.03, 131.25, 131.48, 149.84; DEPT-135 (100 MHz, CDCl₃): δ 42.67 (CH2), 51.13 (CH2), 60.14 (CH2), 122.89 (CH), 123.41 (CH), 129.32 (CH), 130.90 (CH2), 131.15 (CH), 131.41 (CH); MS (ES) m/z 351.0 [M+H]+.

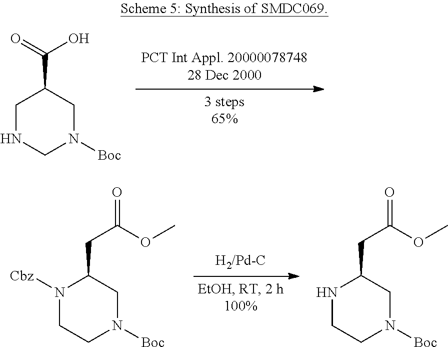

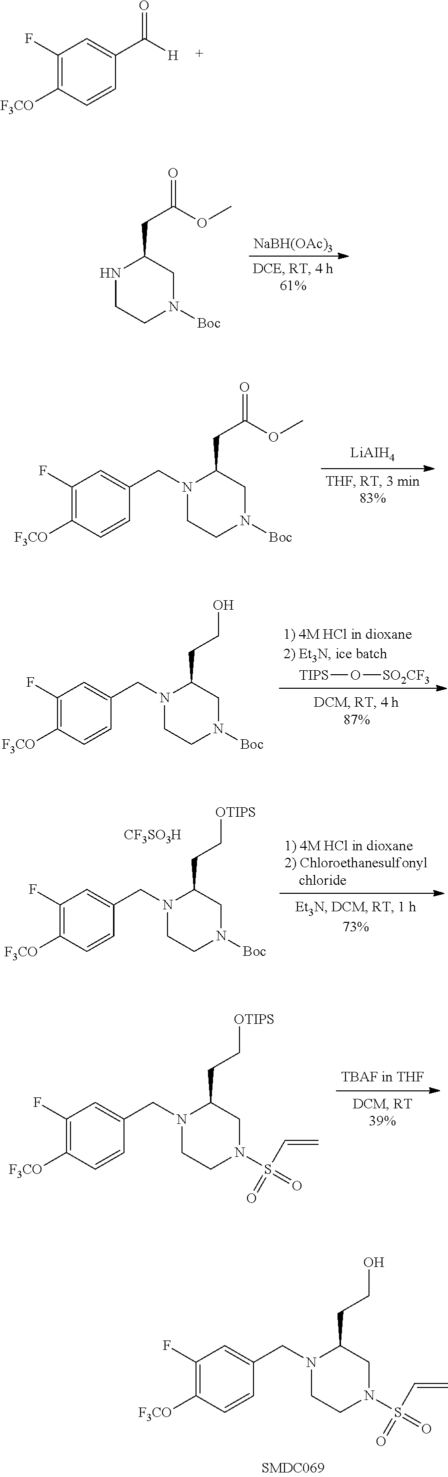

tert-butyl (3S)-3-(2-methoxy-2-oxoethyl)piperazine-1-carboxylate: 1-benzyl 4-tert-butyl (25)-2-(2-methoxy-2-oxoethyl)piperazine-1,4-dicarboxylate (1.23 g, 3.13 mmol, 1 eq) was dissolved in Ethanol (116 ml) and Palladium 10% in Carbon (465 mg) was added. The flask was fitted with a balloon adaptor, evacuated, and purged with hydrogen 3 times. The reaction mixture was then stirred under hydrogen for 2 hours, then filtered through celite and concentrated to afford a sooty-colored oil which was used in the next step. ¹H NMR (400 MHz, CHLOROFORM-d) δ ppm 7.93-10.19 (brs, 1H) 4.05-4.28 (m, 1H) 3.93-4.06 (m, 1H) 3.72 (s, 2H) 3.69-3.78 (m, 1H) 3.53-3.63 (m, 1H) 3.19-3.54 (m, 1H) 3.07-3.18 (m, 1H) 2.95-3.06 (m, 1H) 2.69-2.85 (m, 1H) 1.43 (s, 5H).

tert-butyl (3 S)-4-{[3-fluoro-4-(trifluoromethoxy)phenyl]methyl}-3-(2-methoxy-2-oxoethyl)piperazine-1-carboxylate: tert-butyl (3S)-3-(2-methoxy-2-oxoethyl)piperazine-1-carboxylate (509 mg, 1.97 mmol, 1 eq) was reacted with 3-fluoro-(trifluoromethoxy)benzaldehyde (0.341 ml, 1.97 mmol, 1 eq) in DCE (15 mL). Half an hour later, sodium triacetoxyborohydride (1.4 equiv.) was added to the above reaction mixture. The reaction mixture was monitored by LCMS and TLC. Next day, 12 h later, 100 mg more sodium triacetoxyborohydride was added to push the reaction mixture to the completion. 12 h later, the reaction mixture was treated with sat. 10% saturated solution of sodium bicarbonate solution (100 mL). Then the organic layer was extracted with dichloromethane (2×80 mL), the combined layers was washed with water (2×100 mL), dried over Na₂SO₄, and concentrated under reduced pressure to afford a residue which was purified on a 25 g Silicycle cartridge in a gradient of 0-100% Ethyl acetate in hexane to afford a product (546 mg, 1.21 mmol) 61% yield. ¹H NMR (400 MHz, CHLOROFORM-d) δ ppm 7.08-7.22 (m, 1H) 6.94-7.07 (m, 1H) 3.61 (br. s, 2H) 3.49-3.79 (m, 4H) 2.92-3.48 (m, 3H) 2.34-2.58 (m, 2H) 2.13-2.34 (m, 1H) 1.37 (s, 6H) 1.25-1.47 (m, 6H) 1.25-1.47 (m, 6H); 13C NMR (100 MHz, CHLOROFORM-d) δ ppm 172.06 (s, 1 C) 155.56 (s, 1 C) 154.59 (s, 1 C) 153.05 (s, 1 C) 139.86 (s, 1 C) 134.97 (s, 1 C) 124.04 (s, 1 C) 123.21 (s, 1 C) 121.57 (s, 1 C) 118.99 (s, 1 C) 116.66 (s, 1 C) 79.54 (s, 1 C) 57.11 (s, 1 C) 55.31 (s, 1 C) 51.45 (s, 1 C) 47.50 (s, 1C) 46.83 (s, 1 C) 42.39 (s, 1 C) 29.93 (s, 1 C) 28.03 (s, 1 C); 19F NMR (376 MHz, CHLOROFORM-d) δ ppm −59.04 (d, J=4.09 Hz, 3 F) 129.27-−129.16 (m, 1 F); LCMS (ESI+) m/z 451.16 (M+H+)

tert-butyl (3 S)-4-{[3-fluoro-4-(trifluoromethoxy)phenyl]methyl}-3-(2-hydroxyethyl) piperazine-1-carboxylate: Lithium aluminum hydride (54 mg, 1.42 mmol, 1.33 eq) was stirred in dry Tetrahydrofuran (15 ml) under dry argon with stirring. To this was added a solution of tert-butyl (3 S)-4-{[3-fluoro-4-(trifluoromethoxy)phenyl]methyl}-3-(2-methoxy-2-oxoethyl)piperazine-1-carboxylate (480 mg, 1.07 mmol, 1.00 eq) in 10 ml tetrahydrofuran via syringe over a 3 minute period at room temperature. The reaction was monitored to be complete by TLC. If not complete a small amount of LiAlH₄ may be added. The reaction was then cooled in an icebath with rapid stirring quenched by careful addition of 1.5 ml water, then 5 ml 2M NaOH, then 3 ml more water. The resulting mixture was filtered through celite then extracted twice with Diethyl ether. The combined organics were dried with brine and sodium sulfate, filtered and concentrated. The residue was dissolved in minimal Dichloromethane and eluted on a 25 g Silicycle cartridge in a gradient of Ethyl acetate in Hexane 20-100% to afford the product (371 mg, 0.880 mmol) 83% yield. ¹H NMR (400 MHz, CHLOROFORM-d) δ ppm 7.12-7.22 (m, 1H) 7.02-7.08 (m, 1H) 3.77-3.95 (m, 1H) 3.59-3.77 (m, 2H) 3.34 (br. s., 3H) 2.53-2.71 (m, 1H) 2.10-2.24 (m, 1H) 1.75-1.89 (m, 1H) 1.62-1.75 (m, 1H) 1.39 (s, 6H); 13C NMR (100 MHz, CHLOROFORM-d) δ ppm 155.58 (s, 1 C) 154.85 (s, 1 C) 153.07 (s, 1 C)

(s, 1 C) 139.95 (s, 1 C) 135.13 (s, 1 C) 135.10 (s, 1 C) 135.08 (s, 1 C) 134.99 (s, 1 C) 134.98 (s, 1 C) 134.96 (s, 1 C) 124.23 (s, 1 C) 124.20 (s, 1 C) 123.29 (s, 1 C) 121.60 (s, 1 C) 119.02 (s, 1 C) 117.03 (s, 1 C) 116.84 (s, 1 C) 116.45 (s, 1 C) 79.87 (s, 1 C) 79.73 (s, 1 C) 77.32 (s, 1 C) 76.69 (s, 1 C) 59.81 (s, 1 C) 56.99 (s, 1 C) 56.61 (s, 1 C) 48.57 (s, 1 C) 47.73 (s, 1 C) 45.95 (s, 1 C) 43.13 (s, 1 C) 41.84 (s, 1 C) 29.42 (s, 1 C) 28.15 (s, 1 C); 19F NMR (376 MHz, CHLOROFORM-d) δ ppm −58.97 (d, J=4.09 Hz, 1 F) −129.19-−128.97 (m, 1 F); LCMS (ESI+) m/z 423.11 (M+H+).

(2S)-1-{[3-fluoro-4-(trifluoromethoxy)phenyl]methyl}-2-(2-{[tris(propan-2-yl)silyl]oxy}ethyl)piperazine; trifluoromethanesulfonic acid salt: tert-butyl (3S)-4-{[3-fluoro-4-(trifluoromethoxy)phenyl]methyl}-3-(2-hydroxyethyl)piperazine-1-carboxylate (443 mg, 1.05 mmol, 1.00 eq) was dissolved in dry dichloromethane (3 ml) and treated with 4M HCl in dry 1,4-dioxane (6 ml). The mixture was stirred for 2 hours under dry argon, then evaporated to afford a gummy solid which was immediately redissolved and stirred in dry dichloromethane (30 ml). To this was added Triethylamine (439 ul, 3.15 mmol, 3.00 eq) and the mixture was cooled in an icebath. Triisopropylsilyl trifluoromethanesulfonate (353 ul, 1.31 mmol, 1.25 equiv.) was added and the reaction mixture was allowed to warm to room temperature and checked for completion by TLC Upon completion (4 hours), the reaction was quenched by addition of saturated Sodium bicarbonate, partitioned between water and ethyl acetate with the water layer extracted twice with ethyl acetate, the combined organics were dried with brine, sodium sulfate, filtered and concentrated. The residue was redissolved in a small amount of Dichloromethane then eluted in a gradient of 0-10% Methanol in Dichloromethane to afford the product (435 mg, 0.909 mol) 87% yield. ¹H NMR (400 MHz, CHLOROFORM-d) δ ppm 7.16-7.26 (m, 1H) 7.09 (d, J=8.52 Hz, 1H) 6.24 (s, 1H) 3.96 (d, J=14.12 Hz, 1H) 3.71-3.83 (m, 1H) 3.30 (d, J=14.37 Hz, 1H) 3.19-3.27 (m, 1H) 3.06-3.16 (m, 1H) 2.94-3.06 (m, 1H) 2.85 (br. s., 1H) 2.74-2.81 (m, 1H) 2.38 (br. s., 1H) 1.85-1.97 (m, 1H) 1.72-1.85 (m, 1H) 0.94-1.13 (m, 23H); 13C NMR (100 MHz, CHLOROFORM-d) δ ppm 155.77 (s, 1 C) 153.26 (s, 1 C) 139.80 (s, 1 C) 139.74 (s, 1 C) 135.32 (s, 1 C) 135.31 (s, 1 C) 135.20 (s, 1 C) 135.18 (s, 1 C) 124.77 (s, 1 C) 124.27 (s, 1 C) 124.08 (s, 1 C) 124.05 (s, 1 C) 123.48 (s, 1 C) 121.70 (s, 1 C) 121.59 (s, 1 C) 119.12 (s, 1 C) 118.42 (s, 1 C) 116.89 (s, 1 C) 116.70 (s, 1 C) 116.55 (s, 1 C) 115.25 (s, 1 C) 59.76 (s, 1 C) 56.39 (s, 1 C) 55.81 (s, 1 C) 48.22 (s, 1 C) 48.08 (s, 1 C) 44.11 (s, 1 C) 31.19 (s, 1 C) 17.85 (s, 1 C) 11.76 (s, 1 C); 19F NMR (376 MHz, CHLOROFORM-d) δ ppm −58.99 (d, J=4.09 Hz, 3 F) 78.53 (s, 1 F) −129.24-−128.60 (m, 1 F); LCMS (ESI+) m/z 479.26 (M+H+).

(2S)-4-(ethenesulfonyl)-1-{[3-fluoro-4-(trifluoromethoxy)phenyl]methyl}-2-(2-{[tris(propan-2-yl)silyl]oxy}ethyl)piperazine: (2S)-1-{[3-fluoro-4-(trifluoromethoxy)phenyl]methyl}-2-(2-{[tris(propan-2-yl)silyl]oxy}ethyl)piperazine; trifluoromethanesulfonic acid salt (217 mg, 0.345 mmol, 1.00 eq) was dissolved in DCM and cooled to 0° C. The reaction mixture was treated with trimethylamine (2.00 equiv). To it was added 98.0% 2-Chloroethanesulfonyl chloride (1.0 equiv.). The reaction mixture was stirred for 2 h at cold. Then was added Trimethylamine (2 equiv.) at 0° C. The reaction was stirred for 2 h. The reaction mixture was evaporated to afford a crude product which was eluted on a 12 g Silicycle cartridge in a gradient of ethyl acetate in hexane to give the product (143 mg, 0.252 mmol) 73% yield. ¹H NMR (400 MHz, CHLOROFORM-d) δ ppm 7.18-7.25 (m, 1H) 7.09 (dd, J=8.52, 0.73 Hz, 1H) 6.43 (dd, J=9.98, 6.57 Hz, 1H) 6.23 (d, J=16.56 Hz, 1H) 6.05 (d, J=9.98 Hz, 1H) 3.77-3.85 (m, 1H) 3.75 (s, 1H) 3.46 (d, J=14.37 Hz, 1H) 3.20 (d, J=4.63 Hz, 1H) 3.03-3.11 (m, 1H) 2.90-2.95 (m, 1H) 2.72-2.80 (m, 1H) 2.36-2.44 (m, 1H) 1.80-1.89 (m, 1H) 0.96-1.12 (m, 10H); 13C NMR (100 MHz, CHLOROFORM-d) δ ppm 155.77 (s, 1 C) 153.26 (s, 1 C) 140.25 (s, 1 C) 140.19 (s, 1 C) 135.15 (dq, J=12.47, 1.71 Hz, 1 C) 132.09 (s, 1 C) 128.80 (s, 1 C) 124.01 (d, J=3.67 Hz, 1 C) 123.41 (s, 1 C) 121.71 (s, 1 C) 120.41 (q, J=258.00 Hz, 1 C) 116.86 (s, 1 C) 60.14 (s, 1C) 56.63 (s, 1 C) 56.61 (s, 1 C) 55.57 (s, 1 C) 48.81 (s, 1 C) 47.88 (s, 1 C) 45.30 (s, 1 C) 29.04 (s, 1 C) 17.94 (s, 1 C) 11.81 (s, 1 C); 19F NMR (376 MHz, CHLOROFORM-d) δ ppm −58.90 (d, J=5.45 Hz, 3F) −129.07-−129.00 (m, 1F).

2-[(2S)-4-(ethenesulfonyl)-1-{[3-fluoro-4-(trifluoromethoxy)phenyl]methyl}piperazin-2-yl]ethan-1-ol (SMDC069): (2S)-4-(ethenesulfonyl)-1-{[3-fluoro-4-(trifluoromethoxy)phenyl]methyl}-2-(2-{[tris(propan-2-yl)silyl]oxy}ethyl)piperazine (143 mg, 0.252 mmol, 1.00 eq) was dissolved in dry Dichloromethane (4 ml) and stirred under dry argon. Tetrabutyl ammonium fluoride solution in THE (7 ml) was added and the progress of the reaction was monitored frequently by TLC to prevent formation of the cyclic product. As soon as the starting material disappeared, the reaction was quenched by the addition of 50% saturated Sodium bicarbonate in water. The mixture was diluted with Ethyl acetate and water and the water layer was extracted twice. The combined organic layer was dried with brine then Sodium sulfate, filtered and concentrated to afford a residue which was re-dissolved in Dichloromethane and purified on a 4 g Silicycle cartridge. Further purification was achieved by HPLC on C18 in a gradient of 30-80% Methanol in Water 12 min 18 total each 0.05% Formic acid. Since the UV absorbance of the compound was low in the 200-300 nm UV range, a sensitive collection method was used to obtain the product (41 mg, 0.098 mmol) 39% yield. ¹H NMR (400 MHz, CHLOROFORM-d) δ ppm 7.18-7.27 (m, 2H) 7.10 (d, J=8.28 Hz, 1H) 6.44 (dd, J=16.68, 9.86 Hz, 1H) 6.25 (d, J=16.56 Hz, 1H) 6.08 (d, J=9.98 Hz, 1H) 3.92 (d, J=13.88 Hz, 1H) 3.77-3.88 (m, 1H) 3.73 (ddd, J=10.84, 7.18, 5.60 Hz, 1H) 3.47 (d, J=13.88 Hz, 1H) 3.23-3.29 (m, 1H) 3.04-3.23 (m, 3H) 2.88-2.95 (m, 1H) 2.79-2.88 (m, 1H) 2.64 (s, 1H) 2.42 (ddd, J=12.30, 5.97, 3.65 Hz, 1H) 1.82-2.03 (m, 2H); 13C NMR (100 MHz, CHLOROFORM-d) δ ppm 155.74 (s, 1 C) 153.23 (s, 1 C) 139.58 (d, J=5.87 Hz, 1 C) 135.31 (d, J=10.27 Hz, 1 C) 131.95 (s, 1C) 129.31 (s, 1 C) 129.12 (s, 1 C) 124.26 (d, J=3.67 Hz, 1 C) 123.51 (s, 1 C) 121.67 (s, 1 C) 120.39 (q, J=258.00 Hz, 1 C) 117.08 (s, 1 C) 116.89 (s, 1 C) 116.52 (s, 1 C) 60.02 (s, 1 C) 56.55 (d, J=1.47 Hz, 1 C) 56.42 (s, 1 C) 48.32 (s, 1 C) 47.70 (s, 1 C) 44.67 (s, 1 C) 28.72 (s, 1 C); LCMS (ESI+) m/z 413.04 (M+H+).

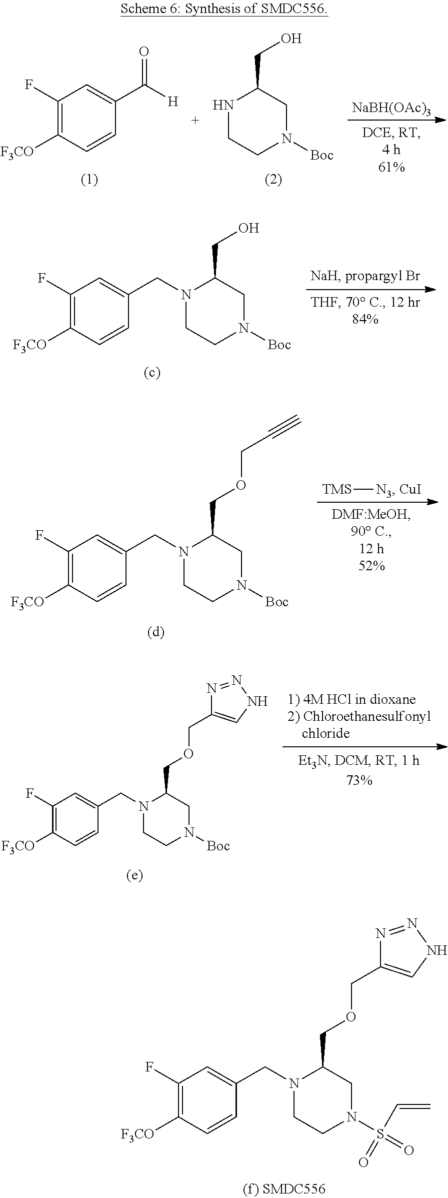

tert-butyl (R)-4-(3-fluoro-4-(trifluoromethoxy)benzyl)-3-(hydroxymethyl)piperazine-1-carboxylate (c): To a solution of 3-fluoro-4-(trifluoromethoxy)benzaldehyde 1 (2.19 g, 10.2 mmol, 1.0 equiv.) in DCE (60 mL) was added tert-butyl (R)-3-(hydroxymethyl)piperazine-1-carboxylate 2 (2.19 g, 9.65 mmol, 1.0 equiv.). Half an hour later, sodium triacetoxyborohydride (3.02 g, 14.3 mmol, 1.4 equiv.) was added to the above reaction mixture. The reaction mixture was monitored by LCMS and TLC. Next day, 12 h later, 500 mg more sodium triacetoxyborohydride was added to push the reaction mixture to the completion. 12 h later, the reaction mixture was treated with sat. 10% saturated solution of sodium bicarbonate solution (100 mL). Then the organic layer was extracted with dichloromethane (2×80 mL), the combined layers was washed with water (2×100 mL), dried over Na₂SO₄, and concentrated under reduced pressure. The crude liquid was loaded on to a 120 g silica column. The crude was purified by a gradient 100% hexane to 100% ethyl acetate. The yield was 68.4%. ¹H NMR (400 MHz; METHANOL-d4) S: 7.32-7.40 (m, 2H), 7.26 (d, J=8.0 Hz, 1H), 4.86 (s, 1H), 4.06-4.14 (m, 1H), 3.73-3.81 (m, 2H), 3.53-3.68 (m, 2H), 3.46 (d, J=14.2 Hz, 1H), 3.14-3.29 (m, 2H), 2.64-2.72 (m, 1H), 2.45-2.52 (m, 1H), 2.16-2.25 (m, 1H), 1.46 (s, 9H); 13C NMR (100 MHz; METHANOL-d4) δ: 157.3, 156.9, 154.8, 142.9, 136.7, 136.5, 126.5, 125.0, 123.6, 121.0, 118.8, 81.6, 62.8, 61.9, 58.5, 51.3, 50.0, 29.0, 21.2, 14.8; 19F NMR (376 MHz; METHANOL-d4) S: 60.59 (s, 1F), −131.71 (s, 1F); MS m/z=431 as a Na+ salt.

tert-butyl (R)-4-(3-fluoro-4-(trifluoromethoxy)benzyl)-3-((prop-2-yn-1-yloxy)methyl) piperazine-1-carboxylate (d): 60.0% Sodium hydride (482 mg, 12.0 mmol, 4.20 equiv.) was added to a solution of tert-butyl (3R)-4-{[3-fluoro-4-(trifluoromethoxy)phenyl]methyl}-3-(hydroxymethyl)piperazine-1-carboxylate c (1.16 g, 2.80 mmol, 1.00 equiv.) in tetrahydrofuran (6.00 mL) at 0° C. under N₂. The reaction mixture was stirred under nitrogen for 60 min. Then 80.0% propargyl bromide in toluene (9.40 ml, 6.0 mmol, 2.1 equiv.) was added to the reaction mixture at 0° C. The reaction mixture was stirred for 12 h at 70° C. The reaction mixture was cooled to room temperature and treated with ice water. The reaction mixture was poured in a separator funnel, extracted with ethyl acetate (3×50 mL), dried over MgSO₄ and concentrated under reduced pressure. Then the crude mixture was loaded on to a 40 g silica column and purified with a gradient 100% hexane to 100% ethyl acetate. The yield was 80%. ¹H NMR (400 MHz; CHLOROFORM-d) S: 7.28-7.51 (m, 1H), 7.20-7.26 (m, 2H), 7.12 (br d, J=8.4 Hz, 1H), 4.71 (s, 1H), 3.95-4.19 (m, 3H), 3.89-3.91 (m, 1H), 3.52-3.78 (m, 3H), 3.41-3.52 (m, 2H), 3.21-3.37 (m, 1H), 2.77 (s, 1H), 2.57-2.73 (m, 2H), 2.43 (t, J=2.1 Hz, 1H), 2.13-2.34 (m, 1H), 2.05 (s, 1H), 1.64 (br d, J=15.9 Hz, 1H), 1.43-1.50 (m, 9H); 13C NMR (100 MHz; CHLOROFORM-d) δ: 155.7, 154.8, 153.2, 140.4, 124.4, 123.4, 121.8, 117.3, 117.1, 79.8, 79.3, 77.3, 77.2, 77.0, 76.7, 74.8, 68.4, 60.7, 60.4, 58.5, 57.6, 28.4, 14.2; 19F NMR (376 MHz; CHLOROFORM-d) δ: −58.86 (s, 1F), −129.16 (s, 1F); MS m/z=447.4

tert-butyl (R)-3-(((1H-1,2,3-triazol-4-yl)methoxy)methyl)-4-(3-fluoro-4-(trifluoro methoxy)benzyl)piperazine-1-carboxylate (e): tert-butyl (3R)-4-{[3-fluoro-4-(trifluoromethoxy)phenyl]methyl}-3-[(prop-2-yn-1-yloxy)methyl]piperazine-1-carboxylate d (0.239 g, 0.60 mmol, 1.0 equiv.) and copper(I) Iodide (0.005 g, 0.05 equiv.) was added in a mixture of N,N-dimethylformamide (5.69 ml, 73.8 mmol) and methanol (0.46 ml,) at 0° C. Then was added TMS azide (0.1 ml, 0.6 mmol, 1.5 equiv.) dropwise and heat the mixture at 90° C. for 18 hours. There was no starting material left in the reaction mixture and then the reaction mixture was passed via a celite plug. The reaction mass was concentrated under reduced pressure and loaded on to a column. The crude mixture was loaded on to a 12 g silica column and eluted with 50% hexane to 50% ethyl acetate to afford e (0.20 g, 76% yield). ¹H NMR (400 MHz; CHLOROFORM-d) δ: 7.48-7.78 (m, 1H), 7.21 (br d, J=9.5 Hz, 2H), 6.99-7.12 (m, 1H), 4.52-4.82 (m, 2H), 3.84-4.02 (m, 1H), 3.47 (br s, 8H), 2.52-2.86 (m, 2H), 2.07-2.36 (m, 1H), 1.46 (s, 10H); 13C NMR (100 MHz; CHLOROFORM-d) δ: 156.0, 155.3, 153.5, 140.2, 140.2, 135.6, 124.7, 124.7, 123.7, 122.0, 119.5, 117.6, 117.4, 80.5, 77.6, 58.9, 58.9, 57.9, 53.7, 49.4, 30.0, 28.7; 19F NMR (376 MHz; CHLOROFORM-d) δ: −58.84 (s, 1F), −58.86 (s, 1F), −129.07 (s, 1F); MS m/z=490.17.

(R)-2-(((1H-1,2,3-triazol-4-yl)methoxy)methyl)-1-(3-fluoro-4-(trifluoromethoxy) benzyl)-4-(vinyl sulfonyl)piperazine (f) (SMDC556): tert-butyl (R)-3-(((1H-1, 2, 3-triazol-5-yl) methoxy) methyl)-4-(3-fluoro-4-(trifluoromethoxy) benzyl)piperazine-1-carboxylate e (0.1 g, 0.2 mmol, 1.0 equiv.) was treated with 4 M hydrochloric acid (1.502 ml, 6.0 mmol, 30.0 equiv.) in 1,4-dioxane in cold. The reaction mixture was stirred for 1 h and then LCMS was checked. The crude reaction mixture was evaporated under reduced pressure. To it was added N, N-diisopropylethylamine (0.073 ml, 0.4 mmol, 2.0 equiv.) and then was added 2-chloroethanesulfonyl chloride (0.021 ml, 0.2 mmol, 1.0 equiv.) at 0° C. The reaction was stirred for 2 h at cold. Then at cold was added N, N-diisopropylethylamine (0.070 ml, 0.4 mmol, 2.0 equiv.). The reaction mixture was stirred for 2 hr at room temperature. The crude was purified by silica column and then by reverse phase HPLC using 100% water to 100% methanol to afford f (0.01 g, 10% yield). ¹H NMR (400 MHz; CHLOROFORM-d) δ: 7.72 (s, 1H), 7.16-7.25 (m, 2H), 6.99-7.11 (m, 1H), 6.36-6.53 (m, 1H), 6.20-6.33 (m, 1H), 5.93-6.15 (m, 1H), 4.57-4.79 (m, 2H), 3.84-3.95 (m, 1H), 3.64-3.80 (m, 2H), 3.45-3.61 (m, 1H), 3.06-3.31 (m, 4H), 2.86-2.98 (m, 1H), 2.70-2.82 (m, 1H), 2.30-2.51 (m, 1H); 13C NMR (100 MHz; CHLOROFORM-d) δ: 155.8, 153.3, 139.8, 131.9, 131.4, 129.2, 124.2, 123.5, 117.1, 116.9, 77.8, 67.3, 64.1, 58.2, 57.3, 48.4, 47.6, 45.4; 19F NMR (376 MHz; CHLOROFORM-d) δ: −58.84 (s, 1F), −58.85 (s, 1F), −58.86 (s, 1F), −128.75 (s, 1F), −128.76 (s, 1F), MS m/z=480.4.

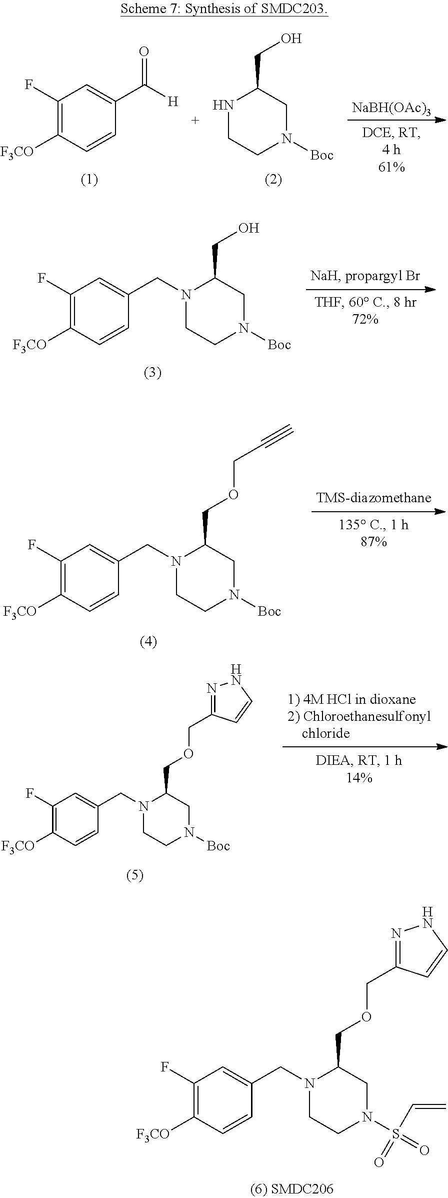

tert-butyl (R)-4-(3-fluoro-4-(trifluoromethoxy)benzyl)-3-(hydroxymethyl)piperazine-1-carboxylate (3): To a solution of 3-fluoro-4-(trifluoromethoxy)benzaldehyde 1 (2.19 g, 10.2 mmol, 1.0 equiv.) in DCE (60 mL) was added tert-butyl (R)-3-(hydroxymethyl)piperazine-1-carboxylate 2 (2.19 g, 9.65 mmol, and 1.00 equiv). Half an hour later, sodium triacetoxyborohydride (3.02 g, 14.3 mmol, 1.4 equiv.) was added to the above reaction mixture. The reaction mixture was monitored by LCMS and TLC. Next day, 12 h later, 500 mg more sodium triacetoxy borrohydride was added to push the reaction mixture to the completion. 12 h later, the reaction mixture was treated with sat. 10% saturated solution of sodium bicarbonate solution (100 mL). Then the organic layer was extracted with dichloromethane (2×80 mL), the combined layers was washed with water (2×100 mL), dried over Na₂SO₄, and concentrated under reduced pressure. The crude liquid was loaded on to a 120 g silica column. The crude was purified by a gradient 100% hexane to 100% ethyl acetate. The yield was 68.4%. ¹H NMR (400 MHz; METHANOL-d4) S: 7.32-7.40 (m, 2H), 7.26 (d, J=8.0 Hz, 1H), 4.86 (s, 1H), 4.06-4.14 (m, 1H), 3.73-3.81 (m, 2H), 3.53-3.68 (m, 2H), 3.46 (d, J=14.2 Hz, 1H), 3.14-3.29 (m, 2H), 2.64-2.72 (m, 1H), 2.45-2.52 (m, 1H), 2.16-2.25 (m, 1H), 1.46 (s, 9H); 13C NMR (100 MHz; METHANOL-d4) δ: 157.3, 156.9, 154.8, 142.9, 136.7, 136.5, 126.5, 125.0, 123.6, 121.0, 118.8, 81.6, 62.8, 61.9, 58.5, 51.3, 50.0, 29.0, 21.2, 14.8; 19F NMR (376 MHz; METHANOL-d4) δ: 60.59 (s, 1F), −131.71 (s, 1F); MS m/z=431 as a Na+ salt.

tert-butyl (R)-4-(3-fluoro-4-(trifluoromethoxy)benzyl)-3-((prop-2-yn-1-yloxy)methyl)-piperazine-1-carboxylate (4): 60.0% Sodium hydride (482 mg, 12.0 mmol, 4.20 equiv.) was added to a solution of tert-butyl (3R)-4-{[3-fluoro-4-(trifluoromethoxy)phenyl]methyl}-3-(hydroxymethyl)piperazine-1-carboxylate 3 (1.16 g, 2.80 mmol, 1.00 equiv.) in tetrahydrofuran (6.00 mL) at 0° C. under N₂. The reaction mixture was stirred under nitrogen for 60 min. Then 80.0% propargyl bromide in toluene (9.40 ml, 6.0 mmol, 2.1 equiv.) was added to the reaction mixture at 0° C. The reaction mixture was stirred for 12 h at 70° C. The reaction mixture was cooled to room temperature and treated with ice water. The reaction mixture was poured in a separator funnel, extracted with ethyl acetate (3×50 mL), dried over MgSO₄ and concentrated under reduced pressure. Then the crude mixture was loaded on to a 40 g silica column and purified with a gradient 100% hexane to 100% ethyl acetate. The yield was 80%. ¹H NMR (400 MHz; CHLOROFORM-d) δ: 7.28-7.51 (m, 1H), 7.20-7.26 (m, 2H), 7.12 (br d, J=8.4 Hz, 1H), 4.71 (s, 1H), 3.95-4.19 (m, 3H), 3.89-3.91 (m, 1H), 3.52-3.78 (m, 3H), 3.41-3.52 (m, 2H), 3.21-3.37 (m, 1H), 2.77 (s, 1H), 2.57-2.73 (m, 2H), 2.43 (t, J=2.1 Hz, 1H), 2.13-2.34 (m, 1H), 2.05 (s, 1H), 1.64 (br d, J=15.9 Hz, 1H), 1.43-1.50 (m, 9H); 13C NMR (100 MHz; CHLOROFORM-d) δ: 155.7, 154.8, 153.2, 140.4, 124.4, 123.4, 121.8, 117.3, 117.1, 79.8, 79.3, 77.3, 77.2, 77.0, 76.7, 74.8, 68.4, 60.7, 60.4, 58.5, 57.6, 28.4, 14.2; 19F NMR (376 MHz; CHLOROFORM-d) δ: −58.86 (s, 1F), −129.16 (s, 1F); MS m/z=447.4.

tert-butyl (R)-3-(((1H-pyrazol-4-yl)methoxy)methyl)-4-(3-fluoro-4-(trifluoro methoxy) benzyl)piperazine-1-carboxylate (5): To a sealed tube was added tert-butyl (3R)-4-{[3-fluoro-4-(trifluoromethoxy)phenyl]methyl}-3-[(prop-2-yn-1-yloxy)methyl]piperazine-1-carboxylate 4 (0.61 g, 1.40 mmol, 1.00 equiv.) and (Trimethylsilyl)diazomethane (2.00 ml, 4.00 mmol, 2.90 equiv.) in hexane. The reaction mixture was heated at 135° C. for 1 h in a sealed tube. [Use a face shield while doing the reaction. Extremely careful while doing this reaction]. LCMS indicated the completion, total consumption of the starting material. The crude reaction mixture was dried under reduced pressure and then was loaded on to a 40 g silica column. The column was purified by 10% methanol in 90% dichloromethane. The yield was 84%. ¹H NMR (400 MHz; METHANOL-d4) δ: 7.61 (br s, 1H), 7.28-7.37 (m, 2H), 7.19 (d, J=8.6 Hz, 1H), 6.31 (br s, 1H), 5.48 (s, 1H), 4.85 (s, 2H), 4.52 (br s, 2H), 4.44 (s, 1H), 4.02-4.19 (m, 1H), 3.96 (br d, J=14.1 Hz, 1H), 3.59-3.73 (m, 2H), 3.33-3.57 (m, 3H), 3.07-3.29 (m, 2H), 2.52-2.74 (m, 2H), 2.22 (br s, 1H), 1.44 (s, 9H); 13C NMR (100 MHz; METHANOL-d4) δ: 157.7, 155.2, 143.3, 137.1, 126.9, 125.4, 124.0, 121.4, 119.0, 106.4, 82.1, 61.1, 59.1, 55.6, 50.0, 29.4; 19F NMR (376 MHz; METHANOL-d4) δ:-60.61 (s, 1F), −131.79 (s, 1F); MS m/z=511 as a Na+ salt.

(R)-2-(((1H-pyrazol-4-yl)methoxy)methyl)-1-(3-fluoro-4-(trifluoromethoxy)benzyl)-4-(vinyl sulfonyl)piperazine (6) (SMDC203): tert-butyl (R)-3-(((1H-pyrazol-4-yl)methoxy) methyl)-4-(3-fluoro-4-(trifluoromethoxy)benzyl)piperazine-1-carboxylate 5 (0.36 g, 0.80 mmol, 1.0 equiv.) was dissolved in dichloromethane (3 mL) and cooled to 0° C. The reaction mixture was treated with hydrochloric acid (0.939 ml, 3.8 mmol, 10 equiv.). The reaction mixture was stirred for 1 h and then LCMS was checked. The reaction was complete, and the reaction mixture was concentrated under reduced pressure. The gummy liquid was triturated with diethyl ether (3×5 mL). Then the reaction mixture was cooled to 0° C. The reaction mixture was treated with trimethylamine (0.71 mL, 4.09 mmol, 2.00 equiv.). To it was added 98.0% 2-Chloroethanesulfonyl chloride (0.062 ml, 0.8 mmol, 1.0 equiv.). The reaction mixture was stirred for 2 h at cold. Then was added Trimethylamine (0.71 ml, 4.09 mmol, 2 equiv.) at 0° C. The reaction was stirred for 2 h. The reaction mixture was evaporated and loaded in a column. The crude was purified by 100% DCM to 100% methanol and finally was purified by reverse phase HPLC using 100% water to 100% methanol. ¹H NMR (400 MHz; METHANOL-d4) δ: 7.58 (s, 1H), 7.24-7.40 (m, 2H), 7.19 (br d, J=8.4 Hz, 1H), 6.51-6.72 (m, 1H), 6.26-6.40 (m, 1H), 6.07-6.23 (m, 1H), 4.43-4.64 (m, 2H), 3.96 (d, J=14.2 Hz, 1H), 3.56-3.85 (m, 2H), 3.33-3.55 (m, 2H), 3.16 (br dd, J=6.3, 3.3 Hz, 1H), 2.90-3.11 (m, 2H), 2.69-2.87 (m, 2H), 2.66 (s, 1H), 2.38 (br dd, J=7.7, 4.2 Hz, 1H); 13C NMR (100 MHz; CHLOROFORM-d) δ: 158.0, 155.5, 143.5, 134.6, 130.8, 127.2, 125.7, 119.4, 106.8, 70.0, 67.6, 61.0, 59.0, 58.3, 51.3, 49.1, 47.5, 43.6, 18.5, 13.8; 19F NMR (376 MHz; METHANOL-d4) δ: −60.62 (s, 1F), −131.74 (s, 1F); MS m/z=479.13 (dimer)

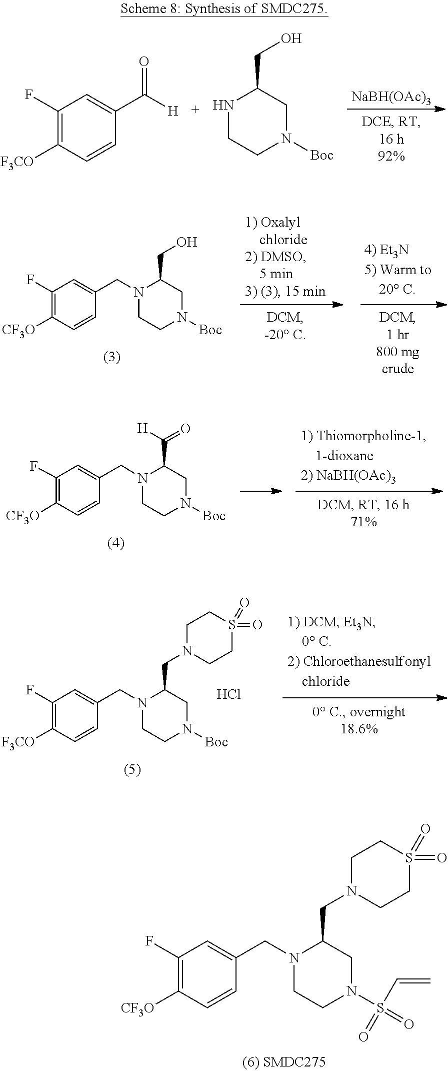

tert-Butyl-(3R)-4-{[3-fluoro-4-(trifluoromethoxy)phenyl]methyl}-3-(hydroxymethyl) piperazine-1-carboxylate (3): tert-Butyl (3R)-3-(hydroxymethyl)piperazine-1-carboxylate (2.5 g, 11.56 mmol, 1.0 eq), dichloroethane (20 mL, 0.58 M), and 3-fluoro-4-(trifluoromethoxy)benzaldehyde (2.4 mL, 17.34 mmol, 1.5 eq) were added to a 100 mL round bottom flask and allowed to stir for 5 minutes at room temperature. NaBH(OAc)₃ (7.4 g, 34.68 mmol. 3.0 eq) was added, and the reaction was allowed to stir for an additional 16 hours, then carefully quenched by the addition of 1 M aqueous NaOH and extracted with CH₂Cl₂ (×3). The combined organics were dried over MgSO₄, filtered, and concentrated in vacuo. The resulting residue was purified on silica (Hexanes/EtOAc, 0-45% gradient) to afford 4.34 g (92% yield) of tert-butyl-(3R)-4-{[3-fluoro-4-(trifluoromethoxy)phenyl]methyl}-3-(hydroxymethyl)piperazine-1-carboxylate. ¹H NMR (400 MHz, CDCl₃): δ 7.26 (m, 2H), 7.10 (d, 1H, J=8.3 Hz), 3.99 (d, 1H, J=13.8 Hz), 3.80 (dd, 1H, J=5.9, 11.5 Hz), 3.64 (dd, 2H, J=4.1, 11.5 Hz), 3.50 (bs, 1H), 3.46 (d, 2H, J=13.9 Hz), 3.27 (bs, 1H), 2.74 (bs, 1H), 2.62 (bs, 1H), 2.39 (bs, 1H), 2.30-2.24 (m, 1H), 1.45 (2, 9H); MS (ES): m/z 409.4 [M+H]+.

tert-Butyl (3R)-4-{[3-fluoro-4-(trifluoromethoxy)phenyl]methyl}-3-formylpiperazine-1-carboxylate (4): A solution containing oxalyl chloride (0.24 mL, 2.7 mmol, 1.1 eq,) in CH₂Cl₂ (30 mL, anhydrous) was cooled to −20° C., after which a solution of DMSO (0.35 mL, 4.9 mmol, 2.0 eq,) in CH₂Cl₂ (6.0 mL, anhydrous) was added and the mixture was allowed to stir for 5 min. A solution of 3 (1.0 g, 2.4 mmol, 1.0 eq) in CH₂Cl₂ (6.0 mL, anhydrous) was then added at −20° C. and the reaction was allowed to stir for 15 min. Et₃N (1.7 mL, 12 mmol, 5.0 eq) was slowly added, and the reaction was allowed to warm to 20° C. and stir for 1 hour. The reaction was diluted with CH₂Cl₂ and then washed sequentially with H2O, 2 M HCl, and saturated aqueous NaHCO₃. The organic layer was dried over MgSO₄, filtered, and concentrated in vacuo to give ˜800 mg of crude material as a clear, reddish oil that was used without any further purification. ¹H NMR (400 MHz, CDCl₃): δ 9.69 (d, 1H, J=2.0 Hz), 7.29-7.23 (m, 2H), 7.11 (d, 1H, J=8.8 Hz), 3.92 (d, 1H, J=14.0 Hz), 3.71 (bs, 1H), 3.62-3.58 (m, 2H), 3.43 (bs, 2H), 3.12 (bs, 1H), 2.96-2.90 (m, 1H), 2.36-2.30 (m, 1H), 1.45 (s, 9H); MS (ES): m/z 407.3 [M+H]+.

4-{[(2R)-1-{[3-Fluoro-4-(trifluoromethoxy)phenyl]methyl}piperazin-2-yl]methyl}-1λ⁶-thiomorpholine-1,1-dione Hydrochloride (5): Thiomorpholine 1,1-dioxide (173 mg, 1.28 mmol, 1.3 eq) was added to a solution of 4 (400 mg, 984 μmol, 1.0 eq,) in CH₂Cl₂ (8 mL) at room temperature. The reaction mixture was allowed to stir for 5 min and NaBH(OAc)₃ (834 mg, 3.94 mmol, 4.0 eq) was added. The reaction mixture was allowed to stir at room temperature for an additional 16 hour, then quenched with H₂O and extracted with CH₂Cl₂ (×3). The combined organic layers were washed with saturated aqueous NaHCO₃, followed by saturated aqueous NaCl. The organic layers were then dried over MgSO₄, filtered, concentrated in vacuo, and purified on silica (ISCO, 24 g, Hexanes/EtOAc, 0-80% gradient) to give 365 mg of carbamate (71% yield) as a clear, colorless oil. ¹H NMR (400 MHz, CDCl₃): δ 7.26-7.22 (m, 2H), 7.09 (d, 1H, J=7.6 Hz), 3.93 (d, 1H, J=13.2 Hz), 3.46-3.38 (m, 5H), 3.03-2.99 (m, 8H), 2.69-2.50 (m, 4H), 2.30 (bs, 1H), 1.45 (s, 9H); MS (ES): m/z 526.4 [M+H]+

4 M HCl in dioxane (6.1 mL, 24.3 mmol, 35 eq) was added to a solution of the above material (365 mg, 0.694 mmol, 1.0 eq) in THE (6 mL). The reaction mixture was allowed to stir at room temperature overnight, then concentrated in vacuo to give 404 mg (93% yield) of crude material that was used in the next step without further purification; MS (ES): m/z 426.4 [M+H]+.

4-{[(2S)-4-(ethenesulfonyl)-1-{[3-fluoro-4-(trifluoromethoxy)phenyl]methyl}piperazin-2-yl]methyl}-1λ⁶-thiomorpholine-1,1-dione (SMDC275): 5 (295 mg, 0.694 mmol, 1.0 eq) and Et₃N (380 uL, 2.78 mmol, 4.0 eq) were dissolved in CH₂Cl₂ (5 mL, 0.14 M) and cooled to 0° C. 2-chloroethane-1-sulfonyl chloride (109 μL, 1.04 mmol, 1.5 eq) was added and the reaction was allowed to stir at 0° C. for 2 hours. LC/MS analysis showed remaining starting material, so additional 2-chloroethane-1-sulfonyl chloride (36 μL, 0.347 mmol, 0.5 eq) was added, and the reaction was allowed to stir overnight. The reaction mixture was quenched with H2O and extracted with CH₂Cl₂ (×3). The combined organic layers were washed with saturated aqueous NaHCO₃, then dried over MgSO₄, filtered, and concentrated in vacuo. The crude material was dissolved in MeOH and purified by HPLC (H2O (0.1% TFA):Acetonitrile, 20-95% gradient) to give 66.6 mg (18.6% yield) of 6. ¹H NMR (400 MHz, MeOD): δ 7.53 (s, 1H) 7.52 (d, 1H, J=8.8), 7.40 (d, 1H, J=8.8 Hz), 6.71 (dd, 1H, J=10.0, 16.4 Hz) 6.26 (d, 1H, J=16.8 Hz), 6.21 (d, 1H, J=10.0 Hz), 4.48 (d, 1H, J=13.6 Hz), 4.20 (d, 1H, J=13.6 Hz), 3.5-3.39 (m, 4H), 3.12 (bs, 11H), 2.93 (dd, 1H, J=7.2, 14.0 Hz); 13C NMR (150 MHz, MeOD): δ 43.52, 45.91, 48.48-51.47, 52.46, 53.02, 56.50, 57.97, 120.13 (d, J=19.5 Hz), 121.84 (q, J=256, 512 Hz), 125.55, 127.95, 130.60, 133.57, 155.81 (d, J=252 Hz); DEPT-135 (150 MHz, MeOD): S 43.23 (CH2), 45.62 (CH2), 48.48 (CH2), 51.18 (CH2), 52.17 (CH2), 52.74 (CH2), 56.21 (CH2), 57.69 (CH), 119.84 (d, J=18 Hz, CH), 125.26 (CH), 127.65 (CH), 130.32 (CH2), 133.28 (CH); MS (ES): m/z 516.3 [M+H]+.

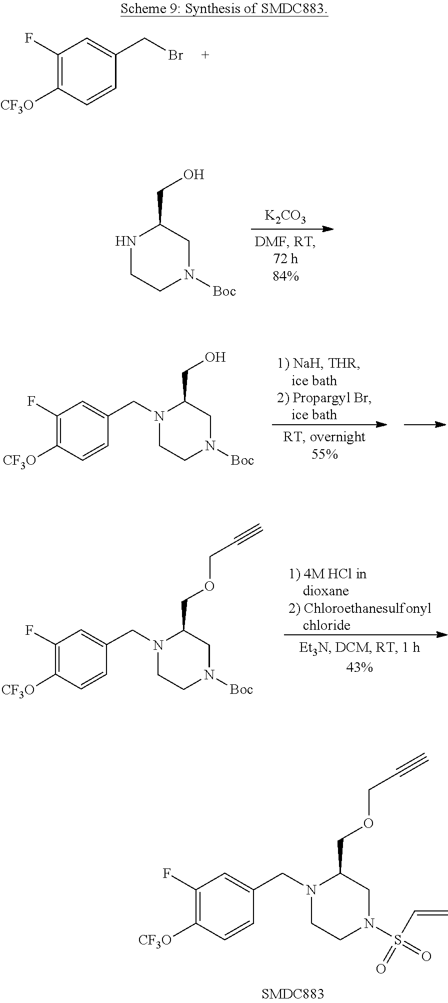

tert-butyl (3R)-4-{[3-fluoro-4-(trifluoromethoxy)phenyl]methyl}-3-(hydroxymethyl) piperazine-1-carboxylate: To a stirred solution of tert-butyl (3R)-3-(hydroxymethyl)piperazine-1-carboxylate (250 mg, 1.2 mol, 1.0 eq) in Dimethylformamide (5 ml) was added Potassium carbonate (200 mg, 1.44 mmol, 1.25 eq) followed by 3-Fluoro-4-(trifluoromethoxy)benzyl bromide (200 ul, 1.18 mmol, 1.02 eq). The reaction mixture was stirred for 72 hours, partitioned between water and Ethyl acetate. The water layer was extracted with Ethyl acetate once again and the organics were dried with brine and Sodium sulfate, decanted, concentrated to afford a residue which was eluted on a 12 g Silicycle cartridge in a gradient of Ethyl acetate in Hexane to afford the product (395 mg, 0.969 mmol) 84% yield. ¹H NMR (300 MHz, CHLOROFORM-d) δ ppm 1.42 (s, 9H) 2.13-2.30 (m, 1H) 2.45-2.62 (m, 1H) 2.62-2.74 (m, 1H) 2.74-3.09 (m, 1H) 3.16-3.56 (m, 4H) 3.63 (dd, J=11.40, 4.05 Hz, 2H) 3.72 (d, J=5.84 Hz, 1H) 3.97 (d, J=13.94 Hz, 1H) 7.02-7.13 (m, 1H) 7.14-7.26 (m, 2H); 19 F NMR (282 MHz, METHANOL-d4) δ ppm −128.93 (s, 1 F)-58.92-58.94 (d, 3 F); 13C NMR (75 MHz, CHLOROFORM-d) δ ppm 13.99 (s, 1 C) 20.84 (s, 1 C) 28.20 (s, 1 C) 44.22 (s, 1 C) 48.79 (s, 1 C) 57.00 (s, 1 C) 59.24 (s, 1C) 59.89 (s, 1 C) 60.28 (s, 1 C) 79.93 (s, 1 C) 115.20 (s, 1 C) 116.85 (s, 1 C) 117.10 (s, 1 C) 118.63 (s, 1 C) 122.05 (s, 1 C) 123.40 (s, 1 C) 124.21 (s, 1 C) 124.26 (s, 1 C) 125.48 (s, 1 C) 135.06 (s, 1 C) 135.09 (s, 1 C) 135.23 (s, 1 C) 135.26 (s, 1 C) 139.81 (s, 1 C) 139.89 (s, 1 C) 152.73 (s, 1 C) 154.99 (s, 1 C) 156.07 (s, 1 C) 171.12 (s, 1 C) 210.98 (s, 1 C); LCMS (ESI+) m/z 409.0 (M+H+).

tert-butyl (3R)-4-{[3-fluoro-4-(trifluoromethoxy)phenyl]methyl}-3-[(prop-2-yn-1-yloxy)methyl]piperazine-1-carboxylate: Sodium Hydride, 60% (19 mg, 0.5 mmol, 1.5 equiv.) was dissolved in dry tetrahydrofuran (5 ml) and cooled in an icebath under dry argon with stirring. A solution of tert-butyl (3R)-4-{[3-fluoro-4-(trifluoromethoxy)phenyl]methyl}-3-(hydroxymethyl)piperazine-1-carboxylate (132 mg, 0.3 mmol, 1.0 equiv.) dissolved in 3 ml THE was added dropwise, and stirring was continued in the icebath for 30 min. Propargyl bromide 80% (0.167 ml, 1.9 mmol, 6.0 equiv.) was added, the reaction mixture was warmed to room temperature and stirred overnight. In the morning, the reaction was quenched by addition of saturated ammonium chloride, then basified with aqueous Sodium hydroxide. Water was added and the mixture was extracted twice with ethyl acetate. The combined organics were dried with brine then Sodium sulfate, filtered then concentrated. The residue was dissolved in Dichloromethane and eluted on a 12 g Silicycle cartridge, in a gradient of Ethyl acetate in hexane to afford the product (78.7 mg, 0.176 mmol) 55% yield ¹H NMR (300 MHz, CHLOROFORM-d) δ ppm 1.37-1.55 (m, 9H) 2.22 (br. s., 1H) 2.43 (br. s., 1H) 2.67 (br. s., 2H) 3.07-3.38 (m, 2H) 3.38-3.54 (m, 2H) 3.58 (dd, J=9.61, 5.27 Hz, 2H) 3.65-3.78 (m, 1H) 3.87-4.06 (m, 1H) 4.14 (br. s., 2H) 7.12 (d, J=8.48 Hz, 1H) 7.15-7.31 (m, 2H); 19F NMR (282 MHz, CHLOROFORM-d) δ ppm −129.20 (s, 1 F)-58.88 (d, J=4.12 Hz, 3 F); LCMS (ESI+) m/z 447.1 (M+H+).

(2R)-4-(ethenesulfonyl)-1-{[3-fluoro-4-(trifluoromethoxy)phenyl]methyl}-2-[(prop-2-yn-1-yloxy)methyl]piperazine (SMDC883): tert-butyl (3R)-4-{[3-fluoro-4-(trifluoromethoxy)phenyl]methyl}-3-[(prop-2-yn-1-yloxy)methyl]piperazine-1-carboxylate (47.4 mg, 0.107 mmol) was dissolved in DCM (10 mL) and cooled to 0° C. The reaction mixture was treated with trimethylamine (2.00 equiv). To it was added 98.0% 2-Chloroethanesulfonyl chloride (1.0 equiv.). The reaction mixture was stirred for 2 h at cold. Then was added Trimethylamine (2 equiv.) at 0° C. The reaction was stirred for 2 h. The reaction mixture was evaporated to afford a crude product which was eluted on a 12 g Silicycle cartridge in a gradient of ethyl acetate in hexane with further purification by HPLC on C18 40-90% Methanol in-Water (all 0.05% formic acid) over 12 min 18 total to afford the product (20.0 mg, 0.046 mmol) 43% yield. ¹H NMR (300 MHz, CHLOROFORM-d) δ ppm 7.19-7.27 (m, 2H) 7.10 (d, J=8.10 Hz, 1H) 6.44 (dd, J=16.58, 9.80 Hz, 1H) 6.26 (d, J=16.58 Hz, 1H) 6.08 (d, J=9.80 Hz, 1H) 4.07-4.24 (m, 2H) 4.00 (d, J=14.32 Hz, 1H) 3.79 (dd, J=9.89, 4.43 Hz, 1H) 3.68 (dd, J=9.89, 5.18 Hz, 1H) 3.49 (d, J=14.13 Hz, 1H) 3.35 (dd, J=11.21, 2.35 Hz, 1H) 3.15-3.27 (m, 1H) 2.98-3.14 (m, 2H) 2.72-2.91 (m, 2H) 2.45 (t, J=2.35 Hz, 1H) 2.35-2.44 (m, 1H) 19F NMR (282 MHz, CHLOROFORM-d) δ ppm −58.84-−58.85 (d, J=4.12 Hz, 3 F) (s, 1 F) −128.84-−128.90 (m, 1 F) LCMS (ESI+) m/z 437.1 (M+H+).

4-(Trifluoromethoxy) phenylacetic acid (46.6 mg, 0.212 mmol) was subjected to the general procedure of Burlingame, et al. to afford N-(3-{[2-(Dimethylamino)ethyl]disulfanyl}propyl)-2-[4-(trifluoromethoxy)phenyl]acetamide; trifluoroacetic acid salt (SMDC673) (7.0 mg, 0.014 mmol) 6.6% yield. ¹H NMR (300 MHz, CHLOROFORM-d) δ ppm 1.90 (quin, J=6.88 Hz, 2H) 2.75 (t, J=6.97 Hz, 2H) 2.88 (s, 5H) 2.96-3.05 (m, 2H) 3.28-3.50 (m, 6H) 3.58 (s, 2H) 6.58 (br. s., 1H) 7.19 (d, J=7.72 Hz, 2H) 7.29-7.37 (m, 2H); ¹⁹F NMR (282 MHz, CHLOROFORM-d) δ ppm −60.70 (s, 3 F), −78.71 (s, 7 F); LCMS (ESI⁺) m/z 397.1 (M+H⁺).

### Example 2: Synthetic Methods

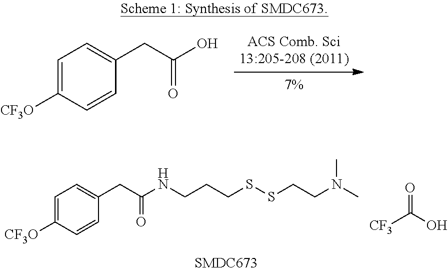

4-(Trifluoromethoxy) phenylacetic acid (46.6 mg, 0.212 mmol) was subjected to the general procedure of Burlingame, et al. to afford N-(3-{[2-(Dimethylamino)ethyl]disulfanyl}propyl)-2-[4-(trifluoromethoxy)phenyl]acetamide; trifluoroacetic acid salt (SMDC673) (7.0 mg, 0.014 mmol) 6.6% yield. ¹H NMR (300 MHz, CHLOROFORM-d) δ ppm 1.90 (quin, J=6.88 Hz, 2H) 2.75 (t, J=6.97 Hz, 2H) 2.88 (s, 5H) 2.96-3.05 (m, 2H) 3.28-3.50 (m, 6H) 3.58 (s, 2H) 6.58 (br. s., 1H) 7.19 (d, J=7.72 Hz, 2H) 7.29-7.37 (m, 2H); ¹⁹F NMR (282 MHz, CHLOROFORM-d) δ ppm −60.70 (s, 3 F), −78.71 (s, 7 F); LCMS (ESI⁺) m/z 397.1 (M+H⁺).

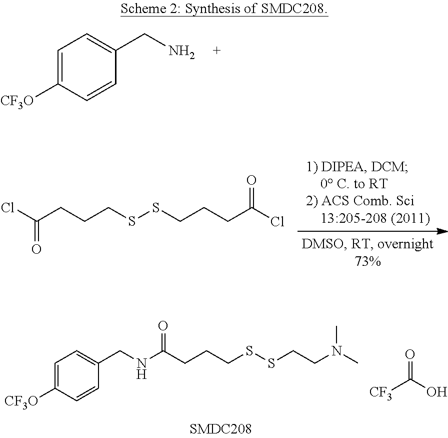

4-{[2-(dimethyl amino)ethyl]disulfanyl}-N-{[4-(trifluoromethoxy)phenyl]methyl}butanamide; trifluoroacetic acid salt (SMDC208):

4-Trifluoromethoxy benzylamine (100 ul, 0.655 mmol, 1.00 eq) was stirred in dry dichloromethane (4 ml) in an icebath. 4-[(4-chloro-4-oxobutyl)disulfanyl]butanoyl chloride (68.4 ul, 0.328 mmol) was added followed by N,N-diisopropylethylamine (171 ul, 0.983 mmol). The reaction was warmed to room temperature stirred until completion was seen by LC-MS [m/z 584.1 (M+H⁺)]. Solvent was removed under vacuum to afford a crude product which was used directly in the next reaction. A portion of the crude product (25 mg, 0.0 mmol, 1.0 equiv.) was dissolved in 300 ul DMSO. Meanwhile a solution of TCEP.HCl (2 mg, 0.0 mmol, 0.2 equiv.) in 100 ul of water was added to a suspension of bis[2-(N,N-dimethylamino)ethyl]disulfide dihydrochloride (60 mg, 0.2 mmol, 5.0 equiv.) in 100 ul DMSO. This was incubated for 10 min then was added to the DMSO solution of starting material, followed by N,N-diisopropylethylamine (0.094 ml, 0.5 mmol, 12.6 equiv.) and a precipitate formed. An additional 200 ul DMSO was added and the reaction mixture clarified and was stirred overnight. In the morning, the reaction mixture was loaded onto a 12 g C18 reverse-phase column that had been pre-equilibrated with water 0.05% TFA. It was then eluted in a gradient as follows: water: 3 CV, 0-80% MeOH water over 24 CV all 0.05% TFA. The fractions bearing product by LC-MS were lyophilized to afford the product (16 mg, 0.031 mmol, 73% yield). ¹H NMR (300 MHz, CHLOROFORM-d) δ ppm 2.06 (quin, J=6.88 Hz, 2H) 2.35-2.44 (m, 2H) 2.76-2.86 (m, 7H) 2.90-2.99 (m, 2H) 3.27-3.37 (m, 2H) 4.43 (d, J=5.84 Hz, 2H) 6.83 (br. s., 1H) 7.17 (d, J=7.91 Hz, 2H) 7.32 (d, J=8.48 Hz, 2H); LCMS (ESI) m/z (m+1) 397.1

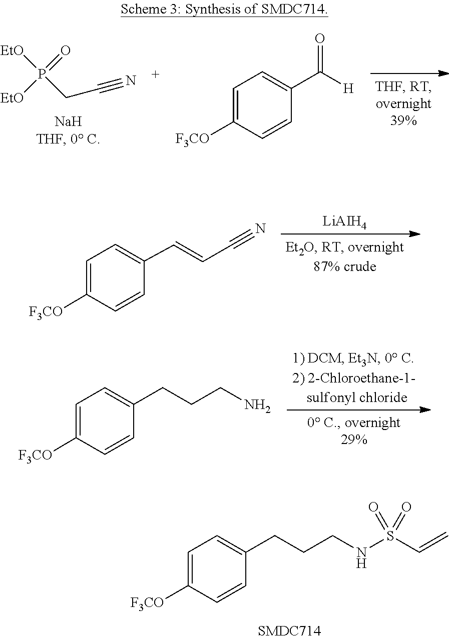

[4-(trifluoromethoxy)phenyl]prop-2-enenitrile: Sodium hydride 60% dispersion in mineral oil (0.558 g, 14.0 mmol 1.00 eq) was dissolved in dry tetrahydrofuran (17 ml) and stirred in an icebath under dry Argon. Diethyl cyanomethyl phosphonate (2.25 ml, 14.0 mmol, 1.00 eq), followed by 4-(trifluoromethoxy)benzaldehyde (1.99 ml, 14.0 mmol, 1.00 eq) in 5 mL THF, was added to the cold NaH/THF solution. The reaction was stirred and allowed to warm to room temperature overnight. The reaction was quenched with water and the reaction mixture was partitioned between water and ethyl acetate with the water layer extracted twice. The combined organic layer was organics were dried with brine and Sodium sulfate, decanted, concentrated to afford a residue which was dissolved in 10:1 hexane ether and hexane was added until 20:1 when crystallization afforded pure intermediate (1.18 g, 5.55 mmol, 39% yield) which was used directly in the next reaction. ¹H NMR (300 MHz, CHLOROFORM-d) δ ppm 5.88 (d, J=16.58 Hz, 1H) 7.26 (d, J=8.67 Hz, 2H) 7.40 (d, J=16.58 Hz, 1H) 7.45-7.55 (m, 2H).

3-[4-(trifluoromethoxy)phenyl]propan-1-amine: Lithium aluminum hydride (304 mg, 8.02 mmol, 3.20 eq) was suspended in dry diethyl ether (15 ml) with stirring under dry argon. This was heated to 50° C. and a solution of [4-(trifluoromethoxy)phenyl]prop-2-enenitrile (534 mg, 2.51 mmol, 1 eq) in diethyl ether (5 ml) was added dropwise with a syringe. Heating was continued for 1.5 hours, the reaction was cooled to room temperature and stirred overnight. The reaction was quenched by slow addition of water (5 ml) in an icebath followed by 1M NaOH (10 ml). The mixture was vigorously stirred with Ethyl acetate for 0.5 hours, partitioned and the water layer was extracted twice more with Ethyl acetate. The combined organics were dried with brine then sodium sulfate, filtered and concentrated to afford crude 3-[4-(trifluoromethoxy)phenyl]propan-1-amine (481 mg, 2.19 mmol) 87% yield, crude which was used crude in the next step. ¹H NMR (300 MHz, CHLOROFORM-d) δ ppm 1.64-1.76 (m, 2H) 2.55-2.63 (m, 2H) 2.66 (t, J=7.06 Hz, 2H) 7.03-7.11 (m, 2H) 7.11-7.17 (m, 2H)

[4-(trifluoromethoxy)phenyl]propyl}ethene-1-Sulfonamide (SMDC714): 3-[4-(trifluoromethoxy)phenyl]propan-1-amine (74.1 mg, 0.385 mmol, 1.00 eq) was dissolved in DCM (10 mL) and cooled to 0° C. The reaction mixture was treated with trimethylamine (2.00 equiv). To it was added 98.0% 2-Chloroethanesulfonyl chloride (1.0 equiv.). The reaction mixture was stirred for 2 h at cold. Then was added Trimethylamine (2 equiv.) at 0° C. The reaction was stirred for 2 h. The reaction mixture was evaporated to afford a crude product which was eluted on a 12 g Silicycle cartridge in a gradient of ethyl acetate 0-30% in Hexane to afford the product (34.8 mg, 0.113 mmol) 29% yield. ¹H NMR (300 MHz, CHLOROFORM-d) δ ppm 1.79-1.94 (m, 2H) 2.63-2.73 (m, 2H) 3.04 (q, J=6.84 Hz, 2H) 4.68 (t, J=5.93 Hz, 1H) 5.94 (d, J=9.80 Hz, 1H) 6.23 (d, J=16.58 Hz, 1H) 6.51 (dd, J=16.48, 9.89 Hz, 1H) 7.08-7.16 (m, 2H) 7.16-7.24 (m, 2H); ¹³C NMR (75 MHz, CHLOROFORM-d) δ ppm 31.45, 31.99, 42.33, 121.10, 122.20, 126.89, 129.66, 135.76, 139.63, 147.62; ¹⁹ F NMR (282 MHz, METHANOL-d4) δ ppm −57.93 (s, 1 F); LCMS (ESI+) m/z 310.30 (M+H⁺).

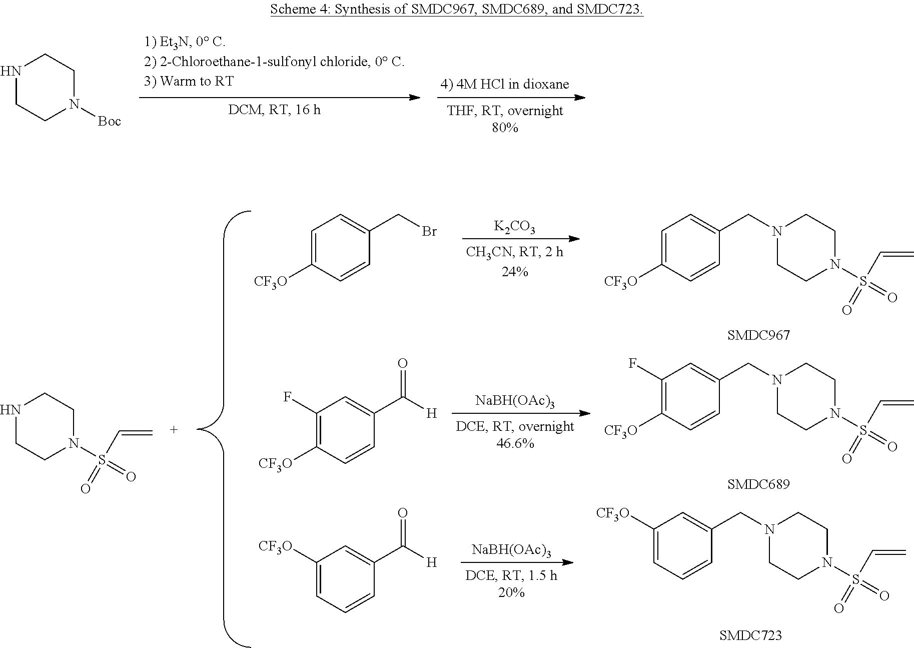

1-(Ethenesulfonyl)piperazine hydrochloride: N-Boc piperazine (10.0 g, 53.7 mmol, 1.0 eq), was dissolved in CH₂Cl₂ (400 mL, 0.13 M), and Et₃N (20 mL, 214 mmol, 4.0 eq) was added. The mixture was cooled to 0° C. in an ice bath and 2-chloroethane sulfonyl chloride (7.86 mL, 75.2 mmol, 1.4 eq) was added. After addition was complete, the reaction mixture was allowed to warm to room temperature and stirred for 16 hours. The reaction was diluted with H₂O and extracted with CH₂Cl₂ (×3). The combined organic layers were washed with saturated aqueous NaCl, dried over MgSO₄, filtered, and concentrated in vacuo. The crude material was purified on silica (0-70% gradient of EtOAc in Hexanes). ¹H NMR (400 MHz, MeOD): δ 6.64 (dd, 1H, J=10.0, 16.5 Hz), 6.19 (d, 1H, J=16.5 Hz), 6.13 (d, 1H, J=10.0 Hz), 3.52 (4H, t, J=4.78 Hz), 3.10 (4H, t, J=5.2 Hz), 1.46 (s, 9H).

This material was dissolved in THF (˜200 mL) and 4 M HCl in dioxane was added (134 mL, 537 mmol, 10 eq) and the reaction was allowed to stir overnight at room temperature. The reaction mixture was then concentrated in vacuo and dried under low pressure to give 9.11 g (80% yield over 2 steps) of 1-(ethenesulfonyl)piperazine hydrochloride (1) as an off-white powder, which was used without further purification. ¹H NMR (400 MHz, MeOD): δ 6.72 (dd, 1H, J=10.0, 16.5 Hz), 6.27 (d, 1H, J=16.5 Hz), 6.21 (d, 1H, J=10.0 Hz), 3.44-3.40 (m, 4H), 3.36-3.30 (m, 4H); MS (ES): m/z 177.1 [M+H]⁺

1-(ethenesulfonyl)-4-{[4-(trifluoromethoxy)phenyl]methyl}piperazine (SMDC967): 1-(ethenesulfonyl)piperazine hydrochloride (122 mg, 0.6 mmol, 1.0 equiv.) was suspended in a septum-capped 20 dram vial in ordinary non-dried reagent grade acetonitrile (3 ml), potassium carbonate (159 mg, 1.1 mmol, 2.0 equiv.) was added, followed by 4-(Trifluoromethoxy)benzyl bromide (97 ul, 0.6 mmol, 1.0 equiv.). The reaction mixture was stirred at room temperature for 2 hours, filtered through celite, concentrated, dissolved in a small amount dichloromethane and eluted on a 12 g Silicycle cartridge in a gradient of Ethyl Acetate 0-100% in Hexane. The product was further purified by HPLC 30-80% Methanol (0.05% Formic acid both) in water over 12 min 18 min total, to afford the product (49 mg, 0.1 mmol, 24% yield) as a clear oil. ¹H NMR (300 MHz, CHLOROFORM-d) δ ppm 2.39-2.72 (m, 4H) 3.10-3.27 (m, 4H) 3.55 (s, 2H) 6.06 (d, J=9.98 Hz, 1H) 6.25 (d, J=16.58 Hz, 1H) 6.44 (dd, J=16.58, 9.80 Hz, 1H) 7.17 (m, J=8.29 Hz, 2H) 7.34 (m, J=8.29 Hz, 2H), ¹⁹F NMR (282 MHz, CHLOROFORM-d) δ ppm 57.87 (s, 1 F)¹³C NMR (75 MHz, CHLOROFORM-d) δ ppm 45.62 (s, 1 C) 52.21 (s, 1 C) 61.68 (s, 1 C) 76.58 (s, 1 C) 77.43 (s, 1 C) 120.89 (s, 1 C) 128.92 (s, 1C) 130.18 (s, 1 C) 132.13 (s, 1 C) 136.38 (s, 1 C); LCMS (ESI+) m/z 351.1 (M+H⁺).

1-(Ethenesulfonyl)-4-{[3-fluoro-4-(trifluoromethoxy)phenyl]methyl}piperazine (SMDC689) (2): 3-Fluoro-4-(trifluoromethoxy)benzaldehyde (78 mg, 0.392 mmol, 1.0 eq.) and 1-(ethenesulfonyl)piperazine hydrochloride (100 mg, 0.47 mmol, 1.2 eq) were dissolved in dichloroethane (2.5 mL, 0.16 M). NaBH(OAc)₃ was added, and the reaction was allowed to stir overnight at room temperature. After confirmation of product formation by LC/MS, the reaction was quenched with 2 M NaOH, then extracted with CH₂Cl₂ (×3). The combined organic layers were then washed with saturated aqueous NaCl, dried over MgSO₄, and concentrated in vacuo. The crude residue was dissolved in MeOH and purified by HPLC to give 66.1 mg (45.6% yield) of the desired product. ¹H NMR (400 MHz, CDCl₃): δ 7.40-7.37 (m, 2H), 7.32 (d, 1H, J=8.0 Hz), 6.44 (dd, 1H, J=9.8, 16.5 Hz), 6.29 (d, 1H, J=16.5 Hz), 6.15 (d, 1H, J=9.8 Hz), 4.15 (s, 1H), 3.57 (bs, 4H), 3.22 (bs, 4H); ¹³C NMR (100 MHz, CDCl₃): δ 42.84, 51.23, 59.64, 120.37 (q, J=249, 518), 120.25 (d, J=20 Hz), 124.56, 127.57 (d, J=2 Hz), 128.69 (d, 7 Hz), 130.98, 131.55, 138.16 (d, 12 Hz), 154.56 (d, J=255 Hz); DEPT-135 (100 MHz, CDCl₃): δ 42.74 (CH₂), 51.14 (CH₂), 59.56 (CH₂), 120.15 (d, J=19 Hz, CH), 124.47 (CH), 127.46 (d, J=4 Hz, CH), 130.88 (CH₂), 131.46 (CH); MS (ES) m/z 369.3 [M+H]⁺.

1-(3-(Trifluoromethoxy)benzyl)-4-(vinyl sulfonyl)piperazine (SMDC723): To 1-(ethenesulfonyl)piperazine hydrochloride (100 mg, 0.47 mmol) in dichloroethane (4.0 mL) was added 3-(trifluoromethoxy)benzaldehyde (70 μL, 0.56 mmol) followed by NaBH(OAc)₃ (350 mg, 1.61 mmol). The reaction vessel capped, purged with Argon, and stirred for 1.5 h at rt. The reaction solution was then diluted with CH₂Cl₂ (20 mL) and washed with 1 M aqueous NaOH (15 mL). The aqueous layer was then extracted 3× with CH₂Cl₂ (20 mL). The combined organics were then washed with brine and dried with MgSO₄. The crude product was absorbed onto celite and purified on a C18 column with 0-50% water (0.1 TFA): acetonitrile to afford 1-(3-(trifluoromethoxy)benzyl)-4-(vinylsulfonyl)piperazine (33.5 mg, 20%). ¹H NMR (400 MHz; MeOD): δ 7.64-7.60 (m, 1H), 7.54-7.51 (m, 2H), 7.45 (d, J=8.2 Hz, 1H), 6.71 (dd, J=16.6, 10.0 Hz, 1H), 6.28 (d, J=16.6 Hz, 1H), 6.22 (d, J=10.0 Hz, 1H), 4.38 (s, 2H), 3.44 (bs, 4H), 3.33 (bs, 4H); ¹³C NMR (100 MHz, CDCl₃): δ 42.76, 51.22, 60.24, 120.38 (q, J=171, 343 Hz), 123.00, 123.51, 129.43, 129.76, 131.03, 131.25, 131.48, 149.84; DEPT-135 (100 MHz, CDCl₃): δ 42.67 (CH₂), 51.13 (CH₂), 60.14 (CH₂), 122.89 (CH), 123.41 (CH), 129.32 (CH), 130.90 (CH₂), 131.15 (CH), 131.41 (CH); MS (ES) m/z 351.0 [M+H]⁺.

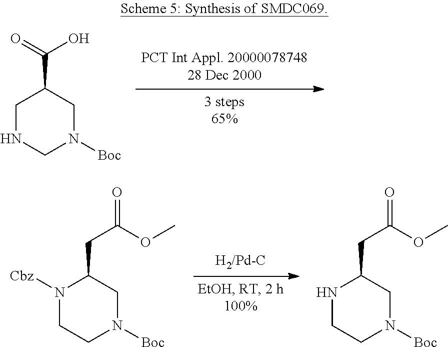

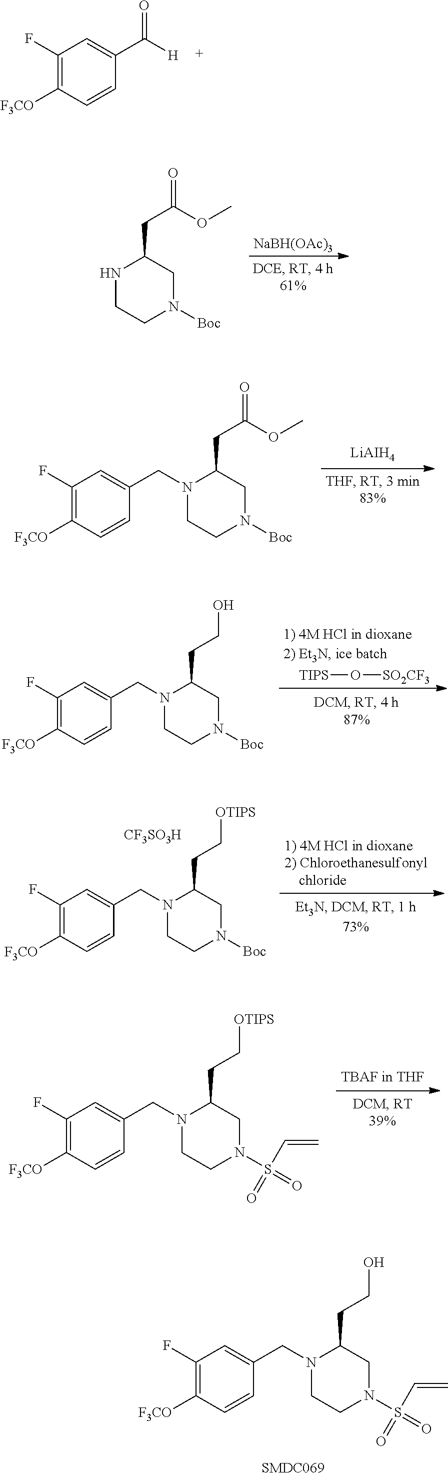

tert-butyl (3S)-3-(2-methoxy-2-oxoethyl)piperazine-1-carboxylate: 1-benzyl 4-tert-butyl (25)-2-(2-methoxy-2-oxoethyl)piperazine-1,4-dicarboxylate (1.23 g, 3.13 mmol, 1 eq) was dissolved in Ethanol (116 ml) and palladium 10% on carbon (465 mg) was added. The flask was fitted with a balloon adaptor, evacuated, and purged with hydrogen 3 times. The reaction mixture was then stirred under hydrogen for 2 hours, then filtered through celite and concentrated to afford a sooty-colored oil which was used in the next step. ¹H NMR (400 MHz, CHLOROFORM-d) δ ppm 7.93-10.19 (brs, 1H) 4.05-4.28 (m, 1H) 3.93-4.06 (m, 1H) 3.72 (s, 2H) 3.69-3.78 (m, 1H) 3.53-3.63 (m, 1H) 3.19-3.54 (m, 1H) 3.07-3.18 (m, 1H) 2.95-3.06 (m, 1H) 2.69-2.85 (m, 1H) 1.43 (s, 5H).

tert-butyl (3 S)-4-{[3-fluoro-4-(trifluoromethoxy)phenyl]methyl}-3-(2-methoxy-2-oxoethyl)piperazine-1-carboxylate: tert-butyl (3S)-3-(2-methoxy-2-oxoethyl)piperazine-1-carboxylate (509 mg, 1.97 mmol, 1 eq) was reacted with 3-fluoro-(trifluoromethoxy)benzaldehyde (0.341 ml, 1.97 mmol, 1 eq) in DCE (15 mL). Half an hour later, sodium triacetoxyborohydride (1.4 equiv.) was added to the above reaction mixture. The reaction mixture was monitored by LCMS and TLC. Next day, 12 h later, 100 mg more sodium triacetoxyborohydride was added to push the reaction mixture to the completion. 12 h later, the reaction mixture was treated with sat. 10% saturated solution of sodium bicarbonate solution (100 mL). Then the organic layer was extracted with dichloromethane (2×80 mL), the combined layers was washed with water (2×100 mL), dried over Na₂SO₄, and concentrated under reduced pressure to afford a residue which was purified on a 25 g Silicycle cartridge in a gradient of 0-100% Ethyl acetate in hexane to afford a product (546 mg, 1.21 mmol) 61% yield. ¹H NMR (400 MHz, CHLOROFORM-d) δ ppm 7.08-7.22 (m, 1H), 6.94-7.07 (m, 1H), 3.61 (br. s, 2H), 3.49-3.79 (m, 4H), 2.92-3.48 (m, 3H), 2.34-2.58 (m, 2H), 2.13-2.34 (m, 1H), 1.37 (s, 6H), 1.25-1.47 (m, 6H), 1.25-1.47 (m, 6H); ¹³C NMR (100 MHz, CHLOROFORM-d) δ ppm 172.06 (s, 1 C), 155.56 (s, 1 C), 154.59 (s, 1 C), 153.05 (s, 1 C), 139.86 (s, 1 C), 134.97 (s, 1 C), 124.04 (s, 1 C), 123.21 (s, 1 C), 121.57 (s, 1 C), 118.99 (s, 1 C), 116.66 (s, 1 C), 79.54 (s, 1 C), 57.11 (s, 1 C), 55.31 (s, 1 C), 51.45 (s, 1 C), 47.50 (s, 1C), 46.83 (s, 1 C), 42.39 (s, 1 C), 29.93 (s, 1 C), 28.03 (s, 1 C); ¹⁹F NMR (376 MHz, CHLOROFORM-d) δ ppm −59.04 (d, J=4.09 Hz, 3 F), −129.27-−129.16 (m, 1 F); LCMS (ESI+) m/z 451.16 (M+H⁺)

tert-butyl (3 S)-4-{[3-fluoro-4-(trifluoromethoxy)phenyl]methyl}-3-(2-hydroxyethyl) piperazine-1-carboxylate: Lithium aluminum hydride (54 mg, 1.42 mmol, 1.33 eq) was stirred in dry Tetrahydrofuran (15 ml) under dry argon with stirring. To this was added a solution of tert-butyl (3 S)-4-{[3-fluoro-4-(trifluoromethoxy)phenyl]methyl}-3-(2-methoxy-2-oxoethyl)piperazine-1-carboxylate (480 mg, 1.07 mmol, 1.00 eq) in 10 ml tetrahydrofuran via syringe over a 3 minute period at room temperature. The reaction was monitored to be complete by TLC. If not complete a small amount of LiAlH₄ may be added. The reaction was then cooled in an icebath with rapid stirring quenched by careful addition of 1.5 ml water, then 5 ml 2M NaOH, then 3 ml more water. The resulting mixture was filtered through celite then extracted twice with diethyl ether. The combined organics were dried with brine and sodium sulfate, filtered and concentrated. The residue was dissolved in minimal Dichloromethane and eluted on a 25 g Silicycle cartridge in a gradient of Ethyl acetate in Hexane 20-100% to afford the product (371 mg, 0.880 mmol, 83% yield). ¹H NMR (400 MHz, CHLOROFORM-d) δ ppm 7.12-7.22 (m, 1H) 7.02-7.08 (m, 1H) 3.77-3.95 (m, 1H) 3.59-3.77 (m, 2H) 3.34 (br. s., 3H) 2.53-2.71 (m, 1H) 2.10-2.24 (m, 1H) 1.75-1.89 (m, 1H) 1.62-1.75 (m, 1H) 1.39 (s, 6H); ¹³C NMR (100 MHz, CHLOROFORM-d) δ ppm 155.58 (s, 1 C) 154.85 (s, 1 C) 153.07 (s, 1 C)

(s, 1 C) 139.95 (s, 1 C) 135.13 (s, 1 C) 135.10 (s, 1 C) 135.08 (s, 1 C) 134.99 (s, 1 C) 134.98 (s, 1 C) 134.96 (s, 1 C) 124.23 (s, 1 C) 124.20 (s, 1 C) 123.29 (s, 1 C) 121.60 (s, 1 C) 119.02 (s, 1 C) 117.03 (s, 1 C) 116.84 (s, 1 C) 116.45 (s, 1 C) 79.87 (s, 1 C) 79.73 (s, 1 C) 77.32 (s, 1 C) 76.69 (s, 1 C) 59.81 (s, 1 C) 56.99 (s, 1 C) 56.61 (s, 1 C) 48.57 (s, 1 C) 47.73 (s, 1 C) 45.95 (s, 1 C) 43.13 (s, 1 C) 41.84 (s, 1 C) 29.42 (s, 1 C) 28.15 (s, 1 C); ¹⁹F NMR (376 MHz, CHLOROFORM-d) δ ppm −58.97 (d, J=4.09 Hz, 1 F)-129.19-−128.97 (m, 1 F); LCMS (ESI⁺) m/z 423.11 (M+H⁺).

(2S)-1-{[3-fluoro-4-(trifluoromethoxy)phenyl]methyl}-2-(2-{[tris(propan-2-yl)silyl]oxy}ethyl)piperazine; trifluoromethanesulfonic acid salt: tert-butyl (3S)-4-{[3-fluoro-4-(trifluoromethoxy)phenyl]methyl}-3-(2-hydroxyethyl)piperazine-1-carboxylate (443 mg, 1.05 mmol, 1.00 eq) was dissolved in dry dichloromethane (3 ml) and treated with 4M HCl in dry 1,4-dioxane (6 ml). The mixture was stirred for 2 hours under dry argon, then evaporated to afford a gummy solid which was immediately redissolved and stirred in dry dichloromethane (30 ml). To this was added Triethylamine (439 ul, 3.15 mmol, 3.00 eq) and the mixture was cooled in an icebath. Triisopropylsilyl trifluoromethanesulfonate (353 ul, 1.31 mmol, 1.25 equiv.) was added and the reaction mixture was allowed to warm to room temperature and checked for completion by TLC. Upon completion (4 hours), the reaction was quenched by addition of saturated sodium bicarbonate, partitioned between water and ethyl acetate with the water layer extracted twice with ethyl acetate, the combined organics were dried with brine, sodium sulfate, filtered and concentrated. The residue was redissolved in a small amount of dichloromethane then eluted in a gradient of 0-10% Methanol in dichloromethane to afford the product (435 mg, 0.909 mol, 87% yield). ¹H NMR (400 MHz, CHLOROFORM-d) δ ppm 7.16-7.26 (m, 1H) 7.09 (d, J=8.52 Hz, 1H) 6.24 (s, 1H) 3.96 (d, J=14.12 Hz, 1H) 3.71-3.83 (m, 1H) 3.30 (d, J=14.37 Hz, 1H) 3.19-3.27 (m, 1H) 3.06-3.16 (m, 1H) 2.94-3.06 (m, 1H) 2.85 (br. s., 1H) 2.74-2.81 (m, 1H) 2.38 (br. s., 1H) 1.85-1.97 (m, 1H) 1.72-1.85 (m, 1H) 0.94-1.13 (m, 23H); ¹³C NMR (100 MHz, CHLOROFORM-d) δ ppm 155.77 (s, 1 C) 153.26 (s, 1 C) 139.80 (s, 1 C) 139.74 (s, 1 C) 135.32 (s, 1 C) 135.31 (s, 1 C) 135.20 (s, 1 C) 135.18 (s, 1 C) 124.77 (s, 1 C) 124.27 (s, 1 C) 124.08 (s, 1 C) 124.05 (s, 1 C) 123.48 (s, 1 C) 121.70 (s, 1 C) 121.59 (s, 1 C) 119.12 (s, 1 C) 118.42 (s, 1 C) 116.89 (s, 1 C) 116.70 (s, 1 C) 116.55 (s, 1 C) 115.25 (s, 1 C) 59.76 (s, 1 C) 56.39 (s, 1 C) 55.81 (s, 1 C) 48.22 (s, 1 C) 48.08 (s, 1 C) 44.11 (s, 1 C) 31.19 (s, 1 C) 17.85 (s, 1 C) 11.76 (s, 1 C); ¹⁹F NMR (376 MHz, CHLOROFORM-d) δ ppm −58.99 (d, J=4.09 Hz, 3 F) 78.53 (s, 1 F)-129.24-−128.60 (m, 1 F); LCMS (ESI+) m/z 479.26 (M+H⁺).

(2S)-4-(ethenesulfonyl)-1-{[3-fluoro-4-(trifluoromethoxy)phenyl]methyl}-2-(2-{[tris(propan-2-yl)silyl]oxy}ethyl)piperazine: (2S)-1-{[3-fluoro-4-(trifluoromethoxy)phenyl]methyl}-2-(2-{[tris(propan-2-yl)silyl]oxy}ethyl)piperazine; trifluoromethanesulfonic acid salt (217 mg, 0.345 mmol, 1.00 eq) was dissolved in DCM and cooled to 0° C. The reaction mixture was treated with trimethylamine (2.00 equiv). To it was added 98.0% 2-Chloroethanesulfonyl chloride (1.0 equiv.). The reaction mixture was stirred for 2 h at cold. Then was added Trimethylamine (2 equiv.) at 0° C. The reaction was stirred for 2 h. The reaction mixture was evaporated to afford a crude product which was eluted on a 12 g Silicycle cartridge in a gradient of ethyl acetate in hexane to give the product (143 mg, 0.252 mmol, 73% yield). ¹H NMR (400 MHz, CHLOROFORM-d) δ ppm 7.18-7.25 (m, 1H) 7.09 (dd, J=8.52, 0.73 Hz, 1H) 6.43 (dd, J=9.98, 6.57 Hz, 1H) 6.23 (d, J=16.56 Hz, 1H) 6.05 (d, J=9.98 Hz, 1H) 3.77-3.85 (m, 1H) 3.75 (s, 1H) 3.46 (d, J=14.37 Hz, 1H) 3.20 (d, J=4.63 Hz, 1H) 3.03-3.11 (m, 1H) 2.90-2.95 (m, 1H) 2.72-2.80 (m, 1H) 2.36-2.44 (m, 1H) 1.80-1.89 (m, 1H) 0.96-1.12 (m, 10H); ¹³C NMR (100 MHz, CHLOROFORM-d) δ ppm 155.77 (s, 1 C) 153.26 (s, 1 C) 140.25 (s, 1 C) 140.19 (s, 1 C) 135.15 (dq, J=12.47, 1.71 Hz, 1 C)_{132.09} (s, 1 C) 128.80 (s, 1 C) 124.01 (d, J=3.67 Hz, 1 C) 123.41 (s, 1 C) 121.71 (s, 1 C) 120.41 (q, J=258.00 Hz, 1 C) 116.86 (s, 1 C) 60.14 (s, 1C) 56.63 (s, 1 C) 56.61 (s, 1 C) 55.57 (s, 1 C) 48.81 (s, 1 C) 47.88 (s, 1 C) 45.30 (s, 1 C) 29.04 (s, 1 C) 17.94 (s, 1 C) 11.81 (s, 1 C); ¹⁹F NMR (376 MHz, CHLOROFORM-d) δ ppm −58.90 (d, J=5.45 Hz, 3F)-129.07-−129.00 (m, 1F).

2-[(2S)-4-(ethenesulfonyl)-1-{[3-fluoro-4-(trifluoromethoxy)phenyl]methyl}piperazin-2-yl]ethan-1-ol (SMDC069): (2S)-4-(ethenesulfonyl)-1-{[3-fluoro-4-(trifluoromethoxy)phenyl]methyl}-2-(2-{[tris(propan-2-yl)silyl]oxy}ethyl)piperazine (143 mg, 0.252 mmol, 1.00 eq) was dissolved in dry Dichloromethane (4 ml) and stirred under dry argon. Tetrabutyl ammonium fluoride solution in THE (7 ml) was added and the progress of the reaction was monitored frequently by TLC to prevent formation of the cyclic product. As soon as the starting material disappeared, the reaction was quenched by the addition of 50% saturated Sodium bicarbonate in water. The mixture was diluted with Ethyl acetate and water and the water layer was extracted twice. The combined organic layer was dried with brine then Sodium sulfate, filtered and concentrated to afford a residue which was re-dissolved in Dichloromethane and purified on a 4 g Silicycle cartridge. Further purification was achieved by HPLC on C18 in a gradient of 30-80% Methanol in Water 12 min 18 total each 0.05% Formic acid. Since the UV absorbance of the compound was low in the 200-300 nm UV range, a sensitive collection method was used to obtain the product (41 mg, 0.098 mmol) 39% yield. ¹H NMR (400 MHz, CHLOROFORM-d) δ ppm 7.18-7.27 (m, 2H) 7.10 (d, J=8.28 Hz, 1H) 6.44 (dd, J=16.68, 9.86 Hz, 1H) 6.25 (d, J=16.56 Hz, 1H) 6.08 (d, J=9.98 Hz, 1H) 3.92 (d, J=13.88 Hz, 1H) 3.77-3.88 (m, 1H) 3.73 (ddd, J=10.84, 7.18, 5.60 Hz, 1H) 3.47 (d, J=13.88 Hz, 1H) 3.23-3.29 (m, 1H) 3.04-3.23 (m, 3H) 2.88-2.95 (m, 1H) 2.79-2.88 (m, 1H) 2.64 (s, 1H) 2.42 (ddd, J=12.30, 5.97, 3.65 Hz, 1H) 1.82-2.03 (m, 2H); ¹³C NMR (100 MHz, CHLOROFORM-d) δ ppm 155.74 (s, 1 C) 153.23 (s, 1 C) 139.58 (d, J=5.87 Hz, 1 C) 135.31 (d, J=10.27 Hz, 1 C) 131.95 (s, 1C) (s, 1 C) 129.12 (s, 1 C) 124.26 (d, J=3.67 Hz, 1 C) 123.51 (s, 1 C) 121.67 (s, 1 C) 120.39 (q, J=258.00 Hz, 1 C) 117.08 (s, 1 C) 116.89 (s, 1 C) 116.52 (s, 1 C) 60.02 (s, 1 C) 56.55 (d, J=1.47 Hz, 1 C) 56.42 (s, 1 C) 48.32 (s, 1 C) 47.70 (s, 1 C) 44.67 (s, 1 C) 28.72 (s, 1 C); LCMS (ESI+) m/z 413.04 (M+H⁺).

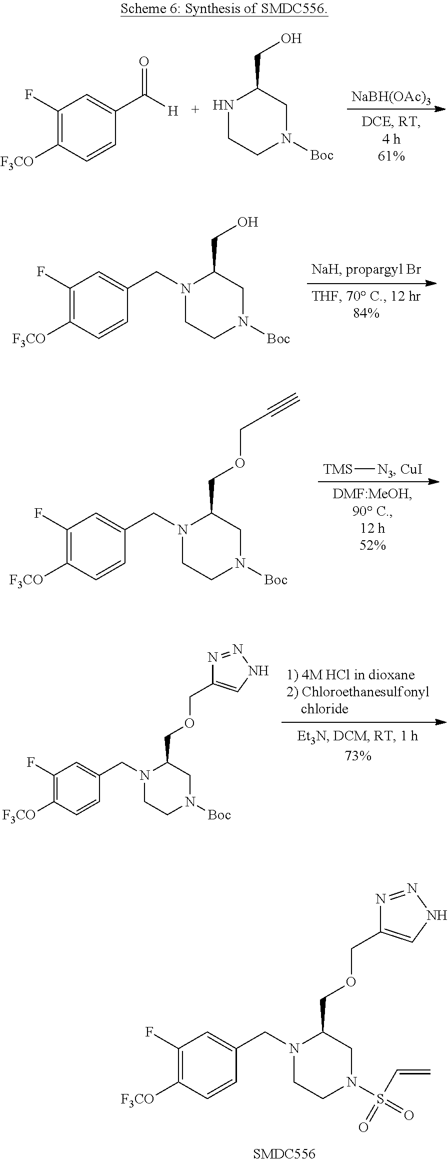

tert-butyl (R)-4-(3-fluoro-4-(trifluoromethoxy)benzyl)-3-(hydroxymethyl)piperazine-1-carboxylate: To a solution of 3-fluoro-4-(trifluoromethoxy)benzaldehyde (2.19 g, 10.2 mmol, 1.0 eq.) in DCE (60 mL) was added tert-butyl (R)-3-(hydroxymethyl)piperazine-1-carboxylate 2 (2.19 g, 9.65 mmol, 1.0 eq.). Half an hour later, sodium triacetoxyborohydride (3.02 g, 14.3 mmol, 1.4 eq.) was added to the above reaction mixture. The reaction mixture was monitored by LCMS and TLC. Next day, 12 h later, 500 mg more sodium triacetoxyborohydride was added to push the reaction mixture to the completion. 12 h later, the reaction mixture was treated with a 10% saturated solution of sodium bicarbonate solution (100 mL). Then the organic layer was extracted with dichloromethane (2×80 mL), the combined layers were washed with water (2×100 mL), dried over Na₂SO₄, and concentrated under reduced pressure. The crude liquid was loaded on to a 120 g silica column. The crude was purified by a gradient 100% hexane to 100% ethyl acetate. The yield was 68.4%. ¹H NMR (400 MHz; METHANOL-d4) δ: 7.32-7.40 (m, 2H), 7.26 (d, J=8.0 Hz, 1H), 4.86 (s, 1H), 4.06-4.14 (m, 1H), 3.73-3.81 (m, 2H), 3.53-3.68 (m, 2H), 3.46 (d, J=14.2 Hz, 1H), 3.14-3.29 (m, 2H), 2.64-2.72 (m, 1H), 2.45-2.52 (m, 1H), 2.16-2.25 (m, 1H), 1.46 (s, 9H); ¹³C NMR (100 MHz; METHANOL-d4) δ: 157.3, 156.9, 154.8, 142.9, 136.7, 136.5, 126.5, 125.0, 123.6, 121.0, 118.8, 81.6, 62.8, 61.9, 58.5, 51.3, 50.0, 29.0, 21.2, 14.8; ¹⁹F NMR (376 MHz; METHANOL-d4) δ: −60.59 (s, 1F), −131.71 (s, 1F); MS m/z=431 as a Na⁺ salt.

tert-butyl (R)-4-(3-fluoro-4-(trifluoromethoxy)benzyl)-3-((prop-2-yn-1-yloxy)methyl) piperazine-1-carboxylate: 60.0% Sodium hydride (482 mg, 12.0 mmol, 4.20 equiv.) was added to a solution of tert-butyl (3R)-4-{[3-fluoro-4-(trifluoromethoxy)phenyl]methyl}-3-(hydroxymethyl)piperazine-1-carboxylate c (1.16 g, 2.80 mmol, 1.00 equiv.) in tetrahydrofuran (6.00 mL) at 0° C. under N₂. The reaction mixture was stirred under nitrogen for 60 min. Then 80.0% propargyl bromide in toluene (9.40 ml, 6.0 mmol, 2.1 equiv.) was added to the reaction mixture at 0° C. The reaction mixture was stirred for an additional 12 h at 70° C. The reaction mixture was cooled to room temperature and treated with ice water. The reaction mixture was poured in a separator funnel, extracted with ethyl acetate (3×50 mL), dried over MgSO₄ and concentrated under reduced pressure. Then the crude mixture was loaded on to a 40 g silica column and purified with a gradient 100% hexane to 100% ethyl acetate. The yield was 80%. ¹H NMR (400 MHz; CHLOROFORM-d) δ: 7.28-7.51 (m, 1H), 7.20-7.26 (m, 2H), 7.12 (br d, J=8.4 Hz, 1H), 4.71 (s, 1H), 3.95-4.19 (m, 3H), 3.89-3.91 (m, 1H), 3.52-3.78 (m, 3H), 3.41-3.52 (m, 2H), 3.21-3.37 (m, 1H), 2.77 (s, 1H), 2.57-2.73 (m, 2H), 2.43 (t, J=2.1 Hz, 1H), 2.13-2.34 (m, 1H), 2.05 (s, 1H), 1.64 (br d, J=15.9 Hz, 1H), 1.43-1.50 (m, 9H); ¹³C NMR (100 MHz; CHLOROFORM-d) δ: 155.7, 154.8, 153.2, 140.4, 124.4, 123.4, 121.8, 117.3, 117.1, 79.8, 79.3, 77.3, 77.2, 77.0, 76.7, 74.8, 68.4, 60.7, 60.4, 58.5, 57.6, 28.4, 14.2; ¹⁹F NMR (376 MHz; CHLOROFORM-d) δ: −58.86 (s, 1F), −129.16 (s, 1F); MS m/z=447.4.

tert-butyl (R)-3-(((1H-1,2,3-triazol-4-yl)methoxy)methyl)-4-(3-fluoro-4-(trifluoro methoxy)benzyl)piperazine-1-carboxylate: tert-butyl (3R)-4-{[3-fluoro-4-(trifluoromethoxy)phenyl]methyl}-3-[(prop-2-yn-1-yloxy)methyl]piperazine-1-carboxylate d (0.239 g, 0.60 mmol, 1.0 eq.) and copper(I) iodide (0.005 g, 0.05 eq.) was added in a mixture of N,N-dimethylformamide (5.69 ml, 73.8 mmol) and methanol (0.46 ml,) at 0° C. TMS azide (0.1 ml, 0.6 mmol, 1.5 equiv.) was then added dropwise and the mixture was heated at 90° C. for 18 hours. Once there was no starting material left, the reaction mixture was passed over a celite plug. The reaction mass was concentrated under reduced pressure and the crude mixture was loaded on to a 12 g silica column and eluted with 50% hexane to 50% ethyl acetate to afford the product (0.20 g, 76% yield). ¹H NMR (400 MHz; CHLOROFORM-d) δ: 7.48-7.78 (m, 1H), 7.21 (br d, J=9.5 Hz, 2H), 6.99-7.12 (m, 1H), 4.52-4.82 (m, 2H), 3.84-4.02 (m, 1H), 3.47 (br s, 8H), 2.52-2.86 (m, 2H), 2.07-2.36 (m, 1H), 1.46 (s, 10H); ¹³C NMR (100 MHz; CHLOROFORM-d) δ: 156.0, 155.3, 153.5, 140.2, 140.2, 135.6, 124.7, 124.7, 123.7, 122.0, 119.5, 117.6, 117.4, 80.5, 77.6, 58.9, 58.9, 57.9, 53.7, 49.4, 30.0, 28.7; ¹⁹F NMR (376 MHz; CHLOROFORM-d) δ: −58.84 (s, 1F), −58.86 (s, 1F), −129.07 (s, 1F); MS m/z=490.17.

(R)-2-(((1H-1,2,3-triazol-4-yl)methoxy)methyl)-1-(3-fluoro-4-(trifluoromethoxy) benzyl)-4-(vinylsulfonyl)piperazine (SMDC556): tert-butyl (R)-3-(((1H-1, 2, 3-triazol-5-yl) methoxy) methyl)-4-(3-fluoro-4-(trifluoromethoxy) benzyl)piperazine-1-carboxylate e (0.1 g, 0.2 mmol, 1.0 equiv.) was treated with 4 M hydrochloric acid (1.502 ml, 6.0 mmol, 30.0 equiv.) in 1,4-dioxane in cold. The reaction mixture was stirred for 1 h and then checked by LC-MS. The crude reaction mixture was evaporated under reduced pressure. To it was added N, N-diisopropylethylamine (0.073 ml, 0.4 mmol, 2.0 equiv.) then 2-chloroethanesulfonyl chloride (0.021 ml, 0.2 mmol, 1.0 equiv.) at 0° C. The reaction was stirred for 2 hours (cold). N, N-diisopropylethylamine (0.070 ml, 0.4 mmol, 2.0 equiv.) was then added (cold). The reaction mixture was stirred for 2 hours at room temperature. The crude was purified by silica column and then by reverse phase HPLC using 100% water to 100% methanol to afford the product (0.01 g, 10% yield). ¹H NMR (400 MHz; CHLOROFORM-d) δ: 7.72 (s, 1H), 7.16-7.25 (m, 2H), 6.99-7.11 (m, 1H), 6.36-6.53 (m, 1H), 6.20-6.33 (m, 1H), 5.93-6.15 (m, 1H), 4.57-4.79 (m, 2H), 3.84-3.95 (m, 1H), 3.64-3.80 (m, 2H), 3.45-3.61 (m, 1H), 3.06-3.31 (m, 4H), 2.86-2.98 (m, 1H), 2.70-2.82 (m, 1H), 2.30-2.51 (m, 1H); ¹³C NMR (100 MHz; CHLOROFORM-d) δ: 155.8, 153.3, 139.8, 131.9, 131.4, 129.2, 124.2, 123.5, 117.1, 116.9, 77.8, 67.3, 64.1, 58.2, 57.3, 48.4, 47.6, 45.4; ¹⁹F NMR (376 MHz; CHLOROFORM-d) δ: −58.84 (s, 1F), −58.85 (s, 1F), −58.86 (s, 1F), −128.75 (s, 1F), −128.76 (s, 1F), MS m/z=480.4.

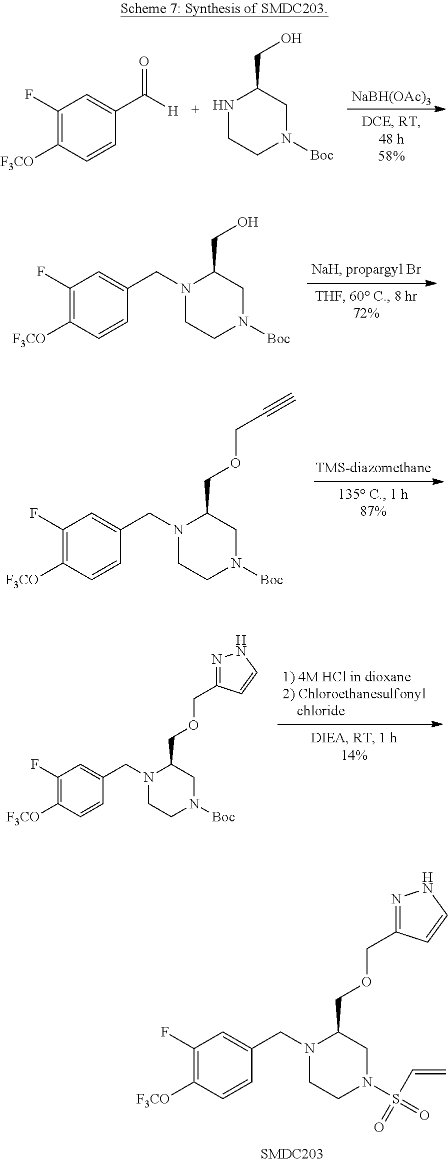

tert-butyl (R)-4-(3-fluoro-4-(trifluoromethoxy)benzyl)-3-(hydroxymethyl)piperazine-1-carboxylate: To a solution of 3-fluoro-4-(trifluoromethoxy)benzaldehyde (2.19 g, 10.2 mmol, 1.0 equiv.) in DCE (60 mL) was added tert-butyl (R)-3-(hydroxymethyl)piperazine-1-carboxylate 2 (2.19 g, 9.65 mmol, and 1.00 equiv). Half an hour later, sodium triacetoxyborohydride (3.02 g, 14.3 mmol, 1.4 equiv.) was added to the above reaction mixture. The reaction mixture was monitored by LCMS and TLC. The next day (12 h later), 500 mg more sodium triacetoxy borrohydride was added to push the reaction mixture to the completion. 12 h later, the reaction mixture was treated with sat. 10% saturated solution of sodium bicarbonate solution (100 mL). Then the organic layer was extracted with dichloromethane (2×80 mL), the combined layers was washed with water (2×100 mL), dried over Na₂SO₄, and concentrated under reduced pressure. The crude liquid was loaded on to a 120 g silica column. The crude was purified by a gradient 100% hexane to 100% ethyl acetate. The yield was 68.4%. ¹H NMR (400 MHz; METHANOL-d4) δ: 7.32-7.40 (m, 2H), 7.26 (d, J=8.0 Hz, 1H), 4.86 (s, 1H), 4.06-4.14 (m, 1H), 3.73-3.81 (m, 2H), 3.53-3.68 (m, 2H), 3.46 (d, J=14.2 Hz, 1H), 3.14-3.29 (m, 2H), 2.64-2.72 (m, 1H), 2.45-2.52 (m, 1H), 2.16-2.25 (m, 1H), 1.46 (s, 9H); ¹³C NMR (100 MHz; METHANOL-d4) δ: 157.3, 156.9, 154.8, 142.9, 136.7, 136.5, 126.5, 125.0, 123.6, 121.0, 118.8, 81.6, 62.8, 61.9, 58.5, 51.3, 50.0, 29.0, 21.2, 14.8; ¹⁹F NMR (376 MHz; METHANOL-d4) δ: −60.59 (s, 1F), −131.71 (s, 1F); MS m/z=431 as a Na+ salt.

tert-butyl (R)-4-(3-fluoro-4-(trifluoromethoxy)benzyl)-3-((prop-2-yn-1-yloxy)methyl)-piperazine-1-carboxylate: 60.0% Sodium hydride (482 mg, 12.0 mmol, 4.20 equiv.) was added to a solution of tert-butyl (3R)-4-{[3-fluoro-4-(trifluoromethoxy)phenyl]methyl}-3-(hydroxymethyl)piperazine-1-carboxylate 3 (1.16 g, 2.80 mmol, 1.00 equiv.) in tetrahydrofuran (6.00 mL) at 0° C. under N₂. The reaction mixture was stirred under nitrogen for 60 min. Then 80.0% propargyl bromide in toluene (9.40 ml, 6.0 mmol, 2.1 equiv.) was added to the reaction mixture at 0° C. The reaction mixture was stirred for 12 h at 70° C. The reaction mixture was cooled to room temperature and treated with ice water. The reaction mixture was poured in a separator funnel, extracted with ethyl acetate (3×50 mL), dried over MgSO₄ and concentrated under reduced pressure. Then the crude mixture was loaded on to a 40 g silica column and purified with a gradient 100% hexane to 100% ethyl acetate. The yield was 80%. ¹H NMR (400 MHz; CHLOROFORM-d) δ: 7.28-7.51 (m, 1H), 7.20-7.26 (m, 2H), 7.12 (br d, J=8.4 Hz, 1H), 4.71 (s, 1H), 3.95-4.19 (m, 3H), 3.89-3.91 (m, 1H), 3.52-3.78 (m, 3H), 3.41-3.52 (m, 2H), 3.21-3.37 (m, 1H), 2.77 (s, 1H), 2.57-2.73 (m, 2H), 2.43 (t, J=2.1 Hz, 1H), 2.13-2.34 (m, 1H), 2.05 (s, 1H), 1.64 (br d, J=15.9 Hz, 1H), 1.43-1.50 (m, 9H); ¹³C NMR (100 MHz; CHLOROFORM-d) δ: 155.7, 154.8, 153.2, 140.4, 124.4, 123.4, 121.8, 117.3, 117.1, 79.8, 79.3, 77.3, 77.2, 77.0, 76.7, 74.8, 68.4, 60.7, 60.4, 58.5, 57.6, 28.4, 14.2; ¹⁹F NMR (376 MHz; CHLOROFORM-d) δ: −58.86 (s, 1F), −129.16 (s, 1F); MS m/z=447.4.

tert-butyl (R)-3-(((1H-pyrazol-4-yl)methoxy)methyl)-4-(3-fluoro-4-(trifluoro methoxy) benzyl)piperazine-1-carboxylate: To a sealed tube was added tert-butyl (3R)-4-{[3-fluoro-4-(trifluoromethoxy)phenyl]methyl}-3-[(prop-2-yn-1-yloxy)methyl]piperazine-1-carboxylate (0.61 g, 1.40 mmol, 1.00 equiv.) and (Trimethylsilyl)diazomethane (2.00 ml, 4.00 mmol, 2.90 equiv.) in hexane. The reaction mixture was heated at 135° C. for 1 h in a sealed tube. LCMS indicated completion, of the reaction. The crude reaction mixture was dried under reduced pressure and then was loaded on to a 40 g silica column. The column was purified by 10% methanol in 90% dichloromethane. The yield was 84%. ¹H NMR (400 MHz; METHANOL-d4) δ: 7.61 (br s, 1H), 7.28-7.37 (m, 2H), 7.19 (d, J=8.6 Hz, 1H), 6.31 (br s, 1H), 5.48 (s, 1H), 4.85 (s, 2H), 4.52 (br s, 2H), 4.44 (s, 1H), 4.02-4.19 (m, 1H), 3.96 (br d, J=14.1 Hz, 1H), 3.59-3.73 (m, 2H), 3.33-3.57 (m, 3H), 3.07-3.29 (m, 2H), 2.52-2.74 (m, 2H), 2.22 (br s, 1H), 1.44 (s, 9H); ¹³C NMR (100 MHz; METHANOL-d4) δ: 157.7, 155.2, 143.3, 137.1, 126.9, 125.4, 124.0, 121.4, 119.0, 106.4, 82.1, 61.1, 59.1, 55.6, 50.0, 29.4; ¹⁹F NMR (376 MHz; METHANOL-d4) δ: −60.61 (s, 1F), −131.79 (s, 1F); MS m/z=511 as a Na+ salt.

(R)-2-(((1H-pyrazol-4-yl)methoxy)methyl)-1-(3-fluoro-4-(trifluoromethoxy)benzyl)-4-(vinyl sulfonyl)piperazine (SMDC203): tert-butyl (R)-3-(((1H-pyrazol-4-yl)methoxy) methyl)-4-(3-fluoro-4-(trifluoromethoxy)benzyl)piperazine-1-carboxylate (0.36 g, 0.80 mmol, 1.0 equiv.) was dissolved in dichloromethane (3 mL) and cooled to 0° C. The reaction mixture was treated with hydrochloric acid (0.939 ml, 3.8 mmol, 10 equiv.). The reaction mixture was stirred for 1 h and then LCMS was checked. The reaction was complete, and the reaction mixture was concentrated under reduced pressure. The gummy liquid was triturated with diethyl ether (3×5 mL). Then the reaction mixture was cooled to 0° C. The reaction mixture was treated with trimethylamine (0.71 mL, 4.09 mmol, 2.00 equiv.). To the mixture was added 98.0% 2-chloroethanesulfonyl chloride (0.062 ml, 0.8 mmol, 1.0 equiv.). The reaction mixture was stirred for 2 h at cold. Then was added trimethylamine (0.71 ml, 4.09 mmol, 2 equiv.) at 0° C. The reaction was stirred for 2 h. The reaction mixture was evaporated and loaded in a column. The crude was purified by 100% DCM to 100% methanol. And finally was purified by reverse phase HPLC using 100% water to 100% methanol. ¹H NMR (400 MHz; METHANOL-d4) δ: 7.58 (s, 1H), 7.24-7.40 (m, 2H), 7.19 (br d, J=8.4 Hz, 1H), 6.51-6.72 (m, 1H), 6.26-6.40 (m, 1H), 6.07-6.23 (m, 1H), 4.43-4.64 (m, 2H), 3.96 (d, J=14.2 Hz, 1H), 3.56-3.85 (m, 2H), 3.33-3.55 (m, 2H), 3.16 (br dd, J=6.3, 3.3 Hz, 1H), 2.90-3.11 (m, 2H), 2.69-2.87 (m, 2H), 2.66 (s, 1H), 2.38 (br dd, J=7.7, 4.2 Hz, 1H); ¹³C NMR (100 MHz; CHLOROFORM-d) δ: 158.0, 155.5, 143.5, 134.6, 130.8, 127.2, 125.7, 119.4, 106.8, 70.0, 67.6, 61.0, 59.0, 58.3, 51.3, 49.1, 47.5, 43.6, 18.5, 13.8; 19F NMR (376 MHz; METHANOL-d4) δ: −60.62 (s, 1F), −131.74 (s, 1F); MS m/z=479.13 (dimer).

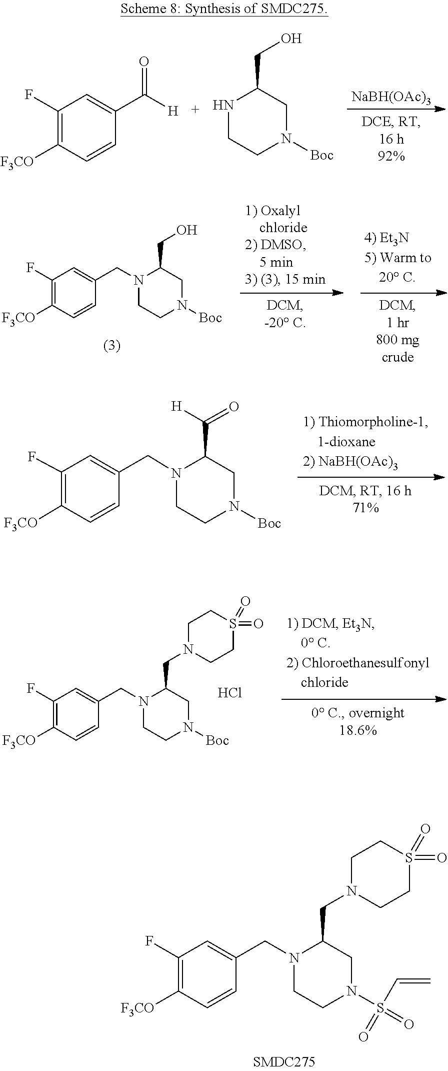

tert-Butyl-(3R)-4-{[3-fluoro-4-(trifluoromethoxy)phenyl]methyl}-3-(hydroxymethyl) piperazine-1-carboxylate: tert-Butyl (3R)-3-(hydroxymethyl)piperazine-1-carboxylate (2.5 g, 11.56 mmol, 1.0 eq), dichloroethane (20 mL, 0.58 M), and 3-fluoro-4-(trifluoromethoxy)benzaldehyde (2.4 mL, 17.34 mmol, 1.5 eq) were added to a 100 mL round bottom flask and allowed to stir for 5 minutes at room temperature. NaBH(OAc)₃ (7.4 g, 34.68 mmol. 3.0 eq) was added, and the reaction was allowed to stir for an additional 16 hours, then carefully quenched by the addition of 1 M aqueous NaOH and extracted with CH₂Cl₂ (×3). The combined organics were dried over MgSO₄, filtered, and concentrated in vacuo. The resulting residue was purified on silica (Hexanes/EtOAc, 0-45% gradient) to afford 4.34 g (92% yield) of tert-butyl-(3R)-4-{[3-fluoro-4-(trifluoromethoxy)phenyl]methyl}-3-(hydroxymethyl)piperazine-1-carboxylate. ¹H NMR (400 MHz, CDCl₃): δ 7.26 (m, 2H), 7.10 (d, 1H, J=8.3 Hz), 3.99 (d, 1H, J=13.8 Hz), 3.80 (dd, 1H, J=5.9, 11.5 Hz), 3.64 (dd, 2H, J=4.1, 11.5 Hz), 3.50 (bs, 1H), 3.46 (d, 2H, J=13.9 Hz), 3.27 (bs, 1H), 2.74 (bs, 1H), 2.62 (bs, 1H), 2.39 (bs, 1H), 2.30-2.24 (m, 1H), 1.45 (2, 9H); MS (ES): m/z 409.4 [M+H]⁺.

tert-Butyl (3R)-4-{[3-fluoro-4-(trifluoromethoxy)phenyl]methyl}-3-formylpiperazine-1-carboxylate: A solution containing oxalyl chloride (0.24 mL, 2.7 mmol, 1.1 eq,) in CH₂Cl₂ (30 mL, anhydrous) was cooled to −20° C., after which a solution of DMSO (0.35 mL, 4.9 mmol, 2.0 eq,) in CH₂Cl₂ (6.0 mL, anhydrous) was added and the mixture was allowed to stir for 5 min. A solution of 3 (1.0 g, 2.4 mmol, 1.0 eq) in CH₂Cl₂ (6.0 mL, anhydrous) was then added at −20° C. and the reaction was allowed to stir for 15 min. Et₃N (1.7 mL, 12 mmol, 5.0 eq) was slowly added, and the reaction was allowed to warm to 20° C. and stir for 1 hour. The reaction was diluted with CH₂Cl₂ and then washed sequentially with H2O, 2 M HCl, and saturated aqueous NaHCO₃. The organic layer was dried over MgSO₄, filtered, and concentrated in vacuo to give ˜800 mg of crude material as a clear, reddish oil that was used without any further purification. ¹H NMR (400 MHz, CDCl₃): δ 9.69 (d, 1H, J=2.0 Hz), 7.29-7.23 (m, 2H), 7.11 (d, 1H, J=8.8 Hz), 3.92 (d, 1H, J=14.0 Hz), 3.71 (bs, 1H), 3.62-3.58 (m, 2H), 3.43 (bs, 2H), 3.12 (bs, 1H), 2.96-2.90 (m, 1H), 2.36-2.30 (m, 1H), 1.45 (s, 9H); MS (ES): m/z 407.3 [M+H]⁺.

4-{[(2R)-1-{[3-Fluoro-4-(trifluoromethoxy)phenyl]methyl}piperazin-2-yl]methyl}-1λ⁶-thiomorpholine-1,1-dione Hydrochloride: Thiomorpholine 1,1-dioxide (173 mg, 1.28 mmol, 1.3 eq) was added to a solution of tert-Butyl (3R)-4-{[3-fluoro-4-(trifluoromethoxy)phenyl]methyl}-3-formylpiperazine-1-carboxylate (400 mg, 984 μmol, 1.0 eq,) in CH₂Cl₂ (8 mL) at room temperature. The reaction mixture was allowed to stir for 5 min and NaBH(OAc)₃ (834 mg, 3.94 mmol, 4.0 eq) was added. The reaction mixture was allowed to stir at room temperature for an additional 16 hours, then quenched with H₂O and extracted with CH₂Cl₂ (×3). The combined organic layers were washed with saturated aqueous NaHCO₃, followed by saturated aqueous NaCl. The organic layers were then dried over MgSO₄, filtered, concentrated in vacuo, and purified on silica (ISCO, 24 g, Hexanes/EtOAc, 0-80% gradient) to give 365 mg of carbamate (71% yield) as a clear, colorless oil. ¹H NMR (400 MHz, CDCl₃): δ 7.26-7.22 (m, 2H), 7.09 (d, 1H, J=7.6 Hz), 3.93 (d, 1H, J=13.2 Hz), 3.46-3.38 (m, 5H), 3.03-2.99 (m, 8H), 2.69-2.50 (m, 4H), 2.30 (bs, 1H), 1.45 (s, 9H); MS (ES): m/z 526.4 [M+H]⁺

4 M HCl in dioxane (6.1 mL, 24.3 mmol, 35 eq) was added to a solution of the above material (365 mg, 0.694 mmol, 1.0 eq) in THE (6 mL). The reaction mixture was allowed to stir at room temperature overnight, then concentrated in vacuo to give 404 mg (93% yield) of crude material that was used in the next step without further purification; MS (ES): m/z 426.4 [M+H]⁺.

4-{[(2S)-4-(ethenesulfonyl)-1-{[3-fluoro-4-(trifluoromethoxy)phenyl]methyl}piperazin-2-yl]methyl}-1λ⁶-thiomorpholine-1,1-dione (SMDC275): 4-{[(2R)-1-{[3-Fluoro-4-(trifluoromethoxy)phenyl]methyl}piperazin-2-yl]methyl}-1λ⁶-thiomorpholine-1,1-dione hydrochloride (295 mg, 0.694 mmol, 1.0 eq) and Et₃N (380 uL, 2.78 mmol, 4.0 eq) were dissolved in CH₂Cl₂ (5 mL, 0.14 M) and cooled to 0° C. 2-chloroethane-1-sulfonyl chloride (109 μL, 1.04 mmol, 1.5 eq) was added and the reaction was allowed to stir at 0° C. for 2 hours. LC/MS analysis showed remaining starting material, so additional 2-chloroethane-1-sulfonyl chloride (36 μL, 0.347 mmol, 0.5 eq) was added, and the reaction was allowed to stir overnight. The reaction mixture was quenched with H2O and extracted with CH₂Cl₂ (×3). The combined organic layers were washed with saturated aqueous NaHCO₃, then dried over MgSO₄, filtered, and concentrated in vacuo. The crude material was dissolved in MeOH and purified by HPLC (H₂O (0.1% TFA):Acetonitrile, 20-95% gradient) to give 66.6 mg (18.6% yield) of final product. ¹H NMR (400 MHz, MeOD): δ 7.53 (s, 1H) 7.52 (d, 1H, J=8.8), 7.40 (d, 1H, J=8.8 Hz), 6.71 (dd, 1H, J=10.0, 16.4 Hz) 6.26 (d, 1H, J=16.8 Hz), 6.21 (d, 1H, J=10.0 Hz), 4.48 (d, 1H, J=13.6 Hz), 4.20 (d, 1H, J=13.6 Hz), 3.5-3.39 (m, 4H), 3.12 (b s, 11H), 2.93 (dd, 1H, J=7.2, 14.0 Hz); ¹³C NMR (150 MHz, MeOD): δ 43.52, 45.91, 48.48, 51.47, 52.46, 53.02, 56.50, 57.97, 120.13 (d, J=19.5 Hz), 121.84 (q, J=256, 512 Hz), 125.55, 127.95, 130.60, 133.57, 155.81 (d, J=252 Hz); DEPT-135 (150 MHz, MeOD): δ 43.23 (CH₂), 45.62 (CH₂), 48.48 (CH₂), 51.18 (CH₂), 52.17 (CH₂), 52.74 (CH₂), 56.21 (CH₂), 57.69 (CH), 119.84 (d, J=18 Hz, CH), 125.26 (CH), 127.65 (CH), 130.32 (CH₂), 133.28 (CH); MS (ES): m/z 516.3 [M+H]⁺.

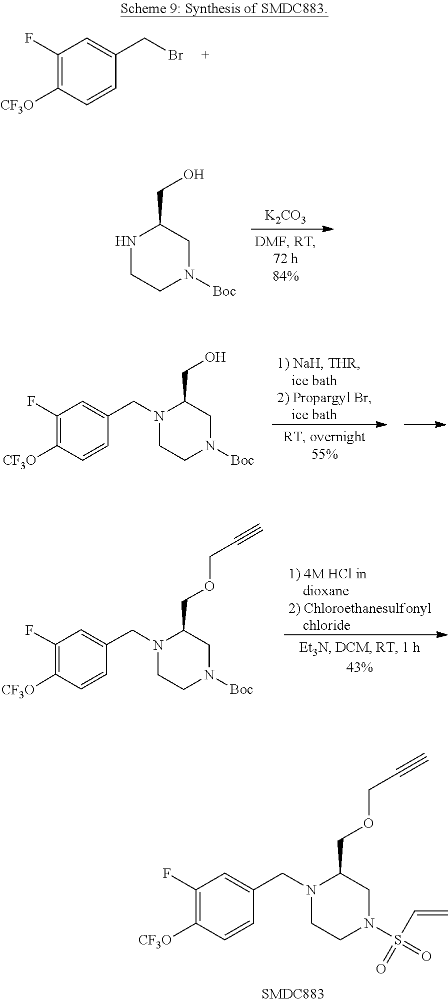

tert-butyl (3R)-4-{[3-fluoro-4-(trifluoromethoxy)phenyl]methyl}-3-(hydroxymethyl) piperazine-1-carboxylate: To a stirred solution of tert-butyl (3R)-3-(hydroxymethyl)piperazine-1-carboxylate (250 mg, 1.2 mol, 1.0 eq) in dimethylformamide (5 ml) was added potassium carbonate (200 mg, 1.44 mmol, 1.25 eq) followed by 3-fluoro-4-(trifluoromethoxy)benzyl bromide (200 ul, 1.18 mmol, 1.02 eq). The reaction mixture was stirred for 72 hours, partitioned between water and Ethyl acetate. The water layer was extracted with ethyl acetate once again and the organics were dried with brine and Sodium sulfate, decanted, concentrated to afford a residue which was eluted on a 12 g Silicycle cartridge in a gradient of ethyl acetate in Hexane to afford the product (395 mg, 0.969 mmol, 84% yield). ¹H NMR (300 MHz, CHLOROFORM-d) δ ppm 1.42 (s, 9H) 2.13-2.30 (m, 1H) 2.45-2.62 (m, 1H) 2.62-2.74 (m, 1H) 2.74-3.09 (m, 1H) 3.16-3.56 (m, 4H) 3.63 (dd, J=11.40, 4.05 Hz, 2H) 3.72 (d, J=5.84 Hz, 1H) 3.97 (d, J=13.94 Hz, 1H) 7.02-7.13 (m, 1H) 7.14-7.26 (m, 2H); ¹⁹ F NMR (282 MHz, METHANOL-d4) δ ppm −128.93 (s, 1 F)-58.92-58.94 (d, 3 F); ¹³C NMR (75 MHz, CHLOROFORM-d) δ ppm 13.99 (s, 1 C) 20.84 (s, 1 C) 28.20 (s, 1 C) 44.22 (s, 1 C) 48.79 (s, 1 C) 57.00 (s, 1 C) 59.24 (s, 1C) 59.89 (s, 1 C) 60.28 (s, 1 C) 79.93 (s, 1 C) 115.20 (s, 1 C) 116.85 (s, 1 C) 117.10 (s, 1 C) 118.63 (s, 1 C) 122.05 (s, 1 C) 123.40 (s, 1 C) 124.21 (s, 1 C) 124.26 (s, 1 C) 125.48 (s, 1 C) 135.06 (s, 1 C) 135.09 (s, 1 C) 135.23 (s, 1 C) 135.26 (s, 1 C) 139.81 (s, 1 C)_{139.89} (s, 1 C) 152.73 (s, 1 C) 154.99 (s, 1 C) 156.07 (s, 1 C) 171.12 (s, 1 C) 210.98 (s, 1 C); LCMS (ESI+) m/z 409.0 (M+H⁺).

tert-butyl (3R)-4-{[3-fluoro-4-(trifluoromethoxy)phenyl]methyl}-3-[(prop-2-yn-1-yloxy)methyl]piperazine-1-carboxylate: sodium hydride, 60% (19 mg, 0.5 mmol, 1.5 equiv.) was dissolved in dry tetrahydrofuran (5 ml) and cooled in an icebath under dry argon with stirring. A solution of tert-butyl (3R)-4-{[3-fluoro-4-(trifluoromethoxy)phenyl]methyl}-3-(hydroxymethyl)piperazine-1-carboxylate (132 mg, 0.3 mmol, 1.0 equiv.) dissolved in 3 ml THE was added dropwise, and stirring was continued in the icebath for 30 min. Propargyl bromide 80% (0.167 ml, 1.9 mmol, 6.0 equiv.) was added, the reaction mixture was warmed to room temperature and stirred overnight. In the morning, the reaction was quenched by addition of saturated ammonium chloride, then basified with aqueous Sodium hydroxide. Water was added and the mixture was extracted twice with ethyl acetate. The combined organics were dried with brine then Sodium sulfate, filtered then concentrated. The residue was dissolved in Dichloromethane and eluted on a 12 g Silicycle cartridge, in a gradient of Ethyl acetate in hexane to afford the product (78.7 mg, 0.176 mmol, 55% yield). ¹H NMR (300 MHz, CHLOROFORM-d) δ ppm 1.37-1.55 (m, 9H) 2.22 (br. s., 1H) 2.43 (br. s., 1H) 2.67 (br. s., 2H) 3.07-3.38 (m, 2H) 3.38-3.54 (m, 2H) 3.58 (dd, J=9.61, 5.27 Hz, 2H) 3.65-3.78 (m, 1H) 3.87-4.06 (m, 1H) 4.14 (br. s., 2H) 7.12 (d, J=8.48 Hz, 1H) 7.15-7.31 (m, 2H); ¹⁹F NMR (282 MHz, CHLOROFORM-d) δ ppm −129.20 (s, 1 F)-58.88 (d, J=4.12 Hz, 3 F); LCMS (ESI+) m/z 447.1 (M+H⁺).

(2R)-4-(ethenesulfonyl)-1-{[3-fluoro-4-(trifluoromethoxy)phenyl]methyl}-2-[(prop-2-yn-1-yloxy)methyl]piperazine (SMDC883): tert-butyl (3R)-4-{[3-fluoro-4-(trifluoromethoxy)phenyl]methyl}-3-[(prop-2-yn-1-yloxy)methyl]piperazine-1-carboxylate (47.4 mg, 0.107 mmol) was dissolved in DCM (10 mL) and cooled to 0° C. The reaction mixture was treated with trimethylamine (2.00 equiv). To it was added 98.0% 2-Chloroethanesulfonyl chloride (1.0 equiv.). The reaction mixture was stirred for 2 h at cold. Then was added Trimethylamine (2 equiv.) at 0° C. The reaction was stirred for 2 h. The reaction mixture was evaporated to afford a crude product which was eluted on a 12 g Silicycle cartridge in a gradient of ethyl acetate in hexane with further purification by HPLC on C18 40-90% methanol in water (all 0.05% formic acid) over 12 min (18 total) to afford the product (20.0 mg, 0.046 mmol, 43% yield). ¹H NMR (300 MHz, CHLOROFORM-d) δ ppm 7.19-7.27 (m, 2H) 7.10 (d, J=8.10 Hz, 1H) 6.44 (dd, J=16.58, 9.80 Hz, 1H) 6.26 (d, J=16.58 Hz, 1H) 6.08 (d, J=9.80 Hz, 1H) 4.07-4.24 (m, 2H) 4.00 (d, J=14.32 Hz, 1H) 3.79 (dd, J=9.89, 4.43 Hz, 1H) 3.68 (dd, J=9.89, 5.18 Hz, 1H) 3.49 (d, J=14.13 Hz, 1H) 3.35 (dd, J=11.21, 2.35 Hz, 1H) 3.15-3.27 (m, 1H) 2.98-3.14 (m, 2H) 2.72-2.91 (m, 2H) 2.45 (t, J=2.35 Hz, 1H) 2.35-2.44 (m, 1H) 19F NMR (282 MHz, CHLOROFORM-d) δ ppm −58.84-−58.85 (d, J=4.12 Hz, 3 F) (s, 1 F)-128.84 128.90 (m, 1 F) LCMS (ESI+) m/z 437.1 (M+H+).

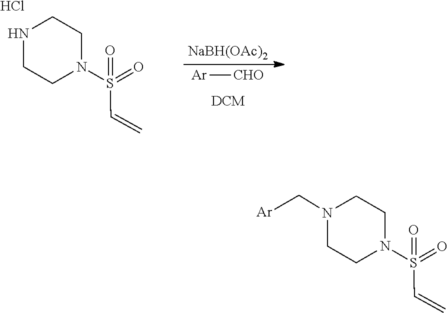

The following analogs were synthesized by reductive amination, in a manner similar to that described in Scheme 4, for compounds SMDC689 and SMDC723.

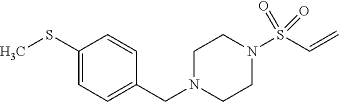 [SMILES: C=CS(=O)(=O)N1CCN(Cc2ccc(SC)cc2)CC1]

1-(4-(methylthio)benzyl)-4-(vinylsulfonyl)piperizine (SMDC739): ¹H NMR (400 MHz, CDCl₃): δ 7.30 (d, J=2.1 Hz, 4H), 6.45 (dd, J=9.6, 16.4 Hz, 1H), 6.30 (d, J=16.6 Hz, 1H), 6.16 (d, J=9.7 Hz, 1H), 4.17 (s, 2H), 3.51 (br mult, 8H), 2.51 (s, 3H); MS (ES) m/z 313.0 [M+H]⁺.

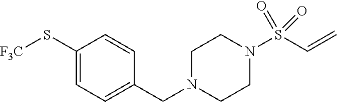 [SMILES: C=CS(=O)(=O)N1CCN(Cc2ccc(SC(F)(F)F)cc2)CC1]

1-(4-((trifluoromethyl)thio)benzyl)-4-(vinyl sulfonyl)piperazine (SMDC807): ¹H NMR (400 MHz, CDCl₃): δ 7.76 (d, J=8.4 Hz, 2H), 7.51 (d, J=8.1 Hz, 2H), 6.45 (dd, J=9.8, 16.4 Hz, 1H), 6.31 (d, J=16.5 Hz, 1H), 6.17 (d, J=9.8 Hz, 1H), 4.23 (s, 2H), 3.55 (br mult, 4H), 3.25 (br mult, 4H); MS (ES): m/z 367.0 [M+H]⁺.

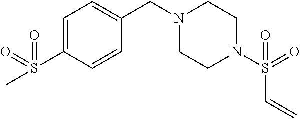 [SMILES: C=CS(=O)(=O)N1CCN(Cc2ccc(S(C)(=O)=O)cc2)CC1]

1-(4-(methylsulfonyl)benzyl)-4-(vinyl sulfonyl)piperazine (SMDC863): ¹H NMR (400 MHz, D20): δ 8.11 (d, 2H, J=6.2 Hz), 7.83 (d, 2H, J=5.8 Hz), 6.78-6.68 (m. 1H), 6.42-6.29 (m, 2H), 4.55 (s, 2H), 3.49 (s, 8H), 3.32 (s, 3H); MS (ES): m/z 345.0 [M+H]⁺.

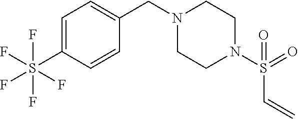 [SMILES: C=CS(=O)(=O)N1CCN(Cc2ccc(S(F)(F)(F)(F)F)cc2)CC1]

1-(4-(pentafluoro-16-sulfanyl)benzyl)-4-(vinylsulfonyl)piperazine (SMDC808). ¹H NMR (400 MHz, CDCl₃): δ 7.88 (d, 2H, J=8.6 Hz), 7.59 (d, 2H, J=8.3 Hz), 6.46 (dd, 1H, J=9.7, 16.5), 6.32 (d, 2H, J=16.5 Hz), 6.18 (d, 1H, J=9.8 Hz), 4.27 (s, 2H), 3.57 (br mult, 4H), 3.28 (br mult, 4H); MS (ES): m/z 393.0 [M+H]⁺.

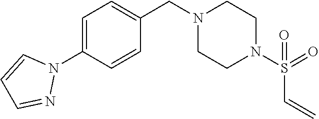 [SMILES: C=CS(=O)(=O)N1CCN(Cc2ccc(-n3cccn3)cc2)CC1]

1-(4-(1H-pyrazol-1-yl)benzyl)-4-(vinylsulfonyl)piperazine (SMDC866). ¹H NMR (400 MHz, MeOD): δ 8.33 (d, 1H, J=2.5 Hz), 7.93 (d, 2H, J=8.6 Hz), 7.78 (d, 1H, J=1.6 Hz), 7.66 (d, 2H, J=8.6 Hz), 6.73 (dd, 1H, J=10.0, 16.4 Hz), 6.58 (dd, 1H, J=1.9, 2.4 Hz), 6.29 (d, 1H, J=16.5 Hz), 6.23 (d, 1H, J=10.0), 4.46 (s, 2H), 3.45 (br mult, 8H); MS (ES): m/z 333.1 [M+H]⁺.

### Example 3

Taspase1 (Tasp1) is a unique threonine aspartyl protease, which is expressed as an inactive 420-residue proenzyme and is structurally homologous to Asparaginase-2. The proenzyme self-activates, undergoing autoproteolysis and forming two subunits (a/13) that associate as a heterodimer (Hsieh, 2003). Tasp1 is classified as a Ntn protease; after cis-activation, as the N-terminal residue of the beta subunit is the catalytic threonine (Thr234). The major substrates of Tasp1 include MLL family of epigenetic regulatory proteins, and the TFIIA family of cell cycle regulatory nuclear proteins (Zhou, 2006; Niizuma, 2015).

As a regulator of both MLL and TFIIA, Tasp1 has been implicated in multiple oncogenic and developmental diseases, (Wunch, 2016; Stauber, 2016). In particular, processing of MLL-1 by Tasp1 splits the protein into two fragments: (1) a 320 kDa N-terminal domain, and (2) a 180 kDa C-terminal domain, which are associated with chromatin binding and histone lysine methylation, respectively. Importantly, the N-terminal MLL fragment is susceptible to forming fusion proteins which influence the aberrant expression of oncogenic proteins including the HOX and cyclin families (Dorrance, 2006; Takeda, 2006). Similarly, Tasp1 cleaves TFIIAa-β, forming a heterotrimer with TFIIAγ, and is critical in the development of the head and neck, liver hematopoetic stem cells, and mammalian germ cell lines (Oyama, 2013; Stauber, 2016) A recent report also demonstrated that Tasp1 processes REV3L, the catalytic subunit of DNA polymerase C, which is involved with DNA lesion repair (Wang, 2020). In this case, Tasp1 stabilizes REV3L against ubiquitination and degradation, potentially allowing for unregulated DNA lesion repair by a polymerase which lacks proofreading functions.

Tasp1 is overexpressed in numerous liquid and solid malignancies and has been termed a ‘non-oncogene addiction’ protease (Hsieh, 2003; Chen, 2010). Loss of Tasp1 sensitizes glioblastoma and melanoma cells to chemotherapy-induced apoptosis, disrupts proliferation of human cancer cell lines in vitro, and reduces growth of tumor xenograft modes of several aggressive tumor types (Takeda, 2006; Scrideli, 2008; Chen, 2010). In addition, loss of Tasp1 strongly inhibits development of HER2-driven breast tumors and EGFR-driven lung cancer, including drug-resistant, EGFR-T790M mutant tumors (Dong, 2014). Other evidence points to a key role of Tasp1 in invasion and metastasis via proteolysis of MLL in HepG2 hepatocellular carcinoma metastasis models in vitro and in vivo (Niehof, 2008). High expression levels of Tasp1 correlated with poor prognosis in patients with gall bladder carcinoma, with an upregulation of FAM49B via the MLL-PI3K/AKT signaling pathway (Zhang, 2020). Another recent study suggested that Tasp1 plays an important role in head and neck squamous cell carcinomas by regulating nuclear localization and transcriptional activity of TFIIA, leading to a reduction of CDK inhibitor expression levels (Gribko, 2017). Therefore, growth factor-driven, drug-resistant cancers represent promising clinical indications for Tasp1 inhibitors. Previous attempts of developing Tasp1 inhibitors have met with limited success. Early peptidic inhibitors designed to target the substrate binding pocket included either electrophilic warheads (Lee, 2009) or hypothesized succinimide intermediates (van den Boom 2014). However, these peptide-based inhibitors demonstrated modest in vitro potency with fluorescently labeled substrates: assays with recombinant Tasp1 yielded IC50 values in the micromolar range. In addition, no further biochemical evaluation in the form of cellular activity assays were performed on these peptides. Small molecule Tasp1 inhibitors have also been reported. One study (Knauer, 2011), used an in silico approach to dock low molecular weight compounds into the crystal structure of activated Tasp1 (Khan, 2005). Two resulting groups, clustered into “deep hole” and “chloride hole” compounds were evaluated using a cell-based fluorescent Tasp1 cleavage assay, but only identified two fragments that demonstrated Tasp1 inhibition at 50 μM when added to cell culture media. A second study (Chen, 2012) likewise used a cell-based proteolysis assay to evaluate the NCI Diversity Set Library. Confirmatory and specificity in vitro screens later identified one arsenate compound that acted as a non-competitive allosteric inhibitor, with an IC50 of 7.5 μM. This compound also demonstrated efficacy in breast cancer and brain tumor xenograft models. Critically, none of these aforementioned examples further evaluated modes of action of their respective lead compounds in order to improve their efficacy, or to determine exact binding poses within hypothetical binding pockets.

In light of this, we have focused on a structure-based approach to develop and validate novel Tasp1 inhibitors. While no tractable hits emerged from an initial high-throughput screen (HTS) performed in our group, strong interest was retained in the potential of Tasp1 as a novel oncology drug discovery target. Thus, we employed an alternative hit-finding approach using a disulfide-trapping tethering screen (Erlanson, 2004; Kathman, 2016) to target and covalently modify surface exposed cysteines. Recently, tethering screens have also been used to successfully discover lead compounds targeting K-Ras G12C (Ostrem 2013; Gentile 2017; Nnadi 2018), Caspases (Hardy, 2004; Gao 2012), PTP1B (Keedy 2018), BRD4 (Olp, 2020), NADH dehydrogenase (Heikal, 2018), and 14-3-3 (Sijbesma, 2019).

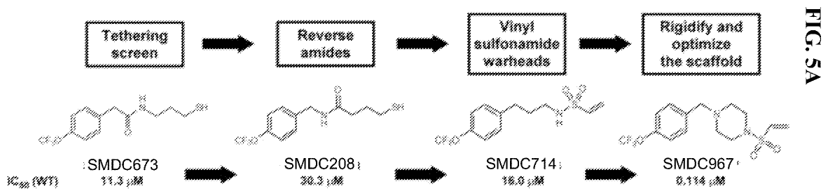

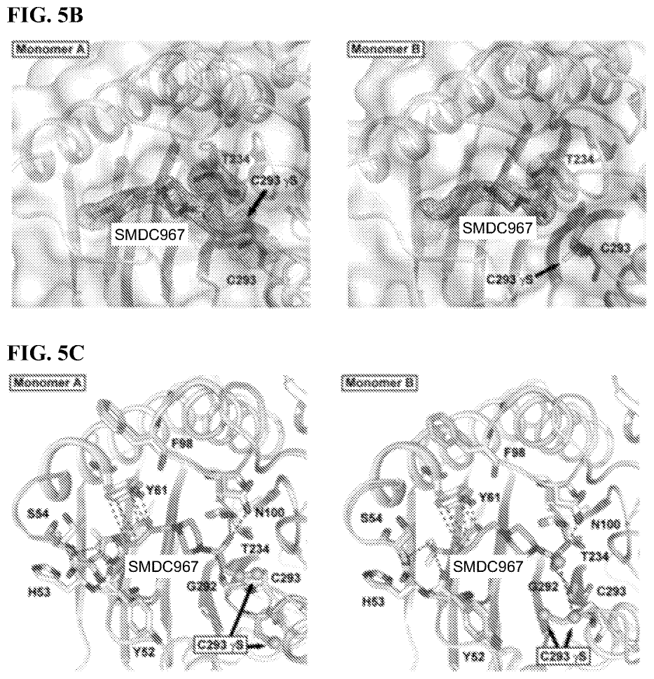

Disulfide-trapping was an especially suited screening method for targeting Tasp1 due to a non-catalytic cysteine residue (Cys293) located in the substrate binding pocket, and in close proximity to the catalytic Thr234 (FIGS. 5A-5C). Importantly, Cys293 is a unique endogenous residue which is surface exposed upon Tasp1 activation. Any compounds targeting Cys293 should be highly selective for Tasp1 against other type-2 asparaginases. For instance, although Tasp1 is structurally homologous to human asparaginase-like protein 1 (hASRGL1), both containing a conserved N-terminal catalytic threonine in their respective beta domains, only Tasp1 contains a native cysteine in the active site. Conceptually, tethering screens illustrate an unusual approach to targeting cysteines: this is in contrast to other strategies which start with non-covalent inhibitors, then add covalency for selectivity and enhanced potency, as in the case of kinases (recently reviewed in Hallenbeck, 2017; Wang, 2017; Abdeldayem, 2020). Our initial tethering screens were accompanied by expanded protein crystallography efforts to drive a structure-based design optimization strategy, followed by biological evaluation. This approach produced the first and only known potent Taspase1 inhibitors, with biochemical IC50 values less than 100 nM and single-digit μM cell-based potency.

Results

Tethering Screen Hits and Evolution to Warheaded Compounds

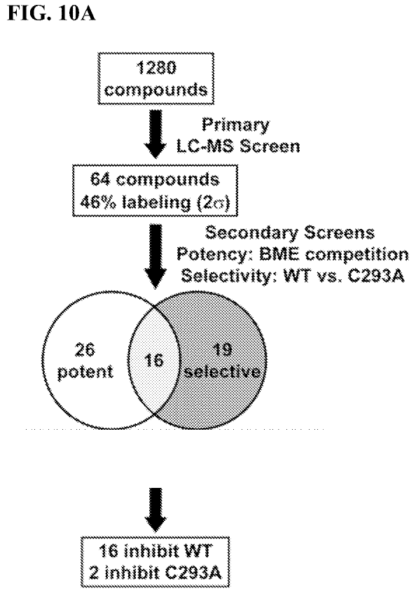

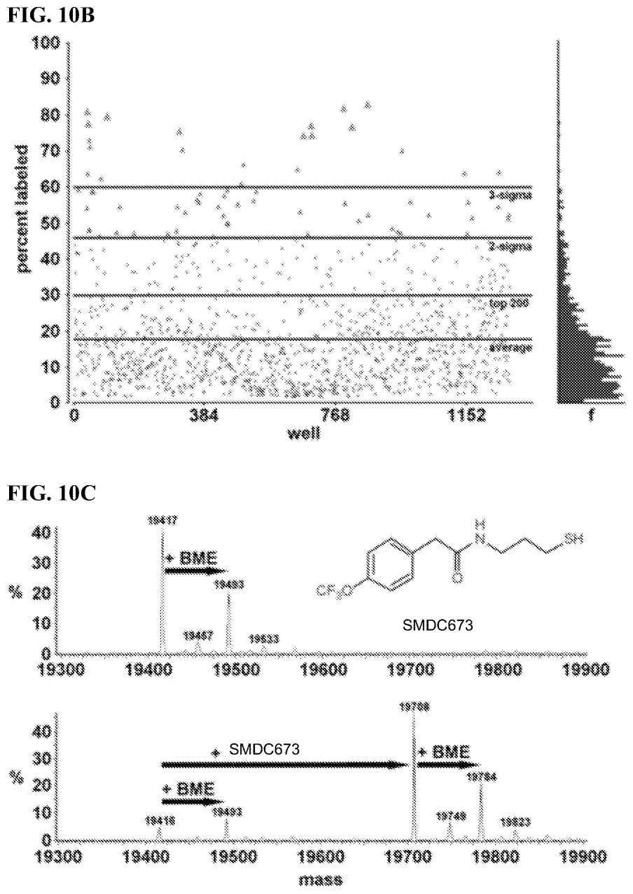

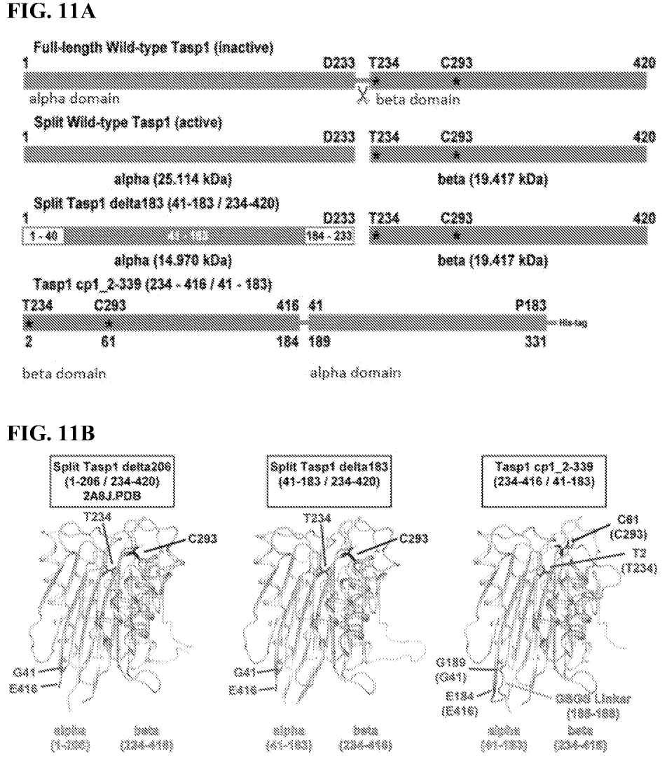

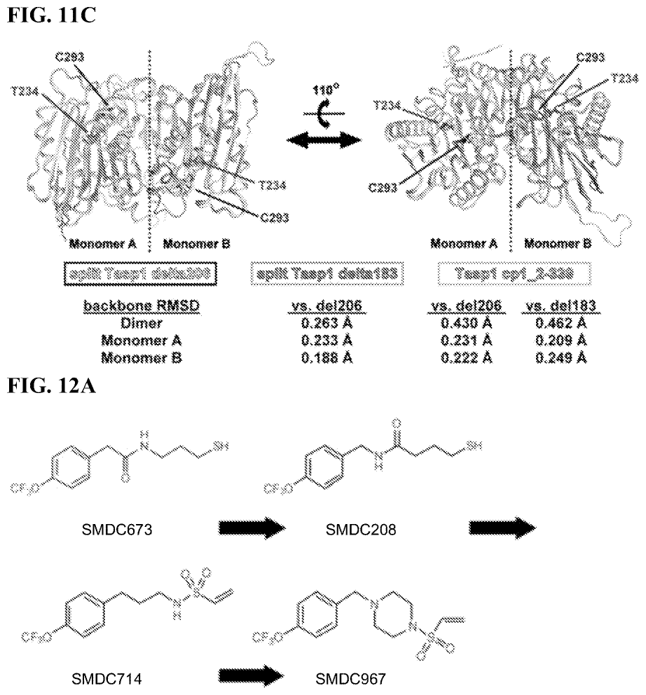

A primary LC-MS-based disulfide-trapping tethering screen using a library containing 1280 thiol-containing compounds (Burlingame, 2011; Turner, 2014; FIGS. 10A-10C) was performed against the active wild-type full-length split Tasp1 construct (henceforth, split Tasp1 WT; FIG. 11) in the presence of 625 μM beta mercaptoethanol (BME). This resulted in 21 compounds with >60% labeling (3 sigma, 1.6% hit rate), with an additional 43 compounds at >46% labeling (2 sigma, 3.4% hit rate). Of the total 64 hits, 26 thiols demonstrated strong labeling in the presence of 1 mM BME. In addition, 19 compounds showed greater than 20-fold higher % labeling for split Tasp1 WT, relative to a split Tasp1 C293A construct. Overlap of these two data sets resulted in 16 unique compounds, all of which exhibited IC₅₀ values less than 100 μM in a Taspase1 inhibition assay. The majority of the most potent compounds contained a three-carbon linker between the reactive thiol and either a substituted benzyl amide (e.g., SMDC633) or phenyl acetamide (e.g., SMDC673).

The 1,4-disubstituted phenyl acetamide series was subsequentially chosen for further structure-activity relationship (SAR) studies due to its high potency against split Tasp1 WT (IC₅₀=11.3 μM for SMDC673 after re-synthesis), lack of inhibition against the split Tasp1 C293A construct, and relative tolerance for multiple linker lengths (FIGS. 5A-5C). Evolution of the SMDC673 structure (FIG. 5A) included reversing the orientation of the amide group (SMDC208, WT IC₅₀=30.3 μM) and adding a vinyl sulfonamide warhead (SMDC 993714, WT IC₅₀=16.0 μM). Importantly, all three compounds maintained the same relative linker length between the reactive functional groups and the substituted phenyl ring.

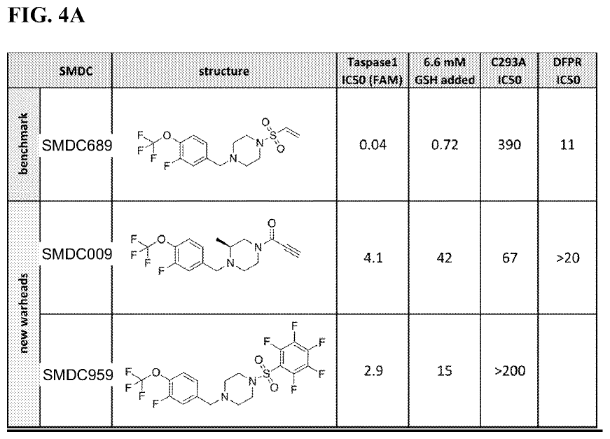

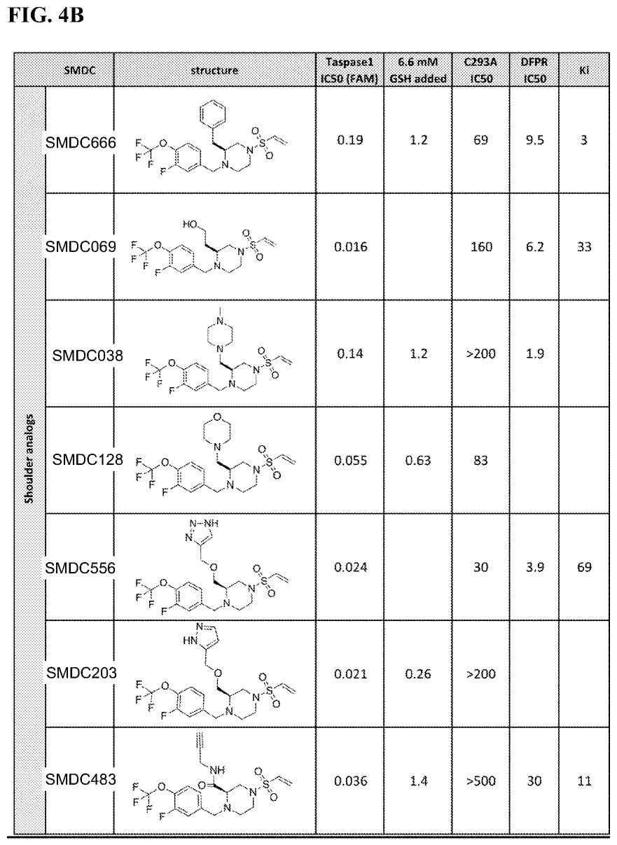

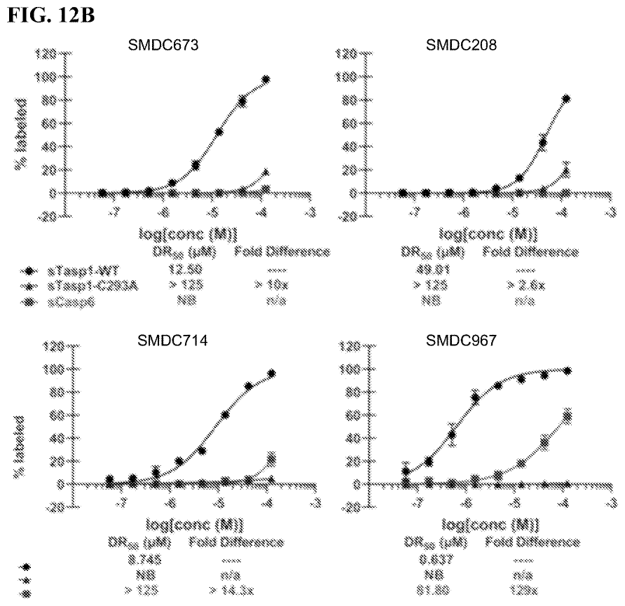

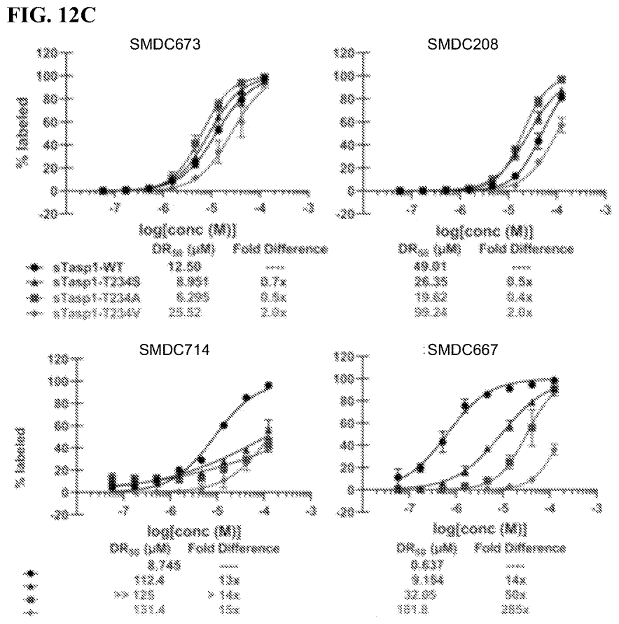

Significant improvement of the inhibitory activity was observed upon rigidifying the scaffold by replacing the flexible linker with a piperazine (SMDC967, WT IC₅₀=0.114 μM, Table 1a and FIG. 4 and FIG. 23). Importantly, all four compounds demonstrated negligible LC-MS binding and enzymatic inhibition against the split Tasp1 C293A construct, indicating binding selectivity for the active site. As measured by LC-MS dose response (DR50) values, SMDC967 demonstrated significant binding selectivity for split Tasp1 WT over split Tasp1 C293A, as well as an activated Caspase-6 (FIGS. 12A-12C). Notably, switching the trifluoromethoxy group from the 4-position to the 3-position on the phenyl ring (SMDC723) significantly reduced both binding affinity (DR₅₀>120 μM) and inhibitory activity (IC₅₀=40 μM) (FIG. 23). To determine which factors of the Thr234 sidechain are important for ligand binding, we examined the T234S, T234A, and T234V constructs of split Tasp1 (FIGS. 12A-12C). The resulting DR50 binding curves indicate that removing the threonine hydroxyl (T234A) and/or replacing it with a methyl group (T234V) greatly reduces binding affinity of SMDC967 by 50- and 285-fold, respectively. Removing the threonine methyl group (T234S) did not exhibit as significant effect on binding affinity, with a 14-fold reduction. These results suggest that both removing a potential hydrogen-bonding partner and steric crowding at the catalytic T234 site have deleterious effects on compound binding, whereas increasing the space around the T234 pocket is more tolerated.

TABLE 1a
Biochemical and Cell-Activity Data for Selected Compounds

| | WT IC₅₀ (nM) | | LC-MS | | | DFPR |
|---|---|---|---|---|---|---|
| | No | 6.6 mM | DR₅₀ | Kᵢ | k_{inact} | EC₅₀ |
| SMDC ID | GSH | GSH | (nM) | (μM) | (s⁻¹) | (μM) |
| SMDC967 | 168 ± 54 | n/a | 384 ± 238 | 30 (1) | 0.004 (1) | 26.5 (1) |
| | (22) | | (9) | | | |
| SMDC689 | 51 ± 47 | 661 ± 152 | 128 ± 20 | 45.8 ± 13.4 | 0.031 ± 0.003 | 8.3 ± 3.4 |
| | (74) | (14) | (6) | (5) | (5) | (4) |
| SMDC883 | 26 ± 8 | 340 ± 30 | 132 ± 73 | 87 (1) | 0.045 (1) | 6.5 ± 1.6 |
| | (6) | (2) | (5) | | | (2) |
| SMDC069 | 25 ± 16 | 273 ± 68 | 61 ± 9 | 33 (1) | 0.112 (1) | 2.1 ± 1.7 |
| | (8) | (3) | (3) | | | (7) |
| SMDC283 | 32 ± 18 | 237 ± 40 | 254 ± 62 | 45 (1) | 0.061 (1) | 2.3 ± 0.6 |
| | (4) | (3) | (4) | | | (6) |
| SMDC275 | 38 ± 16 | 420 ± 80 | 60 ± 28 | n/a | n/a | 6.8 (1) |
| | (5) | (3) | (3) | | | |
| * SMDC723 | 40 × 10³ | n/a | (75 ± 9) × 10³ | n/a | n/a | >40 (1) |
| | (1) | | (3) | | | |

* Negative control
New constructs & Tasp1 cp1 + SMDC967 soaked crystal structure

In order to more accurately define these interactions, we embarked on a crystallography campaign. Previous studies had suggested that both the N- and C-terminal residues of the alpha domain in the activated form of Tasp1 were too dynamic to form a lattice sufficient for crystallography (Khan, 2005). Examination of the full-length inactive single-chain crystal structure (2A8Lpdb) shows a lack of electron density between residues 1-40 and 206-229. The Tong group was subsequently able to crystallize a truncated split Tasp1 construct which deleted residues 206-233 (split Tasp1 delta206, 2A8J.pdb). However, residues 184-205 were still unobserved in the delta206 structure (Khan, 2005). In this study, we designed two truncations to remove both of the conformationally dynamic alpha domain N-terminal and C-terminal tails (FIGS. 11A-11C). Both constructs incorporate residues 41-183 of the alpha domain and the full sequence (residues 234-420) of the beta domain. The first construct (Tasp1 cp1, 6UGK.pdb) employed a circular permutation strategy in which the two domains are reversed, with the catalytically active Thr234 as the first residue in the single-chain sequence. The two domains are linked by a GSGS sequence between a truncated beta domain C-terminus (Glu416) and alpha domain N-terminus (Gly41). For clarity, we will use the split Tasp1 residue numbers for the circularly permuted variant. The second construct (split Tasp1 delta183,) employed a traditional two-chain split Tasp1 sequence. As with a circularly permuted version of human Asparaginase-1 (Li, 2012; Li, 2016), overlays of the dimeric apo split Tasp1 delta206, split Tasp1 delta183, and the Tasp1 cp1 structures did not yield significant backbone RMSD values (≤0.5 Å), indicating high structural homology.

Concurrent with the binding and enzymatic inhibition studies, we crystallized the Tasp1 cp1 construct in the presence of SMDC967 (6VKU.pdb; FIGS. 5B and 5C) using a soaking method. Examination of the active-site indicated close contacts and potential hydrogen bonds between the sulfonyl group of SMDC967 and the backbone amide of the catalytic Thr234, as well as the sidechain amide of Asn100. This observation explains the binding selectivity data of the T234 mutations. Retaining the polar sidechain (T234S) may help maintain binding interactions between the sulfonyl group of SMDC967 and Tasp1, while increasing the hydrophobicity of the residue (T234A or T234V) may disrupt such interactions. In addition, the conformation of the piperazine allows the phenyl ring to adopt a pi-stacking interaction with Tyr61, while the 4-trifluoromethoxy group is buried in a hydrophobic pocket incorporating Ile393-Ser194 and Ser376-Met377-Cys378, and capped by a loop which includes Tyr52-Ala58. Interestingly, although the inhibitor is selective and specifically binds to the active site of Tasp1, we observed partial covalency between the inhibitor and the protein. This is apparent when comparing the two monomeric units of the dimeric structure: within the same unit cell, a covalent bond is observed between the Cys293 thiol and the vinyl sulfonamide warhead in Monomer A, but not in Monomer B. An overlay of the apo Tasp1 cp1 structure with that containing the bound inhibitor shows significant conformational differences of the loop containing Cys293 (FIG. 5C). In Monomer A, the loop shifts “up”, allowing to form the covalent bond between the Cys293 thiol group and the vinyl sulfonamide warhead. Conversely, in Monomer B, the loop remains in the same relative “down” conformation similar to that observed for the apo structure, preventing close contacts between Cys293 and SMDC967. This implies that the covalent bond forms after the non-covalent interactions position the inhibitor within the binding pocket.

Evolution of Warheaded Compounds: Addition of Shoulders

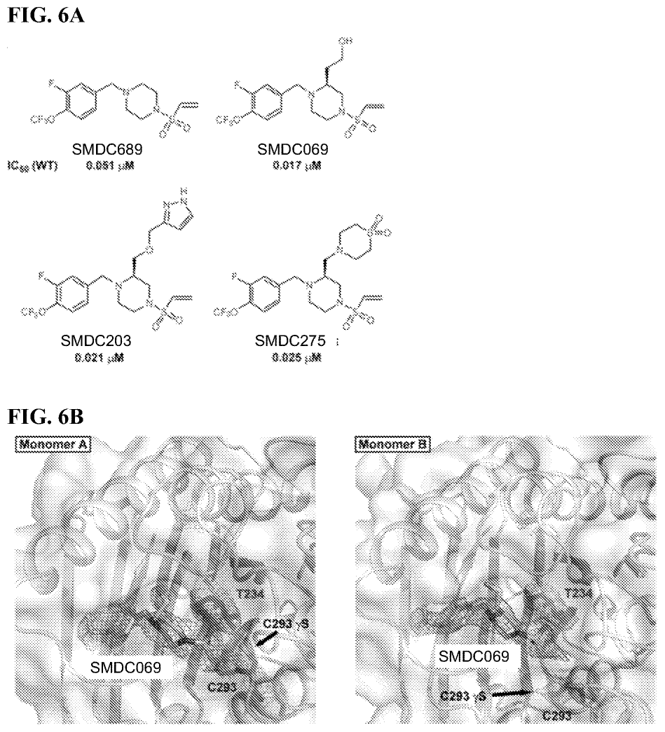

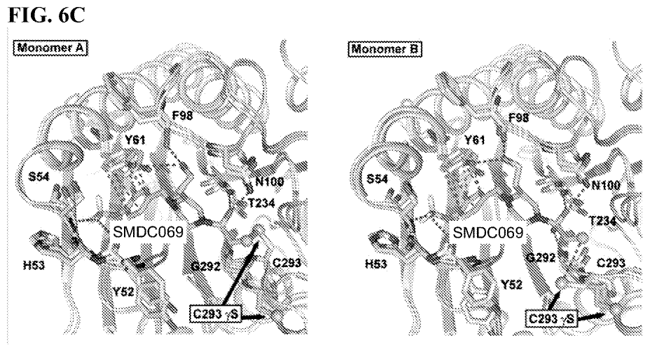

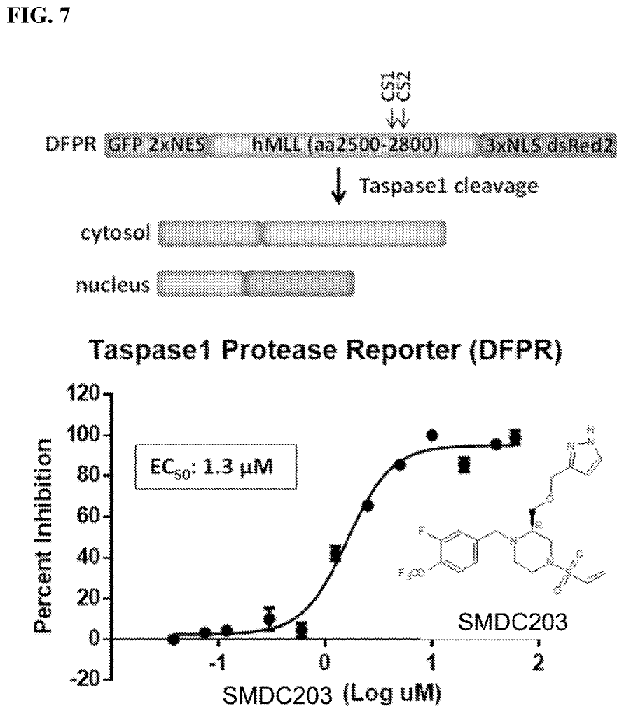

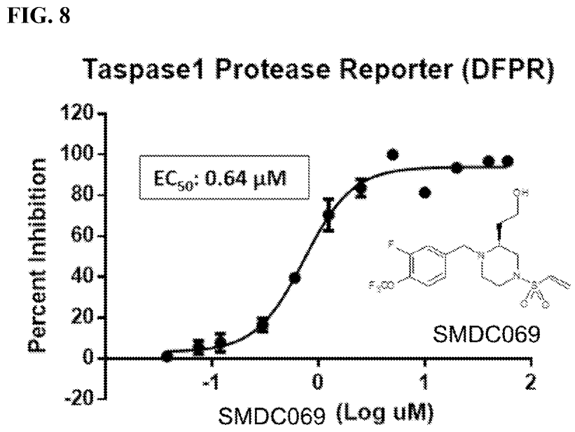

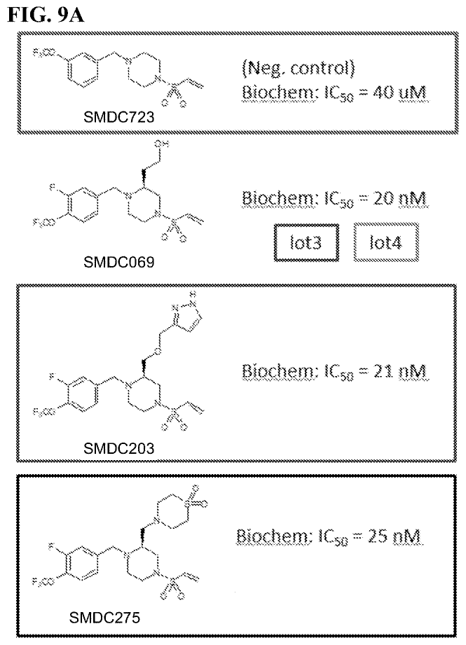

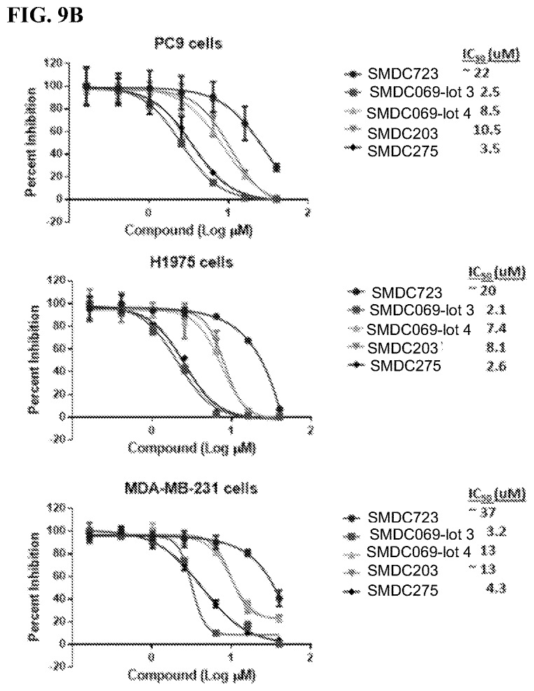

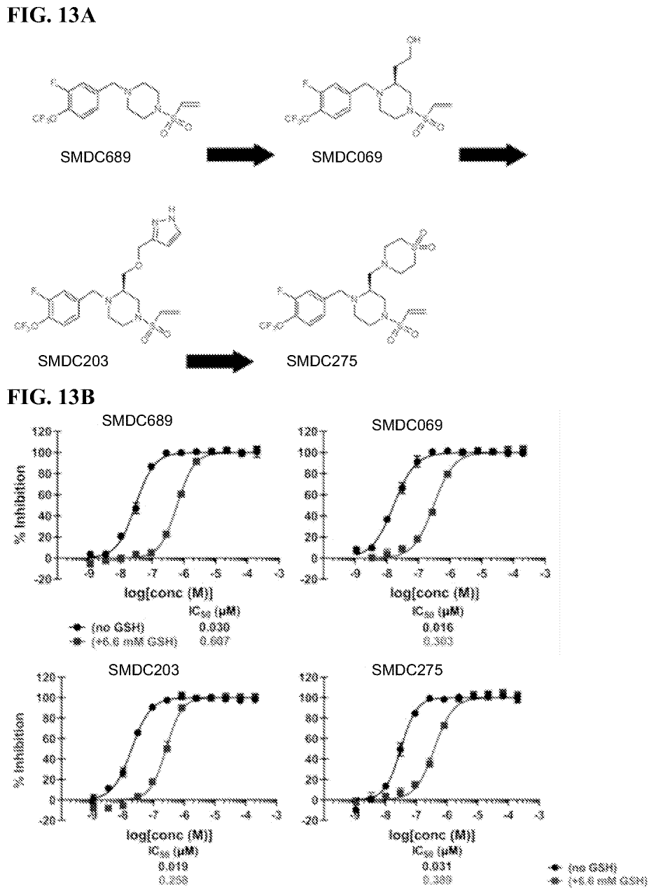

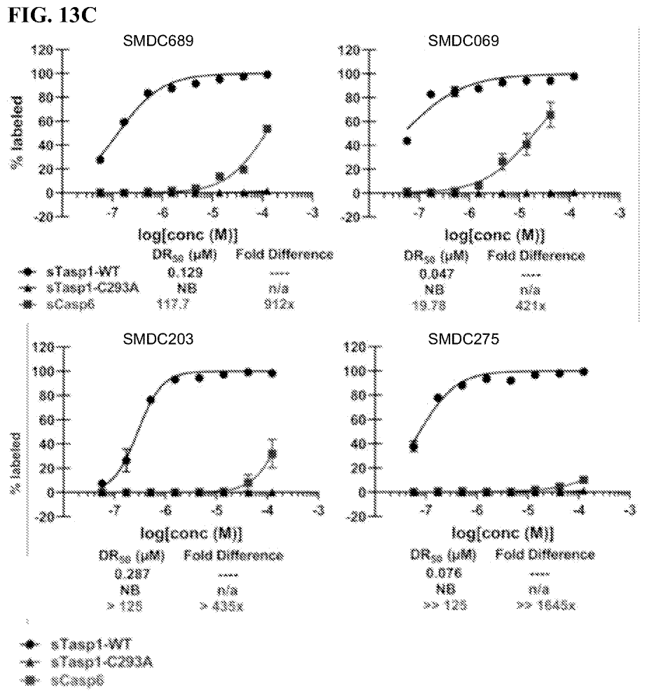

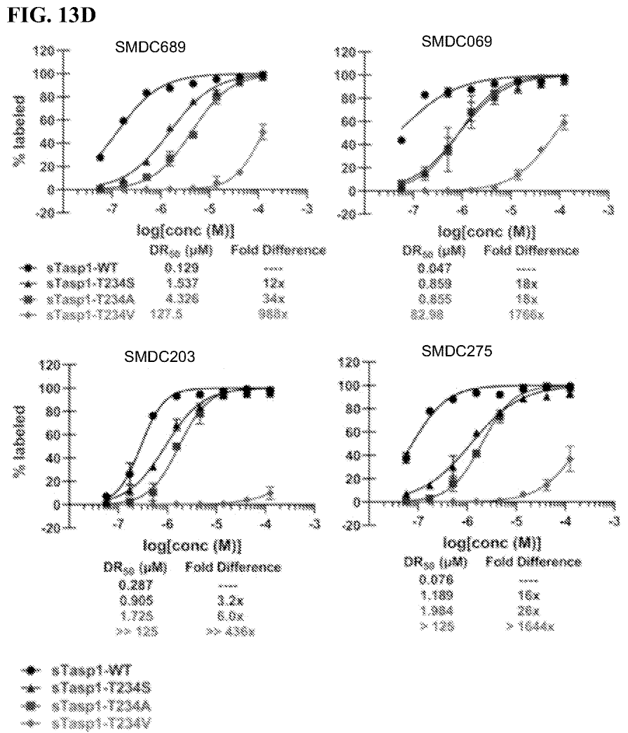

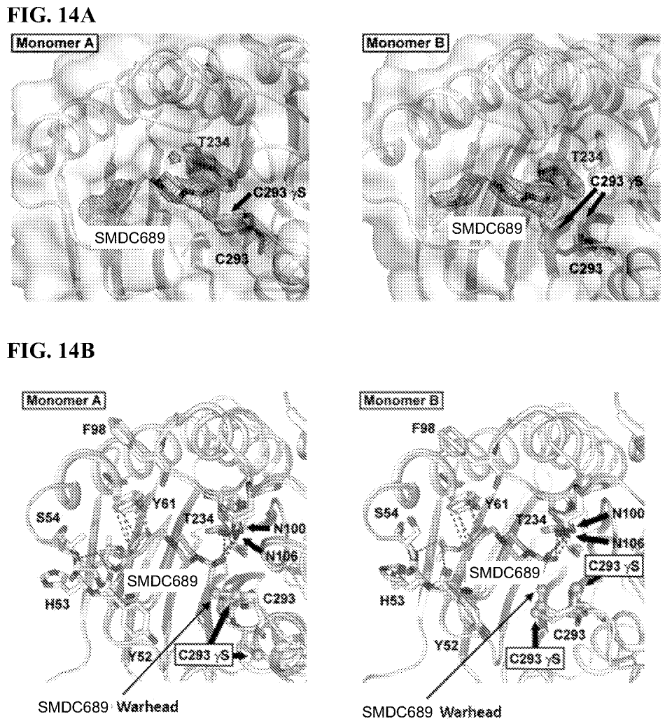

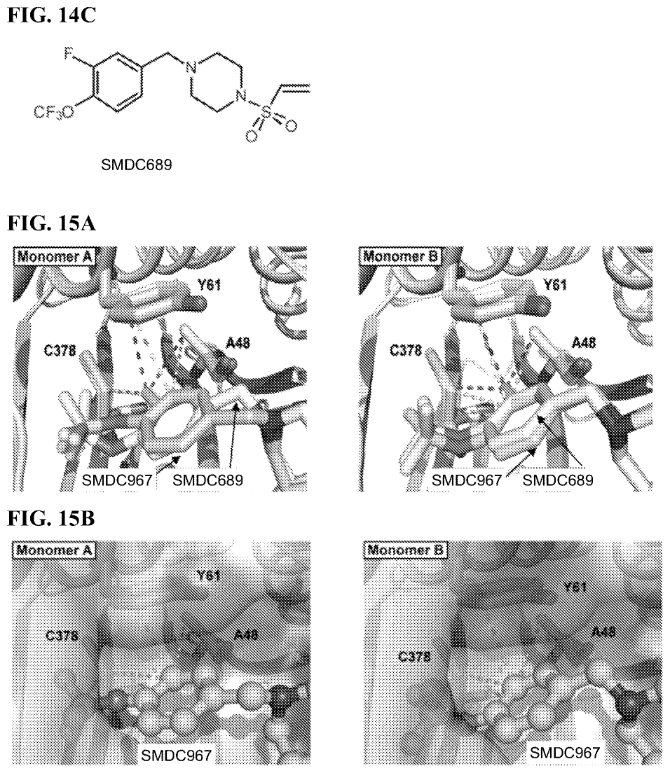

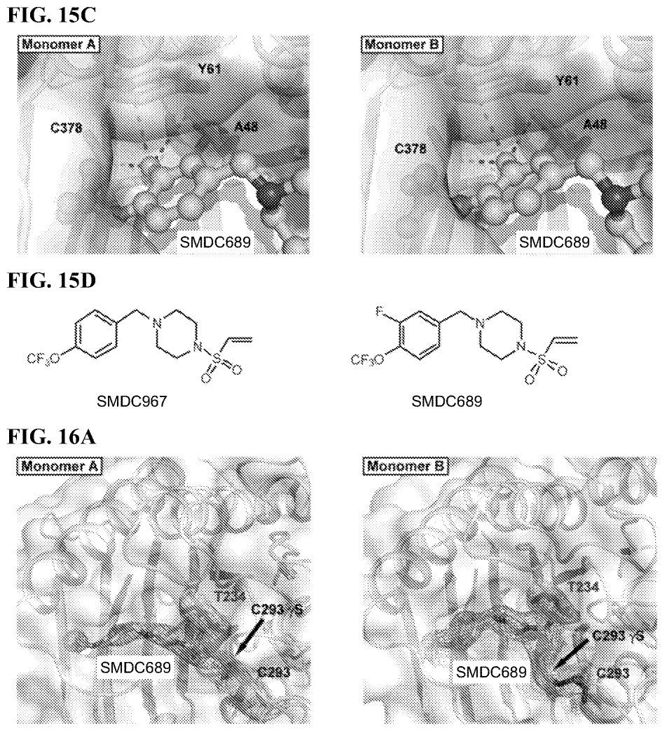

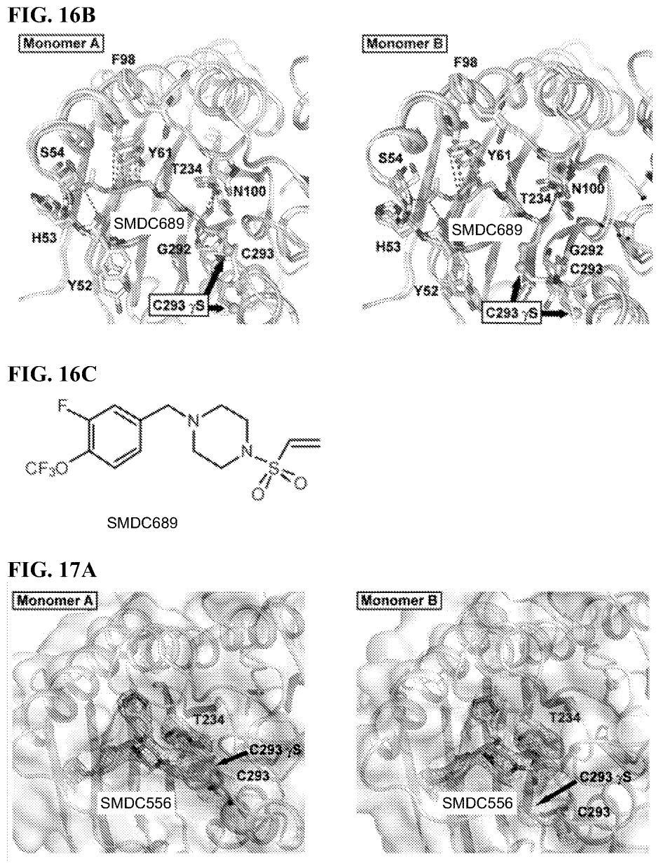

Based on the binding, enzymatic and crystallography results, we performed further SAR studies to improve the biophysical and biochemical characteristics of the SMDC967 scaffold (FIG. 6A, FIG. 23, and Table 1a). Replacing the monosubstituted 4-trifluoromethoxy phenyl ring with a disubstituted 3-fluoro-4-trifluoromethoxy phenyl group (SMDC689, IC₅₀=0.051 μM) yielded greater than 2-fold improvement in inhibitory activity (FIG. 6A), along with enhanced binding selectivity against split Tasp1 C293A and split Casp6 (FIGS. 13A-13D). Examination of the co-crystal structure SMDC689 with Tasp1 cp1 (6VKY.pdb, FIGS. 14A-14C and FIGS. 15A-15D) or split Tasp1 delta183 (FIGS. 16A-16C) indicates similar poses and close interactions with residues within the active site as those observed for SMDC967. Notably, the additional 3-fluoro group of SMDC689 has close contacts with Tyr61, Ala48, and Cys378, helping to fill out an empty gap in the hydrophobic pocket that is present in the SMDC967 structure (FIGS. 15A-15D).

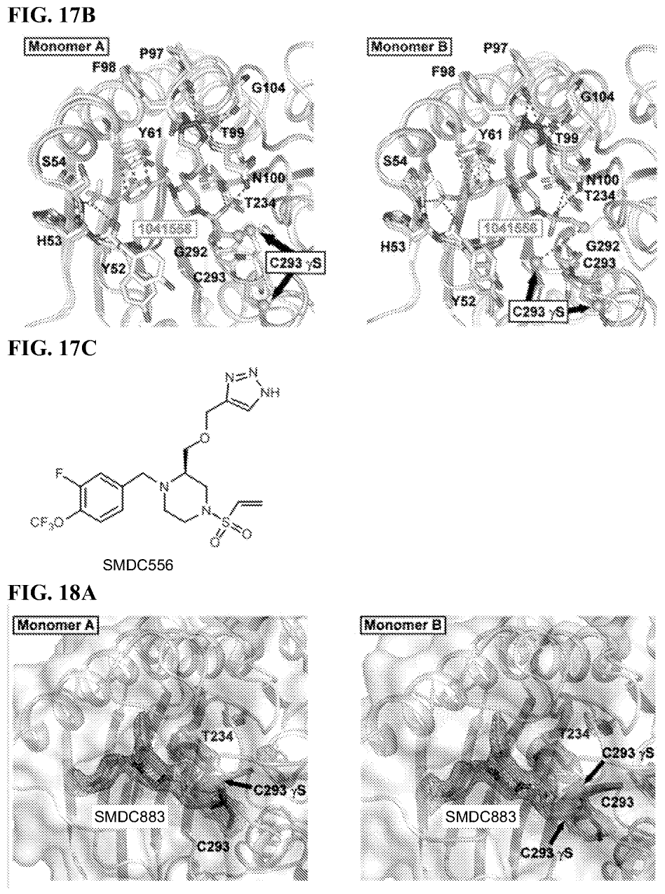

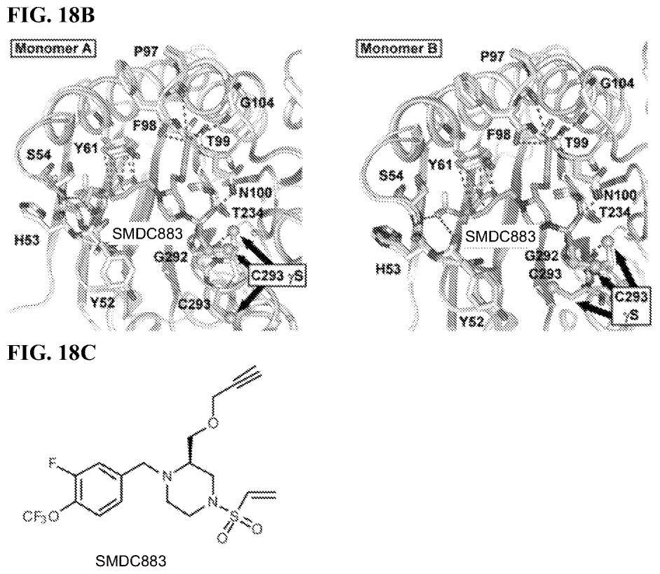

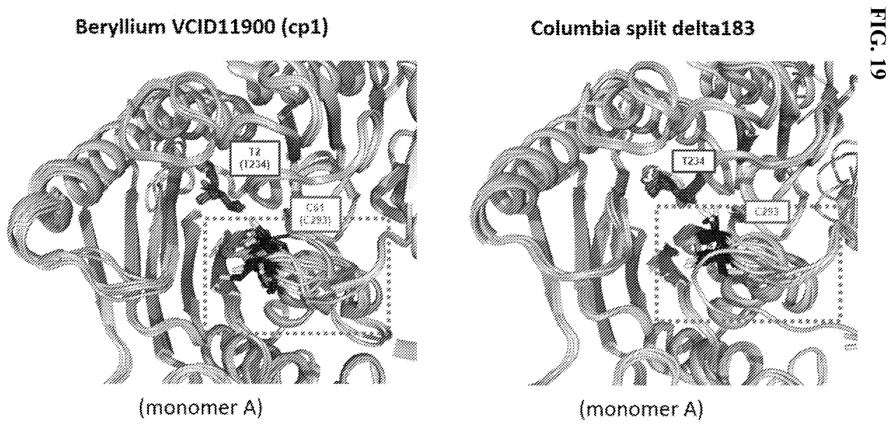

With the phenyl ring optimized, our structure-guided design strategy shifted to the piperazine ring. Efforts yielded a series of compounds (SMDC069, SMDC 883, SMDC 203, and SMDC 275) with an additional 2 to 3-fold increased inhibitory activity (all IC₅₀ values˜0.020 μM) over SMDC689 (FIG. 6A, FIG. 23, and Table 1a). The addition of these “shouldered” functional groups allowed for exploration of interactions between the compounds and the “top-shelf” of the active site, defined by a loop encompassing Phe98-Thr99-Asn100 (FIGS. 6B and 6C, FIGS. 18A-18C and FIGS. 17A-17C). In the case of SMDC069, the shouldered ethanol group displays potential hydrogen-bonding interactions with the sidechain of Tyr61 and the backbone of Phe98 (FIGS. 6B and 6C). The shouldered propargyl variant, SMDC883 also exhibited similar networks in a co-crystal structure with Tasp1 cp1 (6VKW.pdb, FIGS. 18A-18C). These interactions are further stabilized in the co-crystal structure of SMDC556 with split Tasp1 delta183 (FIGS. 17A-17C). In particular, the shouldered triazole group formed potential hydrogen bonds with the backbone carbonyl groups of Pro97, Phe98, Thr99, and Gly104. However, the triazole moiety in the shouldered region proved too unstable, and was replaced with a diazole (SMDC203). It is worth noting that SMDC883 is an intermediate of both SMDC556 and SMDC203, and may act as a “click” chemistry (Kolb, 2001) probe for further ex vivo studies. Rigidifying the shoulder with a thiomorphoroline-1,1-dioxane group (SMDC 275) only slightly improved the inhibitory activity, but showed enhanced binding affinity in the tethering assay relative to the other shouldered piperazine analogs (FIG. 23, Table 1a, FIGS. 6A-6C and FIGS. 13A-13D).

Cell Efficacy (and +Xenografts?)

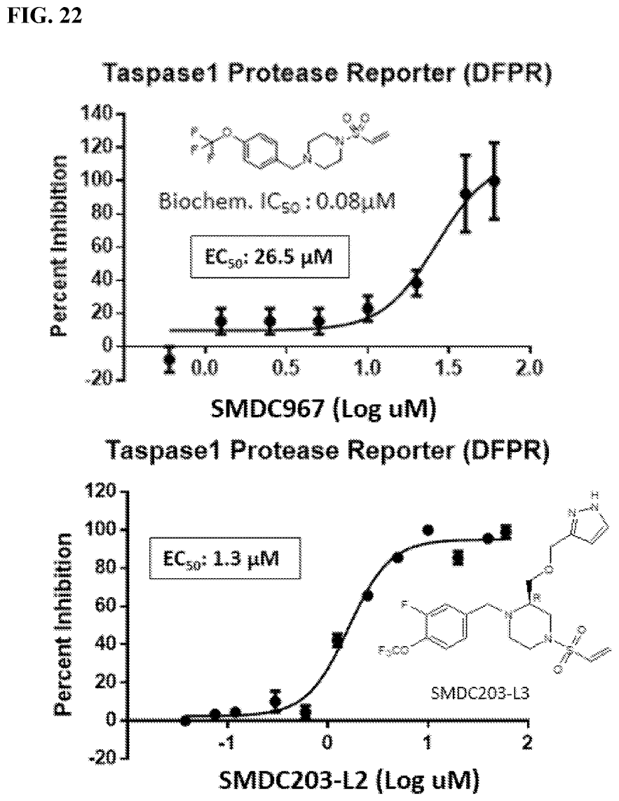

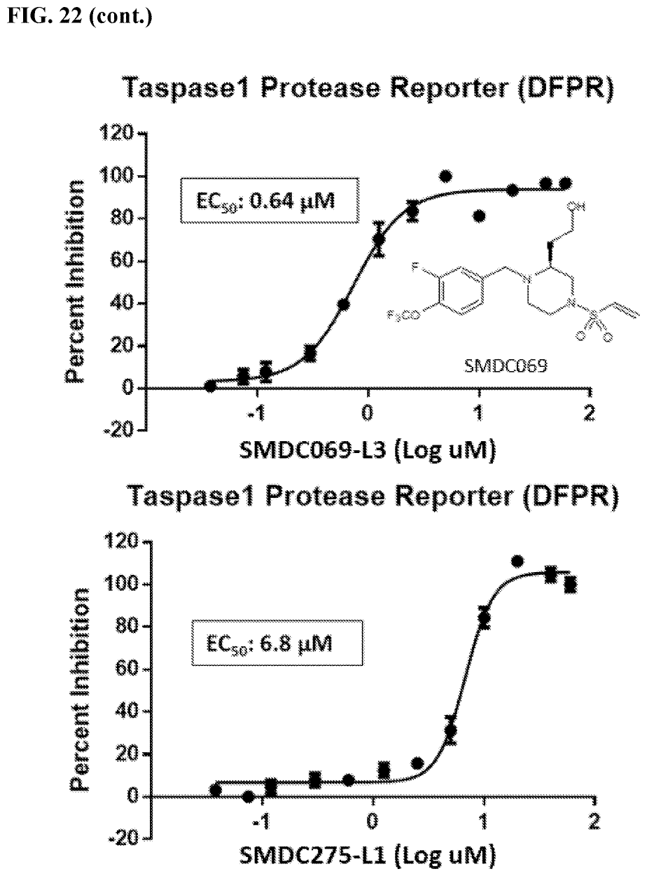

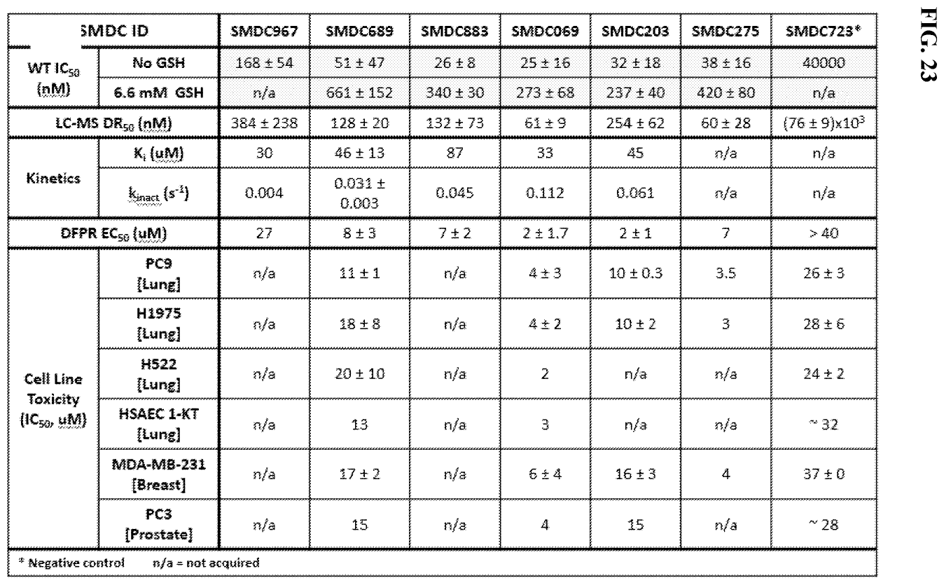

With improvements in the scaffold reaching an IC₅₀ lower limit approaching 20 nM, we forwarded the compounds for further ex vivo cell efficacy and cytotoxicity studies. Using a previously established dual-fluorescent proteolytic reporter (DFPR) assay (Chen/Hsieh, 2012), we examined the efficacy of selected potent inhibitors against Taspase-1 in transfected HEK-293T cells (FIGS. 22A-22C and FIG. 23, and Table 1a). Briefly, the reporter substrate contains a 300-residue segment of human MLL protein incorporating two Taspase-1 cleavage sites. Upon cleavage, the N-terminal region, which contains a GFP-2×NES sequence, will localize in the cytosol resulting in a green fluorescent signal. The C-terminal region, which contains a 3×NLS-dsRed2 sequence, will remain in the nucleus and displays a red fluorescent signal. Inhibition of Taspase-1 activity results in a predominantly yellow fluorescent signal located in the nucleus. The negative control compound (SMDC723) displays segregated red and green signals up to 40 μM concentration, indicating no inhibitory activity. Conversely, SMDC967 exhibits an EC₅₀ of 26.5 μM, with a 3-fold enhancement for SMDC689 (EC₅₀=8.3 μM). Adding shoulder groups to the piperazine ring further enhances ex vivo inhibitory activity (EC₅₀=6.5 μM for SMDC883 to 2.1 μM and 2.3 μM for SMDC069 and SMDC203, respectively), mirroring trends observed for the in vitro binding and activity assays.

Viability of selected compounds (SMDC689, SMDC 069, SMDC 203, and SMDC 275) were evaluated against four lung cancer (PC9, H1975, H522, and HSAEC 1-KT), one triple-negative breast cancer (MDA-MB-231), and one prostate (PC3) cell lines (Table 2a, FIG. 23). All compounds generally exhibited IC₅₀ ranging between 2 and 17 μM, while the negative control for these experiments (SMDC723) exhibited IC₅₀ values >20 μM against all cell lines studied. Although the IC₅₀ and EC₅₀ values for SMDC069 are similar (˜2-6 μM), those for SMDC203 exhibit a larger difference (IC₅₀=2.3 μM; EC₅₀=9.6-15.5 μM).

TABLE 2a
Cancer Cell Line Cytotoxicity Data

| | IC₅₀ (μM) | | | | | |
|---|---|---|---|---|---|---|
| | | | | HSAEC | MDA- | |
| | PC9 | H1975 | H522 | 1-KT | MB-231 | PC3 |
| SMDC ID | (Lung) | (Lung) | (Lung) | (Lung) | (Breast) | (Prostate) |
| SMDC689 | 11 ± 1 | 17.6 ± 8.3 | 20 ± 10 | 13 (1) | 16.5 ± 1.5 | 15 (1) |
| | (2) | (3) | (2) | | (2) | |
| SMDC069 | 3.9 ± 2.6 | 3.6 ± 2.2 | 2 (1) | 3.2 (1) | 6.0 ± 4.1 | 3.4 (1) |
| | (4) | (4) | | | (4) | |
| SMDC203 | 10.3 ± 0.3 | 9.6 ± 1.5 | n/a | n/a | 15.5 ± 2.5 | 15 (1) |
| | (2) | (2) | | | (2) | |
| SMDC275 | 3.5 (1) | 2.6 (1) | n/a | n/a | 4.3 (1) | n/a |
| *1014723 | 25.8 ± 2.7 | 27.8 ± 5.6 | 24.4 ± 1.7 | ~32 (1) | 37.0 ± 0.0 | ~28 |
| | (3) | (4) | (2) | | (2) | |

*Negative control

### Example 4: Methods

Taspase-1 Expression and Purification

Biochemical and Biophysical Constructs (Full-Length Split Tasp1)

Split Taspase-1 wild-type construct (1-233/234-420): DNA sequences were ligated into a pETDuet-1 vector (Novagen/EMD Millipore), with residues 1-233 (alpha domain) in Multiple Cloning Site-1 (MCS-1) and residues 234-420 (beta domain) in MCS-2. The alpha domain also contained a TEV-cleavable N-terminal His6-tag. Vectors were heat-shocked transformed into E. coli Rosetta2(DE3) competent cells (Novagen/EMD Millipore) and grown on LB/Agar plates supplemented with carbenicillin.

Site-directed mutagenesis: All site-directed mutagenesis reactions were performed on the above template DNA with QuikChange Lightning kits (Agilent). Optimized forward primers (C293A, TGT→GCG; T234A, ACG→GCG; T234S, ACG→TCG; T234V, ACG→GTG), and their respective reversed complements were designed using Agilent's QuikChange Primer Design website and purchased from IDT.

Expression and Purification: All full-length split Tasp1 constructs used for biochemical and biophysical assays were expressed in 2×YT media containing carbenicillin. Bacterial pellets were harvested and frozen at −80° C. prior to lysis via sonication. Lysis buffer consisted of 50 mM Tris (pH 8), 500 mM NaCl, 5% glycerol, 5 mM BME, and 30 mM imidazole. Solutions were clarified prior to loading onto HisTrap FF columns (daisy-chained 2×5 mL, GE Healthcare Lifesciences) attached to an AKTA Pure FPLC (GE Healthcare Lifesciences). Proteins were eluted using a step function from 30 mM to 200 mM imidazole. Major fractions were collected and dialyzed overnight using a 6-8 kD MWCO dialysis tubing (Spectra/Por-1, Spectrum Labs) in the presence of 1 mg/mL TEV protease. Dialysis buffer consisted of 20 mM Tris (pH 8), 500 mM NaCl, 5% (v/v) glycerol, 0.5 mM TCEP. Samples were applied through the HisTrap FF columns again to remove the His6-tag and TEV protease. The flow-through from the loading step was concentrated with 30 kD MWCO Amicon Centrifiugal Filter Units (Millipore) to 2 mL prior to injection on a Superdex 200 16/60 or Superdex 200 10/300 Increase column (GE Healthcare Lifesciences). Proteins were eluted using 1.5 CV of fresh dialysis buffer. Major fractions were pooled and concentrated to ˜0.5-1 mL final volume prior to aliquoting and flash freezing. All protein samples were stored at −80° C. until ready for analysis. Protein concentrations for all full length Tasp1 constructs (wild-type, C293 Å, and T234X) were evaluated using a NanoDrop 2000c (Thermo Scientific) using ε₂₈₀=26930 M⁻¹ cm⁻¹.

Crystallography constructs (split truncated Tasp1 and circularly permuted Tasp1)

Split Taspase1 delta183 construct (41-183/234-420): DNA encoding residues 41-183 with a non-cleavable N-terminal His6-Tag (MCS-1) and residues 234-420 (MCS-2) were ligated into pCDFDuet vectors (Novagen/EMD Millipore). Vectors were then transformed into E. coli BL21 (DE3) Star competent cells. Split Tasp1 delta183 was expressed and purified as previously reported (Khan, 2005). Expression was induced with 0.3 mM IPTG at 20° C. for 18 hours and purified with Ni-NTA Superflow (Qiagen) and gel filtration (Sephacryl S-300) chromatography (in a buffer containing 450 mM NaCl, 20 mM Tris-HCl (pH 7.5) and 5 mM DTT). Split Tasp1 delta183 protein stock was supplemented with 5% (v/v) glycerol and concentrated to 5 mg/ml before being flash-frozen in liquid nitrogen.

Circularly permuted Taspase1 (CP-1: CID 11900): The truncated Tasp1 cp-1_2-339 (a.a. 234-416-GSGS-41-183) human Taspase1 constructs with a hexa-histidine tag at the C-terminus was expressed and purified as previously reported (Nagaratnam, 2020).

Dynamic Light Scattering (DLS)

All small molecule inhibitors were subjected to DLS analysis on a DynaPro Plate Reader II (Wyatt) using either one of two buffers: (1, crystallography conditions) 100 mM MES pH 7.5, 250 mM KF, 0.005% (v/v) Tween20, 5% (v/v) DMSO; (2, biophysics and biochemical conditions) 100 mM NH₄OAc pH 8.3, 0.005% (v/v) Tween20, 5% (v/v) DMSO. DLS data were collected in a dose-dependent manner. Small molecule samples in assay buffer alone were serially diluted in 2-fold steps, starting with a 1 mM high concentration (40 μL total volume; 11 steps, with a replicate), and were loaded onto a 384-well clear bottom plate (Corning #3540), with the 2 outside columns containing blank buffer solution. Samples were incubated in the plate reader at 25° C. during the data collection, which consisted of 10 reads per well and a 2 second acquisition time per read. Data was acquired and analyzed using Dynamics 7.1.7.16, with the output reporting estimated particle radius size. Experimental errors were estimated by a sum-of-squares parameter resulting from the 10 reads/well. Samples with estimated particle sizes >1 nm were considered to contain aggregates. All compounds included in this study, with the exception of SMDC556, exhibited estimated particle sizes >1 nm at concentrations up to 125 μM in both buffer conditions (data not shown).

LC-MS Tethering and Dose-Response Assays

The general methodology for the high-content primary screen using a 1280-member disulfide-containing compound library (Burlingame, 2011; Turner, 2014) was as previously described (Hallenbeck, 2018). All LC-MS tethering and dose response assays were collected on a Waters Acquity UPLC with either a (1) Micromass LCT Premier or (2) Xevo G2-XS Q-Tof mass spectrometer. A Waters UPLC Protein BEH-C4 Column (300 Å, 1.7 μm, 2.1 mm×50 mm) was used to desalt the samples prior to application on the mass spectrometer. For the primary tethering assays, 500 nM protein samples in 100 mM NH₄OAc (pH 8.3) and 625 μM BME were loaded onto a 384-well plate. For the follow-up dose-response tethering assays, 200-300 nM protein samples in 100 mM NH₄OAc (pH 8.3) buffer containing either 1 mM BME or glutathione were loaded onto a 384-well plate. A Biomek FX Automation Workstation (Beckman Coulter) equipped with a 384-pin tool was used to simultaneously apply the 50 mM compound stocks in 50 nL increments to individual wells containing protein solution. Final concentration of the compounds was 10011M for the single-dose high content screen. In the case of the 8-point dose response assays, the 50 mM compound stocks were serially diluted in 3-fold increments, with a final high concentration of 125 μM after pinning. Mixed samples were incubated at room temperature for at least 1 hr prior to loading the plates into the LC-MS, and at 10° C. during the data acquisition. All data acquisition, processing, and analysis were performed using MassLynx 4.1 (Waters). Raw m/z spectra were processed with the MaxEnt-1 deconvolution algorithm (Ferrige, 1991) within the MassLynx program suite.

Crystallization, Data Collection and Structure Determination

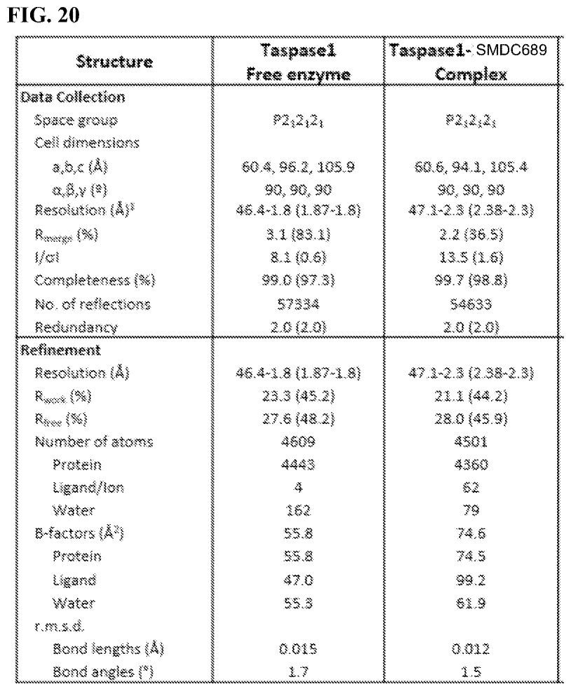

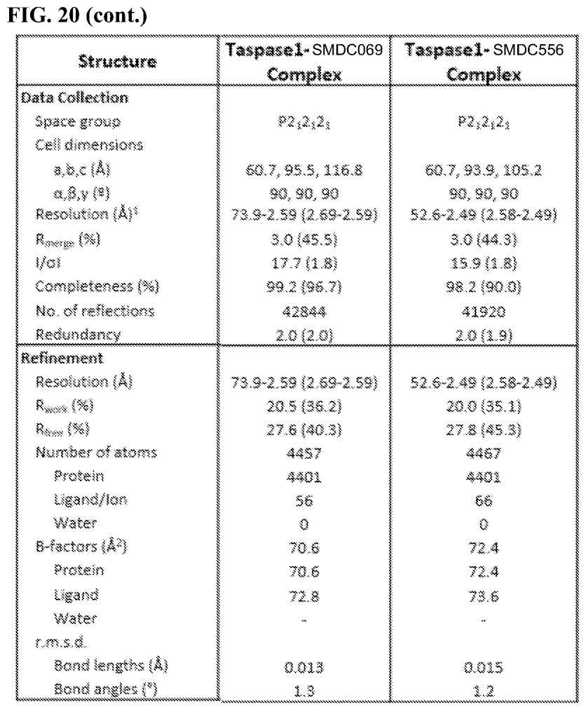

Split Taspase1 delta183. Split Tasp1 delta183 crystals were obtained using the hanging-drop vapor-diffusion method at 20° C. with a reservoir solution containing 20-26% (v/v) MPD and 0.1 M IVIES (pH 6.5). Seeding resulted in faster growth and more consistent good quality crystals. To obtain inhibitor-bound split Tasp1 delta183 structures, crystals were soaked with 5 to 25 mM inhibitor for 5 to 20 hours. Split Tasp1 delta183 crystals were cryoprotected with 40% (w/v) MPD before being flash-frozen in liquid nitrogen for diffraction analysis and data collection at 100 K. X-ray diffraction data were collected at the Advanced Photon Source (APS) beamline 24-ID-C and the diffraction images were processed and scaled using the XDS program (Kabsch, 2010). The structure was solved by molecular replacement with Phaser (McCoy et al., 2007) using Taspase1 (PDB 2A8J) as the initial model. Further structure refinement was performed using PHENIX (Adams et al., 2002) or Refmac (Murshudov, 1997) and the atomic model was rebuilt with the Coot program (Emsley, 2004). The crystallographic information is summarized in FIG. 20.

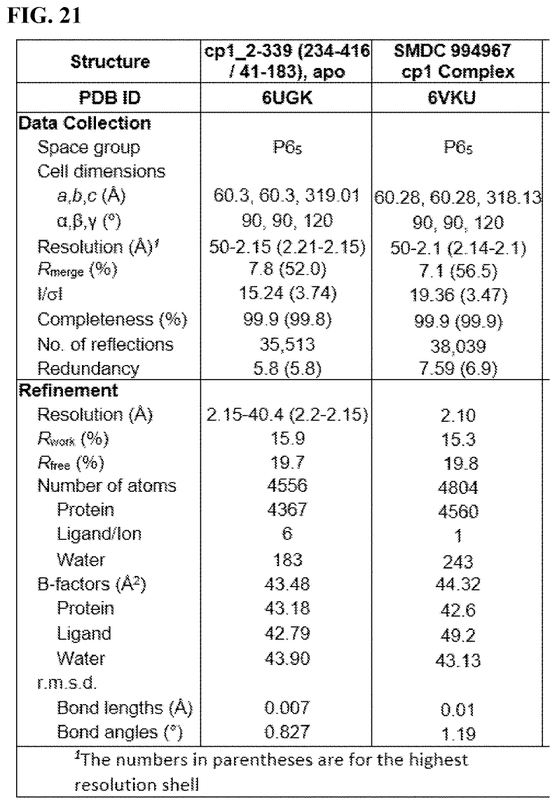

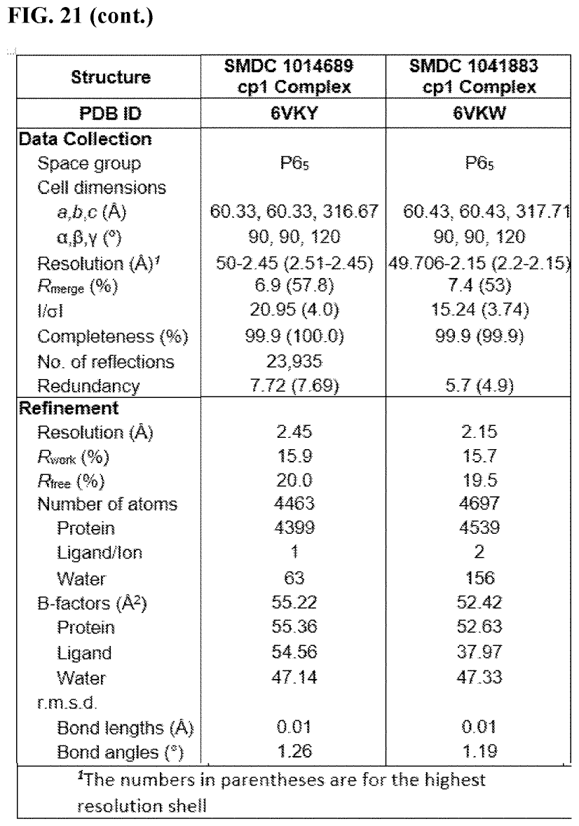

Circularly permuted Taspase1 (CP-1: CID 11900): The crystallization conditions of CP-1 (Tasp1 cp-1_2-339) human Taspase1 at ˜9-10 mg/ml were identified from PACT screen condition (B11: 0.2 M calcium chloride, 0.1 M MES pH 6, 20% (w/v) PEG 6000) at 16° C. using hanging-drop vapor-diffusion method. Ligand bound co-crystals were obtained with a 1 hour incubation at 4° C. with 2.5 mM compound prior to crystal tray set-up. Cryo-protection of 20% ethylene glycol was used for all CP-1 crystals. X-ray diffraction data were collected at the APS beamline 21-ID-F and the diffraction images were processed and scaled as described above. The crystallographic data collection and structure refinement statistics are summarized in FIG. 21.

### REFERENCES

1. Niizuma H, Cheng E H, Hsieh J J. Taspase 1: A protease with many biological surprises. Mol Cell Oncol. 2015 Jan. 23; 2(4):e999513. 2015 October-December PMID: 27308523.
2. Chen DY1, Liu H, Takeda S, et al. Taspase1 Functions as a non-Oncogene Addiction Protease that Coordinates Cancer Cell Proliferation and Apoptosis. Cancer Res., 70, 5358-5367, 2010.
3. Hsieh et al., Taspase1: A Threonine Aspartase Required for Cleavage of MLL and Proper HOXgene expression. Cell, 115, 293-303, 2003.
4. Khan et al., Crystal Structure of Human Taspase1, a Crucial Protease Regulating the Function of MLL. Structure, 13, 1443-1452, 2005.
5. Takeda et al., Proteolysis of MLL family proteins is essential for Taspase1-orchestrated cell cycle progression. Genes and Development, 20, 2397-2409, 2006.
6. Zhou et al., Uncleaved TFIIA Is a Substrate for Taspase1 and Active in Transcription. Molecular and Cellular Biology, 26, 2728-2735, 2006.
7. Chen DY1, Lee Y, Van Tine B A, et al. A Pharmacologic Inhibitor of the Protease Taspase1 Effectively Inhibits Breast and Brain Tumor Growth. Cancer Res., 72, 736-746, 2012.
8. Takeda 51, Liu H, Sasagawa S, et al. HGF-MET signals via the MLL-ETS2 complex in hepatocellular carcinoma; J Clin Invest; 123(7): 3154-3165, 2013.
9. Ostrem J M, Peters U, Sos M L, Wells J A, Shokat, K M. K-Ras(G12C) inhibitors allosterically control GTP affinity and effector interactions; Nature; 503(7477) 548-551, 2013.
10. Lito P, Soloman M, Li L S, Hansen R, Rosen N. Allele-specific inhibitors inactivate mutant KRAS G12C by a trapping mechanism; Science; 351(6273):604-608, 2016.
11. Hallenbeck K K, Turner D M, Renslo A R, Arkin M R. Covalent Strategies in Structure-Guided Drug Discovery. Curr Top Med Chem, 2016, epub.
Abdeldayem, A., Raouf, Y. S., Constantinescu, S. N., Moriggl, R. & Gunning, P. T. Advances in covalent kinase inhibitors. Chem. Soc. Rev. (2020) doi:10.1039/C9CS00720B.
Adams, P. D. et al. PHENIX: building new software for automated crystallographic structure determination. Acta Cryst D 58, 1948-1954 (2002).
Burlingame, M. A., Tom, C. T. M. B. & Renslo, A. R. Simple One-Pot Synthesis of Disulfide Fragments for Use in Disulfide-Exchange Screening. ACS Comb. Sci. 13, 205-208 (2011).
Chen, D. Y. et al. A pharmacologic inhibitor of the protease Taspase1 effectively inhibits breast and brain tumor growth. Cancer Res. 72, 736-746 (2012).
Chen, D. Y. et al. Taspase1 functions as a non-oncogene addiction protease that coordinates cancer cell proliferation and apoptosis. Cancer Res. 70, 5358-5367 (2010).
Dong, Y. et al. Taspase1 cleaves MLL1 to activate cyclin E for HER2/neu breast tumorigenesis. Cell Res. 24, 1354-1366 (2014).
Dorrance, A. M. et al. M11 partial tandem duplication induces aberrant Hox expression in vivo via specific epigenetic alterations. J. Clin. Invest. 116, 2707-2716 (2006).
Emsley, P. & Cowtan, K. Coot: model-building tools for molecular graphics. Acta Cryst D 60, 2126-2132 (2004).
Erlanson, D. A., Wells, J. A. & Braisted, A. C. Tethering: fragment-based drug discovery. Annu Rev Biophys Biomol Struct 33, 199-223 (2004).
Ferrige, A. G. et al. Disentangling electrospray spectra with maximum entropy. Rapid Commun. Mass Spectrom. 6, 707-711 (1992).
Gao, J. & Wells, J. A. Identification of Specific Tethered Inhibitors for Caspase-5. Chem Biol Drug Des 79, 209-215 (2012).
Gentile, D. R. et al. Ras Binder Induces a Modified Switch-II Pocket in GTP and GDP States. Cell Chemical Biology 24, 1455-1466.e14 (2017).
Gribko, A. et al. Disease-relevant signalling-pathways in head and neck cancer: Taspase1's proteolytic activity fine-tunes TFIIA function. Sci Rep 7, 14937 (2017).
Hallenbeck, K. K. et al. A Liquid Chromatography/Mass Spectrometry Method for Screening Disulfide Tethering Fragments. SLAS Discov 23, 183-192 (2018).
Hallenbeck, K. K., Turner, D. M., Renslo, A. R. & Arkin, M. R. Targeting Non-Catalytic Cysteine Residues Through Structure-Guided Drug Discovery. Curr Top Med Chem 17, 4-15 (2017).
Hardy, J. A., Lam, J., Nguyen, J. T., O'Brien, T. & Wells, J. A. Discovery of an allosteric site in the caspases. PNAS 101, 12461-12466 (2004).
Heikal, A. et al. ‘Tethering’ fragment-based drug discovery to identify inhibitors of the essential respiratory membrane protein type II NADH dehydrogenase. Bioorg. Med. Chem. Lett. 28, 2239-2243 (2018).
Hong, V., Presolski, S. I., Ma, C. & Finn, M. G. Analysis and Optimization of Copper-Catalyzed Azide-Alkyne Cycloaddition for Bioconjugation. Angew. Chem. Int. Ed. 48, 9879-9883 (2009).
Hong, V., Steinmetz, N. F., Manchester, M. & Finn, M. G. Labeling Live Cells by Copper-Catalyzed Alkyne-Azide Click Chemistry. Bioconjugate Chem. 21, 1912-1916 (2010).
Hsieh, J. J.-D., Cheng, E. H.-Y. & Korsmeyer, S. J. Taspase1: a threonine aspartase required for cleavage of MLL and proper HOX gene expression. Cell 115, 293-303 (2003).
Kabsch, W. Integration, scaling, space-group assignment and post-refinement. Acta Cryst D 66, 133-144 (2010).
Kathman, S. G. & Statsyuk, A. V. Covalent Tethering of Fragments For Covalent Probe Discovery. Medchemcomm 7, 576-585 (2016).
Keedy, D. A. et al. An expanded allosteric network in PTP1B by multitemperature crystallography, fragment screening, and covalent tethering. eLife 7, e36307 (2018).
Khan, J. A., Dunn, B. M. & Tong, L. Crystal structure of human Taspase1, a crucial protease regulating the function of MLL. Structure 13, 1443-1452 (2005).
Knauer, S. K. et al. Bioassays to monitor Taspase1 function for the identification of pharmacogenetic inhibitors. PLoS ONE 6, e18253 (2011).
Kolb, H. C., Finn, M. G. & Sharpless, K. B. Click Chemistry: Diverse Chemical Function from a Few Good Reactions. Angew. Chem. Int. Ed. Engl. 40, 2004-2021 (2001).
Lee, J. T. et al. Design, syntheses, and evaluation of Taspase1 inhibitors. Bioorg. Med. Chem. Lett. 19, 5086-5090 (2009).
Li, W. et al. Intramolecular Cleavage of the hASRGL1 Homodimer Occurs in Two Stages. Biochemistry 55, 960-969 (2016).
Li, W. et al. Uncoupling Intramolecular Processing and Substrate Hydrolysis in the N-Terminal Nucleophile Hydrolase hASRGL1 by Circular Permutation. ACS Chem. Biol. 7, 1840-1847 (2012).
McCoy, A. J. et al. Phaser crystallographic software. J Appl Crystallogr 40, 658-674 (2007).
Murshudov, G. N., Vagin, A. A. & Dodson, E. J. Refinement of Macromolecular Structures by the Maximum-Likelihood Method. Acta Crystallogr. D Biol. Crystallogr. 53, 240-255 (1997).
Nagaratnam, N. et al. Enhanced X-ray diffraction of in vivo-grown [mu]NS crystals by viscous jets at XFELs. Acta Crystallographica Section F 76, 278-289 (2020).
Niehof, M. & Borlak, J. EPS15R, TASP1, and PRPF3 are novel disease candidate genes targeted by HNF4alpha splice variants in hepatocellular carcinomas. Gastroenterology 134, 1191-1202 (2008).
Niizuma, H., Cheng, E. H. & Hsieh, J. J. Taspase 1: A protease with many biological surprises. Mol Cell Oncol 2, e999513 (2015).
Nnadi, C. I. et al. Novel K-Ras G12C Switch-I I Covalent Binders Destabilize Ras and Accelerate Nucleotide Exchange. J. Chem. Inf. Model. 58, 464-471 (2018).
Olp, M. D. et al. Covalent-Fragment Screening of BRD4 Identifies a Ligandable Site Orthogonal to the Acetyl-Lysine Binding Sites. ACS Chem. Biol. 15, 1036-1049 (2020).
Ostrem, J. M., Peters, U., Sos, M. L., Wells, J. A. & Shokat, K. M. K-Ras(G12C) inhibitors allosterically control GTP affinity and effector interactions. Nature 503, 548-551 (2013).
Oyama, T. et al. Cleavage of TFIIA by Taspase1 activates TRF2-specified mammalian male germ cell programs. Dev. Cell 27, 188-200 (2013).
Presolski, S. I., Hong, V. P. & Finn, M. G. Copper-Catalyzed Azide-Alkyne Click Chemistry for Bioconjugation. Current Protocols in Chemical Biology 3, 153-162 (2011).
Scrideli, C. A. et al. Gene expression profile analysis of primary glioblastomas and non-neoplastic brain tissue: identification of potential target genes by oligonucleotide microarray and real-time quantitative PCR. J Neurooncol 88, 281-291 (2008).
Sijbesma, E. et al. Site-Directed Fragment-Based Screening for the Discovery of Protein-Protein Interaction Stabilizers. J. Am. Chem. Soc. 141, 3524-3531 (2019).
Stauber, R. H., Hahlbrock, A., Knauer, S. K. & Wunsch, D. Cleaving for growth: threonine aspartase 1—a protease relevant for development and disease. FASEB J. 30, 1012-1022 (2016).
Takeda, S. et al. Proteolysis of MLL family proteins is essential for taspase1-orchestrated cell cycle progression. Genes Dev. 20, 2397-2409 (2006).
Turner, D. M., Tom, C. T. M. B. & Renslo, A. R. Simple Plate-Based, Parallel Synthesis of Disulfide Fragments using the CuAAC Click Reaction. ACS Comb. Sci. 16, 661-664 (2014). van den Boom, J. et al. Peptidyl succinimidyl peptides as taspase 1 inhibitors. Chembiochem 15, 2233-2237 (2014).
Wang, F. et al. Site-specific proteolytic cleavage prevents ubiquitination and degradation of human REV3L, the catalytic subunit of DNA polymerase ζ Nucleic Acids Res 48, 3619-3637 (2020).
Wang, L. et al. Covalent binding design strategy: A prospective method for discovery of potent targeted anticancer agents. Eur J Med Chem 142, 493-505 (2017).
Wunsch, D. et al. Taspase1: a ‘misunderstood’ protease with translational cancer relevance. Oncogene 35, 3351-3364 (2016).
Zhang, Y., Ji, T., Ma, S. & Wu, W. MLL1 promotes migration and invasion of fibroblast-like synoviocytes in rheumatoid arthritis by activating the TRIF/NF-κB signaling pathway via H3K4me3 enrichment in the TLR4 promoter region. Int. Immunopharmacol. 82, 106220 (2020).
Zhou, H. et al. Uncleaved TFIIA Is a Substrate for Taspase 1 and Active in Transcription. Mol Cell Biol 26, 2728-2735 (2006).

What is claimed is:

1. A compound having the formula: 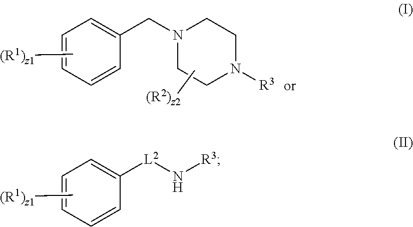
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1B}, —NHC(O)NR^{1A}R^{1B}N(O)ₘ₁, —NR^{1A}R^{1B}, —C(O)R^{1C}, —C(O)—OR^{1C}, —C(O) NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
L² is substituted or unsubstituted alkylene;
R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O) NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents may optionally be joined to form a substituted or unsubstituted cycloalkyl, substituted or unsubstituted heterocycloalkyl, substituted or unsubstituted aryl, or substituted or unsubstituted heteroaryl;
R³ is independently —CN, 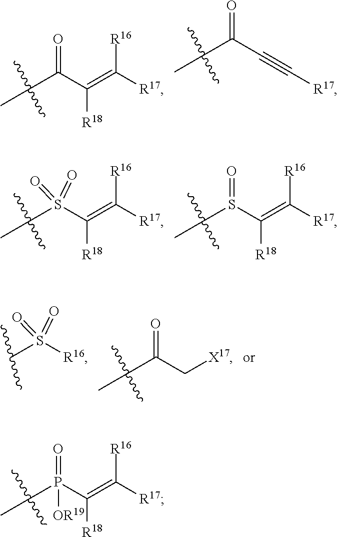
wherein R¹⁶ is independently hydrogen, halogen, —CX¹⁶₃, —CHX¹⁶₂, —CH₂X¹⁶, —CN, —SOₙ₁₆R^{16A}—SOᵥ₁₆NR^{16A}R^{16B}, —NHNR^{16A}R^{16B}, —ONR^{16A}R^{16B}, —NHC(O)NHNR^{16A}R^{16B}, —NHC(O)NR^{16A}R^{16B}, —N(O)ₘ₁₆, —NR^{16A}R^{16B}, —C(O)R^{16A}, —C(O)—OR^{16A}, —C(O)NR^{16A}R^{16B}, —OR^{16A}, —NR^{16A}SO₂R^{16B}, —NR^{16A}C(O)R^{16B}, —NR^{16A}C(O)OR_{16B}, —NR^{16A}OR^{16B}, —OCX¹⁶₃, —OCHX¹⁶₂, —OCH₂X¹⁶, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁷ is independently hydrogen,
halogen, —CX¹⁷₃, —CHX¹⁷₂, —CH₂X¹⁷, —CN, —SOⁿ¹⁷R^{17A}, —SOᵥ₁₇NR^{17A}R^{17B}, —NHNR^{17A}R^{17B}, —ONR^{17A}R^{17B}, —NHC(O)NHNR^{17A}R^{17B},
—NHC(O)NR^{17A}R^{17B}, —N(O)ₘ₁₇, —NR^{17A}R^{17B}, —C(O)R^{17A}, —C(O)—OR^{17A}, —C(O)NR^{17A}R^{17B}, —OR^{17A}, —NR^{17A}SO₂R^{17B}, —NR^{17A}C(O)R^{17B}, —NR^{17A}C(O)OR^{17B}, —NR^{17A}OR^{17B}, —OCX¹⁷₃, —OCHX¹⁷₂, —OCH₂X¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁸ is independently hydrogen,
halogen, —CX¹⁸₃, —CHX¹⁸₂, —CH₂X¹⁸, —CN, —SOₙ₁₈R^{18A}, —SOᵥ₁₈NR^{18A}R^{18B}, —NHNR^{18A}R^{18B}, —ONR^{18A}R^{18B}, —NHC(O)NHNR^{18A}R^{18B},
—NHC(O)NR^{18A}R^{18B}, —N(O)ₘ₁₈, —NR^{18A}R^{18B}, —C(O)R^{18A}, —C(O)—OR^{18A}, —C(O)NR^{18A}R^{18B}, —OR^{18A}, —NR^{18A}SO₂R^{18B}, —NR^{18A}C(O)R^{18B}, —NR^{18A}C(O)OR^{18B}—NR^{18A}OR^{18B}, —OCX¹⁸₃, —OCHX¹⁸₂, —OCH₂X¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁹ is independently hydrogen,
halogen, —CX¹⁹₃, —CHX¹⁹₂, —CH₂X¹⁹, —CN, —SOₙ₁₉R^{19A}, —SO^{v19}NR^{19A}R^{19B}, —NHNR^{19A}R^{19B}, —ONR^{19A}R^{19B}, —NHC(O)NHNR^{19A}R^{19B},
—NHC(O)NR^{19A}R^{19B}, —N(O)ₘ₁₉, —NR^{19A}R^{19B}, —C(O)R^{19A}, —C(O)—OR^{19A}, —C(O)NR^{19A}R^{19B}, —OR^{19A}, —NR^{19A}SO₂R^{19B}, —NR^{19A}C(O)R^{19B}, —NR^{19A}C(O)OR^{19B}, —NR^{19A}OR^{19B}, —OCX¹⁹₃, —OCHX¹⁹₂, —OCH₂X¹⁹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R^{1A}, R^{1B}, R^{1C}, R^{1D}, R^{2A}, R^{2B}, R^{2C}, R^{2D}, R^{16A}, R^{16B}, R^{17A}, R^{17B}, R^{18A}, R^{18B}, R^{19A}, and R^{19B} are independently hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl;
X, X¹, X², X¹⁶, X¹⁷, X¹⁸, and X¹⁹ are independently —F, —Cl, —Br, or —I;
n1, n2, n16, n17, n18, and n19 are independently an integer from 0 to 4;
m1, m2, m16, m17, m18, m19, v1, v2, v16, v17, v18, and v19 are independently 1 or 2;
z1 is an integer from 0 to 5; and
z2 is an integer from 0 to 8.

2. The compound of claim 1, having the formula: 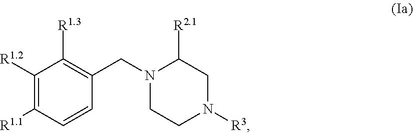
wherein,
R^{1.1}, R^{1.2}, and R^{1.3} are independently hydrogen, halogen, —CX¹₃, —CHX¹₂, —CH₂X¹, —OCX¹₃, —OCH₂X¹, —OCHX¹₂, —CN, —SOₙ₁R^{1D}, —NR^{1A}R^{1B}, —OR^{1D}, —SF₅, substituted or unsubstituted C1-C6 alkyl, substituted or unsubstituted 2 to 6 membered heteroalkyl, or substituted or unsubstituted 5 to 6 membered heteroaryl;
R^{2.1} is independently hydrogen, oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl;
R³ is independently —CN, 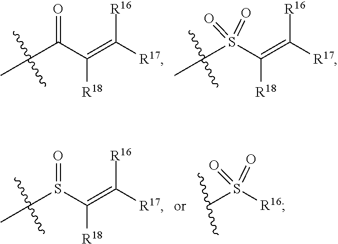
R¹⁶, R¹⁷, and R¹⁸ are independently hydrogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₇ cycloalkyl, or substituted or unsubstituted C₆-C₁₂ aryl;
R^{1A}, R^{1B}, and R^{1D} are independently hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; and
X is independently —F, —Cl, —Br, or —I.

3. The compound of claim 1, having the formula: 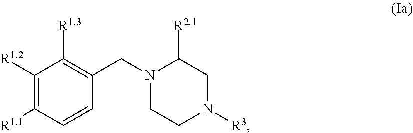
wherein,
R^{1.1}, R^{1.2}, and R^{1.3} are independently hydrogen, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, —SH, —SCH₃, —SCF₃, —SCHF₂, —SCH₂F, —SCCl ₃, —SCHCl₂, —SCH₂Cl, —SCBr₃, —SCHBr₂, —SCH₂Br, —SCl₃, —SCHI₂, —SCH₂I, —SOCH₃, —SO₂CH₃, —NH₂, —NHCH₃, —OH, —SF₅, substituted or unsubstituted C₁-C₆ alkyl, substituted or unsubstituted 2 to 6 membered heteroalkyl, or substituted or unsubstituted 5 to 6 membered heteroaryl;
R^{2.1} is independently hydrogen, oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
—NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₂ aryl, or substituted or unsubstituted 5 to 12 membered heteroaryl;
R³ is independently —CN, 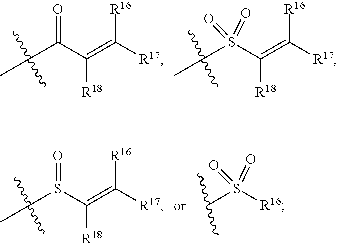
and
R¹⁶, R¹⁷, and R¹⁸ are independently hydrogen, —CCl₃, —CBr₃, —CF₃, —Cl₃, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₆ cycloalkyl, or substituted C₆ aryl.

4. The compound of claim 1, wherein
R^{2.1} is independently hydrogen, substituted or unsubstituted C₁-C₆ alkyl, substituted or unsubstituted 2 to 6 membered heteroalkyl, substituted or unsubstituted C₆-C₁₂ aryl, or substituted or unsubstituted 5 to 12 membered heteroaryl.

5. The compound of claim 1, having the formula: 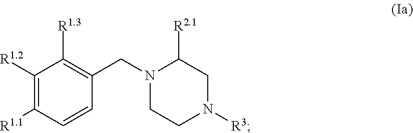
wherein
R^{2.1} is independently —CH₂O—CH₂CCH, —CH₂O—CH₂CN, —CH₂O—CH₂-heterocycloalkyl, substituted or unsubstituted C₆-C₁₂ aryl, or substituted or unsubstituted 5 to 12 membered heteroaryl.

6. The compound of claim 1, wherein
R^{2.1} is independently hydrogen, substituted or unsubstituted C₁-C₆ alkyl, or substituted or unsubstituted 2 to 6 membered heteroalkyl.

7. The compound of claim 1, wherein
R^{2.1} is independently hydrogen, R²⁰-substituted or unsubstituted C₁-C₆ alkyl, or R²⁰-substituted or unsubstituted 2 to 6 membered heteroalkyl;
R²⁰ is independently —OH, R²¹-substituted or unsubstituted 5 to 6 membered heterocycloalkyl or R²¹-substituted or unsubstituted 5 to 6 membered heteroaryl; and
R²¹ is independently oxo.

8. The compound of claim 1, having the formula: 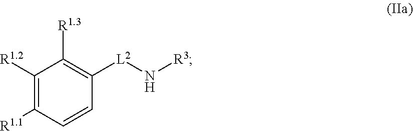
wherein
R^{1.1}, R^{1.2}, and R^{1.3} are independently hydrogen, halogen, —CX¹₃, —CHX¹₂, —CH₂X¹, —OCX¹₃, —OCH₂X¹, —OCHX¹₂, —CN, —SOₙ₁R^{1D}, —NR^{1A}R^{1B}, —OR^{1D}, —SF₅, substituted or unsubstituted C₁-C₆ alkyl, substituted or unsubstituted 2 to 6 membered heteroalkyl, or substituted or unsubstituted 5 to 6 membered heteroaryl;
L² is unsubstituted C₁-C₆ alkylene;
R³ is independently —CN, 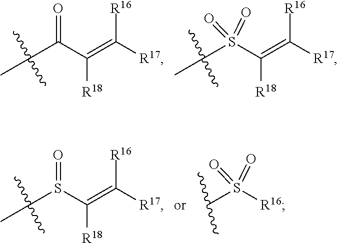
R¹⁶, R¹⁷, and R¹⁸ are independently hydrogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₇ cycloalkyl, or substituted or unsubstituted C₆-C₁₂ aryl;
R^{1A}, R^{1B}, and R^{1D} are independently hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; and
X is independently —F, —Cl, —Br, or —I.

9. The compound of claim 1, having the formula: 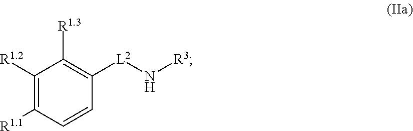
wherein,
R^{1.1}, R^{1.2}, and R^{1.3} are independently hydrogen, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, —SH, —SCH₃, —SCF₃, —SCHF₂, —SCH₂F, —SCCl ₃, —SCHCl₂, —SCH₂Cl, —SCBr₃, —SCHBr₂, —SCH₂Br, —SCl₃, —SCHI₂, —SCH₂I, —SOCH₃, —SO₂CH₃, —NH₂, —NHCH₃, —OH, —SF₅, substituted or unsubstituted C₁-C₆ alkyl, substituted or unsubstituted 2 to 6 membered heteroalkyl, or substituted or unsubstituted 5 to 6 membered heteroaryl;
L² is unsubstituted C₁-C₆ alkylene;
R³ is independently —CN, 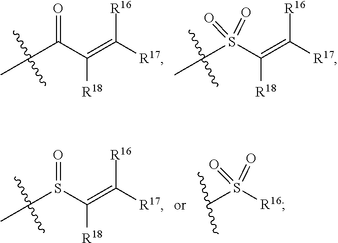
and
R¹⁶, R¹⁷, and R¹⁸ are independently hydrogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —CN, substituted or unsubstituted C₁-C₆ alkyl, substituted or unsubstituted C₃-C₆ cycloalkyl, or substituted C₆ aryl.

10. The compound of claim 9, wherein L² is unsubstituted n-propylene or unsubstituted n-butylene.

11. The compound of claim 2, wherein
R^{1.1} is independently
hydrogen, —OCCl₃, —OCF₃, —OCBr₃, —OCl₃, —OCHCl₂, —OCHBr₂, —OCHI₂, —OCHF₂, —OCH₂Cl, —O CH₂Br, —OCH₂I, —OCH₂F, —CN, —SH, —SCH₃, —SCF₃, —SCHF₂, —SCH₂F, —SCCl₃, —SCHCl₂, —SCH₂Cl, —SCBr₃, —SCHBr₂, —SCH₂Br, —SCl₃, —SCHI₂, —SCH₂I, —SOCH₃, —SO₂CH₃, —NH₂, —NHCH₃, —OH, —SF₅, alkenyl, alkynyl, unsubstituted methoxy, unsubstituted ethoxy, unsubstituted n propoxy, unsubstituted isopropoxy, unsubstituted n-butoxy, unsubstituted t-butoxy, unsubstituted sec-butoxy, unsubstituted isobutoxy, or unsubstituted pyrazolyl;
R^{1.2} is independently hydrogen, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, or unsubstituted C₁-C₄ alkyl; and
R^{1.3} is independently hydrogen,
halogen, —OCCl₃, —OCF₃, —OCBr₃, —OCl₃, —OCHCl₂, —OCHBr₂, —OCHI₂, —OCHF₂, —OCH₂Cl, —OC H₂Br, —OCH₂I, —OCH₂F, —CN, unsubstituted methoxy, unsubstituted ethoxy, unsubstituted n propoxy, unsubstituted isopropoxy, unsubstituted n-butoxy, unsubstituted t-butoxy, unsubstituted sec-butoxy, or unsubstituted isobutoxy.

12. The compound of claim 2, wherein
R^{1.1} is independently
hydrogen, —OCF₃, —CN, —SCH₃, —SCF₃, —SOCH₃, —SO₂CH₃, —NHCH₃, —SF₅, unsubstituted C₂-C₄ alkenyl, unsubstituted C₂-C₄ alkynyl, unsubstituted isopropoxy, or unsubstituted pyrazolyl;
R^{1.2} is independently hydrogen, —F, —Br, or —CF₃; and
R^{1.3} is independently hydrogen, —F, or —OCF₃.

13. The compound of claim 1, wherein
R³ is independently —CN.

14. The compound of claim 1, wherein
R³ is independently

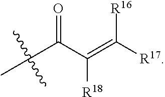

15. The compound of claim 1, wherein
R³ is independently

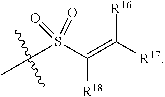

16. The compound of claim 1, wherein
R³ is independently

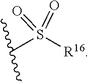

17. The compound of claim 1, wherein
R³ is independently

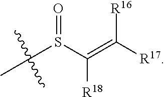

18. The compound of claim 1, wherein
R¹⁶ is hydrogen;
R¹⁷ is independently hydrogen, unsubstituted C₁-C₄ alkyl, or unsubstituted C₃-C₆ cycloalkyl; and
R¹⁸ is independently hydrogen, unsubstituted C₁-C₄ alkyl, or unsubstituted C₃-C₆ cycloalkyl.

19. The compound of claim 1, wherein
R¹⁶ is hydrogen;
R¹⁷ is independently hydrogen or unsubstituted C₁-C₄ alkyl; and
R¹⁸ is independently hydrogen or unsubstituted C₁-C₄ alkyl.

20. The compound of claim 1, wherein
R¹⁶ is hydrogen;
R¹⁷ is independently hydrogen, unsubstituted methyl, or unsubstituted cyclopropyl; and
R¹⁸ is independently hydrogen, unsubstituted methyl, or unsubstituted cyclopropyl.

21. The compound of claim 1, wherein
R¹⁶ is hydrogen;
R¹⁷ is independently hydrogen or unsubstituted methyl; and
R¹⁸ is independently hydrogen or unsubstituted methyl.

22. The compound of claim 1, wherein
R¹⁶, R¹⁷ and R¹⁸ are hydrogen.

23. The compound of claim 1, having the formula:

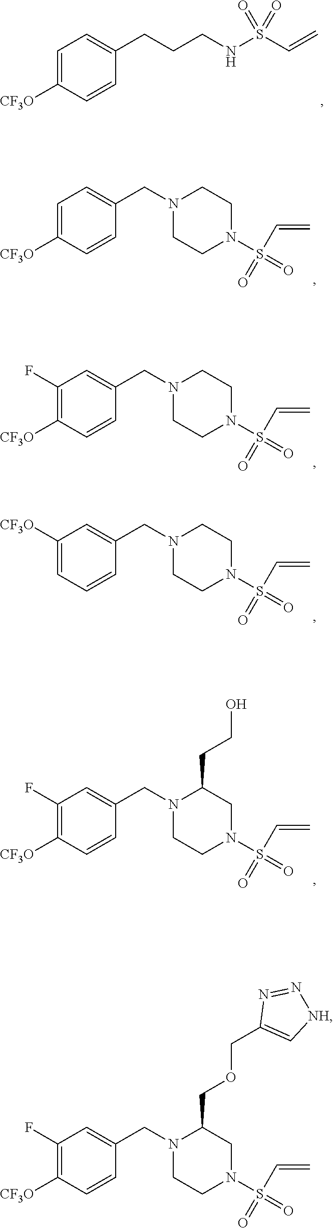

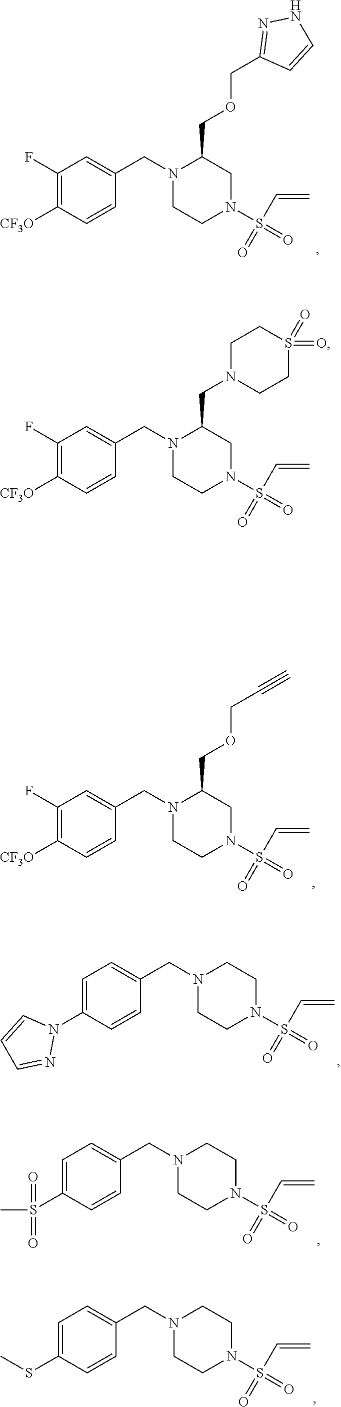

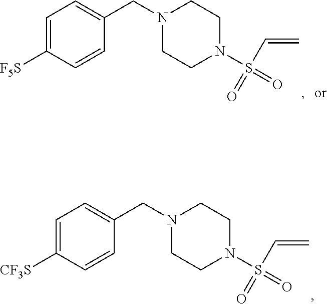

24. A pharmaceutical composition comprising the compound of any one of claims 1 to 23 and a pharmaceutically acceptable excipient.

25. A method of inhibiting Taspase1 protein activity, said method comprising: contacting the Taspase1 protein with a compound of one of claims 1 to 23.

26. A method of treating cancer, said method comprising administering to a subject in need thereof an effective amount of a compound of one of claims 1 to 23.

27. The method of claim 26, wherein the cancer is glioblastoma, melanoma, leukemia, lymphoma, ovarian cancer, renal cancer, colon cancer, prostate cancer, lung cancer, brain cancer, or breast cancer.

28. The method of claim 26, wherein the cancer is sensitive to Taspase1 inhibition.

29. A Taspase1 protein covalently bonded to a compound of one of claims 1 to 23.

30. The Taspase1 protein of claim 29, wherein the compound is bonded to a cysteine residue of the protein.

31. A Taspase protein covalently bonded to a portion of a compound of one of claims 1 to 23.

32. A compound of any one of claims 1 to 23, or a pharmaceutically acceptable salt therof, for use in a method of treating cancer, comprising administering to a subject in need thereof an effective amount of the compound.

33. A compound for the use of claim 32, wherein the cancer is glioblastoma, melanoma, leukemia, lymphoma, ovarian cancer, renal cancer, colon cancer, prostate cancer, lung cancer, brain cancer, or breast cancer.

34. A compound for the use of claim 32, wherein the cancer is sensitive to Taspase1 inhibition.
